(19) United States
(12) Patent Application Publication
(10) Pub. No.: US 2004/0110228 A1
(43) Pub. Date: Jun. 10, 2004

(54) Combinatorial organic synthesis of unique biologically active compounds

(76) Inventors: Shelli R. McAlpine, San Diego, CA (US); Rachel E. Taylor, La Jolla, CA (US); Megan L. Bolla, Irvine, CA (US); Anca M. Segall, San Diego, CA (US)

Correspondence Address: McDermott, Will & Emery, 7th Floor, 4370 La Jolla Village Drive, San Diego, CA, 92122, US

(21) Appl. No.: 10/407,851

(22) Filed: Apr. 1, 2003

Related U.S. Application Data
(60) Provisional application No. 60/369,420, filed on Apr. 1, 2002.

(51) Int. Cl. G01N 33/53; C08B 37/00; C07K 7/64

(52) U.S. Cl. 435/7.1; 530/317; 536/53

(57) ABSTRACT

The invention provides a method for making a combinatorial library of cyclic compounds, such as Holliday junction-trapping compounds, comprising the steps of (a) obtaining a plurality of trimers according to the generic structure X₁₋X₂₋X₃, wherein X₁, X₂ and X₃ can be independently any naturally or nonnaturally occurring amino acids or peptidomimetics thereof; (b) optionally coupling a spacer S to the trimer at either end; (c) cyclizing two trimers or trimer-spacer conjugates in a head-to-tail orientation; thereby obtaining a combinatorial library of compounds, wherein the library does not include an unmodifed or naturally occurring Holliday Junction-trapping compounds. The invention additionally provides methods macrocyclic compounds that are synergimycin derivatives.

This application claims benefit of the filing date of U.S. Provisional Application No. 60/369,420, filed Apr. 1, 2002, and which is incorporated herein by reference.

This invention was made with government support under solicited grant RFA-GM-01-006, awarded by the National Institutes of Health. The government has certain rights in the invention.

### BACKGROUND OF THE INVENTION

The present invention relates generally to macrocyclic compounds and to combinatorial libraries of mactrocyclic coumpounds. The present invention also relates to the generation of macrocyclic and peptidomimetic antibiotics and combinatorial libraries containing such compounds.

Antibiotic resistance of pathogenic bacteria has become an increasing public health concern. As more pathogenic bacteria become resistant to first and second line antibiotics, formerly easily treatable infectious diseases can become life-threatening and lethal. As resistance mutations spread in a population, particular antibodies become ineffective due to the concomitant spread of viability-improving mutations. In order to combat increasing numbers of pathogenic bacteria species with antibiotic resistance, new antibiotics will be required, including new antibiotics that target alternative genes and pathways.

One approach for developing new antibiotics is to start with old and ineffective antibiotics and to alter them to make them potent again. This approach has been taken with a number of drugs. For example, Synercid is a cocktail of two previously “retired” synergimycin antibiotics that have been structurally altered to attain renewed antibiotic activity.

Due to the structural complexity of antibiotics, altering the structure in a specific way can be difficult and often the most desirable changes are not possible. Thus, there is a need for methods for generating more potent and specific antibiotics. The present invention satisfies this need and provides related advantages as well.

### SUMMARY OF THE INVENTION

The invention provides a method for making a combinatorial library of cyclic compounds, such as Holliday junction-trapping compounds, comprising the steps of (a) obtaining a plurality of trimers according to the generic structure X₁-X₂-X₃, wherein X₁, X₂ and X₃ can be independently any naturally or nonnaturally occurring amino acids or peptidomimetics thereof; (b) optionally coupling a spacer S to the trimer at either end; (c) cyclizing two trimers or trimer-spacer conjugates in a head-to-tail orientation; thereby obtaining a combinatorial library of compounds, wherein the library does not include an unmodifed or naturally occurring Holliday Junction-trapping compounds.

The invention also provides a method for making a combinatorial library of compounds that are derivatives of class B synergimycins. The method involves (a) obtaining precursors of subunits designated as 1, 2, 3, 4, 5, 6 and 7, or any combination of a plurality of contiguous subunits thereof and (b) joining contiguous subunit precursors in a combinatorial manner; thereby obtaining a combinatorial library.

The invention further provides a method for making a combinatorial library of compounds that are derivatives of class A synergimycins. The method involves (a) obtaining precursors, of subunits designated 8, 9, 10, 11, 12 and 13, or any combination of a plurality of contiguous subunits or peptidomimetics thereof and (b) joining contiguous subunit precursors in a combinatorial manner; thereby obtaining a combinatorial library.

The invention provides macrocyclic compounds that are Holliday junction-trapping compounds, as well as, macrocyclic compounds that are Synergimycin Class A or B derivatives. Also provided are libraries containing such compounds and methods of use.

### BRIEF DESCRIPTION OF THE DRAWINGS

FIG. 1 shows Class A and Class B synergimycins and Synercid®.

FIG. 2 shows two DNA substrates (solid and dotted lines) are synapsed, and one strand of each is cleaved, exchanged with the partner substrate, and ligated to form a Holliday junction.

FIG. 3 shows an exemplary Class B synergimycin-derived compounds of the invention.

FIG. 4 shows low energy conformations of class B synergimycin (FIG. 4a) and a derivative (FIG. 4b), where residue 4 in the natural product is replaced by monomer 4a.

FIG. 5 shows reactions schemes for the preparation of (a) Class B synergimycin derivatives and (b) fragments of class B synergimycin.

FIG. 6 shows exemplary second fragments of the class B synergimycin derivatives of the invention.

FIG. 7 shows exemplary third fragments of the class B synergimycin derivatives of the invention.

FIG. 8 shows 3-4-5-6-7 pentamers of the class B synergimycin derivatives of the invention.

FIG. 9 shows pentamers, and (b) a linear heptamer of the class B synergimycin derivatives of the invention.

FIG. 10 shows a strategy and assay for identifying peptide effecters of recombination among the peptide libraries. The O represents a defined amino acid (for example, 20 total) while the X represents mixtures of all amino acids except Cys. At each step, the most active mixtures are ranked further by dose response curves to identify the most potent among them. The second step, which tests the effect of neighbors on the activity of the peptide mixture, reduces the number of specific peptides that need to be synthesized in the third step.

FIG. 11 shows co-crystal structures. The co-crystal structure on the left was obtained by mixing the mutant CreR173K protein with pre-formed Holliday junction substrates. The co-crystal structure on the right was obtained by mixing wild type Cre with wild type loxP substrates in the presence of peptide WKHYNY. The crystal was peptide dependent.

FIG. 12 shows inhibition of bacterial cell growth in the presence of antibiotics peptides in the medium. Left panel, Salmonella enterica, serovar Typhimurium Ames strain (gram-positive). Right panel, Salmonella enterica, Serovar Typhimurium LT2 strain (gram-negative). Peptide 8=WRWYCR; peptide 52=WKHYNY; peptide 56=KWWWRW.

FIG. 13 shows an exemplary synthesis approach for macrocyclic peptides and peptidomimetics.

FIG. 14 shows an exemplary syntheses of “pentamers” and “dimers” in each grid of “pentamers” ranging from 1-2-3-4a-5a to 1-2-3-4a-5e and “dimers” ranging from 6a-7a to 6e-7a.

FIG. 15 shows a reaction scheme for preparing exemplary macrocyclic compounds designed to trap the Holliday Junction.

FIG. 16 shows a reaction scheme for preparing macrocyclic compounds containing a spacer, designed to trap the Holliday Junction.

FIG. 17 shows a reaction scheme for preparing macrocyclic compounds that can involve: A) exchanging ester; B) exchanging ester; C) exchanging two bonds; and D) exchanging three bonds.

FIG. 18 shows a reaction scheme for preparing an initial 1-2 fragment and a second 1-2 fragment.

FIG. 19 shows a reaction scheme for preparing a peptidomimetic, designed from a peptide.

FIG. 20 shows a reaction scheme for preparing a peptidomimetic in which an amide and ester have been exchanged for ketones.

FIG. 21 shows a reaction scheme for preparing a peptidomimetic in which two amides and an ester bond have been exchanged.

FIG. 22 shows a reaction scheme for preparing peptidomimetics containing chiral secondary alcohols.

FIG. 23 shows a reaction scheme for preparing a peptidomimetic.

FIG. 24 shows a reaction scheme for preparing peptidomimetics with a spacer designed to trap the Holliday Junction.

FIG. 25 shows a matrix for preparing macrocyclic compound libraries of the invention using a combinatorial approach.

FIG. 26 shows comparison of resistance to peptide WRWYCR of a parental strain and 2 mutant derivatives.

FIG. 27 shows exemplary acid chlorides useful in the synthetic methods of the invention (X=Cl).

FIG. 28 shows exemplary organozinc reagents useful in the synthetic methods fo the invention.

FIG. 29 shows a reaction scheme for preparing a first fragment of a macrocyclic compound (Yield=75%).

FIG. 30 shows a reaction scheme for preparing “dimers” or “trimers”.

FIG. 31 shows a solid phase reaction scheme in which (a) acid chloride is bound to solid-phase; (b) aldehyde is bound to solid-phase.

FIG. 32 shows a reaction scheme in which organozinc is bound to the solid-phase.

FIG. 33 shows a reaction scheme for synthesis of aldehydes on solid support.

[FIG. 34 shows a reaction scheme for an initial 1-2 fragment (1-2A) and a second 1-2 fragment (1-2B).

FIG. 35 shows a reaction scheme for formation of peptidomimetic designed from a peptide.

FIG. 36 shows a reaction scheme for formation of peptidomimetic where an amide and ester have been exchanged for ketones.

FIG. 37 shows a reaction scheme for formation of peptidomimetic, in which two amides and an ester have been exchanged.

FIG. 38 shows a reaction scheme for the synthesis of peptidomimetics containing chiral secondary alcohols.

FIG. 39 shows a reaction scheme for the synthesis of peptidomimetic libraries designed to trap the Holliday Junction.

FIG. 40 shows a reaction scheme for synthesis of peptidomimetic libraries with a spacer designed to trap the Holliday Junction.

FIG. 41 shows a reaction scheme for preparing a Class A derivative using a combinatorial approach.

FIG. 42 shows a variety of monomers using for preparing a Class A synergimycin macrocyclic derivative.

FIG. 43 shows reaction schemes for macrocyclization using organozinc chemistry.

FIG. 44 shows justification of monomers in Class A synergimycin derivatives.

FIG. 45 shows a reaction scheme for preparing a Class A synergimycin derivative.

FIG. 46 shows a reaction scheme for macrocyclization of a Class A synergimycin derivative.

FIG. 47 shows reaction schemes for preparing Class A synergimycin derivatives and percentage yields.

FIG. 48 shows an exemplary macrocyclic hexamer of the invention.

FIG. 49 shows an exemplary macrocyclic compound of the invention with a spacer.

FIG. 50 shows a strategy for preparing peptidomimetic libraries of the invention.

FIG. 51 shows a reaction scheme for preparing Class B synergimycin derivatives.

FIG. 52 shows an exemplary reaction scheme for preparing macrocyclic compounds designed to trap the Holliday junction.

FIG. 53 shows an exemplary reaction scheme for preparing fragment 1 of a macrocyclic compound. (a) is TBTU (1.2 equiv.), Hunig's base (3 equiv.) CH₂Cl₂; (b) is TFA (20%), CH₂Cl₂, Anisole (2 equiv.)

FIG. 54 shows a reaction scheme for preparing C-2 symmetric cyclic hexapeptides.

FIG. 55 shows exemplary macrocyclic compounds of the invention.

FIG. 56 shows in vitro assay results, which indicate that C-2 symmetric macrocycles can bind to the Holliday junction.

FIG. 57 shows in vivo assay results, which indicate that C-2 symmetric macrocycles of the invention can inhibit bacterial growth.

FIG. 58 shows a reaction scheme for preparing cyclic hexapepeptides.

FIG. 59 shows a reaction scheme for preparing cylic octapeptides.

FIG. 60 shows a reaction scheme for solid-phase synthesis of C-2 symmetric macrocycles. (a) is HATU, TBTU, PyBOP and/or DEPBT (3 equiv), free acid, Hunig's base (3 equiv), CH₂CL₂; (b) TFA (20%), CH₂Cl₂, Anisole (2 eqiv); (c) HF; (d) HATU (3 equiv), Hunig's base (3 equiv.), CH₂Cl₂.

FIG. 61 shows a reaction scheme for preparing Class B synergimycin derivatives.

FIG. 62 shows a reaction scheme for preparing Class B synergimycin derivatives.

FIG. 63 shows a variety of monomers for synthesis of Class B synergimycin derivatives.

FIG. 64 shows a reaction scheme for preparing Class B synergimycin peptidomimetics.

### DETAILED DESCRIPTION OF THE INVENTION

The present is directed to macrocyclic compounds and the development of combinatorial libraries associated with these macrocyclic compounds. The invention also is directed to methods for preparing macrocyclic compounds, as well as combinatory libraries of macrocyclic compounds. As described herein, the macrocyclic compounds of the invention can be used to inhibit bacterial growth, for example, to treat patients having bacterial infections, and the combinatory libraries of the invention can be used, for example, for screening to identify additional useful compounds, including antibiotic compounds.

Some of the macrocyclic compounds of the invention have antibiotic activity based on their ability to stabilize the Holliday junction, which forms during bacterial replication. Other macrocyclic compounds, for which methods of preparation are provided, are based on known synergimycin A and B antibiotics. The methods provided allow efficient and high-throughput preparation of macrocyclic compounds that can be potent antibiotics.

The present invention relates to an improved approach for synthesizing potent antibiotics and other biologically active compounds. The synthetic methods of the invention involve building libraries of compounds based on previously known natural products, but containing new structural variations that cannot be found using previously-described methods of derivatization. In certain instances, the methods involve substituting specific “building blocks” into desirable positions, thereby placing selected functional groups into any position in the target structure.

As described herein, libraries are prepared by selecting building blocks at various positions in a template molecule. The building blocks are selected based on chemical, structural or biological properties desired at a particular position in the template. For example, if a water-soluble compound is desired, building blocks containing water-soluble groups are used. However, to examine the effects of water-solubility versus hydrophobicity, building blocks that contain hydrophobic groups are substituted for building blocks that contain water-soluble groups, or visa versa, and the two compounds are compared. There can be a particular building block in the compound that requires a water-soluble group to interact with its biological target, while another building block in the compound may require a more hydrophobic group to interact with its biological target. By building libraries, the ability to explore such options in natural product-based structures can be achieved. Architecturally different building blocks have been selected to obtain diverse structures, which can have diverse biological activities.

The invention provides a method for synthesizing derivatives. The method can be used, for example, to prepare a derivative of a peptide or natural product. Rather than using traditional modification of a chosen peptide or natural product, the approach described herein employs building blocks, or monomers, to build the derivatives from “scratch.” In addition, the approach described herein can employ an organic compound, such as a specific monomer, attached to zinc metal, which allows selective attachment of the monomer to another building block.

The approach taken in various embodiments of the present invention differs from typical approaches to building derivatives in that the derivatives are built using a combinatorial chemistry approach. In a method of the invention, the scaffolds of two types, for example class A and class B synergimycins, are figuratively “broken down” to monomers or building blocks that are interchangeable. The use of diverse building blocks leads to a structurally diverse set of derivatives. By comparison, other approaches begin with a molecule scaffold and then selectively functionalize that scaffold. In contrast, the inventors have used complex building blocks to build an entire molecule. The combinatorial libraries provide structurally diverse derivatives that cannot be synthesized using any other methods.

As shown herein, synthetic methods employing organozinc chemisty are useful for building complex libraries. The resultant libraries can contain compounds having sensitive functional groups. The organozinc chemistry methods described herein are also useful because this chemistry forms carbon-carbon bonds, which are more stable than amide bonds in polypeptides. Further, the organozinc chemistry methods described herein allow for formation of chiral centers on the carbons as well as preservation of a desired chirality. These chiral centers can play critical roles in biological activity. For these reasons, the organozinc chemistry methods described herein are useful for preparing peptidomimetic compounds based on peptides and cyclic peptides.

In several embodiments, the invention involves synthesizing new structures that are based on a known structure, such as a peptide or antibiotic. It is recognized that modification of existing non-active drugs has successfully renewed antibiotic potency against resistant strains of bacteria. The approaches described herein employ non-amino acid building blocks can be used to build, for example, a Class A or B synergimycin derivative, and these building blocks are constructed with carbon-carbon bonds rather than amide or ester bonds. Derivatives that contain non-amino acids are referred to as peptidomimetics because they mimic peptides but do not have the unfavorable amide and esters bonds. The advantage of the approaches described herein is that they allow exchange of amino acids on an individual basis for a non-amino acid, such as exchange of individual amide and ester bonds for carbon-carbon bonds using organozinc chemistry.

Peptides are convenient initial synthetic targets as they can be assembled and modified relatively easily. There are a large number of commercially available amino acids useful for efficient synthesis of peptides. Using peptides as initial leads allows rapid identification of the structural requirements of an active biological inhibitor. Using methods described herein, a macrocyclic compound, such as a cyclic peptide or libraries containing such compounds, can be prepared to contain carbon-carbon bonds of desired chirality. Organozinc-based methods described herein can be used to convert a peptide into a peptidomimetic. Peptidomimetics are derived from the amino acids used to synthesize the active structures and they are converted into organozinc reagents and coupled to an appropriately modified monomer. In one example, the organozinc monomers come from organo-halides, which then couple to an acid chloride. This methodology replaces an amide or ester bond in the original lead peptide with a ketone (which is a hydrogen-bond acceptor). In another example, organozinc monomers couple to aldehydes, thereby replacing the amide or ester bond with a chiral alcohol (a hydrogen-bond donor).

The invention provides peptidomimetic compounds, methods for making them, and libraries containing them. One approach for preparing peptidomimetics from peptides described herein uses several fragments of a peptide, but converts an ester functionality between two “monomers” to a ketone. Although this approach creates a macrocycle with one less atom in the ring than the original peptide, it is the fastest method for replacing the ester functionality. A second approach uses replacement of an ester bond to give the same number of atoms in a peptide ring as the original peptide. A third approach uses replacement of several amide bonds with appropriate ketones and alcohols. These methods are described herein below, and are applicable to preparing peptidomimetics of peptide Holliday junction-trapping compounds and Class A or B synergimycins.

In one embodiment, the invention is directed to compounds that can be used for trapping Holliday junctions, libraries of such compounds and related methods. Holliday junction-trapping compounds can function as antibiotics by inhibiting recombination reactions involved in bacterial proliferation. Site-specific recombination reactions are wide-spread in nature and control gene expression, amplify episome copy number, create genetic diversity, and separate chromosomes at bacterial cell division. Tyrosine recombinases are necessary for appropriate segregation of bacterial chromosomes that have been dimerized by homologous recombination. These enzymes, exemplified by the E. coli Xer C and D proteins, are conserved in both gram-positive and gram-negative bacteria, and present an attractive target for new antibiotics. The enzymes must act on the chromosome of each bacterium, and inhibitors can be found that trap intermediates which can act as poisons, much in the manner of topoisomerase inhibitors.

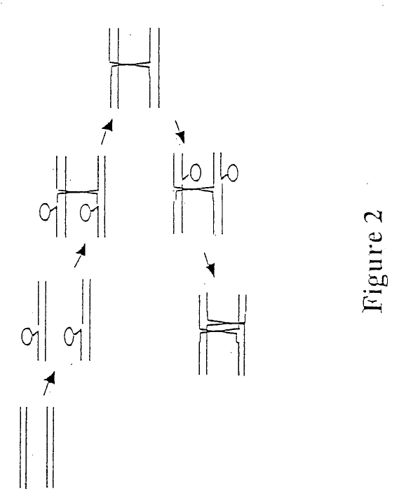

Two features distinguish the tyrosine recombinase family from other recombination proteins: first, the tyrosine nucleophile in the active site, and second, the independent cleavage and ligation of the two DNA strands, leading to the formation of a Holliday junction (FIG. 2). Cleavage and ligation are accomplished using a type I topoisomerase mechanism. One strand of each DNA substrate is cleaved by the protein using a tyrosine nucleophile, generating a transient 3′-phosphotyrosyl linkage between the enzyme and the DNA. The free 5′-OH group of each strand is exchanged between substrates and acts as a nucleophile to cleave the phosphotyrosyl linkage.

Previous work has identified a family of peptides that stabilize the Holliday junction (Cassell, et al. J. Mol. Bio. 299: 1193-1202 (2000); and Klemm, M et al. J. Mol. Bio. 299: 1203-1216 (2000)). One active structure was a dimer of the peptide sequence Lys-Trp-Trp-Cys-Arg-Trp, where the cysteine residue forms a disulfide bridge to give the active structures. As shown herein, the identified peptides can be used as a starting point for designing peptidomimetics that bind to and subsequently stabilize the Holliday junction.

As shown in Examples II and III, macrocyclic Holliday junction-trapping compounds of the invention both stabilized the formation of Holliday junctions in vitro and inhibited bacterial growth in vivo.

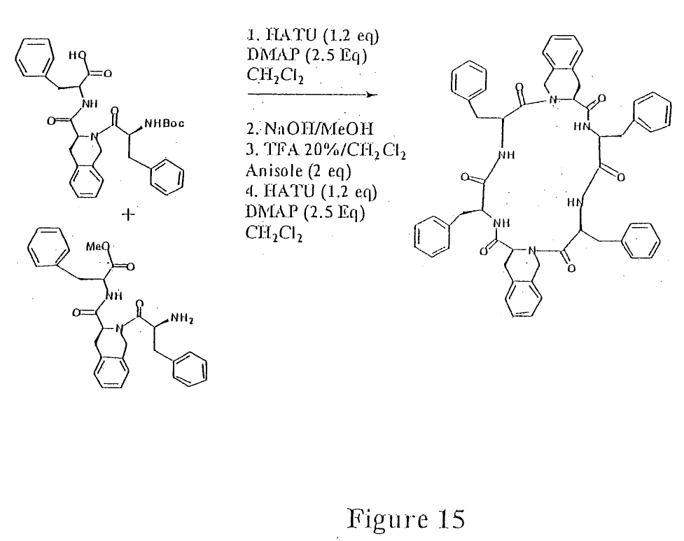

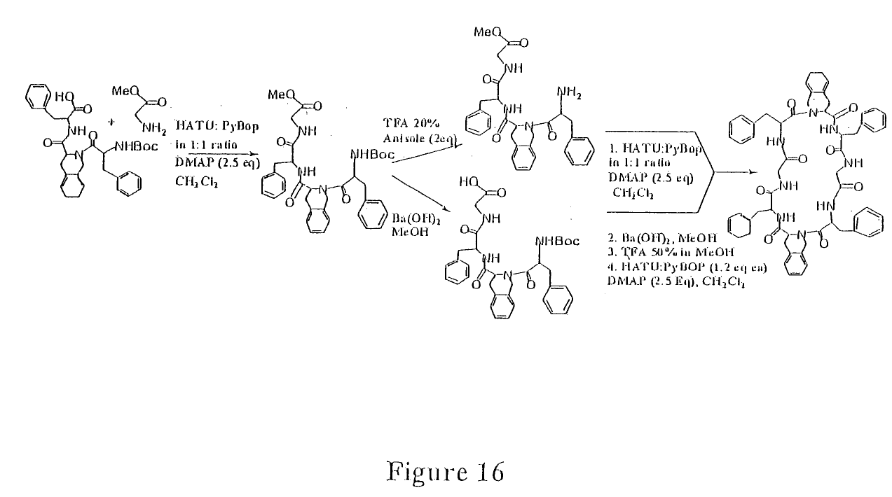

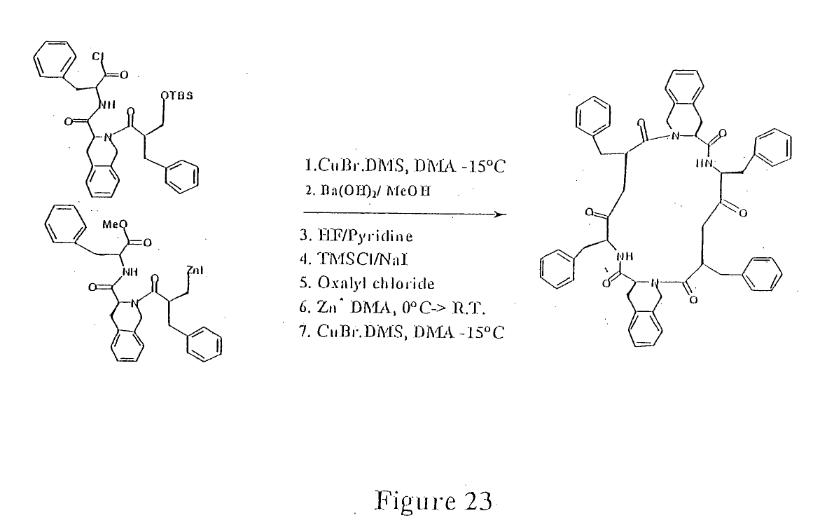

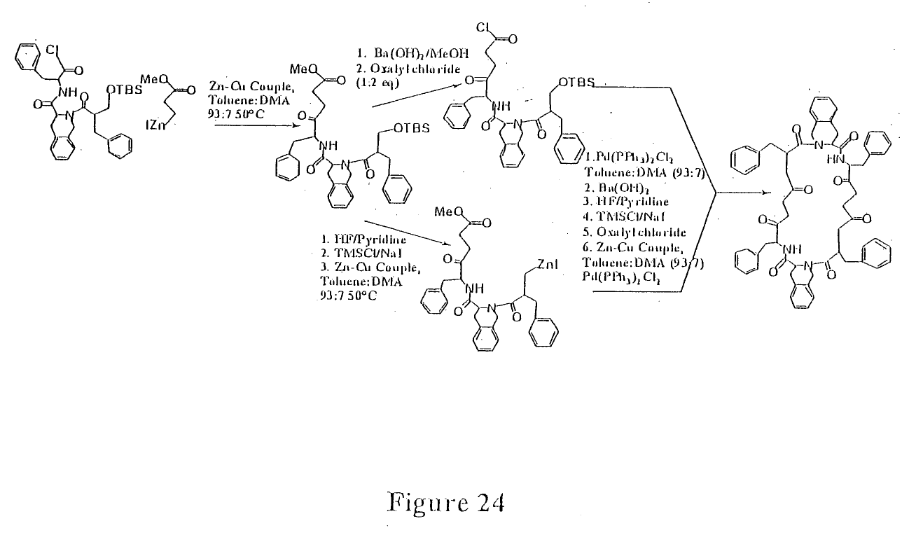

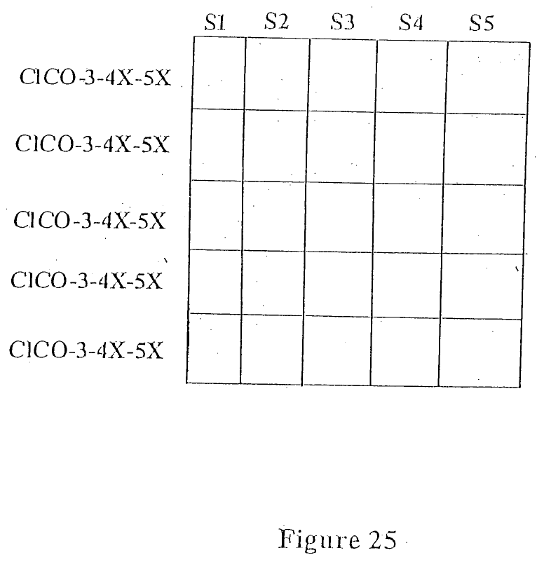

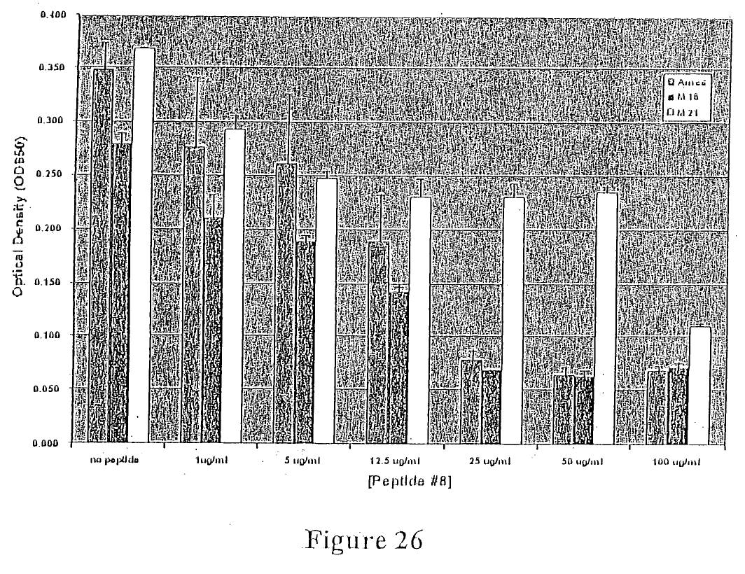

The methods of the invention can be used, for example, to prepare a library of macrocyclic compounds, such as cyclic peptides or peptidomimetics, or a combination thereof, designed to block the Holliday junction in site-specific recombination reactions. In one embodiment, the target of the compounds is a C-2 symmetric site. The invention provides several C-2 symmetric peptidomimetics based on the structure of linear peptide Holliday junction-trapping compounds. A library of hexamers can be prepared using, for example, the synthetic scheme shown in FIGS. 15 and 23. As shown in FIGS. 16 and 24, another synthetic scheme includes a spacer to be incorporated into the hexamer in a C-2 symmetric fashion (head-to-tail connectivity).

In one method for preparing a cyclic peptide library, organozinc chemistry is used to make hexamers. As shown in FIG. 23, the 5 monomer is exchanged from the amino acid to the acid-alcohol. The synthesis of the first precursor is accomplished by converting the acid to the acid chloride. The second trimer precursor is synthesized from the same peptide intermediate. By using reaction conditions such as those described below with respect to synergimycin derivates, the alcohol on monomer 5 is deprotected, converted to the iodide and then to the desired organozinc. These two trimer intermediates are coupled using organozinc conditions. The acid on the linear precursor is then deprotected with barium hydroxide, while the alcohol is deprotected with HF pyridine. This alcohol is then converted into the iodide, and generation in-situ of the acid chloride from the free acid prepares the hexamer for macrocyclization. Immediate addition of the appropriate activated zinc reagent can provide the C-2 symmetric peptidomimetic.

Another method for preparing a cyclic peptide of the invention is shown in FIG. 15. These methods are applicable to producing macrocyclic compounds from precursors smaller or larger than trimers. For example, as described in Example I, tetrameric precursors can be used to prepare octomeric macrocycles.

A Holliday junction-trapping compound, or library containing such a compound, can contain a spacer. A variety of spacers can be used, such as one or more amino acids or derivatives. Five exemplary spacers that have been incorporated into Holliday junction-trapping compounds include glycine, tryptophan, arginine, histidine and phenylalanine. A reaction scheme for coupling a spacer to a macrocyclic Holliday junction-trapping compound is shown in FIGS. 16 and 24. As shown in FIG. 24, upon coupling of the spacer to a trimer, the resulting tetramer is divided into two portions, and the acid is deprotected on one allotment of the tetramer then converted to the acid chloride. The other allotment has the alcohol deprotected, converted to the iodide (the liability of this iodo derivative can require in-situ generation and immediate conversion) and then converted to the organozinc reagent, following the below outlined synthesis conditions. The two tetramers are then reacted to form the octamer. Deprotection of both the acid and alcohol, then conversion of the alcohol to the iodide in-situ generation of the acid chloride prepare the octamer for macrocyclization. Formation of the organozinc reagent from the octamer iodide and macrocyclization with the acid chloride can provide the desired peptidomimetic.

In addition to the strategy of replacing the amide functional groups with ketones, amides can be replaced with chiral alcohols if they improve binding to their biological target. This can be done using a chiral catalyst as described below with respect to the Class B synergimycin derivative libraries.

The invention provides a compound having a formula selected from:

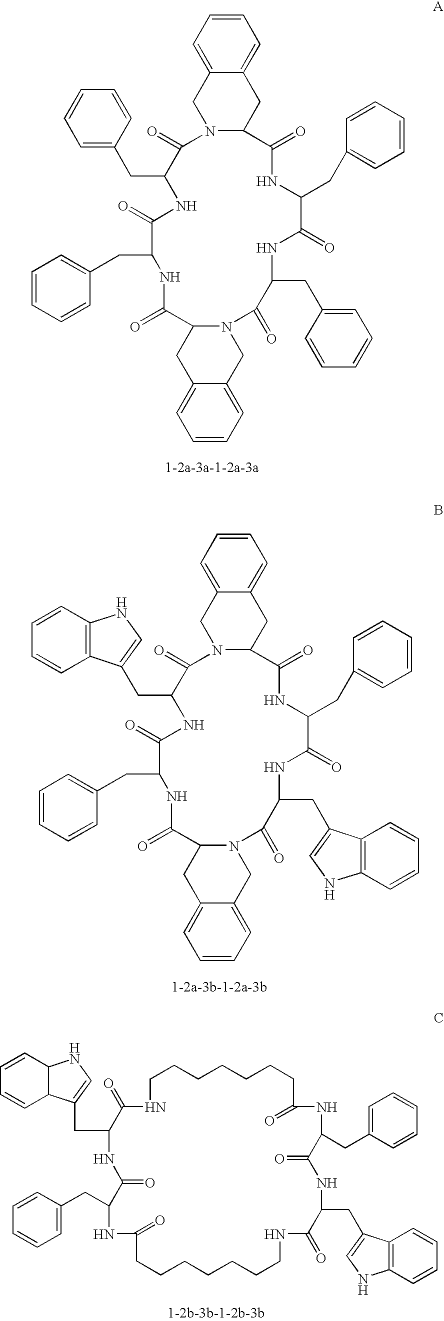

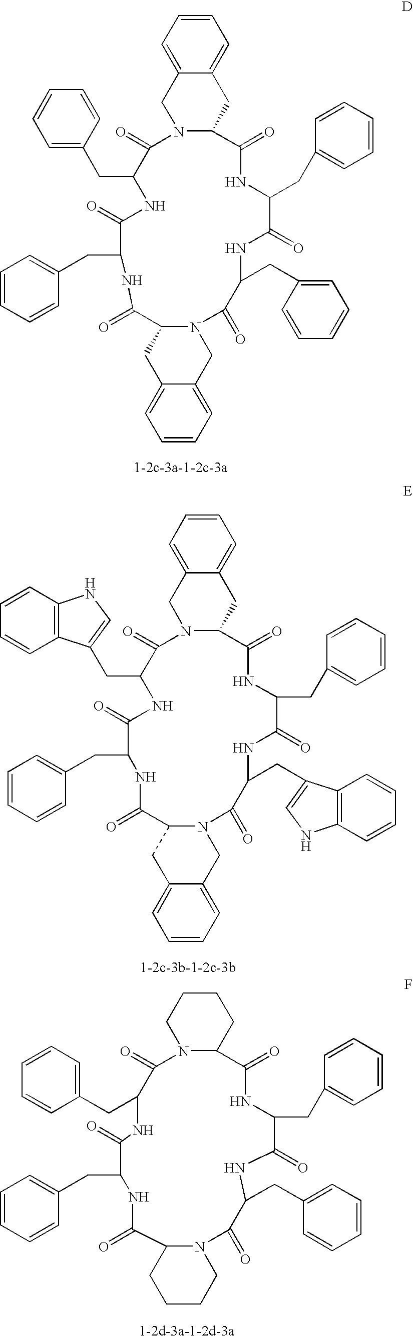

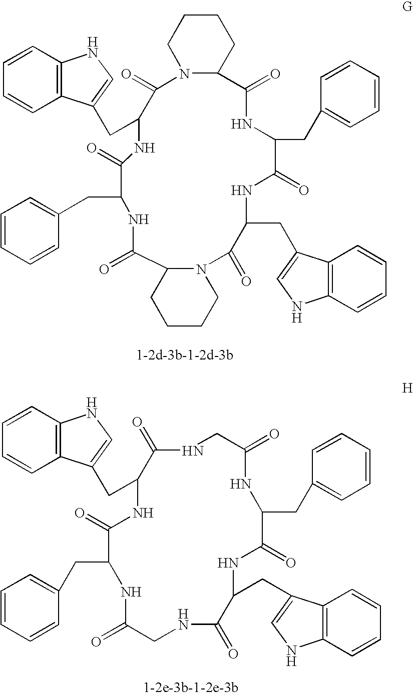

The invention also provides a combinatorial library containing a compound having a formula selected from the above macrocyclic compounds.

In one embodiment, the invention provides a method for making a combinatorial library of compounds that have the generic structure:

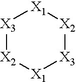

comprising the steps of (a) obtaining a plurality of trimers according to the generic structure X₁-X₂-X₃, wherein X₁, X₂ and X₃ are independently any naturally or nonnaturally occurring amino acids or peptidomimetics thereof; (b) optionally coupling a spacer S to the trimer at either end; (c) cyclizing two trimers or trimer-spacer conjugates in a head-to-tail orientation C-2 symmetry; thereby obtaining a combinatorial library of compounds, wherein the library does not include an unmodifed or naturally occurring Holliday Junction-trapping compounds.

The side group of X₃ or X₅ independently can have a C₁ to C₃ alkyl connecting a cycloalkyl, which can be substituted, or aryl group, which can be a heteroaryl, substituted aryl or substituted heteroaryl. The side group of X₄ incorporates a mono or bicyclic moiety that can be a heterocycle, which can be a substituted heterocycle. The side group of X₄ can provide conformational constraint to the trimer.

When a spacer S is employed, the spacer can be of a length, for example, of 5 to 25, 5 to 15, or 5 to 10 Å. An exemplary S can be selected from glycine, tryptophan, arginine, histidine, phenylalanine, or any other naturally occurring or non-naturally occurring amino acid. The spacer can be from an organozinc spacer precursor, for example, as shown in FIG. 24.

A library of the invention does not include an unmodifed or naturally occurring Holliday Junction-trapping compound. A library of the invention can include a compound at least one subunit contains a detectable moiety, and can be, for example, radio-labeled.

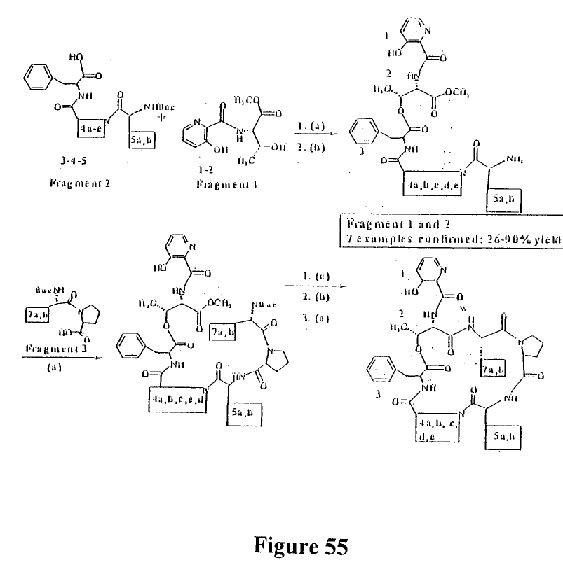

Exemplary compounds that can be contained in a macrocyclic compound library of the invention include those shown in FIG. 55. Exemplary synthetic schemes that can be used to prepare a macrocyclic compound library of the invention are shown in FIGS. 15, 16, 23, and 24.

Synergimycin Derivative Compounds, Libraries and Related Methods

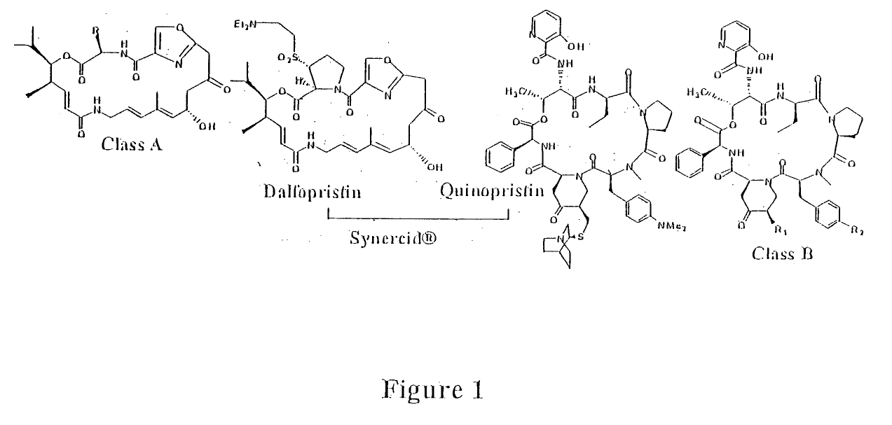

The Class A and Class B synergimycin antibiotics (FIG. 1) inhibit protein synthesis in bacteria. The target sites for the two classes are specific bases in the peptidyltransferase center of the 50S ribosomal subunit. Class A or Class B synergimycins, used independently, transiently block protein synthesis by forming a relatively unstable binary complex with the peptidyltransferase domain. However, when used together, each antibiotic enhances the inhibitory power of its partner at least one hundred fold. This synergistic effect is explained by the initial binding of a Class A structure to the ribosome, whereupon this binary complex undergoes a conformational change that increases its binding affinity for a Class B synergimycin structure. This ternary complex is extremely stable and, unlike the binary complex, the ternary complex is bactericidal.

In an embodiment of the invention, the invention provides methods for preparing new Class B synergimycin macrocyclic derivative compounds, such as cyclic peptides and peptidomimetics, and libraries containing such compounds (FIG. 1). Class B synergimycin has a depsipeptide structure and was selected as a scaffold due to the ease with which it can be broken into appropriate building blocks, as well as its usefulness as an antibiotic in Synercid® (FIG. 1). The Class B synergimycin peptidomimetic compounds, which can be contained in libraries, in combination with a Class A active compound, can provide new structures that exhibit synergistic bacterial growth inhibition.

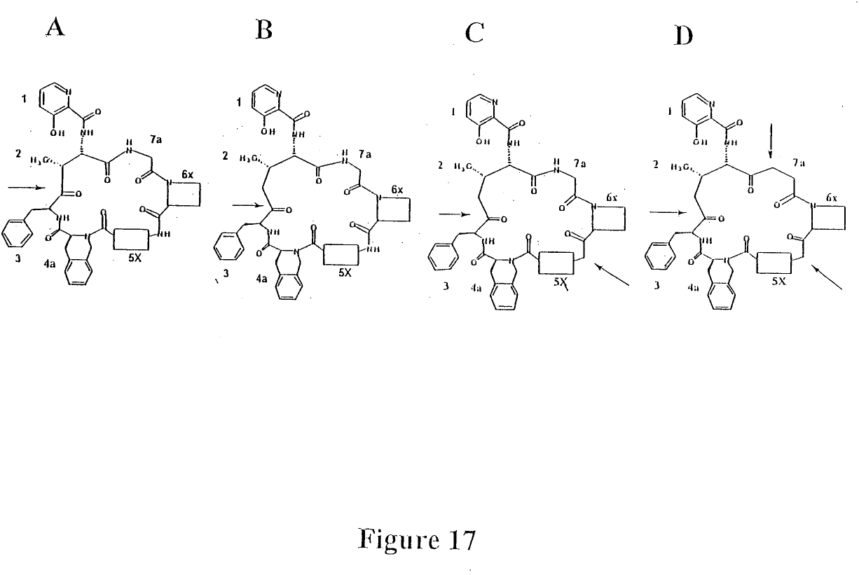

Examples of peptidomimetic macrocyclic compounds of the invention are shown (FIG. 17). The first peptidomimetic macrocyclic compound (FIG. 17A) has a ketone replacing the labile ester functionality between 2 and 3. This approach has one less atom than the original peptide. The second macrocycle also replaces the labile ester bond with a ketone, but has the same number of atoms in the macrocycle as the original peptide. The third macrocyclic compound is designed to exchange not only the ester bond between 2 and 3, but also the amide bond between 5 and 6. The fourth macrocyclic compound is designed to exchange the bonds between 2 and 3, 5 and 6, and 7 and 2, the latter of which is the site of macrocyclization.

In another embodiment, the methods of the invention for synthesizing Class B synergimycin derivatives represent a convergent strategy that allows easy exchange of specific monomers and conversion of these monomers into peptidomimetic building blocks. By substituting a specific monomer, functional groups can be displayed at a desired orientation in the chosen ring size. In addition, the convergent approach allows substitution of organozinc reagents that form carbon-carbon bonds, as well as radio labeled monomers that are useful for biological detection.

Previous studies have shown that a number of natural product Class B synergimycins have modified residues 4, 5, 6, and 7 indicating that fidelity of this portion of the ring is not necessary for synergistic activity. In addition, the macrocyclic conformation created by these four residues has been shown to be important for activity (Cocito, C., Microbiol. Rev. 43:145 (1979)).

The specific binding site for both Class A and B synergimycins on the ribosome has been determined. The first three residues (1, 2, and 3) remain the same for this family of the synergimycins and it is thought that picolinic reside 1 is responsible for binding to the ribosome (DiGiambattista et al., Bull. Soc. Chim. Belg. 99:195-211 (1990); DiGiambattista et al., Biol. Chem. 259:334-6339 (1984)). Conformational studies of Class B synergimycin molecules show that the solution conformation of these compounds have several characteristics. These include: a cis peptide configuration between the 4 and 5 residue, a type VI β-turn, reinforced by an intramolecular hydrogen bond between 6 and 3, and a biased rotational state where the aromatic ring of 5 is oriented towards residue 4. Studies on analogs have demonstrated the complexity involved in the synergistic mechanism. For example when residue 5 is altered from the L-amino acid residue to the enantiomeric D residue, the ring conformation is subtly altered. Although the macrocycle maintains both the cis peptide configuration between 4 and 5, and the intramolecular hydrogen bond, it reorients the aromatic ring of 5 towards 6 rather than towards 4. This causes the drug to lose all synergistic activity. As residue 1 is known to be the residue in direct contact with the ribosome, the loss of activity due to the exchange of 5 is attributed to changes in the ring conformation. It is known that residue 4 is embedded into a lipophilic region of the bacteria and that binding to the ribosome does not involve this residue. It has also been shown that the alteration of residue 4 to a more hydrophobic residue was one of the small changes that led to increased synergistic activity in Synercid®.

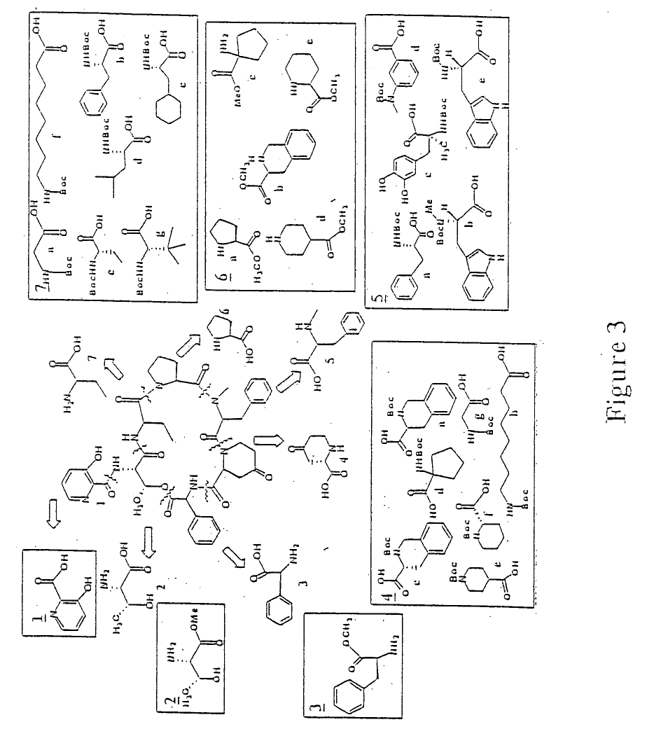

The invention provides macrocyclic compounds containing hydrophobic building blocks, as shown in FIG. 3, some mimicking the oxopipecolinic acid fragment (such as 4a, 4c, 4d, 4e, and 4f) while others such as 4b and 4g having little rigidity. In particular, 4b increases the size and flexibility of the macrocycle.

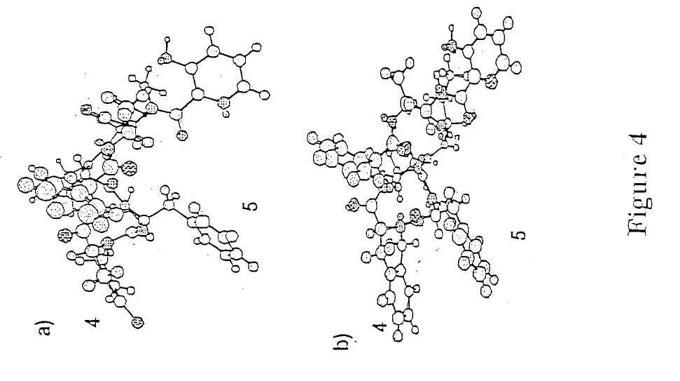

Studies indicate the importance of π-stacking between residues 4 and where the aromatic moiety of 5 sits underneath 4, and therefore the alteration from 4 to 4a for example, can induce this highly favored π-stacking. The low energy conformation for class B synergimycin and a derivative where residue 4 in the natural product is replaced by monomer 4a are shown in FIGS. 4A and 4B, respectively. The similarity in structure, particularly the preferred conformation of residue 5 being ri stacked beneath residue 4 illustrates how replacement of a key monomer can enforce a desired conformation.

As residue 5 is known to π-stack below residue 4, monomers with the same stereochemistry were chosen to mimic the natural product so that the orientation of the aromatic group was under residue 4. Residue 6, although it does not play a role in directly binding to the ribosome, is crucial as it forces a β-turn element into the macrocyclic architecture, and is responsible for hydrogen bonding to residue 3. Thus, secondary amines that varied β-turn elements and contained different degrees of rigidity are chosen to incorporate into the structure at position 6. Monomers to replace residue 6 have the potential to hydrogen bond with 3, known to be an important attribute. The systematic change in ring size and structure probe the importance of the β-turn and its relationship to the intramolecular hydrogen bond. Residue 7 appears to play a small role in the conformation of the ring insert (Anteunis and Sharma, Bull. Soc. Chim. Belg. 97:281-292 (1988); Anteunis et al., Bull. Soc. Chim. Belg. 97:135-148 (1988)) and a number of synergistic class B synergimycin compounds vary in the type of alkyl chain on this residue. A diverse number of amino acids were chosen to replace residue 7 including the enantiomer of the natural product, to probe the relationship between this residue and the macrocycle conformation.

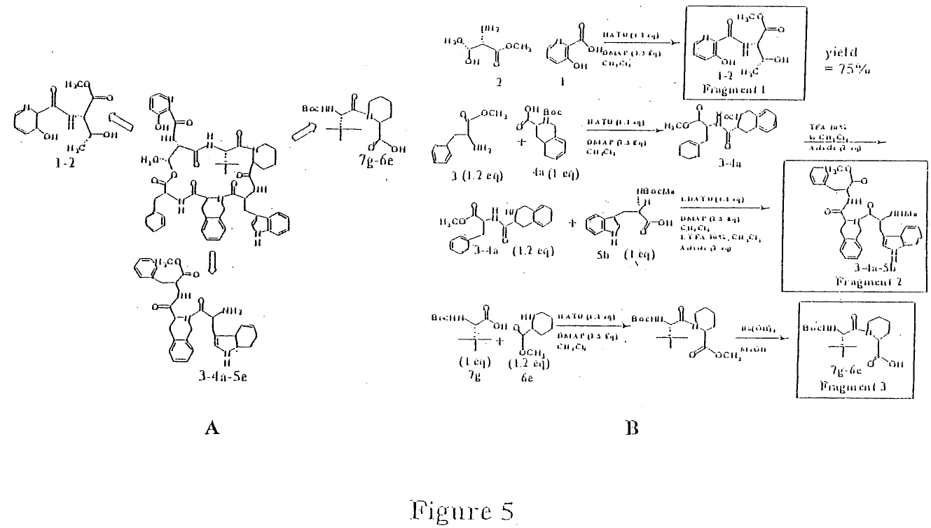

As shown in FIG. 5, Class B synergimycin derivative libraries were synthesized in three fragments (FIGS. 5a and 5b). These three fragments were selected to facilitate the exchange of specific monomers during peptidomimetic library synthesis. In the reaction scheme in FIG. 5, three monomer connections (one ester and two amide bonds) were exchanged for carbon-carbon bonds. The connection point between 2 and 3 was exchanged because it is an ester bond, which is labile. Modifications to monomer 2 and 3 (ie fragment 1 and fragment 2) allowed the bond between them to be changed to a carbon-carbon bond. In addition, it is known that an important hydrogen bond exists between the nitrogen-hydrogen on 3 and the carbonyl on 6 (Anteunis and Sharma, Bull. Soc. Chim. Belg. 97:281-292 (1988); Zhang et al., Bull. Soc. Chim. Belg. 97:419-429 (1988); Anteunis et al., Bull. Soc. Chim. Belg. 97:135-148 (1988)). Therefore, the amide between 5 and 6 was exchanged to a ketone. The selected ring-closing reaction was between a primary amine and acid (Bodanszky, M., Principles of Peptide Synthesis, 2nd ed; Springer-Verlag: Berlin (1993); Bodanszky and Bodanszky, Int. J. Pept. Protein Res. 23:565 (1984); Humphrey and Chamberlin, R. Chem. Rev. 97:2243-2266 (1997)).

The invention provides a method for making a combinatorial library of compounds that have the generic structure

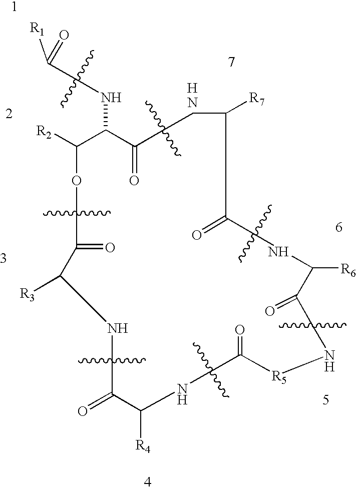

The method involves (a) obtaining precursors of subunits 1, 2, 3, 4, 5, 6 and 7, or any combination of a plurality of contiguous subunits or peptidomimetics thereof and (b) joining contiguous subunit precursors in a combinatorial manner; thereby obtaining a combinatorial library

In various embodiments, subunits 3 and 6 are capable of hydrogen bonding with each other. Subunit 1 can be, for example, hydroxy acid, wherein R is a linear or branched alkyl or aryl, which can be substituted; an aromatic hydroxy acid; or a hydroxy iodide.

Subunit 2 can be, for example, a hydroxy amine, wherein R is a linear or branched alkyl or aryl, which can be substituted; a hydroxy acid, wherein R is a linear or branched alkyl or aryl, which can be substituted; a hydroxy iodide, wherein R is a linear or branched alkyl or aryl, which can be substituted; a hydroxy amino acid, wherein R is a linear or branched alkyl or aryl, which can be substituted; an iodo amino acid, wherein R is a linear or branched alkyl or aryl, which can be substituted; or an iodo hydroxy amino acid, wherein R is a linear or branched alkyl or aryl, which can be substituted.

Subunit 3 can be, for example, an amino acid having the following structure, wherein R is a linear or branched alkyl or aryl, which can be substituted; an iodo acid, wherein R is a linear or branched alkyl or aryl, which can be substituted; a hydroxyacid, wherein R is a linear or branched alkyl or aryl, which can be substituted; an aromatic amino acid; an aromatic iodo acid; or an aromatic hydroxy acid. Subunit 3 can also contains a hydrogen and 6 can contain an electronegative atom (such as N or O, as in a carbonyl). In addition, subunit 6 can be, for example, a hydrogen and 3 can be an electronegative atom (such as N or O, as in a carbonyl).

Subunit 4 can be, for example, an amino acid; an aromatic amino acid; a hydroxy acid; an iodo acid, natural or nonnatural; and can have a hydrophobic moiety. Exemplary hydrophobic moieties include an unsubstituted or substituted alkyl, aryl, napthyl or polycyclic group. Subunits 4 and 5 can be in a cis peptide configuration; and can be capable of pi-stacking.

Subunit 5 can be, for example, an unsubstituted or substituted aryl, napthyl or polycyclic group; an amino acid; a hydroxy acid; an aromatic amino acid; anaromatic hydroxy amino acid; a hydroxy acid capable of pi-stacking; an iodo acid, natural or nonnatural; an aromatic iodo acid; an iodo acid capable of pi-stacking; or an L-isomer amino acid. Subunit 5 can have an aromatic ring and the ring can be oriented toward 4.

Subunit 6 can be, for example, an amino acid, including a natural or non-natural amino acid; an iodo acid; or a hydroxy acid. Subunit 6 can provides a (type-VI) beta-turn element into the compound. The beta-turn element can be, for example, a type-VI beta-turn element.

Subunit 7 can be an amino acid including a natural or non-natural amino acid; an iodo acid; or a hydroxy acid. Subunit 7 can have a linear or branched alkyl chain (such as t-butyl), or a large aromatic or hydrophobic group, any of which can be substituted. Subunits 2, 5 and 7 can independently be selected from the derivatives and groups in Table 3.

The combinatorial library of the invention does not include unmodifed or naturally occurring Class B Synergimycin. A library of the invention can contain at least one subunit that is labeled with a detectable moiety, such as a subunit that is radiolabeled. A library of the invention can be a linear or cyclic library and can contain peptides, peptidomimetics or both.

Submits 1-2 can be combination of contiguous subunits; subunits 3-4-5 can be a combination of contiguous subunits; and subunits 6-7 can be a combination of contiguous subunits. The first, second or third fragments can be joined via an organozinc reaction such that, for example, the bond between 5 and 6 is a ketone.

A macrocyclic compound of the invention can contain subunits 1 to 7, selected from the subunits shown in FIG. 3.

Class A Synergimycin Derivatives

In one embodiment, the invention provides a method for making a combinatorial library of compounds that have the structure

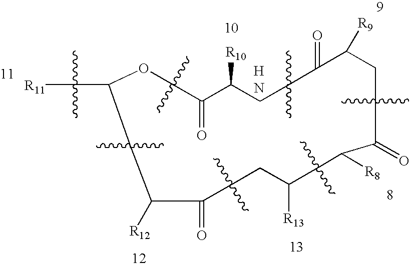

, comprising the steps of (a) obtaining precursors of subunits 8, 9, 10, 11, 12 and 13, or any combination of a plurality of contiguous subunits or peptidomimetics thereof, and (b) joining contiguous subunit precursors in a combinatorial manner; thereby obtaining a combinatorial library.

The claimed library does not include unmodifed or naturally occurring Class A Synergimycin. In the library, the side groups (R) of 8, 9, 10, 11, 12 or 13 can be the side group of a naturally or nonnatural amino acid. The side group (R) of 11 contains one of or a combination of a (substituted) C₁ to C₈ alkyl, cycloalkyl or aryl.

The side groups (R) 8, 9, 10, 11, 12 or 13 can be independently a hydroxy acid, or derivative thereof, an aromatic hydroxy acid, a hydroxy iodide, a hydroxy amine, a hydroxy amino acid, an iodo amino acid or an iodo hydroxy amino acid. For example, 8 can be a hydryoxy acid, hydroxy ester or hydroxy aldehyde; 9 can be an iodo acid or hydroxy acid; 10 can be a hydroxy acid or iodo acid; 11 can be a haloalkyl, halo aryl or hydrophobic halogen group; 12 can be a hydroxy acid, hydroxy ester, hydroxy aldehyde, iodo acid, iodo ester or iodo aldehyde; 13 can be a halo amine, hydroxy acid or iodo acid.

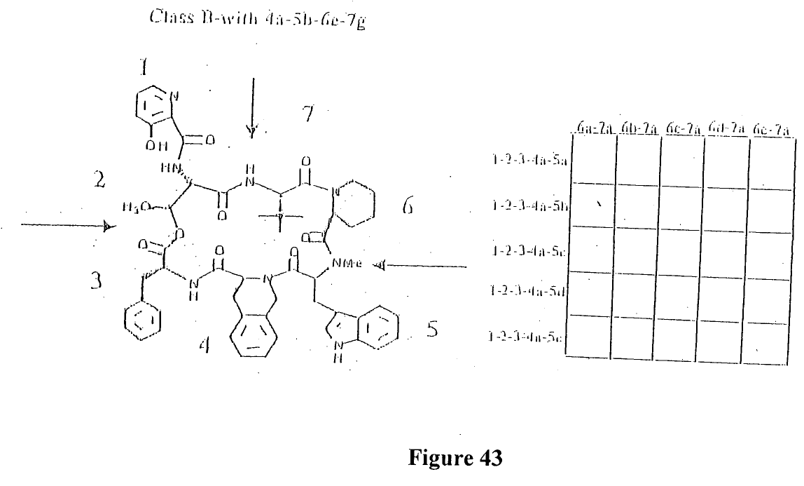

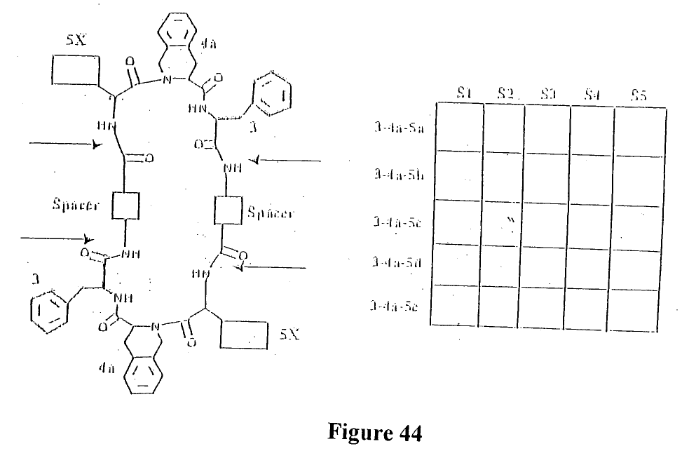

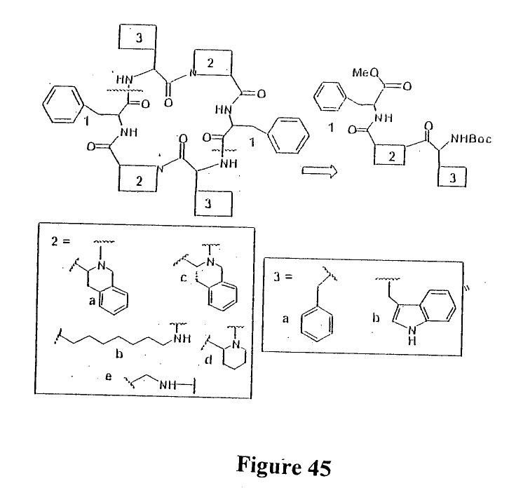

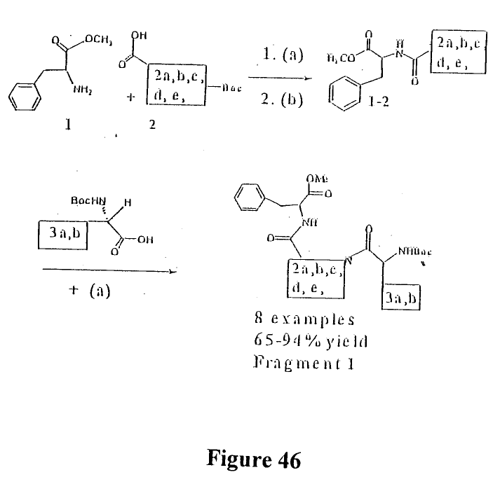

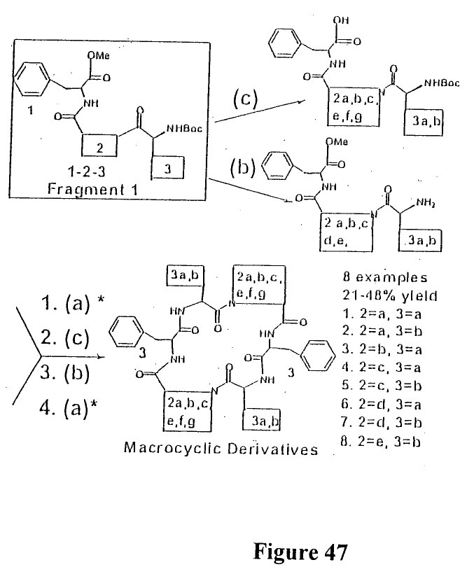

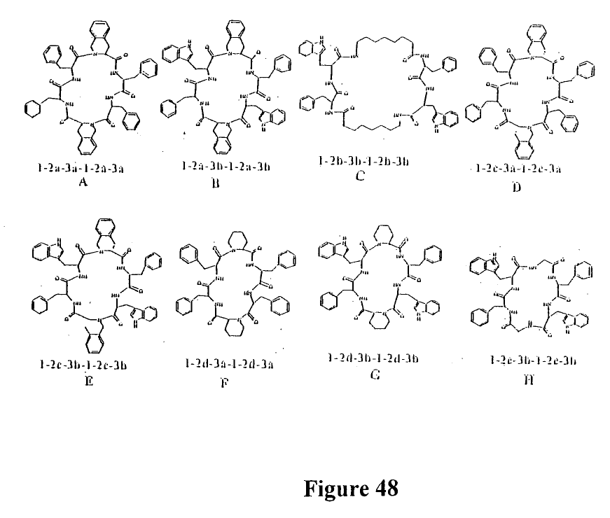

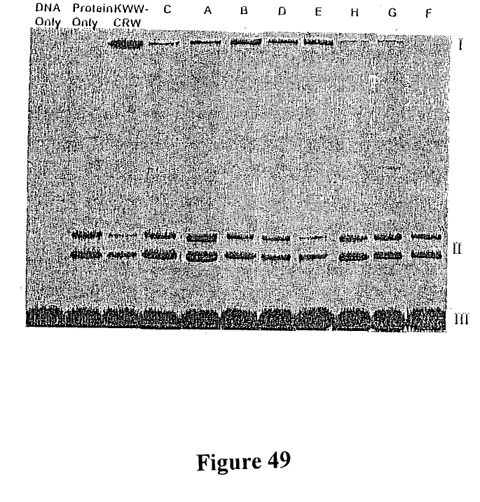

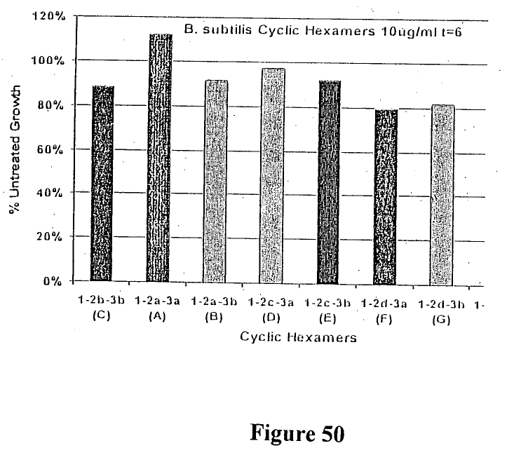

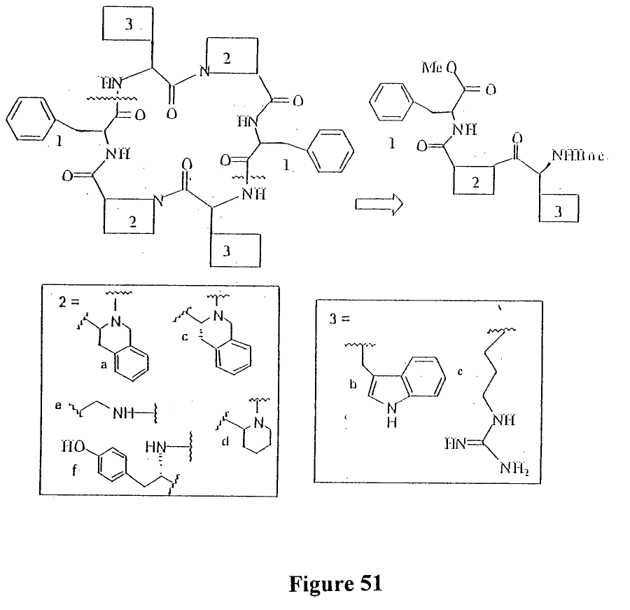

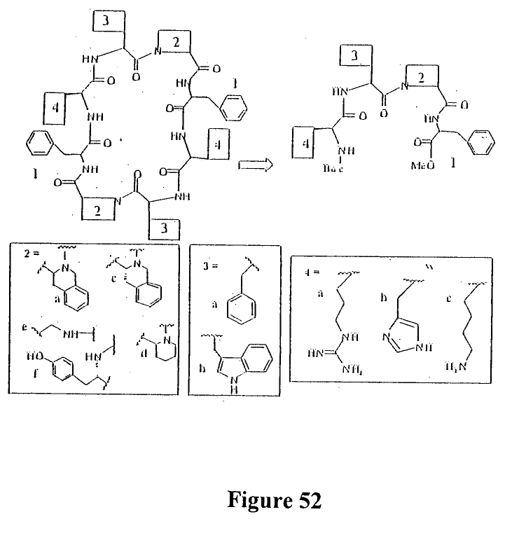

Macrocyclization can occur, for example, according to FIG. 43 or 46. A compound contained in a library of the invention can have at least one subunit is radiolabelled.

In various synthetic methods of the invention, organozinc reagents are used for preparing compounds and combinatorial libraries. A variety of methods can be used to generate either the organozinc halide or the diorganozinc (Pelter et al., C. Tetrahedron Lett. 24:1433-1436 (1983); Knochel, P., Comprehensive Organometallic Chemistry II Abel, E. W., Gordon, F., Stone, A., Wilkinson, G. (editors), 1st ed., Pergamon Press: New York (1995); Erdik, E., Tetrahedron 43:2203 (1987); Knochel et al., Tetrahedron 54:8275-8319 (1998); Soai and Niwa Chem. Rev. 92:833-856 (1992); Noyori, R. In Asymmetric catalysis in organic synthesis; 1st ed.; Noyori, R., Ed.; John Wiley & Sons, Inc.: New York, 1994; pp 255-297 Lutz and Knochel J. Org. Chem. 62:7895-7898 (1997)). Two common methods for generating organozinc halides are using activated zinc dust or Rieke's activating method (Hanson and Rieke, J. Am. Chem. Soc. 117:10775-10776 (1995)). A halogen lithium exchange using lithium metal (Peterson et al., J. Org. Chem. 51:2381-2382 (1986); Tucker et al., J. Am. Chem. Soc. 114:3983-3984 (1992)) or t-butyl lithium (Rozema et al., J. Org. Chem. 57:1956-1958 (1992)) and a transmetalation to the organozinc via addition of zinc bromide is another general approach.

Several methods can be used to generate diorganozincs. A method for preparing functionalized dialkylzinces proceeds via an iodine-zinc exchange reaction allowing the synthesis of a large number of polyfunctionalized diorganozincs from the readily available organohalides (Micouin et al., Synlett 327-328 (1997)). A mild method developed recently uses isopropyl Grignard and zinc bromide generating the diisopropylzinc in situ, which subsequently reacts to form the isopropylorganozinc (Brown and Coleman, J. Org. Chem. 44:2328-2329 (1979)). Another mild method generates an organozinc reagent via hydroboration of an alkene and subsequent halogen-metal exchange to yield a diorganozinc. This method leaves intact a number of sensitive functionalities that are typically difficult to include when generating these reagents using other methods (Jones and Knochel, J. Chem. Soc., Perkin Trans. I 13117-3118 (1997)). A relatively new method for synthesizing diorganozincs involves reaction of an organohalide and activated zinc to form an intermediate organozinc halide, which is allowed to react with trimethylsilylmethyl (TMSM) lithium to form the organo-trimethylsilylmethyl zinc. This mixed organozinc has been successfully used in Michael additions to enones and chiral additions to aldehydes.

Amino acid derivatives have been converted into organozinc reagents and successfully coupled to a range of electrophiles using palladium catalysts (Deboves et al., J. Chem. Soc. Perkin Trans. I 1:1876-1884 (2001); Dunn et al., J. Chem. Soc., Perkin Trans. I 13:1639-1640 (1995); Jackson et al., J. Chem. Soc., Chem Commun. 10:644-645 (1989); Jackson et al., J. Org. Chem. 60:2210-2215 (1995); Dunn and Jackson Tetrahedron 53:13905-13914 (1997); Jackson et al., J. Org. Chem. 57:3397-3404 (1992); Jackson et al., J. Org. Chem. 63:7875-7884 (1998)). For example, Jackson et al. have successfully demonstrated that protected amino acids can be converted into organozinc reagents, and then effectively coupled to amino acid chlorides. As shown herein, organozinc reagents are coupled to acid chlorides, thereby replacing the amide or ester with a ketone or chiral alcohol.

A variety of methods can be used for enantioselective addition of zinc reagents to aldehydes. High enantioselectivities and chemical yields can be obtained, for example, upon addition of a wide variety of organozinc reagents to both aromatic and aliphatic aldehydes. Two general chelators for asymmetric addition of functionalized organozincs to aldehydes have been successfully employed: (a) chiral aminoalcohols (b) chiral titanium complexes (Knochel et al., Tetrahedron 54:8275-8319 (1998); Soai and Niwa, Chem. Rev. 92:833-856 (1992); Noyori, R., In Asymmetric catalysis in organic synthesis (1st ed.), Noyori, R., Ed., John Wiley & Sons, Inc.: New York, 255-297 (1994); Knochel and Singer, Chem. Rev. 93:2117-2188 (1993)). Not only do we use organozinc reagents made from amino acid precursors and their addition to acid chlorides, but also apply their addition to amino aldehydes. The development of this methodology allows for replacement functionality of the amide or ester from a hydrogen-bond donor (alcohol) to a hydrogen-bond acceptor (ketone).

In one embodiment, the methods of the invention involve preparing peptidomimetics using organozinc chemistry. One exemplary approach uses acid chloride electrophiles with organozinc reagents; a second exemplary approach uses aldehydes as electrophiles; a further exemplary approach is significantly more complicated as it forms a chiral center upon addition of an organozinc reagent to an aldehyde.

In a method of the invention, organozinc reagents derived from amino acids are prepared and employed in reactions with peptide derivatives to generate ketones and alcohols. Part of this methodology involves organozinc ring-closing reactions using peptidomimetic precursors (Oppolzer et al., Tetrahedron Lett. 36:2607-2610 (1995); Oppolzer and Radinov, J. Am. Chem. Soc. 115:1593-1594 (1993)).

A number of reports have described successful couplings of organozinc reagents with acid chlorides (Deboves et al., J. Chem. Soc., Perkin Trans. 1 1:1876-1884 (2001); Jackson et al., J. Org. Chem. 57:3397-3404 (1992); Jackson et al., J. Org. Chem. 60:2210-2215 (1995)). Herein is described a general organozinc method for preparing protected amino acid organozinc reagents. These reagents are useful for combinatorial approaches and can be used with any amino acids. As shown herein, eight iodo-derivatized protected amino acids and iodo esters were reacted with a single acid chloride (N-protected phenylalanine acid chloride).

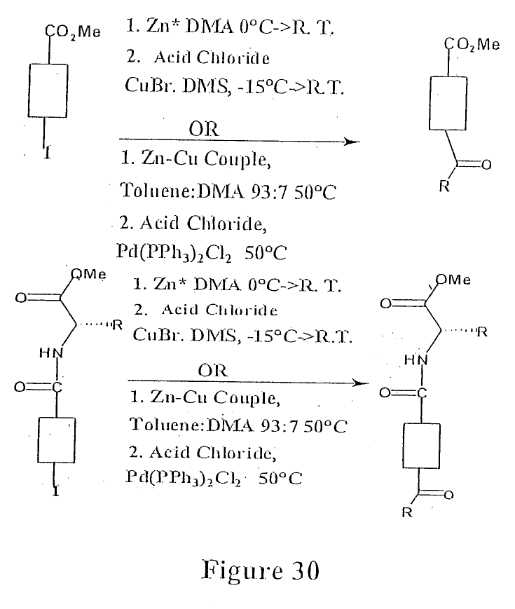

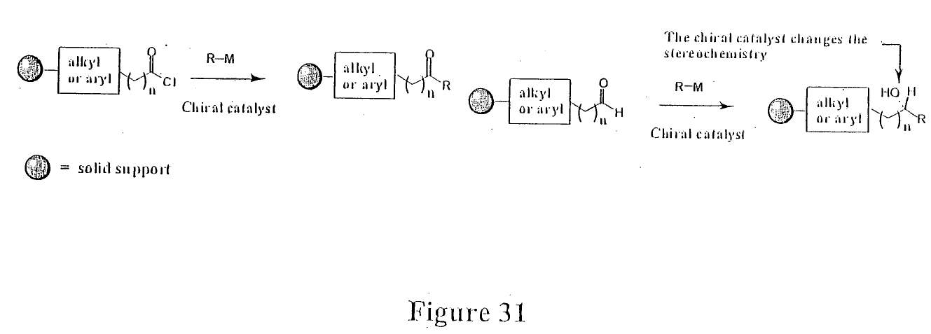

One set of conditions for conversion of iodo compounds to organozinc reagents uses the procedures developed by Jackson and co-workers (Deboves et al., J. Chem. Soc., 1:1876-1884 (2001); Dunn et al., J. Chem. Soc. 13:1639-1640 (1995); Dunn et al., Tetrahedron 53:13905-13914 (1997); Jackson et al., J. Org. Chem. 57:3397-3404 (1992); Jackson et al., J. Org. Chem. 60:2210-2215 (1995)). Zinc dust was activated via addition of 1,2 dibromoethane (Knochel and Singer, Chem. Rev. 93:2117-2188 (1993) and Knochel et al., J. Org. Chem. 53:2390-2391 (1998)) in dimethylacetamide (DMA). After heating to 65° C., trimethylchlorosilane was added. The reaction was then be cooled to room temperature and sonicated for 30 minutes. A solution of the iodide in DMA was then added to the activated zinc and the reaction was heated to 35° C. for approximately 30 minutes. The supernatant was removed as the reaction went to completion, and was cooled to −10° C., whereupon a solution of copper (I) cyanide-lithium chloride in DMA was added. The reaction was stirred at 0° C. for 10 minutes and then cooled to −25° C. Addition of the acid chloride can give the desired ketone as either the “dimer”, which is defined as two monomers coupled (See FIG. 30), or the “trimer”, which is defined as three monomers coupled (See FIG. 30). These “dimers” and “trimers” no longer contain an amide or ester bond. Another method for converting a peptide to a peptidomimetic involves conversion of an iodo-derivatized amino acid to the zinc reagent via addition of zinc-copper to couple to the iodide in toluene:DMA at 50° C. Addition of this reagent to bis(triphenylphosphine) dichloropalladium and the acid chloride at 50° C. can provide the desired ketone.

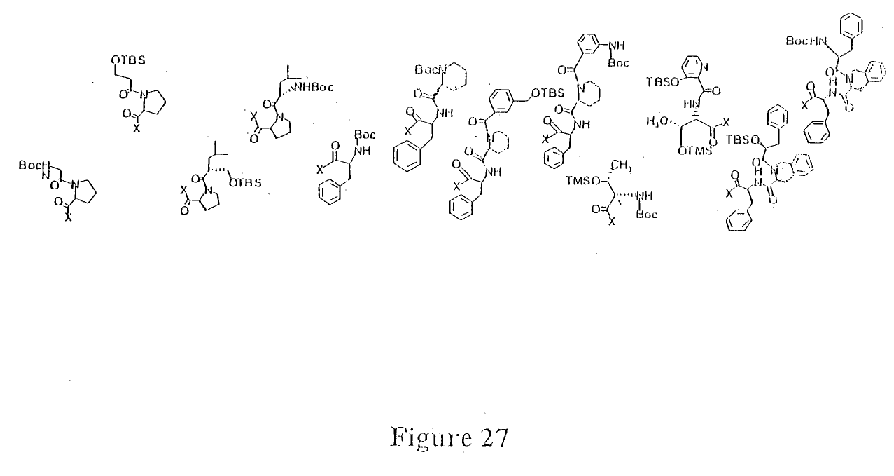

In one embodiment, the invention provides methods for preparing peptidomimetics and libraries containing peptidomimetics using aldehydes and chiral catalysts. Chiral alcohols can be employed under similar conditions as those described above where the organozinc reagent react with aldehydes rather than acid chlorides (FIG. 27 X=H).

A comparison can be drawn between the biological activity of the ketone (a hydrogen-bond acceptor), which was synthesized using acid chloride precursors, to that of the alcohol (a hydrogen-bond donor), synthesized using aldehyde precursors. Thus, the features are most important for binding to the biological target can be determined. Another benefit is the ability to compare enantiomers of the chiral alcohol by using the opposite enantiomer of the chiral catalyst in the organozinc reaction with the aldehyde. This allows determination of specific interactions responsible for the biological activity and compare enantiomeric monomers in the target, as well as compare the difference in binding between these two enantiomers of a hydrogen-bond donor with that of a hydrogen-bond acceptor.

A variety of ring-closing schemes can be employed to prepare a compound or library of the invention. The mild reaction conditions necessary for organozinc chemistry and the ability to generate a chiral center are useful for ring-closing reactions (Oppolzer et al., Tetrahedron Lett. 36:2607-2610 (1995); Oppolzer and Radinov, N. J. Am. Chem. Soc. 115:1593-1594 (1993); Oppolzer et al., J. Org. Chem. 66:4766-4770 (2001)). Specific ring closing reactions have been have been described, for example, in Oppolzer et al., J. Org. Chem. 66:4766-4770 (2001). As shown herein, acid chlorides and organozinc reagents can be used for ring-closing to prepare macrocyclic ketones. The general approach employs a number of linear hepta- and hexapeptides that contain an acid chloride at one end and an iodide at the other end.

Organozincs are synthetically useful reagents as they provide a mild method for enantioselective addition to aldehydes. Enantioselective addition of zinc reagents to aldehydes is described, for example, in Knochel et al., Tetrahedron 54:8275-8319 (1998). High enantioselectivities and chemical yields have been achieved upon addition of a wide variety of organozinc reagents to both aromatic and aliphatic aldehydes. This is also true of organozinc additions to 1,2 and 1,4 a,b-unsaturated aldehydes. Rozema et al., J. Org. Chem. 57:1956-1958 (1992). Several enantioselective additions of dialkylzincs to aldehydes using polymer-supported catalysts have been reported (Soai and Niwa, S. Chem. Rev. 92:833-856 (1992); and Itsuno and Frechet, J. Org. Chem. 52:4140-4142 (1987)) and recently Kondo et al. reported the synthesis of an organozinc reagent on solid-phase via the successful halogen-metal exchange of aryl iodides (Micouin, and Knochel, Synlett 327-328 (1997)). Transmetalation of the solid-phase organozinc reagent to give the immobilized cuprate led to the desired 1,4 addition product. The palladium-catalyzed cross-coupling reaction was also investigated and successfully provided unsymmetrical biaryl systems, while the Reformatsky-type reaction yielded the α-hydroxy ester.

Attachment of the electrophile to solid support and then adding solutions of diorganozincs or organozinc halides can give the desired ketone or aldehyde. Unlike other organometallic reactions such as the Stille, Heck, and Suzuki, (Bunin, The combinatorial Indexr1 ed.; Academic Press: San Diego, 1998 and Lorsbach and Kurth, Chem. Rev. 99:1549-1581 (1999); Andres et al., Current Opinion in Chemical Biology 2:353-362 (1998); Booth et al., Tetrahedron 54:15385-15443 (1998)) this route has not been extensively investigated, with only one paper to date on organozinc additions to electrophiles on solid support (Davis et al., Advanced Bacterial Genetics: A Manual for Genetic Engineering.; Cold Spring Harbor Press,: New York., 1980). Earlier work describing solid-phase catalysts used to promote enantioselective additions to aldehydes, illustrate the potential success of these strategies (See references within, particularly for alkenyl and alkynyl additions: Soai and Niwa, Chem. Rev. 92:833-856 (1992); Itsuno et al., J. Org. Chem. 55:304-310 (1990); Soai et al., J. Org. Chem. 53:927-928 (1998); Soai et al., J. Chem. Soc. Perkin Trans. 1109-113 (1990); Soai and Watanabe, Tetrahedron: Asymmetry 2:97-100 (1991); and Itsuno and Frechet, J. Org. Chem. 52:4140-4142 (1997)).

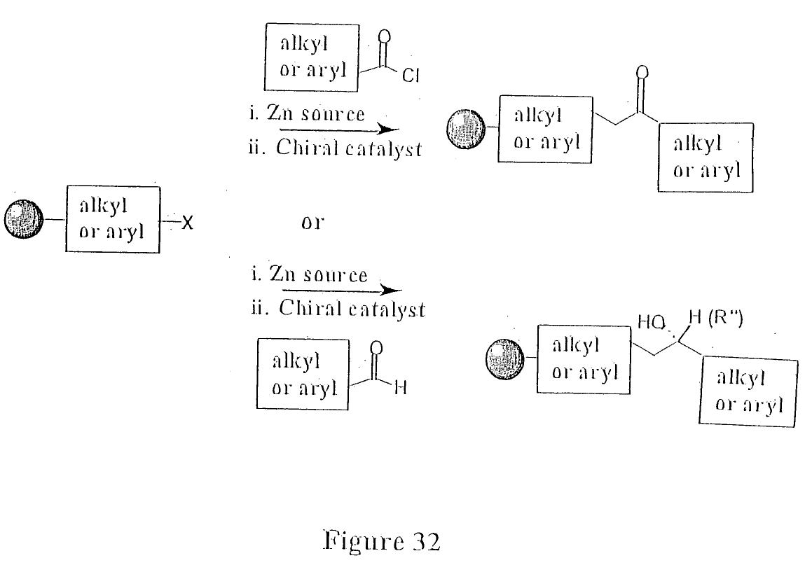

A variety of solid-phase synthesis approaches can be used to prepare a macrocyclic compound using organozinc chemistry. Exemplary solid phase synthesis schemes are shown in FIGS. 32 and 34. The methods for solid phase synthesis can employ a linker that is robust to the synthesis of organozincs and the ensuing chemistry. Such linkers can include, for example, silyl linkers (Randolph et al., J. Am. Chem. Soc. 117:5712-5719 (1995); Woolard et al., J. Org. Chem. 62:6102-6103 (1997); Farrell and Fréchet, J. Org. Chem. 41:3877-3879 (1976); Chan and Huang, J. Chem. Soc. Chem. Commun. 909-910 (1985); Boehm and Showalter, J. Org. Chem. 61:6498-6499 (1996); Hu and Porco, Tetrahedron Lett., 40:3289-3292 (1999); Plunkett and Ellman, J. Org. Chem. 60:6006-6007 (1995); Plunkett and Ellman, J. Org. Chem. 62:2885-2893 (1997); Smith, Tetrahedron Lett. 40:3285-3288 (1999); and Hu et al., J. Org. Chem. 63:4518-4521 (1998)) and all-carbon diisopropyl silyl linkers (Woolard et al., J. Org. Chem. 62:6102-6103 (1997)). Silyl linkers have a robust character and are inert to a wide range of synthetic transformations, including the formation of zinc reagents and their subsequent reactions. They are easily removed using a fluoride source. In addition, they are readily attached to the solid support via lithiation of polystyrene, followed by trapping of the lithiated aryl intermediate using dialkyldichlorosilanes (Randolph et al., J. Am. Chem. Soc. 117:5712-5719 (1995); Farrell and Fréchet, J. Org. Chem. 41:3877-3879 (1976); and Chan and Huang, J. Chem. Soc., Chem. Commun. 909-910 (1985)) or via a Suzuki coupling with bromophenyl substituted resin (Woolard et al., J. Org. Chem. 62:6102-6103 (1997) and Farrell and Fréchet, J. Org. Chem. 41:3877-3879 (1976).

The method of organozinc generation can vary depending on the aldehydes and acid chlorides that are on the solid-phase. The organozinc and the organozinc halides are generally selected to achieve high enantioselectivity when reacting with an aldehyde (Noyori et al., J. Organomet. Chem. 382:19-37 (1990) and Tombo et al., Synlett 547-548 (1990)).

Combinatorial Libraries of Compounds

The methods of the invention for synthesizing combinatorial libraries allow for the ability to substitute an amino acid that forms an amide bond, for a monomer that forms a carbon-carbon bond. This exchange allows a number of advantages to the current method for synthesizing peptides and converting them to peptidomimetics. The synthesis of peptides is well established and relatively rapid (Bodanszky and Bodanszky, Int. J. Pept. Protein Res. 23:565 (1984); Bodanszky and Bodanszky, A., The practise of peptide synthesis (2nd ed.), Springer: New York, N.Y. (1994)). Therefore, when targeting a biological site of interest, it can be ideal, in a synthetic sense, if a relatively rigid yet rapidly built structure could be initially constructed using the computational core and available crystal structure information. Then as specific interactions between the peptide and the biological target have been optimized, the peptide can be converted to a peptidomimetic by replacing some, if not all of the amide and ester functional groups with appropriately designed ketones or alcohols. Not only can organozinc reagents produce both ketones and alcohols, they are unreactive to a large diversity of functional groups and are therefore ideal reagents to be used in a combinatorial library synthesis.

With the development of the organozinc methodology using amino acids describe herein, the design of peptidomimetics based on a known active peptide can be straightforward. That is, the individual exchange of amino acids within an active peptide, for monomers containing appropriate functional groups and consisting of carbon-carbon bonds simplify the peptidomimetic building process. nces of success when using these reagents on solid support.

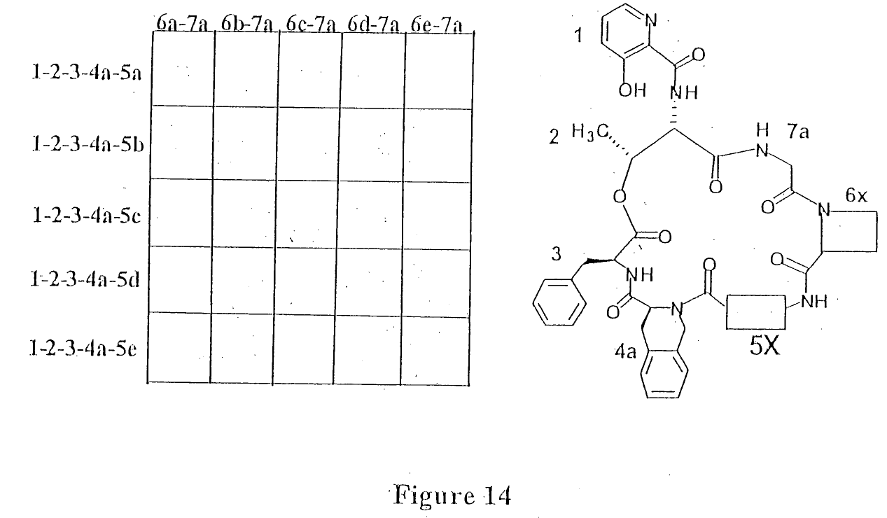

An example of the general strategy for the combinatorial approach is shown in FIG. 14. The figure shows an example lay-out for plates of compounds, where each plate can have a specific 3 and 4, but varies the 5 monomer in each row. For the first directed library, the amount of monomer 6 was varied in each column, and have a specific 7 was used. The choices of these monomers can depend on the biological activity of the initial peptides and the structure of the monomers within these peptides. For a second directed library each plate has a specific 3 and 4, and the 5 monomer was varied by row. In each column was a different organozinc spacer designed to couple to each trimer.

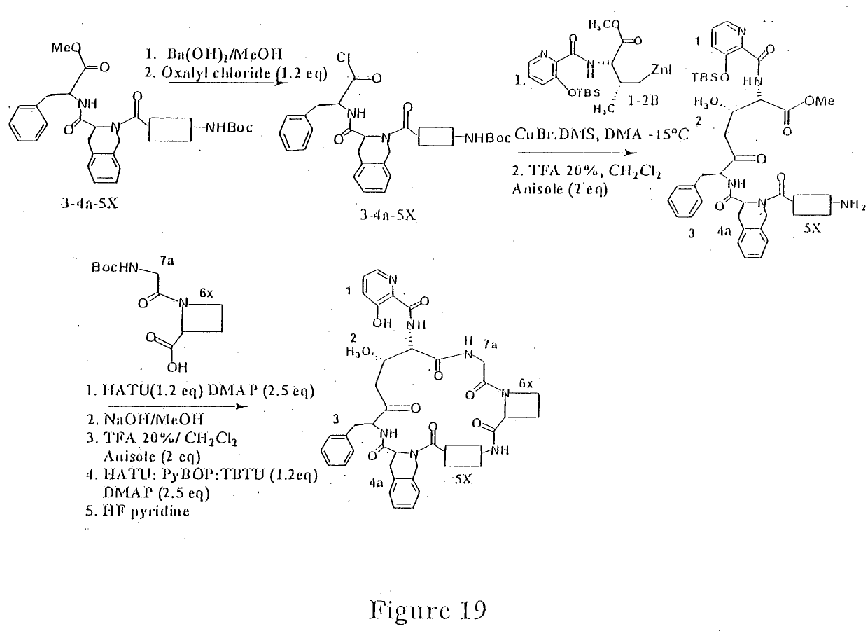

Another example of a synthesis approach for a peptidomimetic macrocyclic compound is shown in FIG. 19. The first peptidomimetic macrocycle has a ketone to replace the labile ester bond. It also has one less atom than the original peptide, but utilizes our current intermediates from our convergent peptide synthesis strategy. The second macrocycle also replaces the labile ester bond with a ketone but has the same number of atoms on the macrocycle as the original peptide. The third macrocycle is designed to exchange not only the ester bond between 2 and 3, but also the amide bond between 5 and 6. The fourth macrocycle is designed to exchange the bonds between 2 and 3, 5 and 6, and 7 and 2. The exchange between 7 and 2 are the site of macrocyclization.

Assays for Testing Compound Activities

A compound synthesized using a method of the invention can be tested for an in vitro or in vivo activity appropriate for its particular desired activity. For example, a Holliday junction-trapping compound can be tested in a variety of in vitro assays for determining the ability of a compound to stabilize a Holliday junction intermediate. Such assays include, for example, determining the ability of a compound to interact with recombinase enzymes, such as XerC/D, and Int; determining the ability of a compound to reduce DNA replication in a cell; the ability of a compound to reduce DNA transcription; and the ability of a compound to reduce or prevent accumulation of DNA damage in a cell. Exemplary assays for testing Holliday junction-trapping compounds are provided in Examples III and IV.

A variety of in vivo assays can be used for determining the ability of a compound to inhibit cell growth, such as bacterial cell growth. Such assays include, for example, measurement of culture growth by density and counting, and measurement of bacterial cell death by detection of dying, degenerating or otherwise non-growing cells. Exemplary assays for determining the ability of a compound to inhibit cell growth are provided in Examples IX and XIV.

In the formulas shown in the figures and text of this application, the stereochemistry of chiral centers associated with the R groups can independently be in the R or S configuration, or a mixture of the two. These can be designated as R or S or R,S or d,D, l,L or d,l, D,L.

R can be a “C₁ to C₁₀ alkyl,” such as methyl, ethyl, n-propyl, isopropyl, n-butyl, sec-butyl, tert-butyl, amyl, tert-amyl, hexyl and the like. The preferred “C₁ to C₁₀alkyl” group is methyl.

R can be a “C₂ to C₁₀alkenyl” such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl, as well as dienes and trienes of straight and branched chains.

R can be a “C₂ to C₁₀alkynyl” such as ethynyl, propynyl, butynyl, pentynyl, hexynyl, heptynyl, as well as di- and tri-ynes of straight and branched chains.

R can be a “C₁ to C₁₀alkylene,” which means a C₁ to C₁₀alkyl group where the alkyl radical is bonded at two positions connecting together two separate additional groups. Examples of C₁ to C₁₀alkylene include methylene, 1,2-ethyl, 1,1-ethyl, 1,3-propyl and the like. The term “C₂ to C₁₀ alkenylene” means a C₂ to C₁₀ alkenyl radican which is bonded at two positions connecting together two separate additional groups.

R can be a “C₁ to C₁₀ substituted alkyl,” “C₂ to C₁₀ substituted alkenyl,” and “C₂ to C₁₀ substituted alkynyl,” denote that the above C₁ to C₁₀ alkyl groups and C₂ to C₁₀ alkenyl and alkynyl groups are substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, C₁ to C₇ alkyl ester, carboxy, protected carboxy, carbamoyl, carboxamide, protected carboxamide, N-(C₁ to C₆ alkyl)carboxamide, protected N-(C₁ to C₆ alkyl)carboxamide, N,N-di(C₁ to C₆alkyl)carboxamide, cyano, methylsulfonylamino, thio, C₁ to C₄ alkylthio or C₁ to C₄ alkyl sulfonyl groups. The substituted alkyl, alkenyl or alkynyl groups may be substituted once or more, and preferably once or twice, with the same or with different substituents.

Examples of the above substituted alkyl groups include the 2-oxo-prop-1-yl, 3-oxo-but-1-yl, cyanomethyl, nitromethyl, chloromethyl, hydroxymethyl, tetrahydropyranyloxymethyl, trityloxymethyl, propionyloxymethyl, amino, methylamino, aminomethyl, dimethylamino, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-aminopropyl, chloroethyl, bromoethyl, fluoroethyl, iodoethyl, chloropropyl, bromopropyl, fluoropropyl, iodopropyl, 2-aminoethyl, 1-aminoethyl, N-benzoyl-2-aminoethyl, N-acetyl-2-aminoethyl, N-benzoyl-1-aminoethyl, N-acetyl-1-aminoethyl and the like.

Examples of the above substituted alkenyl groups include styrenyl, 3-chloro-propen-1-yl, 3-chloro-buten-1-yl, 3-methoxy-propen-2-yl, 3-phenyl-buten-2-yl, 1-cyano-buten-3-yl and the like. The geometrical isomerism is not critical, and all geometrical isomers for a given substituted alkenyl can be used.

Examples of the above substituted alkynyl groups include phenylacetylen-1-yl, 1-phenyl-2-propyn-1-yl and the like.

C₁ to C₁₀ alkyl, C₂ to C₁₀alkenyl, C₂ to C₁₀ alkynyl, C₁ to C₁₀ substituted alkyl, C₂ to C₁₀ substituted alkenyl, or C₂ to C₁₀ substituted alkynyl are preferably C₁ to C₇ or C₂ to C₈, respectively, and more preferably, C₁ to C₆ and C₂ to C₇. However, it should be appreciated by those of skill in the art that one or a few carbons could be added to an alkyl, alkenyl, alkynyl, substituted or unsubstituted, without substantially modifying the structure and function of the subject compounds and that such additions would not depart from the spirit of the invention.

R can be a “C₁ to C₁₀ substituted alkylene” meaning a C₁ to C₁₀ alkyl group where the alkyl radical is bonded at two positions connecting together two separate additional groups and further bearing an additional substituent. Examples of C₁ to C₁₀ substituted alkylene include aminomethylene, 1-(amino)-1,2-ethyl, 2-(amino)-1,2-ethyl, 1-(acetamido)-1,2-ethyl, 2-(acetamido)-1,2-ethyl, 2-hydroxy-1,1-ethyl, 1-(amino)-1,3-propyl. Similarly, the term “C₂ to C₁₀ substituted alkenylene” means a C₂ to C₁₀ substituted alkenyl group where the alkenyl radical is bonded at two positions connecting together two separate additional groups and further bearing an additional substituent.

The term “oxo” denotes a carbon atom bonded to two additional carbon atoms substituted with an oxygen atom doubly bonded to the carbon atom, thereby forming a ketone moiety.

The term “protected oxo” denotes a carbon atom bonded to two additional carbon atoms substituted with two alkoxy groups or twice bonded to a substituted diol moiety, thereby forming an acyclic or cyclic ketal moiety.

R can be a “C₁ to C₇ alkoxy” meaning groups such as methoxy, ethoxy, n-propoxy, isopropoxy, n-butoxy, t-butoxy and like groups. A preferred alkoxy is methoxy. The term “C₁ to C₇ substituted alkoxy” means the alkyl portion of the alkoxy can be substituted in the same manner as in relation to C₁ to C₆ substituted alkyl.

R can be a “C₁ to C₇ acyloxy” meaning groups such as formyloxy, acetoxy, propionyloxy, butyryloxy, pentanoyloxy, hexanoyloxy, heptanoyloxy and the like.

R can be a “C₁ to C₇ acyl” meaning groups such as formyl, acetyl, propionyl, butyryl, pentanoyl, pivaloyl, hexanoyl, heptanoyl, benzoyl and the like. Preferred acyl groups are acetyl and benzoyl.

R can be a “C₁ to C₇ substituted acyl” meaning the acyl group substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, C₁ to C₇ alkyl ester, carboxy, protected carboxy, carbamoyl, carboxamide, protected carboxamide, N(C₁ to C₆ alkyl)carboxamide, protected N-(C₁ to C₆ alkyl)carboxamide, N,N-di(C₁ to C₆ alkyl) carboxamide, cyano, methylsulfonylamino, thio, C₁ to C₄ alkylthio or C₁ to C₄ alkyl sulfonyl groups. The substituted acyl groups may be substituted once or more, and preferably once or twice, with the same or with different substituents.

Examples of C₁ to C₇ substituted acyl include 4-phenylbutyroyl, 3-phenylbutyroyl, 3-phenylpropanoyl, 2-cyclohexanylacetyl, cyclohexanecarbonyl, 2-furanoyl and 3-(dimethylamino)benzoyl.

R can be a “C₃ to C₇ cycloalkyl” including the cyclopropyl, cyclobutyl, cyclopentyl, cyclohexyl or cycloheptyl rings. The substituent term “C₃ to C₇ substituted cycloalkyl” indicates the above cycloalkyl rings substituted by one or two halogen, hydroxy, protected hydroxy, C₁ to C₆ alkyl, C₁ to C₇ alkoxy, oxo, protected oxo, (monosubstituted)amino, (disubstituted)amino, trifluoromethyl, carboxy, protected carboxy, phenyl, substituted phenyl, amino, or protected amino groups.

R can be a “C₁ to C₇ cycloalkenyl” meaning a 1,2, or 3-cyclopentenyl ring, a 1,2,3 or 4-cyclohexenyl ring or a 1,2,3,4 or 5-cycloheptenyl ring, while the term “C₅ to C₇ substituted cycloalkenyl” denotes the above C₅ to C₇ cycloalkenyl rings substituted by a C₁ to C₆ alkyl radical, halogen, hydroxy, protected hydroxy, C₁ to C₇ alkoxy, trifluoromethyl, carboxy, protected carboxy, oxo, protected oxo, (monosubstituted)amino, protected (monosubstituted)amino, (disubstituted)amino, phenyl, substituted phenyl, amino, or protected amino.

The term “heterocyclic ring” denotes optionally substituted five-membered or six-membered rings that have 1 to 4 heteroatoms, such as oxygen, sulfur and/or nitrogen, in particular nitrogen, either alone or in conjunction with sulfur or oxygen ring atoms. These five-membered or six-membered rings may be saturated, fully saturated or partially unsaturated, with fully saturated rings being preferred. A “substituted heterocyclic ring” means any of the above-described heterocycles substituted with any of the substituents as referred to above in relation to substituted phenyl. An “amino-substituted heterocyclic ring” means any one of the above-described heterocyclic rings is substituted with at least one amino group. Preferred heterocyclic rings include morpholino, piperidinyl, piperazinyl, tetrahydrofurano, pyrrolo, and tetrahydrothiophen-yl.

The abbreviation “Ar” stands for an aryl group. Aryl groups which can be used with present invention include phenyl, substituted phenyl, as defined above, heteroaryl, and substituted heteroaryl. The term “heteroaryl” or “heteroaryl ring” means a heterocyclic aromatic derivative which is a five-membered or six-membered ring system having from 1 to 4 heteroatoms, such as oxygen, sulfur and/or nitrogen, in particular nitrogen, either alone or in conjunction with sulfur or oxygen ring atoms. Examples of heteroaryls include pyridinyl, pyrimidinyl, and pyrazinyl, pyridazinyl, pyrrolo, furano, oxazolo, isoxazolo, thiazolo and the like.

R can be a “substituted heteroaryl” or “substituted heteroaryl ring” meaning the above-described heteroaryl is substituted with, for example, one or more, and preferably one or two, substituents which are the same or different which substituents can be halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino carboxamide, protected carboxamide, N(C₁ to C₆alkyl)carboxamide, protected N-(C₁ to C₆ alkyl)carboxamide, N,N-di(C₁ to C₆ alkyl)carboxamide, trifluoromethyl, N-((C₁ to C₆ alkyl)sulfonyl)amino or N-(phenylsulfonyl)amino groups.

R can be a “C₇ to C₁₂ phenylalkyl” meaning a C₁ to C₆ alkyl group substituted at any position by a phenyl ring. Examples of such a group include benzyl, 2-phenylethyl, 3-phenyl(n-propyl), 4-phenylhexyl, 3-phenyl(n-amyl), 3-phenyl(sec-butyl) and the like. Preferred C₇ to C₁₂ phenylalkyl groups are the benzyl and the phenylethyl groups.

R can be a “C₇ to C₁₂ substituted phenylalkyl” meaning a C₇ to C₁₂ phenylalkyl group substituted on the C₁ to C₆ alkyl portion with one or more, and preferably one or two, groups chosen from halogen, hydroxy, protected hydroxy, oxo, protected oxo, amino, protected amino, (monosubstituted)amino, protected (monosubstituted)amino, (disubstituted)amino, guanidino, heterocyclic ring, substituted heterocyclic ring, C₁ to C₇ alkoxy, C₁ to C₇ substituted alkoxy, C₁ to C₇ acyl, C₁ to C₇ acyloxy, nitro, carboxy, protected carboxy, carbamoyl, carboxamide, protected carboxamide, N-(C₁ to C₆ alkyl)carboxamide, protected N-C₁ to C6 alkyl)carboxamide, N,N-(C₁ to C₆ dialkyl)carboxamide, cyano, N-((C1 to C6 alkylsulfonyl)amino, thiol, C₁ to C₄ alkylthio, C₁ to C₄ alkylsulfonyl groups; and/or the phenyl group may be substituted with one or more, and preferably one or two, substituents chosen from halogen, hydroxy, protected hydroxy, cyano, nitro, C1 to C6 alkyl, C₁ to C₇ alkoxy, C₁ to C₇ substituted alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C₁ to C₆ alkyl) carboxamide, protected N-(C₁ to C₆ alkyl) carboxamide, N,N-di(C₁ to C₆ alkyl)carboxamide, trifluoromethyl, N-((C₁ to C₆ alkyl)sulfonyl)amino, N-(phenylsulfonyl)amino, cyclic C₂ to C₇ alkylene or a phenyl group, substituted or unsubstituted, for a resulting biphenyl group. The substituted alkyl or phenyl groups may be substituted with one or more, and preferably one or two, substituents which can be the same or different.

Examples of the term “C₇ to C₁₂ substituted phenylalkyl” include groups such as 2-phenyl-1-chloroethyl, 2-(4-methoxyphenyl)ethyl, 4-(2,6-dihydroxy phenyl)n-hexyl, 2-(5-cyano-3-methoxyphenyl)n-pentyl, 3-(2,6-dimethylphenyl)n-propyl, 4-chloro-3-aminobenzyl, 6-(4-methoxyphenyl)-3-carboxy(n-hexyl), 5-(4-aminomethylphenyl)-3-(aminomethyl)ₙ-pentyl, 5-phenyl-3-oxo-n-pent-1-yl and the like.

R can be a “substituted phenyl” meaning a phenyl group substituted with one or more, and preferably one or two, moieties chosen from the groups consisting of halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted)amino, carboxamide, protected carboxamide, N(C₁ to C₆ alkyl)carboxamide, protected N-(C₁ to C₆ alkyl)carboxamide, N,N-di (C₁ to C₆ alkyl)carboxamide, trifluoromethyl, N-((C₁ to C₆ alkyl)sulfonyl)amino, N-(phenylsulfonyl)amino or phenyl, substituted or unsubstituted, such that, for example, a biphenyl results.

Examples of the term “substituted phenyl” include a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3 or 4-hydroxyphenyl, 2,4-dihydroxyphenyl, the protected-hydroxy derivatives thereof and the like; a nitrophenyl group such as 2, 3 or 4-nitrophenyl; a cyanophenyl group, for example, 2, 3 or 4-cyanophenyl; a mono- or di(alkyl)phenyl group such as 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl and the like; a mono or di(alkoxyl)phenyl group, for example, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono-or di(hydroxymethyl)phenyl or (protected hydroxymethyl)phenyl such as 2, 3, or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy 4-chlorophenyl and the like.

R can be a “phenylene” meaning a phenyl group where the phenyl radical is bonded at two positions connecting together two separate additional groups. Examples of phenylene include 1,2-phenyl, 1,3-phenyl, and 1,4-phenyl.

R can be a “substituted phenylene” meaning a substituted phenyl group where the phenyl radical is bonded at two positions connecting together two separate additional groups. Examples of substituted phenylene include 3-chloro-1,2-phenyl, 4-amino-1,3-phenyl, and 3-hydroxy-1,4-phenyl.

R can be a “phenoxy” meaning a phenyl bonded to an oxygen atom provided that the phenoxy is bonded to the quinoline ring through the oxygen atom as opposed to a carbon atom of the phenyl ring. The term “substituted phenoxy” specifies a phenoxy group substituted with one or more, and preferably one or two, moieties chosen from the groups consisting of halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C₁ to C₆ alkyl)carboxamide, protected N-(C₁ to C₆ alkyl)carboxamide, N,N-di(C₁ to C₆ alkyl)carboxamide, trifluoromethyl, N-((C₁ to C₆ alkyl)sulfonyl)amino and N-(phenylsulfonyl)amino.

Examples of substituted phenoxy include 2-methylphenoxy, 2-ethylphenoxy, 2-propylphenoxy, 2-isopropylphenoxy, 2-sec-butylphenoxy, 2-tert-butylphenoxy, 2-allylphenoxy, 2-propenylphenoxy, 2-cyclopentylphenoxy, 2-fluorophenoxy, 2-(trifluoromethyl)phenoxy, 2-chlorophenoxy, 2-bromophenoxy, 2-methoxyphenoxy, 2-ethoxyphenoxy, 2-isopropoxyphenoxy, 3-methylphenoxy, 3-ethylphenoxy, 3-isopropylphenoxy, 3-tert-butylphenoxy, 3-pentadecylphenoxy, 3-(trifluoromethyl)phenoxy, 3-fluorophenoxy, 3-chlorophenoxy, 3-bromophenoxy, 3-iodophenoxy, 3-methoxyphenoxy, 3-(trifluoromethoxy)phenoxy, 4-methylphenoxy, 4-ethylphenoxy, 4-propylphenoxy, 4-isopropylphenoxy, 4-sec-butylphenoxy, 4-tert-butylphenoxy, 4-tert-amylphenoxy, 4-nonylphenoxy, 4-dodecylphenoxy, 4-cyclopenylphenoxy, 4-(trifluoromethyl)phenoxy, 4-fluorophenoxy, 4-chlorophenoxy, 4-bromophenoxy4-iodophenoxy, 4-methoxyphenoxy, 4-(trifluoromethoxy)phenoxy, 4-ethoxyphenoxy, 4-propoxyphenoxy, 4-butoxyphenoxy, 4-hexyloxyphenoxy, 4-heptyloxyphenoxy, 2,3-dimethylphenoxy, 5,6,7,8-tetrahydro-1-naphthoxy, 2,3-dichlorophenoxy, 2,3-dihydro-2,2-dimethyl-7-benzofuranoxy, 2,3-dimethoxyphenoxy, 2,6-dimethylphenoxy, 2,6-diisopropylphenoxy, 2,6-di-sec-butylphenoxy, 2-tert-butyl-6-methylphenoxy, 2,6-di-tert-butylphenoxy, 2-allyl-6-methylphenoxy, 2,6-difluorophenoxy, 2,3-difluorophenoxy, 2,6-dichlorophenoxy, 2,6-dibromophenoxy, 2-fluoro-6-methoxyphenoxy, 2,6-dimethoxyphenoxy, 3,5-dimethylphenoxy, 5-isopropyl-3-methylphenoxy, 3,5-di-tert-butylphenoxy, 3,5-bis(trifluoromethyl)phenoxy, 3,5-difluorophenoxy, 3,5-dichlorophenoxy, 3,5-dimethoxyphenoxy, 3-chloro-5-methoxyphenoxy, 3,4-dimethylphenoxy, 5-indanoxy, 5,6,7,8-tetrahydro-2-naphthoxy, 4-chloro-3-methylphenoxy, 2,4-dimethylphenoxy, 2,5-dimethylphenoxy, 2-isopropyl-5-methylphenoxy, 4-isopropyl-3-methylphenoxy, 5-isopropyl-2-methylphenoxy, 2-tert-butyl-5-methylphenoxy, 2-tert-butyl-4-methylphenoxy, 2,4-di-tert-butylphenoxy, 2,4-di-tert-amylphenoxy, 4-fluoro-2-methylphenoxy, 4-fluoro-3-methylphenoxy, 2-chloro-4-methylphenoxy, 2-chloro-5-methylphenoxy, 4-chloro-2-methylphenoxy, 4-chloro-3-ethylphenoxy, 2-bromo-4-methylphenoxy, 4-iodo-2-methylphenoxy, 2-chloro-5-(trifluoromethyl)phenoxy, 2,4-difluorophenoxy, 2,5-difluorophenoxy, 3,4-difluorophenoxy, 4-chloro-2-fluorophenoxy, 3-chloro-4-fluorophenoxy, 4-chloro-3-fluorophenoxy, 2-bromo-4-fluorophenoxy, 4-bromo-2-fluorophenoxy, 2-bromo-5-fluorophenoxy, 2,4-dichlorophenoxy, 3,4-dichlorophenoxy, 2,5-dichlorophenoxy, 2-bromo-4-chlorophenoxy, 2-chloro-4-fluorophenoxy, 4-bromo-2-chlorophenoxy, 2,4-dibromophenoxy, 2-methoxy-4-methylphenoxy, 4-allyl-2-methylphenoxy, trans-2-ethoxy-5-(1-propenyl)phenoxy, 2-methoxy-4-propenylphenoxy, 3,4-dimethoxyphenoxy, 3-ethoxy-4-methoxyphenoxy, 4-allyl-2,6-dimethoxyphenoxy, 3,4-methylenedioxyphenoxy, 2,3,6-trimethylphenoxy, 2,4-dichloro-3-methylphenoxy, 2,3,4-trifluorophenoxy, 2,3,6-trifluorophenoxy, 2,3,5-trifluorophenoxy, 2,3,4-trichlorophenoxy, 2,3,6-trichlorophenoxy, 2,3,5-trimethylphenoxy, 3,4,5-trimethylphenoxy, 4-chloro-3,5-dimethylphenoxy, 4-bromo-3,5-dimethylphenoxy, 2,4,6-trimethylphenoxy, 2,6-bis(hydroxymethyl)-4-methylphenoxy, 2,6-di-tert-butyl-4-methylphenoxy, 2,6-di-tert-butyl-4-methoxyphenoxy, 2,4,5-trifluorophenoxy, 2-chloro-3,5-difluorophenoxy, 2,4,6-trichlorophenoxy, 3,4,5-trimethoxyphenoxy, 2,3,5-trichlorophenoxy, 4-bromo-2,6-dimethylphenoxy, 4-bromo-6-chloro-2-methylphenoxy, 2,6-dibromo-4-methylphenoxy, 2,6-dichloro-4-fluorophenoxy, 2,6-dibromo-4-fluorophenoxy, 2,4,6-tribromophenoxy, 2,4,6-triiodophenoxy, 2-chloro-4,5-dimethylphenoxy, 4-chloro-2-isopropyl-5-methylphenoxy, 2-bromo-4,5-difluorophenoxy, 2,4,5-trichlorophenoxy, 2,3,5,6-tetrafluorophenoxy and the like.

R can be a “C₇ to C₁₂ phenylalkoxy” meaning a C₇ to C₁₂ phenylalkoxy group, provided that the phenylalkoxy is bonded to the quinoline ring through the oxygen atom. By “C₇ to C₁₂ substituted phenylalkoxy” is meant C₇ to C₁₂ phenylalkoxy group which can be substituted on the C₁ to C₆ alkyl portion with one or more, and preferably one or two, groups chosen from halogen, hydroxy, protected hydroxy, oxo, protected oxo, amino, protected amino, (monosubstituted) amino, protected (monosubstituted) amino, (disubstituted)amino, guanidino, heterocyclic ring, substituted heterocyclic ring, C₁ to C₇ alkoxy, C₁ to C₇ acyl, C₁ to C₇ acyloxy, nitro, carboxy, protected carboxy, carbamoyl, carboxamide, protected carboxamide, N-(C₁ to C₆ alkyl)carboxamide, protected N-C₁ to C₆ alkyl)carboxamide, N,N-(C₁ to C₆ dialkyl)carboxamide, cyano, N-((C₁ to C₆ alkylsulfonyl)amino, thiol, C₁ to C₄ alkylthio, C₁ to C₄ alkylsulfonyl groups; and/or the phenyl group can be substituted with one or more, and preferably one or two, substituents chosen from halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N(C₁ to C₆ alkyl) carboxamide, protected N-(C₁ to C₆ alkyl) carboxamide, N,N-di(C₁ to C₆ alkyl)carboxamide, trifluoromethyl, N-((C₁ to C₆ alkyl)sulfonyl)amino, N-(phenylsulfonyl)amino or a phenyl group, substituted or unsubstituted, for a resulting biphenyl group. The substituted alkyl or phenyl groups may be substituted with one or more, and preferably one or two, substituents which can be the same or different.

Examples of the term “C₇ to C₁₂ substituted phenylalkoxy” include groups such as 2-(4-hydroxyphenyl)ethoxy, 4-(4-methoxyphenyl)butoxy, (2R)-3-phenyl-2-amino-propoxy, (2S)-3-phenyl-2-amino-propoxy, 2-indanoxy, 6-phenyl-1-hexanoxy, cinnamyloxy, (+/−)-2-phenyl-1-propoxy, 2,2-dimethyl-3-phenyl-1-propoxy and the like.

The term “phthalimide” means a cyclic imide which is made from phthalic acid, also called 1, 2 benezene-dicarboxylic acid. The term “substituted phthalimide” specifies a phthalimide group substituted with one or more, and preferably one or two, moieties chosen from the groups consisting of halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C₁ to C₆ alkyl)carboxamide, protected N-(C₁ to C₆ alkyl)carboxamide, N,N-di(C₁ to C₆ alkyl)carboxamide, trifluoromethyl, N-((C₁ to C₆ alkyl)sulfonyl)amino and N-(phenylsulfonyl)amino.

Examples of substituted phthalimides include 4,5-dichlorophthalimido, 3-fluorophthalimido, 4-methoxyphthalimido, 3-methylphthalimido, 4-carboxyphthalimido and the like.

R can be a “substituted naphthyl” meaning a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted) amino, (disubstituted) amino, carboxamide, protected carboxamide, N-(C₁ to C₆ alkyl)carboxamide, protected N-(C₁ to C₆alkyl)carboxamide, N,N-di(C₁ to C₆ alkyl)carboxamide, trifluoromethyl, N-((C₁ to C₆ alkyl) sulfonyl)amino or N-(phenylsulfonyl) amino.

Examples of substituted naphthyl include a mono or di(halo)naphthyl group such as 1, 2, 3, 4, 5, 6, 7 or 8-chloronaphthyl, 2,6-dichloronaphthyl, 2, 5-dichloronaphthyl, 3,4-dichloronaphthyl, 1, 2, 3, 4, 5, 6, 7 or 8-bromonaphthyl, 3,4-dibromonaphthyl, 3-chloro-4-fluoronaphthyl, 1, 2, 3, 4, 5, 6, 7 or 8-fluoronaphthyl and the like; a mono or di(hydroxy)naphthyl group such as 1, 2, 3, 4, 5, 6, 7 or 8-hydroxynaphthyl, 2,4-dihydroxynaphthyl, the protected-hydroxy derivatives thereof and the like; a nitronaphthyl group such as 3- or 4-nitronaphthyl; a cyanonaphthyl group, for example, 1, 2, 3, 4, 5, 6, 7 or 8-cyanonaphthyl; a mono- or di(alkyl)naphthyl group such as 2, 3, 4, 5, 6, 7 or 8-methylnaphthyl, 1, 2, 4-dimethylnaphthyl, 1, 2, 3, 4, 5, 6, 7 or 8-(isopropyl)naphthyl, 1, 2, 3, 4, 5, 6, 7 or 8-ethylnaphthyl, 1, 2, 3, 4, 5, 6, 7 or 8-(n-propyl)naphthyl and the like; a mono or di(alkoxy)naphthyl group, for example, 2,6-dimethoxynaphthyl, 1, 2, 3, 4, 5, 6, 7 or 8-methoxynaphthyl, 1, 2, 3, 4, 5, 6, 7 or 8-ethoxynaphthyl, 1, 2, 3, 4, 5, 6, 7 or 8(isopropoxy)naphthyl, 1, 2, 3, 4, 5, 6, 7 or 8-(t-butoxy)naphthyl, 3-ethoxy-4-methoxynaphthyl and the like; 1, 2, 3, 4, 5, 6, 7 or 8-trifluoromethylnaphthyl; a mono- or dicarboxynaphthyl or (protected carboxy)naphthyl group such as 1, 2, 3, 4, 5, 6, 7 or 8-carboxynaphthyl or 2,4-di(protected carboxy)naphthyl; a mono- or di(hydroxymethyl)naphthyl or (protected hydroxymethyl)naphthyl such as 1, 2, 3, 4, 5, 6, 7 or 8-(protected hydroxymethyl)naphthyl or 3, 4-di(hydroxymethyl)naphthyl; a mono- or di(amino)naphthyl or (protected amino)naphthyl such as 1, 2, 3, 4, 5, 6, 7 or 8-(amino)naphthyl or 2,4-(protected amino)-naphthyl, a mono- or di(aminomethyl)naphthyl or (protected aminomethyl)naphthyl such as 2, 3, or 4-(aminomethyl)naphthyl or 2,4-(protected aminomethyl)naphthyl; or a mono- or di-(N-methylsulfonylamino) naphthyl such as 1, 2, 3, 4, 5, 6, 7 or 8-(N-methylsulfonylamino)naphthyl. Also, the term “substituted naphthyl” represents disubstituted naphthyl groups wherein the substituents are different, for example, 3-methyl-4-hydroxynaphth-1-yl, 3-chloro-4-hydroxynaphth-2-yl, 2-methoxy-4-bromonaphth-1-yl, 4-ethyl-2-hydroxynaphth-1-yl, 3-hydroxy-4-nitronaphth-2-yl, 2-hydroxy-4-chloronaphth-1-yl, 2-methoxy-7-bromonaphth-1-yl, 4-ethyl-5-hydroxynaphth-2-yl, 3-hydroxy-8-nitronaphth-2-yl, 2-hydroxy-5-chloronaphth-1-yl and the like.

The terms “halo” and “halogen” refer to the fluoro, chloro, bromo or iodo groups. There can be one or more halogen, which are the same or different. Preferred halogens are chloro and fluoro.

R can be a “(monosubstituted)amino” meaning an amino group with one substituent chosen from the group consisting of phenyl, substituted phenyl, C₁ to C₆ alkyl, C₁ to C₆ substituted alkyl, C₁ to C₇ acyl, C₂ to C₇ alkenyl, C₂ to C₇ substituted alkenyl, C₂ to C₇ alkynyl, C₂ to C₇ substituted alkynyl, C₇ to C₁₂ phenylalkyl, C₇ to C₁₂ substituted phenylalkyl and heterocyclic ring. The (monosubstituted)amino can additionally have an amino-protecting group as encompassed by the term “protected (monosubstituted)amino.”

R can be a “(disubstituted)amino” meaning amino groups with two substituents chosen from the group consisting of phenyl, substituted phenyl, C₁ to C₆ alkyl, C₁ to C₆ substituted alkyl, C₁ to C₇ acyl, C₂ to C₇ alkenyl, C₂ to C₇ alkynyl, C₇ to C₁₂ phenylalkyl, and C₇ to C₁₂ substituted phenylalkyl. The two substituents can be the same or different.

R can be a “amino-protecting group” meaning substituents of the amino group commonly employed to block or protect the amino functionality while reacting other functional groups of the molecule. The term “protected (monosubstituted)amino” means there is an amino-protecting group on the monosubstituted amino nitrogen atom. In addition, the term “protected carboxamide” means there is an amino-protecting group on the carboxamide nitrogen.

Examples of such amino-protecting groups include the formyl (“For”) group, the trityl group, the phthalimido group, the trichloroacetyl group, the chloroacetyl, bromoacetyl, and iodoacetyl groups, urethane-type blocking groups, such as t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl (“Ddz”), 2-(p-toluyl)propyl-2-oxycarbonyl, cyclopentanyloxycarbonyl, 1-methylcyclopentanyloxycarbonyl, cyclohexanyloxy-carbonyl, 1-methylcyclohexanyloxycarbonyl, 2-methylcyclohexanyloxycarbonyl, 2-(4-toluylsulfonyl)-ethoxycarbonyl, 2-(methylsulfonyl)ethoxycarbonyl, 2-(triphenylphosphino)-ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloroethoxycarbonyl, 2-ethynyl-2-propoxycarbonyl, cyclopropylmethoxycarbonyl, isobornyloxycarbonyl, 1-piperidyloxycarbonyl, benzyloxycarbonyl (“Cbz”), 4-phenylbenzyloxycarbonyl, 2-methylbenzyloxy-carbonyl, α-2,4,5,-tetramethylbenzyloxycarbonyl (“Tmz”), 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyl-oxycarbonyl, 4-bromobenzyloxycarbonyl, 3-bromobenzyloxycarbonyl, 4-nitrobenzyloxy-carbonyl, 4-cyanobenzyloxycarbonyl, 4-(decyloxy)benzyloxycarbonyl and the like; the benzoylmethylsulfonyl group, dithiasuccinoyl (“Dts”), the 2-(nitro)phenylsulfenyl group (“Nps”), the diphenyl-phosphine oxide group and like amino-protecting groups. The species of amino-protecting group employed is not critical so long as the derivatized amino group is stable to the conditions of the subsequent reaction(s) and can be removed at the appropriate point without disrupting the remainder of the compounds. Preferred amino-protecting groups are Boc, Cbz and Fmoc. Further examples of amino-protecting groups embraced by the above term are well known in organic synthesis and the peptide art and are described by, for example, T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, each of which is incorporated herein by reference. The related term “protected amino” defines an amino group substituted with an amino-protecting group discussed above.

R can be a “carboxy-protecting group” meaning one of the ester derivatives of the carboxylic acid group commonly employed to block or protect the carboxylic acid group while reactions are carried out on other functional groups on the compound. Examples of such carboxylic acid protecting groups include t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, β-(trimethylsilyl)ethyl, β-(di(n-butyl)methylsilyl)ethyl, p-toluenesulfonylethyl, 4-nitrobenzylsulfonylethyl, allyl, cinnamyl, 1-(trimethylsilylmethyl)propenyl and like moieties. The species of carboxy-protecting group employed is not critical so long as the derivatized carboxylic acid is stable to the conditions of subsequent reaction(s) and can be removed at the appropriate point without disrupting the remainder of the molecule. Further examples of these groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5, each of which is incorporated herein by reference. A related term is “protected carboxy,” which refers to a carboxy group substituted with one of the above carboxy-protecting groups.

R can be a “hydroxy-protecting group” meaning readily cleavable groups bonded to hydroxyl groups, such as the tetrahydropyranyl, 2-methoxypropyl, 1-ethoxyethyl, methoxymethyl, 2-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, 2,2,2-trichloroethoxycarbonyl groups and the like. The species of hydroxy-protecting group is not critical so long as the derivatized hydroxyl group is stable to the conditions of subsequent reaction(s) and can be removed at the appropriate point without disrupting the remainder of the molecule. Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3. Related terms are “protected hydroxy,” and “protected hydoxymethyl” which refer to a hydroxy or hydroxymethyl substituted with one of the above hydroxy-protecting groups.

R can be a “C₁ to C₄ alkylthio” refering to sulfide groups such as methylthio, ethylthio, n-propylthio, isopropylthio, n-butylthio, t-butylthio and like groups.

R can be a “C₁ to C₄ alkylsulfoxide” indicating sulfoxide groups such as methylsulfoxide, ethylsulfoxide, npropylsulfoxide, isopropylsulfoxide, n-butylsulfoxide, sec-butylsulfoxide and the like.

R can be a “C₁ to C₄ alkylsulfonyl” encompassing groups such as methylsulfonyl, ethylsulfonyl, n-propylsulfonyl, isopropylsulfonyl, n-butylsulfonyl, t-butylsulfonyl and the like.

R can be a “C₁ to C₄ substituted alkylthio,” “C₁ to C₄ substituted alkylsulfoxide,” and “C₁ to C₄ substituted alkylsulfonyl,” denoting the C₁ to C₄ alkyl portion of these groups may be substituted as described above in relation to “substituted alkyl.”

R can be a “phenylthio,”phenylsulfoxide,” and “phenylsulfonyl” specify a thiol, a sulfoxide, or sulfone, respectively, containing a phenyl group. The terms “substituted phenylthio,” “substituted phenylsulfoxide,” and “substituted phenylsulfonyl” mean that the phenyl of these groups can be substituted as described above in relation to “substituted phenyl.”

R can be a “C₁ to C₆ alkylaminocarbonyl” meaning a C₁ to C₆ alkyl attached to an aminocarbonyl group, where the C₁ to C₆ alkylaminocarbonyl groups are the resulting urea when an isocyanate is used in the reaction scheme. Examples of C₁ to C₆ alkylaminocarbonyl include methylaminocarbonyl (from methylisocyanate), ethylaminocarbonyl (from ethylisocyanate), propylaminocarbonyl (from propylisocyanate), butylaminocarbonyl (from butylisocyatate). The term “C₁ to C₆ substituted alkylaminocarbonyl” denotes a substituted alkyl bonded to an aminocarbonyl group, which alkyl may be substituted as described above in relation to C₁ to C₆ substituted alkyl. Examples of C₁ to C₆ substituted alkylaminocarbonyl include, for example, methoxymethylaminocarbonyl (from methoxymethylisocyanate), 2-chloroethylaminocarbonyl (from 2-chloroethylisocyanate), 2-oxopropylaminocarbonyl (from 2-oxopropylisocyanate), and 4-phenylbutylaminocarbonyl (from phenylbutylisocyanate).

R can be a “phenylaminocarbonyl” meaning a phenyl attached to an aminocarbonyl group, where the phenylaminocarbonyl groups are the result of using a phenylisocyanate in the reaction scheme. The term “substituted phenylaminocarbonyl” denotes a substituted phenyl bonded to an aminocarbonyl group, which phenyl may be substituted as described above in relation to substituted phenyl. Examples of substituted phenylaminocarbonyl include 2-chlorophenylaminocarbonyl (from 2-chlorophenylisocyanate), 3-chlorophenylaminocarbonyl (from 3-chlorophenylisocyanate), 2-nitorphenylaminocarbonyl (from 2-nitrophenylisocyanate), 4-biphenylaminocarbonyl (from 4-biphenylisocyanate), and 4-methoxyphenylaminocarbonyl (from 4-methoxyphenylisocyanate).

R can be a “cyclic C₂ to C₇ alkylene,” “substituted cyclic C₂ to C₇ alkylene,” “cyclic C₂ to C7 heteroalkylene,” and “substituted cyclic C₂ to C₇ heteroalkylene,” defining such a cyclic group bonded (“fused”) to the phenyl radical resulting in a bicyclic ring system. The cyclic group may be saturated or contain one or two double bonds. Furthermore, the cyclic group may have one or two methylene or methine groups replaced by one or two oxygen, nitrogen or sulfur atoms which are the cyclic C₂ to C₇ heteroalkylene.

The cyclic alkylene or heteroalkylene group may be substituted once or twice by the same or different substituents selected from the group consisting of the following moieties: hydroxy, protected hydroxy, carboxy, protected carboxy, oxo, protected oxo, C₁ to C₄ acyloxy, formyl, C₁ to C₇ acyl, C₁ to C₆ alkyl, C₁ to C₇ alkoxy, C₁ to C₄ alkylthio, C₁ to C₄ alkylsulfoxide, C₁ to C₄ alkylsulfonyl, halo, amino, protected amino, (monosubstituted)amino, protected (monosubstituted)amino, (disubstituted)amino, hydroxymethyl and a protected hydroxymethyl.

The cyclic alkylene or heteroalkylene group fused onto the benzene radical can contain two to ten ring members, but it preferably contains three to six members. Examples of such saturated cyclic groups are when the resultant bicyclic ring system is 2,3-dihydro-indanyl and a tetralin ring. When the cyclic groups are unsaturated, examples occur when the resultant bicyclic ring system is a naphthyl ring or indolyl. Examples of fused cyclic groups which each contain one nitrogen atom and one or more double bond, preferably one or two double bonds, are when the phenyl is fused to a pyridino, pyrano, pyrrolo, pyridinyl, dihydropyrrolo, or dihydropyridinyl ring. Examples of fused cyclic groups which each contain one oxygen atom and one or two double bonds are when the phenyl ring is fused to a furo, pyrano, dihydrofurano, or dihydropyrano ring. Examples of fused cyclic groups which each have one sulfur atom and contain one or two double bonds are when the phenyl is fused to a thieno, thiopyrano, dihydrothieno or dihydrothiopyrano ring. Examples of cyclic groups which contain two heteroatoms selected from sulfur and nitrogen and one or two double bonds are when the phenyl ring is fused to a thiazolo, isothiazolo, dihydrothiazolo or dihydroisothiazolo ring. Examples of cyclic groups which contain two heteroatoms selected from oxygen and nitrogen and one or two double bonds are when the benzene ring is fused to an oxazolo, isoxazolo, dihydrooxazolo or dihydroisoxazolo ring. Examples of cyclic groups which contain two nitrogen heteroatoms and one or two double bonds occur when the benzene ring is fused to a pyrazolo, imidazolo, dihydropyrazolo or dihydroimidazolo ring or pyrazinyl.

One or more of the compounds, even within a given library, may be present as a salt. The term “salt” encompasses those salts that form with the carboxylate anions and amine nitrogens and include salts formed with the organic and inorganic anions and cations discussed below. Furthermore, the term includes salts that form by standard acid-base reactions with basic groups (such as amino groups) and organic or inorganic acids. Such acids include hydrochloric, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and like acids.

The term “organic or inorganic cation” refers to counterions for the carboxylate anion of a carboxylate salt. The counter-ions are chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge et al., J. Pharm. Sci., 66:1-19 (1977), which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is referred to by this term. For example, a cation for a carboxylate anion will exist when R₂ or R₃ is substituted with a (quaternary ammonium)methyl group. A preferred cation for the carboxylate anion is the sodium cation.

The compounds of the above Formulae can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

One or more compounds, even when in a library, can be in the biologically active ester form, such as the nontoxic, metabolically-labile ester-form. Such ester forms induce increased blood levels and prolong the efficacy of the corresponding non-esterified forms of the compounds. Ester groups which can be used include the lower alkoxymethyl groups, for example, methoxymethyl, ethoxymethyl, isopropoxymethyl and the like; the α-(C₁ to C₇) alkoxyethyl groups, for example methoxyethyl, ethoxyethyl, propoxyethyl, isopropoxyethyl and the like; the 2-oxo-1,3-diooxlen-4-ylmethyl groups, such as 5-methyl-2-oxo-1,3-dioxolen-4-ylmethyl, 5-phenyl-2-oxo-1,3-dioxolen-4-ylmethyl and the like; the C₁ to C₄ alkylthiomethyl groups, for example methylthiomethyl, ethylthiomethyl, isopropylthiomethyl and the like; the acyloxymethyl groups, for example pivaloyloxymethyl, pivaloyloxyethyl, α-acetoxymethyl and the like; the ethoxycarbonyl-1-methyl group; the α-acetoxyethyl; the 1-(C₁ to C₇ alkyloxycarbonyloxy)ethyl groups such as the 1-(ethoxycarbonyloxy)ethyl group; and the 1-(C₁ to C₇ alkylaminocarbonyloxy)ethyl groups such as the 1-(methylaminocarbonyloxy)ethyl group.

The term “amino acid” includes any one of the twenty naturally-occurring amino acids or the D-form of any one of the naturally-occurring amino acids. In addition, the term “amino acid” also includes other non-naturally occurring amino acids besides the D-amino acids, which are functional equivalents of the naturally-occurring amino acids. Such non-naturally-occurring amino acids include, for example, norleucine (“Nle”), norvaline (“Nva”), β-Alanine, L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art.

The amino acids are indicated herein by either their full name or by the commonly known three letter code. Further, in the naming of amino acids, “D-” or “d-” designates an amino acid having the “D” configuration, as opposed to the naturally occurring L-amino acids. Where no specific configuration is indicated, one skilled in the art would understand the amino acid to be an L-amino acid. The amino acids can, however, also be in racemic mixtures of the D- and L-configuration or the D-amino acid can readily be substituted for that in the L-configuration.

Amino acids can be described in terms of having a peptide backbone segment and having an R-group, as commonly understood in the art. This R group can any of the compounds described above, such as an alkyl, alkoyxy, acyloxy, acyl, cycloalkyl heterocyclic ring, phenylalkyl and their substituted versions. Various amino acids, amino acid derivatives and R-groups are illustrated and exemplified in Tables 2, 3 and 4.

As used herein, a chemical or combinatorial “library” is an intentionally created collection of differing molecules which can be prepared by the synthetic means provided below or otherwise and screened for biological activity in a variety of formats (e.g., libraries of soluble molecules, libraries of compounds attached to resin beads, silica chips or other solid supports). The libraries can be screened in any variety of assays, such as those detailed below as well as others useful for assessing the biological activity. The libraries are useful in their ability to rapidly synthesize and screen a diverse number or compounds. Moreover, the libraries will generally have at least one active compound and are generally prepared in such that the compounds are in equimolar quantities. A library of the invention can contain 2, 5, 10, 20, 50, 100, 200, 500, 1000, 2000, 5000, 10,000 and even 24,000 or more compounds.

“Combinatorial chemistry” or “combinatorial synthesis” refers to the parallel synthesis of diverse compounds by sequential addition of reagents which leads to the generation of large chemical libraries having molecular diversity. Combinatorial chemistry, therefore, involves the systematic and repetitive, covalent connection of a set of different “building blocks” of varying structures to yield large arrays of diverse molecular entities.

The compounds and the libraries of compounds can be prepared as set forth herein. It should be appreciated that the substituents discussed herein are merely exemplary and other groups can be used.

The generic and substituent structures shown can reflect their actual structure in the compound or in on the precursor elements. For example, it common practice in the peptide arts to represent or disclose different R groups by providing the R group as attached to a precursor molecule prior to addition to a main compound that will receive the R group. Thus, disclosure of a listing of amino acid derivatives—such as acid chlorides, acid iodides, Boc-derivatives and acid alcohols in Table 2—should be understood to encompass the disclosure of the amino acids themselves, as well as their R groups as functional groups and as incorporated in a larger peptide structure.

A Holliday junction-trapping compound of the invention has a variety of activities, including binding to a ribosome; modulating the function of a ribosome; and inhibiting the growth of bacteria. Therefore, the invention provides a method for using a Holliday junction-trapping compound, such as those exemplified in FIG. 55 to bind to a ribosome; to modulate the function of a ribosome, for example, by inhibiting translation, and to inhibit the growth of, or kill, bacteria.

A Holliday junction-trapping compound of the invention also can bind to or modulate the activity of a tyrosine recombinase, such as the XerC/D site-specific recombinases or topoisomerases, such as a type I topoisomerases. Such a compound can modulate recombinase activity, for example, by blocking recombination before strand cleavage or blocking ligation without blocking strand cleavage; or by binding to or stabilize replication or recombination intermediates that have a structure substantially similar to a Holliday junction. Therefore, the invention provides a method for using a Holliday junction-trapping compound, such as those exemplified in FIG. 55, to modulate recombinase activity.

As shown herein above, the Holliday junction-trapping compounds exemplified in FIG. 55 are effective in inhibiting bacterial growth. As described herein above, compounds that are peptide synergimycin derivatives also can be used for inhibiting bacterial growth (see Example XVI). Therefore, the invention provides a method for using a compound of the invention to treat a pathology caused by the presence of bacteria. The method involves contacting bacteria with a compound of the invention. Bacteria treated with a compound can be, for example, present in an individual, in a sample, such as food or waste, or on a surface that requires sanitization.

The invention further provides a method for treating a subject. The method for treating a condition comprises administering to the subject an effective dose of a pharmaceutical composition, which comprises a pharmaceutically acceptable carrier and a compound obtained by screening a compound library, as described above.

An “effective dose” of the compound herein means an amount of the composition that is sufficient to therapeutically alleviate the condition.

The amount of a therapeutically effective dose depends on a variety of factors, including the particular characteristics of the compound, the type and severity of the condition to be treated and the physical condition of the individual to be treated. Based on such factors, those skilled in the art can readily determine a therapeutically effective dose of the compound, which can be about 0.0001 to 100 mg/kg body weight per administration. For example, the compound can be administered at 0.0001, 0.0005, 0.001, 0.005, 0.01, 0.05, 0.1, 0.5, 1, 5, 10, 50 or 100 mg/kg body weight.

TABLE 2
Amino Acids and Derivatives

Boc—MeVal—OH (DCHA)
Boc-D-MeVal—OH
Boc—Tyr(Me)—OH
Boc-D-Tyr(Me)—OH
Valine
Boc—Val—OH
Boc-D-Val—OH
Boc—Val—OSu
Boc—Val—N(OCH₃)CH₃ (oil)
Boc—MeVal—OH
Boc—Tyr(2—Br—Z)—N(OCH₃)CH₃(oil)
Boc—Tyr(tBu)—OH
Boc—Tyr(2,6—di—Cl—Bzl)—OH
Boc-D-Tyr(2,6—di—Cl—Bzl)—OH
Boc—Tyr(Et)—OH
Boc-D-Tyr(Et)—OH
Boc—MeTyr(Bzl)—OH
Boc—MeTyr(Me)—OH (DCHA)
Tyrosine
Boc—Tyr—OH
Boc-D-Tyr—OH
Boc—Tyr—OSu
Boc—Tyr(Bzl)—OH
Boc-D-Tyr(Bzl)—OH
Boc—Tyr(Bzl)—OSu
Boc—Tyr(2—Br—Z)—OH
Boc-D-Tyr(2—Br—Z)—OH
Boc—Trp(For)—OH
Boc-D-Trp(For)—OH
Boc—Trp(Mts)—OH (DCHA)
Boc—Trp—ψ—[CH₂NH]—Gly—OH
Boc—Thr(Bzl)—N(OCH₃)CH₃(oil)
Boc—Thr(tBu)—OH
Tryptophan
Boc—Trp—OH
Boc-D-Trp—OH
Boc—Trp—ONp
Boc—Trp—N(OCH₃)CH₃
Boc—Trp(Boc)—OH
Tetrahydroisoquinoline-3-carboxylic acids
Boc—Tic—OH
Boc-D-Tic—OH
Threonine
Boc—Thr—OH
Boc-D-Thr—OH
Boc—Thr(Bzl)—OH
Boc-D-Thr(Bzl)—OH
Boc—Thr(Bzl)—OSu
Boc—Ser(Bzl)—OSu
Boc—Ser(tBu)—OH (DCHA)
Boc—Ser(Me)—OH
Statine & derivatives
Boc—ACHPA
Boc—AHPPA
Boc—Sta—OH
Boc-3,4-dehydro-Pro—OH
Sarcosine
Boc—Sar—OH
Boc—Sar—OSu
Serine
Boc—Ser—OH
Boc-D-Ser—OH
Boc—Ser(Bzl)—OH
Boc-D-Ser(Bzl)—OH
Phenylglycine
Boc—Phg—OH
Boc-D-Phg—OH
Proline
Boc—Pro—OH
Boc-D-Pro—OH
Boc—Pro—OSu
Boc-D-Pro—OSu
Boc—Pro—ONp
Boc-D-Phe—Osu
Boc—Phe—N(OCH₃)CH₃ (oil)
Boc—Phe—ONp
Boc—Phe(pCl)—OH
Boc-D-Phe(pCl)—OH
Boc—MePhe—OH (DCHA)
Boc-D-MePhe—OH (DCHA)
Norvaline
Boc—Nva—OH (syrup)
Boc-D-Nva—OH (syrup)
Ornithine
Boc—Orn(Z)—OH
Phenylalanine
Boc—Phe—OH
Boc-D-Phe—OH
Boc—Phe—OSu
Methionine
Boc—Met—OH
Boc-D-Met—OH
Boc—Met—OSu
Boc—Met(O)—OH
Boc—Met(O₂)—OH
Norleucine
Boc—Nle—OH (DCHA)
Boc—MeNle—OH (oil)
Boc—Lys(Z)—OH (cryst.)
Boc—Lys(Z)—OSu
Boc—Lys(Boc)—OH (DCHA)
Boc—Lys(Boc)—OSu
Boc—Lys(2—Cl—Z)—OH (cryst.)
Boc-D-Lys(2—Cl—Z)—OH (cryst.)
Boc—Lys(Fmoc)—OH
Boc—Lys(Tfa)—OH
Boc—Leu—OSu
Boc—Leu—ψ—[CH₂NH]—Leu—OH
Boc—Leu—ψ—[CH₂N(2—Cl—Z)]—Leu—OH (oil)
Boc—Leu—ψ—[CH₂NH]—Val—OH
Boc—Leu—ψ—[CH₂N(2—Cl—Z)]—Val—OH
Boc—MeLeu—OH
Lysine
Boc—Lys—OH
Boc—Lys(Ac)—OH
Hydroxyproline
Boc—Hyp—OH (cryst.)
Isoleucine
Boc—Ile—OH (0.5 H₂O)
Boc—Ile—OSu
Boc—Ile—N(OCH₃)CH₃ (oil)
Leucine
Boc—Leu—OH (H₂O)
Boc-D-Leu—OH (H₂O)
Boc—His(Tos)—OH (DCHA)
Boc-D-His(Tos)—OH (DCHA)
Boc—His(Trt)—OH
Homocitrulline
Boc—Hci—OH
Boc-D-Hci—OH
Boc—His(Boc)—OH (DCHA)
Boc—His(Bom)—OH
Boc—His(Dnp)—OH (isopropanol)
Boc—His(Tos)—OH
Boc-D-His(Tos)—OH
Boc—Gln(Xan)—OH
Glycine
Boc—Gly—OH
Boc—Gly—ONp
Boc—Gly—OSu
Histidine
Boc—His—OH
Boc-D-His—OH
Glutamine
Boc—Gln—OH
Boc-D-Gln—OH
Boc—Gln—ONp
Boc—Gln—OSu
Boc—Gln(Trt)—OH
Boc-D-Gln(Trt)—OH
Boc—Glu(OBzl)—OH (cryst.)
Boc-D-Glu(OBzl)—OH
Boc—Glu(OBzl)—OSu
Boc—Glu—OtBu
Boc—Glu(OtBu)—OH
Boc—Glu(OtBu)—OSu
Boc—Glu(OcHx)—OH
Boc—Cys(pMeOBzl)—OH
Boc—Cys(pMeBzl)—OH
Boc—Cys(Trt)—OH
(Boc—Cys—OH)₂
Glutamic acid
Boc—Glu—OH
Boc—Glu—NH₂
Boc—Glu—OBzl (cryst.)
Boc-D-Glu—OBzl
Cyclohexylalanine
Boc—Cha—OH (DCHA)
Boc-D-Cha—OH (DCHA)
Cysteine
Boc—Cys—OH (cryst.)
Boc—Cys(Acm)—OH
Boc-D-Cys(Acm)—OH
Boc—Cys(Bzl)—OH
Boc—Asp—OtBu
Boc—Asp(OtBu)—OH (DCHA)
Boc—Asp(OtBu)—OSu
Boc—Asp(OcHx)—OH
Butylglycine
Boc-L-α-t-butylglycine
Boc-L-α-t-butylglycine
Aspartic acid
Boc—Asp—OH
Boc—Asp(O-1-Ada)—OH
Boc—Asp(O-2-Ada)—OH
Boc—Asp—OBzl
Boc—Asp—(OBzl)—OH
Boc-D-Asp(OBzl)—OH
Boc—Asp(OBzl)—OSu
Boc-D-Asn—ONp
Boc—Asn(Trt)—OH
Boc—Asn(Trt)—OH
Boc-D-Asn(Trt)—OH
Boc—Asn(Xan)—OH
Boc-D-Arg(NO₂)—OH
Boc—Arg(Tos)—OH
Boc-D-Arg(Tos)—OH
Asparagine
Boc—Asn—OH
Boc-D-Asn—OH
Boc—Asp—NH₂
Boc—Asn—ONp
Arginine
Boc—Arg—OH (HCl H₂O)
Boc-D-Arg—OH (HCl H₂O)
Boc—Arg(Mts)—OH
Boc—Arg(di-Z)—OH
Boc—Arg(Mtr)—OH
Boc-D-Arg(Mtr)—OH
Boc—Arg(NO₂)—OH
Boc—β—Ala—OH
Boc—β—Ala—OSu
Aminobutyric acid
Boc—Abu—OH
Boc—Υ—Abu—OH
Aminohexanoic acid
Boc—εAhx—OH
Aminoisobutyric acid
Boc—Aib—OH
Alanine
Boc—Ala—OH
Boc-D-Ala—OH
Boc—Ala—OSu
Boc-D-Ala—OSu
Boc—Ala—N(OCH₃)CH₃
Boc—MeAla—OH
Other amino acid derivatives:
N-(tert-Butoxycarbonyl)-L-valine N′-methoxy-N′-methylamide-
(tert-Butoxycarbonyl)-L-valine methyl ester
N-(tert-Butoxycarbonyl)-D-valinol-
(tert-Butoxycarbonyl)-L-valinol
6′-Butoxy-2,6-diamino-3,3′-azodipyridine
2-Butoxy-N-(2-diethylaminoethyl)-4-quinolinecarboxamide
hydrochloride
2-Butoxyethan(ol-d)
2-Butoxyethanol
2-Butoxyethanol
2-Butoxyethanol
1-tert-Butoxy-2-ethoxyethane
2-(2-Butoxyethoxy)ethanol
2-(2-Butoxyethoxy)ethyl acetate
2-Butoxyethyl acetate
2-Butoxyethyl acrylate
2-Butoxyethyl methacrylate
2-Butoxyethyl oleate
(S)-(−)-1(tert-Butoxycarbonyl)-2-pyrrolidinemethanol
(R)-(+)-1-(tert-Butoxycarbonyl)-2-pyrrolidinemethanol
1-(tert-Butoxycarbonyl)-2-pyrrolidinone-
(tert-Butoxycarbonyl)-3-[4-(1-pyrrolyl)phenyl]-L-alanine
N-(tert-Butoxycarbonyl)-D-serine methyl ester
N-(tert-Butoxycarbonyl)-L-serine methyl ester
N-(tert-Butoxycarbonyl)-L-threonine
N-(tert-Butoxycarbonyl)-L-threonine methyl ester
N-(tert-Butoxycarbonyl)-p-toluenesulfonamide-
(tert-Butoxycarbonyl)-S-trityl-L-cysteine
Nα-(tert-Butoxycarbonyl)-L-tryptophan
Nα-(tert-Butoxycarbonyl)-L-tryptophanol-
(tert-Butoxycarbonyl)-L-tyrosine
N-(tert-Butoxycarbonyl)-L-tyrosine methyl ester
N-(tert-Butoxycarbonyl)-L-valine-¹⁵N
N-(tert-Butoxycarbonyl)-L-valine
N-(tert-Butoxycarbonyl)-L-phenylalanine
N-(tert-Butoxycarbonyl)-L-phenylalanine methyl ester
N-(tert-Butoxycarbonyl)-D-phenylalaninol
N-(tert-Butoxycarbonyl)-L-phenylalaninol
(R)-(−)-N-(tert-Butoxycarbonyl)-2-phenylglycinol
(S)-(+)-N-(tert-Butoxycarbonyl)-2-phenylglycinol
N-(tert-Butoxycarbonyl)-β-phenyL-D-phenylalanine
N-(tert-Butoxycarbonyl)-β-phenyl-L-phenylalanine
N-(tert-Butoxycarbonyl)-β-phenyl-D-phenylalaninol
N-(tert-Butoxycarbonyl)-β-phenyl-L-phenylalaninol
(R)-(+)-1-(tert-Butoxycarbonyl)-2-piperidinecarboxylic acid
1-(tert-Butoxycarbonyl)-2-piperidinecarboxylic acid
(S)-(−)-1-(tert-Butoxycarbonyl)-2-piperidinecarboxylic acid
1-(tert-Butoxycarbonyl)-3-piperdinecarboxylic acid
N-(tert-Butoxycarbonyl)-D-prolinal
N-(tert-Butoxycarbonyl)-D-proline
N-(tert-Butoxycarbonyl)-L-proline
N-(tert-Butoxycarbonyl)-L-proline N′-methoxy-N′-methylamide
N-(tert-Butoxycarbonyl)-L-prolinal
N-(tert-Butoxycarbonyl)-1H-pyrazole-1-carboxamidine
Nα-(tert-Butoxycarbonyl)-L-lysine
Nε-(tert-Butoxycarbonyl)-L-lysine
N-(tert-Butoxycarbonyl)metaraminol
N-(tert-Butoxycarbonyl)-D-methionine, dicyclohexylammonium
salt
N-(tert-Butoxycarbonyl)-L-methionine
Butoxycarbonylmethyl butyl phthalate
(tert-Butoxycarbonylmethylene)triphenylphosphorane
(S)-(+)-N-tert-(Butoxycarbonyl)-2-methylpiperidine
(tert-Butoxycarbonylmethyl)triphenylphosphonium bromide
N-(tert-Butoxycarbonyl)-3-(1-naphthyl)-D-alanine
N-(tert-Butoxycarbonyl)-3-(2-naphthyl)-L-alanine
2-(tert-Butoxycarbonyloxyimino)-2-phenylacetonitrile
N-(tert-Butoxycarbonyloxy)phthalimide
4-(tert-Butoxycarbonyloxy)styrene
N-(tert-Butoxycarbonyl-carbonyl-¹³C)-L-phenylalanine
N-(tert-Butoxycarbonyl)-L-phenylalanine-carboxy-¹³C
N-(tert-Butoxycarbonyl)-L-phenylalanine-β-¹³C
N-(tert-Butoxycarbonyl)-L-phenylalanine-α,β,β,2,3,4,5,6-d₈
N-(tert-Butoxycarbonyl)-L-phenylalanine-¹⁵N
N-(tert-Butoxycarbonyl)glycine-2-¹³C-¹⁵N
N-(tert-Butoxycarbonyl)glycine-¹⁵N
N-(tert-Butoxycarbonyl)glycine
N-(tert-Butoxycarbonyl)glycine tert-butyl ester
N-(tert-Butoxycarbonyl)glycine N′-methoxy-N′-methylamide
N-(tert-Butoxycarbonyl)glycine methyl ester
Nα-(tert-Butoxycarbonyl)-L-histidine
N-tert-Butoxycarbonylhydroxylamine
1-(tert-Butoxycarbonyl)imidazole
N-(tert-Butoxycarbonyl)-3-iodo-D-alanine benzyl ester
N-(tert-Butoxycarbonyl)-3-iodo-L-alanine benzyl ester
N-(tert-Butoxycarbonyl)-3-iodo-D-alanine methyl ester
N-(tert-Butoxycarbonyl)-3-iodo-L-alanine methyl ester
N-(tert-Butoxycarbonyl)-L-isoleucine
N-(tert-Butoxycarbonyl)-L-leucine-1-¹³C monohydrate
N-(tert-Butoxycarbonyl)-L-leucine-¹⁵N monohydrate
N-(tert-Butoxycarbonyl)-L-leucine monohydrate
N-(tert-Butoxycarbonyl)-L-leucine N-hydroxysuccinimide ester
N-(tert-Butoxycarbonyl)-L-leucine N′-methoxy-N′-methylamide
N-(tert-Butoxycarbonyl)-L-leucine methyl ester
N-(tert-Butoxycarbonyl)-L-leucinol
(R)-(+)-2-(tert-Butoxycarbonylamino)-1-propanol
(S)-(−)-2-(tert-Butoxycarbonylamino)-1-propanol
5-(tert-Butoxycarbonylamino)valeric acid
Nα-(tert-Butoxycarbonyl)-L-arginine
Nα-(tert-Butoxycarbonyl)-L-asparagine
N-(tert-Butoxycarbonyl)-L-aspartic acid
N-(tert-Butoxycarbonyl)-L-aspartic acid 4-benzyl ester
N-(tert-Butoxycarbonyl)-O-benzyl-L-threonine
(R)-(+)-1-(tert-Butoxycarbonyl)-2-tert-butyl-3-methyl-4-
imidazolidinone
(S)-(−)-1-(tert-Butoxycarbonyl)-2-tert-butyl-3-methyl-4-
imidazolidinone
Nα-(tert-Butoxycarbonyl)-Nε-(carbobenzyloxy)-L-lysine-ε-¹⁵N
N-(tert-Butoxycarbonyl)-3-cyclohexyl-L-alanine methyl ester
N-(tert-Butoxycarbonyl)-L-cysteine methyl ester
N-(tert-Butoxycarbonyl)ethanolamine
N-(tert-Butoxycarbonyl)ethylenediamine
N-(tert-Butoxycarbonyl)-D-glucosamine
Nα-(tert-Butoxycarbonyl)-L-glutamine
N-(tert-Butoxycarbonyl)glycine-1-¹³C
N-(tert-Butoxycarbonyl)glycine-2-¹³C
tert-Butoxybis(dimethylamino)methane
3-(tert-Butoxy)-1-butyne
N-(tert-Butoxycarbonyl)
N-(tert-Butoxycarbonyl)-L-alanine-¹³C₃-¹⁵N
N-(tert-Butoxycarbonyl)-L-alanine-1-¹³C
N-(tert-Butoxycarbonyl)-L-alanine-3-¹³C
N-(tert-Butoxycarbonyl)-L-alanine-¹²C₃, ¹³C-depleted
N-(tert-Butoxycarbonyl)-L-alanine-3,3,3-d₃
N-(tert-Butoxycarbonyl)-L-alanine-¹⁵N
N-(tert-Butoxycarbonyl)-L-alanine
N-(tert-Butoxycarbonyl)-L-alanine N′-methoxy-N′-methylamide
N-(tert-Butoxycarbonyl)-D-alanine methyl ester
N-(tert-Butoxycarbonyl)-L-alanine methyl ester
N-(tert-Butoxycarbonyl)-2-aminoacetonitrile
N-(tert-Butoxycarbonyl)-4-aminobenzylamine
4-(tert-Butoxycarbonylamino)butyric acid
(S)-(−)-2-(tert-Butoxycarbonylamino)-3-cyclohexyl-1-propanol
(R)-(+)-2(tert-Butoxycarbonylamino)-3-methyl-1-butanol
(R)-(+)-2-(tert-Butoxycarbonylamino)-3-phenylpropanal
(S)-(−)-2-(tert-Butoxycarbonylamino)-3-phenylpropanal
(R)-(+)-2-(tert-Butoxycarbonylamino)-3-phenyl-1-propanol
(S)-(−)-2-(tert-Butoxycarbonylamino)-3-phenyl-1-propanol
Blue Tetrazolium
BMS
BN
t-BOC
N-BOC-L-2-aminoadipic acid
3-(BOC-amino)benzoic acid
4-(BOC-amino)benzoic acid
4-(BOC-amino)-1-butanol
6-(BOC-amino)caproic acid
7-(BOC-amino)heptanoic acid
6-(BOC-amino)-1-hexanol
(3R,4S)-4-(BOC-amino)-3-hydroxy-6-methylheptanoic acid
(3S,4S),-4-(BOC-amino)-3-hydroxy-6-methylheptanoic acid
(2S,3R)-3-(BOC-amino)-2-hydroxy-5-methylhexanoic acid
(2S,3R)-3-(BOC-amino)-2-hydroxy-4-(4-nitrophenyl)butyric
acid
(2R,3R)-3-(BOC-amino)-2-hydroxy-4-phenylbutyric acid
(2S,3R)-3-(BOC-amino)-2-hydroxy-4-phenylbutyric acid
3-(BOC-aminomethyl)benzoic acid
4-(BOC-aminomethyl)phenyl isothiocyanate
8-(BOC-amino)octanoic acid
(BOC-aminooxy)acetic acid
5-(BOC-amino)-1-pentanol
BOC-BMI
Nα-BOC-D-citrulline
Nα-BOC-L-citrulline
N-BOC-1,4-diaminobutane
N-BOC-2,5-diaminopentane
Nα-BOC-L-2,3-diaminopropionic acid
N-BOC-diethanolamine
N-BOC-N′-FMOC-diaminoacetic acid
BOC-L-β-homoproline
N-BOC-iminodiacetic acid
N-BOC-4-isothiocyanatoaniline
N-BOC-4-isothiocyanatobutylamine
N-BOC-2-isothiocyanatoethylamine
N-BOC-6-isothiocyanatohexylamine
N-BOC-5-isothiocyanatopentylamine
N-BOC-4-isothiocyanatopropylamine
BOC-S-(4-methoxybenzyl)-D-penicillamine
BOC-S-(4-methoxybenzyl)-L-penicillamine
N-BOC-N-methylethylenediamine
N-BOC-nortropinone
BOC-ON
N-t-BOC-phenylalaninal
N-BOC-1,4-phenylene diamine
1-BOC-piperidine-4-carboxylic acid
N-t-BOC-prolinol
N-t-BOC-pyrrole
N-t-BOC-pyrrolidine
(R)-N-BOC-1,2,3,4-tetrahydroisoquinoiine-3-carboxylic acid
(S)-N-BOC-1,2,3,4-tetrahydroisoquinoline-3-carboxylic acid
N-t-BOC-D-valinol
O-t-BOC-vanillin

TABLE 3
Amino acids, derivatives and R groups for use as
monomers, particularly in positions 2, 5 and 7
(available as acid iodides)

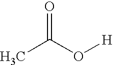

| | |
|---|---|
| | 3-IODOBENZOIC ACID] |
| N-ACETYL-L-PHENYLALANYL- | 4-IODOBENZOIC ACID |
| | [4-IODOBENZOIC ACID |
| 3,5-DIIODO-L-TYROSINE | 4-IODOBENZOIC ACID |
| O-IODOSOBENZOIC ACID | 4-IODOBENZOIC ACID] |
| 2-IODOBENZOIC ACID | 3,3′,5-TRIIODO-L- |
| [2-IODOBENZOIC ACID | |
| 2-IODOBENZOIC ACID | THYRONINE |
| 2-IODOBENZOIC ACID | L-THYROXINE |
| 2-IODOBENZOIC ACID | L-4-IODOPHE |
| | 3-IODO-L-TYROSINE |
| 2-IODOBENZOIC ACID] | [3-IODO-L-TYROSINE |
| 2,3,5-TRIIODOBENZOIC | 3-IODO-L-TYROSINE |
| ACID | 3-IODO-L-TYROSINE |
| 3,5-DIIODOSALICYLIC ACID | 3-IODO-L-TYROSINE |
| 5-IODOSALICYLIC ACID | 3-IODO-L-TYROSINE |
| [5-IODOSALICYLIC ACID | 3-IODO-L-TYROSINE |
| 5-IODOSALICYLIC ACID | 3-IODO-L-TYROSINE |
| 5-IODOSALICYLIC ACID] | 3-IODO-L-TYROSINE] |
| 3-IODOBENZOIC ACID | IODOACETIC ACID |
| [3-IODOBENZOIC ACID | [IODOACETIC ACID |
| 3-IODOBENZOIC ACID | IODOACETIC ACID |
| 3-IODOBENZOIC ACID | IODOACETIC ACID |
| 3-IODOBENZOIC ACID | IODOACETIC ACID |
| IODOACETIC ACID] | 3,3′- |
| O-IODOHIPPURIC ACID | HEXAMETHYLENEDI- |
| 5 3-IODOPROPIONIC ACID | UREIDOBIS |
| 3,5-DIIODO-4-PYRIDONE-1- | (2,4,6-TRIIODOBENZOIC |
| ACETIC ACID | ACID) |
| 3-AMINO-2,4,6- | 3-ACETAMIDO-2,4,6- |
| TRIIODOBENZOIC ACID | TRIIODOBENZOIC ACID, |
| 2-AMINO-3,5- | BIS (2-HYDROXYETHYL)- |
| DIIODOBENZOIC ACID | AMMONIUM SALT |
| 4-AMINO-3,5- | 3-HYDROXY-2,4,6- |
| DIIODOBENZOIC ACID | TRIIODOBENZOIC ACID |
| 2-AMINO-5-IODOBENZOIC | N,N′-ADIPOYLBIS-(3- |
| ACID | AMINO-ALPHA-ETHYL-2,4,6- |
| [2-AMINO-5-IODOBENZOIC | |
| | TRIIODOHYDROCINNAMIC |
| ACID | |
| 2-AMINO-5-IODOBENZOIC | ACID) |
| | N-(4-IODOPHENYL)MALE- |
| ACID] | AMIC |
| 3-IODO-4-METHYLBENZOIC | ACID |
| | 2-PHENYL-2-(2,4,6- |
| ACID | |
| 4-IODOPHENOXYACETIC ACID | TRIIODOPHENOXY)ACETIC |
| 4-IODOBUTYRIC ACID | ACID |
| ACETRIZOIC ACID | 3-(3,5-DIIODO-4- |
| 3,4,5-TRIIODOBENZOIC | HYDROXYPHENYL)-2- |
| ACID | |
| 3,5,3′,5′- | PHENYLPROPIONIC ACID |
| | 2-IODOXYBENZOIC ACID |
| TETRAIODOTHYROFORMIC | 4-AMINO-3,5-DIIODO- |
| ACID | ALPHA- (4-METHOXYPHE- |
| 3,5-DIIODO-4- | NYL)-HYDROCINNAMIC ACID |
| HYDROXYBENZOIC ACID | CIS-HEXAHYDRO-N-(4- |
| 3,5-DIIODO-L-TYROSINE | |
| 3-IODO-4-METHOXYBENZOIC | IODOPHENYL)PHTHALAMIC |
| ACID | ACID |
| 3-(4- | 4′-IODOSUCCINANILIC ACID |
| | N-(4- |
| IODOPHENYL)PROPIONIC | IODOPHENYL)GLUTARAMIC |
| ACID | |
| N-ACETYL-3,5-DIIODO-L- | ACID |
| | N-(4- |
| TYROSINE | |
| 3,5-DIMETHYL-2- | IODOPHENYL)PHTHALAMIC |
| IODOBENZOIC ACID | ACID |
| 2′-CARBOXY-2-CHLORO-4′- | TETRATODOTHYRO-ACETIC |
| IODOACETANILIDE | ACID |
| 4- | 5-IODOOROTIC ACID |
| (IODOMETHYLARSINO)BENZO- | N-FORMYL-MET-LEU-P-IODO |
| IC ACID | PHE |
| 3,3′,5′-TRIIODO-L- | 4-HYDROXY-3-IODO-5- |
| | NITRO-PHENYLACETIC ACID |
| THYRONINE | |
| BOC-3,5-DIIODO-TYR(3′- | 4-HYDROXY-3-IODO-5- |
| BROMO-BZL)-OH | NITROBENZOIC ACID |
| 9(10)-IODOSTEARIC ACID | |
| BOC-PHE(4-I)-OH | BOC-3,5-DIIODO-TYR-OH |
| [BOC-PHE(4-I)-OH] | IODIPAMIDE |
| IOPANOIC ACID | |
| BOC-4-AMINO-3,5-DIIODO- | 4-CHLORO-2-IODOBENZOIC |
| | ACID |
| PHE-OH | |
| 2-FLUORO-6-IODOBENZOIC | 3,3′,5-TRIIODO-D- |
| | THYRONINE |
| ACID | |
| [2-FLUORO-6-IODOBENZOIC | 3,3′,5-TRIIODO-DL- |
| | THYRONINE |
| ACID] | D-THYROXINE |
| TETRAIODOBENZOIC ACID | DL-THYROXINE |
| 2,5-DIIODOBENZOIC ACID | H-P-IODO-DL-PHE-OH |
| 3,5-DIIODO-4′-(4- | D-4-IODOPHE |
| HYDROXYPHENOXY)BENZOIC | 3,5-DIIODO-DL-TYROSINE |
| | FMOC-3,5-DIIODO-TYR-OH |
| ACID | 4-(P-IODOPHENYL)BUTYRIC |
| 3,5-DIIODOTHYROPROPIONIC | |
| | ACID |
| ACID | 4-AMINO-3,5-DIIODO-L- |
| 2-IODOPHENYLACETIC ACID | |
| M-IODOPHENYLACETIC ACID | PHENYLALANINE |
| P-IODOCINNAMIC ACID | 3,5-DIIODO-L-THYRONINE |
| 5-(IODOACETAMIDO)- | 1,4-DIHYDRO-3,5-DIIODO- |
| FLUORESCEIN | 4-OXO-1-PYRIDINEACETIC |
| 4-IODOACETAMIDOSALICYLIC | ACID, DIETHYLAMINE SALT |
| ACID | 3,5-DIIODO-DL-THYRONINE |
| 3,3′,5- | DIATRIZOIC ACID |
| | 2,3-DIMETHOXY-5- |
| TRIIODOTHYROPROPIONIC | |
| | IODOBENZOIC ACID |
| ACID | 5-IODOFERULIC ACID |
| 4′-HYDROXY-3,5,3′-TRI | 8-IODO-1-NAPHTHOIC ACID |
| IODO PHENOXY-4-PHENYL | (3,5-DIIODO-TYR1)- |
| ACETIC ACID | DYNORPHIN A (1-8) |
| 3,3′,5,5′- | (3,5-DIIODO-TYR1)- |
| DYNORPHIN A (1-9) | CARBOXYLIC ACID |
| EXPERIMENTAL ALLERGIC | 4-AMINO-3,5- |
| ENCEPHALITOGENIC PEPTIDE | DIIODOSALICYLIC ACID |
| (HUMAN) (IODINATED) | 1,4-DIHYDRO-3,5-DIIODO- |
| BOC-3,5-DIIODO- | 4-OXO-ALPHA-PHENYL-1- |
| TYR (2′,6′-DICHLORO-BZL)- | PYRIDINEACETIC ACID |
| OH | 2-[(2- |
| BOC-D-4-IODOPHE | CARBOXYPHENYL)THIO]-5- |
| 2-BROMO-5-IODOBENZOIC | IODOBENZOIC ACID |
| ACID | 2-[(1-IODO-2- |
| 5-BROMO-2-IODOBENZOIC | NAPHTHYL)OXY]PROPANOIC |
| ACID | |
| 2-CHLORO-5-IODOBENZOIC | ACID |
| | ALA-SER-THR-THR-THR-ASN- |
| ACID | |
| 4-CHLORO-3-IODOBENZOIC | 3,5-DIIODO-TYR-THR |
| | ALPHA-ETHYL-3-HYDROXY- |
| ACID | |
| 5-CHLORO-2-IODOBENZOIC | 2,4,6- |
| ACID | TRIIODOHYDROCINNAMIC |
| 4,5-DIMETHOXY-2- | ACID |
| IODOBENZOIC ACID | (3,5-DIIODO-TYR1,D- |
| 2-IODO-3-METHYLBENZOIC | THR2)-LEU-ENKEPHALIN-THR |
| ACID | 3-ACETAMIDO-2,4,6- |
| 2-IODO-5-METHYLBENZOIC | TRIIODOBENZOIC ACID, |
| ACID | COMPOUND WITH 1-DEOXY-1- |
| BOC-3-I-TYR(3-BRBZL) | (ME-AMINO)-GLUCIT |
| N,O-DIACETYL-3,5-DIIODO- | N-ACETYL-3-IODO-L- |
| L-TYROSINE | |
| 4-IODOPHENYLACETIC ACID | TYROSINE |
| IODOACETIC ACID-1-13C | N-ACETYL-D-PHENYLALA- |
| IODOACETIC ACID (2-13C) | NYL-3,5-DIIODO-L-TYROSINE |
| 2-(ACETYLAMINO)-3-(4- | N-ACETYL-4-IODO-L- |
| HYDROXY-3,5- | PHENYLALANINE |
| DIIODOPHENYL)PROPANOIC | ATRIAL NATRIURETIC |
| ACID | FACTOR, [125I]-, RAT |
| 125I-BOP | 125I-ALPHA-ATRIAL |
| I-BOP | NATRIURETIC POLYPEPTIDE |
| 3-IODO-4- | 1-28 (HUMAN, CANINE) |
| (ISOPROPYLSULFONYL)-5- | ANGIOTENSIN I, [125I]- |
| (METHYLTHIO)THIOPHENE-2- | TYR4- |
| ANGIOTENSIN II, [125I]- | TRICYCLO(4.2.1.O(3,7))NO |
| TYR4- | NANE-9-CARBOXYLIC ACID |
| 1251-CALCITONIN (SALMON) | 2-IODO-4,6- |
| (3- | DIBENZOFURANDICARBOXY- |
| [125I]IODOTYROSYL)ENDO- | LIC ACID |
| THELIN-1 | (3- |
| GLUCAGON, [125I]- | [125I]-IODOTYROSYL4)SAR1I |
| 125I-NEUROTENSIN | |
| (2R,3R)-1-CARBOXY-4- | LE8, ANGIOTENSIN II |
| IODO-2,3- | IODOACETIC ACID, [1- |
| DIHYDROXYCYCLOHEXA-4,6- | 14C]- |
| | IODOACETIC ACID, [2- |
| DIENE | |
| (2R,3S)-1-CARBOXY-5- | 14C]- |
| | IODOACETIC ACID, [3H]- |
| IODO-4-METHYL-2,3- | 5-AMINO-2,4,6- |
| DIHYDROXYCYCLOHEXA-4,6- | TRIIODOISOPHTHALIC ACID |
| DIENE | 3-BROMO-5-IODOBENZOIC |
| 3,5-DIIODO-L-TYROSINE | ACID |
| DIHYDRATE | 3-BROMO-5-IODOBENZOIC |
| L-703,606 OXALATE | ACID |
| N-(2,2-DICHLOROACETYL)- | 4-HYDROXY-5-IODO-3- |
| 5-IODOANTHRANILIC ACID | METHOXYBENZOIC ACID |
| 4′-IODO-1,2,3,6- | I-SAP |
| | 125I-SAP |
| TETRAHYDROPHTHALANILIC | 2-[[(2- |
| ACID | |
| VASOACTIVE INTESTINAL | IODOPHENYL)SULFONYL]AM- |
| POLYPEPTIDE, [3-(125I)] | INO]ACETIC ACID |
| 2-[2-(4-IODOANILINO)-2- | 2-[[(2-IODO-4- |
| OXOETHOXY]ACETIC ACID | NITROPHENYL)SULFONYL] |
| 5-IODO-2-[(2,2,2- | AMINO]ACETIC ACID |
| TRIFLUOROACETYL)AMINO] | 2-(6-HYDROXY-2,5,7- |
| BENZOIC ACID | TRIIODO-3-OXO-3H- |
| BOC-P-IODO-DL-PHE-OH | XANTHEN-9-YL)-BENZOIC |
| 6-BROMO-3-IODO-2- | ACID |
| METHYLQUINOLINE-4- | 4-HYDROXY-7-IODO-10-OXO- |
| CARBOXYLIC ACID | 9-OXA- |
| 4-IODO-D-PHENYLALANINE, | TRICYCLO(6.2.1.O(1,5))UN- |
| MONOHYDRATE | |
| 2-IODO-5-OXO-4-OXA- | DECANE-2-CARBOXYLIC |
| | ACID |
| (6-IODO- | FMOC-D-TYR(3′,5′-DI-L)- |
| BENZO(1,3)DIOXOL-5-YL)- | OH |
| ACETIC ACID | 3,5′-DIIODO-TYR-ALA-GLY- |
| 2-IODO-5-NITROBENZOIC | GLY |
| ACID | [MONOIODO- |
| BUTTPARK 22\07-100 | TYR3]NEUROTENSIN |
| TRIIODOTHYRONINE, L- | (Z)-(3)-IODOACRYLIC ACID |
| 3,5,3′-[125I]- | 3-IODOPHTHALIC ACID |
| NEUROKININ A, [125I]- | 2-IODO-3-NITROBENZOIC |
| ENDOTHELIN-3, [1251- | ACID |
| TYR6]-RAT | (+)-S-2-AMINO-6- |
| FMOC-3-IODO-TYR-OH | IODOACETAMIDOHEXANOIC |
| TRIIODOTHYRONINE, L- | ACID |
| 3,3′,5, [125I- | (S)-(+)-2-AMINO-5-(IODO- |
| AC-P-IODO-D-PHE-OH | ACETAMIDO)PENTANOIC |
| | ACID |
| (3,5-DIIODO-TYR1, D- | 3,5-DIIODOBENZOIC ACID |
| ALA2)-MET-ENKEPHALIN | TETRAMETHYLRHODAMINE- |
| AMIDE | 5-IODOACETAMIDE |
| FOR-MET-LEU-P-IODO-PHE- | 4-IODO-3-NITROBENZOIC |
| OH | ACID |
| 3,5-DIIODO-D-TYR-ALA- | 4-(3-[2,2,2-TRICHLORO-1- |
| GLY-GLY | (4-IODO-BENZOYLAMINO)- |
| H-3,5-DIIODO-D-TYR-OH | |
| H-GLY-LEU-P-IODO-PHE-OH | ETHYL]-THIOUREIDO)- |
| H-GLY-P-IODO-PHE-TRP-OH | BENZOIC ACID |
| 5-IODO-3- | 4-(4-IODO-2- |
| METHYLBENZO[B]FURAN-2- | METHYLANILINO)-4-OXOBUT- |
| CARBOXYLIC ACID | 2-ENOIC ACID |
| 5-HYDROXY-2-IODOBENZOIC | 4-(3-[2,2,2-TRICHLORO-1- |
| ACID | (3-IODO-BENZOYLAMINO)- |
| 3,5-DIIODO-P- | ETHYL]-THIOUREIDO)- |
| HYDROXYPHENYLPYRUVIC | BENZOIC ACID |
| ACID | 3-(3-[2,2,2-TRICHLORO-1- |
| 3-IODO-2-METHYLBENZOIC | |
| | (4-IODO-BENZOYLAMINO)- |
| ACID | |
| 3-IODO-2-METHYLBENZOIC | ETHYL]-THIOUREIDO)- |
| ACID | BENZOIC ACID |
| 4-IODO-3-METHYLBENZOIC | 3-(3-[2,2,2-TRICHLORO-1- |
| ACID | (3-IODO-BENZOYLAMINO)- |
| ETHYL]-THIOUREIDO)- | ACID |
| | 2-IODO-4-METHYLBENZOIC |
| BENZOIC ACID | |
| (S)-5-IODOWILLARDIINE | ACID |
| [3-H] | ZELINSKY-BB BMA1527 |
| FMOC-PHE(4-I)-OH | 5-(2-FLUORO-4- |
| FMOC-D-PHE(4-I)-OH | IODOANILINO)-5-OXO-3- |
| (3,5-DIIODO-TYR4)- | PHENYLPENTANOIC ACID |
| ANGIOTENSIN II | (3- |
| 2-AMINO-3-BROMO-5- | [125I]IODOTYROSYL11)SOMA |
| IODOBENZOIC ACID | |
| 3-CHLORO-5-IODOBENZOIC | TOSTATIN-14 (TYR11) |
| | 3-AMINO-5-IODO-4-METHYL |
| ACID | |
| GALANIN, PORCINE, | BENZOIC ACID |
| | RBI 257 MALEATE |
| [125I], TYR26- | 3,5-DIIODO-4(4′- |
| (S)-5-IODOWILLARDIINE | METHOXYPHENOXY)BEN- |
| 3,5-DIIODO-4- | ZOIC |
| HYDROXYPHENYLPROPIONIC | ACID |
| | IODOMETHANE-13C, |
| ACID | TRIMETHYL PHOSPHITE, |
| [3H]-(S)-5- | |
| IODOWILLARDIINE | CHROMIUM (III) |
| TRANS-7-HYDROXY-PIPAT | ACETYLACETONATE |
| MALEATE | 5-IODO-2-METHYLBENZOIC |
| BOC-3,5-DIIODO-D-TYR-OH | ACID |
| H-2,5-DIIODO-HIS-OH HCL | 6-[(4- |
| (P-IODO-PHE7)-ACTH (4- | IODOANILINO)CARBONYL]- |
| 10) | |
| [3,5-DIIODO-TYR1, D- | 3,4-DIMETHYLCYCLOHEX-3- |
| | ENE-1-CARBOXYLIC ACID |
| ALA2, N-ME-PHE4, | 5-(2-FLUORO-4- |
| GLYCINOL5]-ENKEPHALIN | IODOANILINO)-5-OXO-3-[4- |
| 2-IODOFLUORENE-5- | |
| CARBOXYLIC ACID | (TRIFLUOROMETHYL)PHE- |
| 4-[[2- | NYL]PENTANOIC ACID |
| | NOCICEPTIN [TYR14] I125 |
| IODOPHENYL)SULFONYL]AM- | BOC-3-I-TYR-OH |
| INO]BENZOIC ACID | 2-(4- |
| METRIZOIC ACID | IODOPHENOXY)PROPANOIC |
| PD 150606 | |
| 2-IODO-6-METHYLBENZOIC | ACID |
| ACID | 4-(2,4-DIIODOANILINO)-4- |
| 3,5-DIIODOTHYROACETIC | OXOBUT-2-ENOIC ACID |
| (Z)-3-[2-(4- | FMOC-THX-OH |
| IODOPHENOXY)PHENYL]-2- | 5,6-DIMETHOXY-2- |
| | IODOBENZOIC ACID |
| PROPENOIC ACID | 2-CHLORO-6-IODO-5- |
| D, L-ALA-3-[2-((5- | |
| IODO)THIAZOLE)] | (TRIFLUOROMETHYL)PY- |
| D, L-ALA-3-[5-((2- | RIDINE-4-CARBOXYLIC ACID |
| IODO)THIAZOLE)] | (2R,3S)-1-CARBOXY-4- |
| D, L-ALA-3-[4-((2- | IODO-2,3- |
| IODO)THIAZOLE)] | DIHYDROXYCYCLOHEXA-4,6- |
| FMOC-D, L-ALA-3-[2-((5- | DIENE |
| IODO)THIAZOLE)] | 3,3′-DIIODO-L-THYRONINE |
| BOC-D, L-ALA-3-[2-((5- | 3-IODO-L-THYRONINE |
| IODO)THIAZOLE)] | TRIIODOACETIC ACID |
| FMOC-D, L-ALA-3-[5-((2- | 3-(4-CYANOPHENYL)-2- |
| IODO)THIAZOLE)] | [[(4- |
| BOC-D, L-ALA-3-[5-((2- | IODOPHENYL)SULFONYL]AM- |
| IODO)THIAZOLE)] | INO]PROPANOIC ACID |
| FMOC-D, L-ALA-3-[4-((2- | 3-[1-(4-IODOPHENYL)-1H- |
| IODO)THIAZOLE)] | PYRROL-2-YL]ACRYLIC ACID |
| BOC-D, L-ALA-3-[4-((2- | 2-[1-[2-(2-FLUORO-4- |
| IODO)THIAZOLE)] | IODOANILINO)-2- |
| (R)-3-AMINO-4-(4-IODO- | OXOETHYL]CYCLOPENTYL] |
| PHENYL)-BUTYRIC ACID HCL | ACETIC ACID |
| BOC-(R)-3-AMINO-4-(4- | 2-[1-[2-(2-FLUORO-4- |
| IODO-PHENYL) -BUTYRIC | IODOANILINO)-2- |
| ACID | |
| FMOC-(R)-3-AMINO-4-(4 | OXOETHYL]CYCLOHEXYL] |
| | ACETIC ACID |
| IODO-PHENYL)-BUTYRIC | 2-[2,4-DIIODO-6- |
| ACID | |
| (S)-3-AMINO-4-(4-IODO- | (METHYLSULFONYL)PHEN- |
| PHENYL)-BUTYRIC ACID HCL | OXY]ACETIC ACID |
| BOC-(S)-3-AMINO-4-(4- | TRIIODOTHYRONINE, N- |
| IODO-PHENYL)-BUTYRIC | SUCCINYL |
| | BOC-TYR(3,5-I2, (BR-Z))- |
| ACID | |
| FMOC-(S)-3-AMINO-4-(4 | OH |
| | FMOC-D-THX-OH |
| IODO-PHENYL)-BUTYRIC | BOC-PHE(4-NHZ,3,5-I2)-OH |
| ACID | FMOC-PHE(4-NHBOC,3,5- |
| | I2)-OH |
| 4-HYDROXY-3-IODO-5- | 1-CARBOXYLIC ACID |
| NITROPHENYLACETYL- | CIS-2-[2-(4-IODOPHENYL)- |
| EPSILON-AMINOCAPROIC | 2-OXOETHYL]CYCLOHEXANE |
| ACID | 1-CARBOXYLIC ACID |
| 5-IODO-2-FUROIC ACID | CIS-3-[2-(2-IODOPHENYL)- |
| 3-(5-IODOFURAN-2- | 2-OXOETHYL]CYCLOHEX- |
| YL)ACRYLIC ACID | ANE-1-CARBOXYLIC ACID |
| 6-IODOIMIDAZO[1,2- | CIS-3-[2-(3-IODOPHENYL)- |
| A]PYRIDINE-2-CARBOXYLIC | 2-OXOETHYL]CYCLOHEX- |
| ACID | ANE-1-CARBOXYLIC ACID |
| CIS-3-[2-(2-IODOPHENYL)- | CIS-3-[2-(4-IODOPHENYL)- |
| 2-OXOETHYL]CYCLOPENTANE- | 2-OXOETHYL CYCLOHEXANE |
| 1-CARBOXYLIC ACID | 1-CARBOXYLIC ACID |
| CIS-3-[2-(3-IODOPHENYL)- | CIS-4-[2-(2-IODOPHENYL)- |
| 2-OXOETHYL]CYCLOPENTANE- | 2-OXOETHYL]CYCLOHEX- |
| 1-CARBOXYLIC ACID | ANE-1-CARBOXYLIC ACID |
| CIS-3-[2-(4-IODOPHENYL)- | CIS-4-[2-(3-IODOPHENYL)- |
| 2-OXOETHYL]CYCLOPENTANE- | 2-OXOETHYL]CYCLOHEX- |
| 1-CARBOXYLIC ACID | ANE-1-CARBOXYLIC ACID |
| TRANS-2-[2-(2- | CIS-4-[2-(4-IODOPHENYL)- |
| IODOPHENYL)-2- | 2-OXOETHYL]CYCLOHEX- |
| OXOETHYL]CYCLOPENTANE-1- | ANE-1-CARBOXYLIC ACID |
| CARBOXYLIC ACID | 4-(2-IODOPHENYL)-4- |
| TRANS-2-[2-(3- | OXOBUTYRIC ACID |
| IODOPHENYL)-2- | 5-(2-IODOPHENYL)-5- |
| | OXOVALERIC ACID |
| OXOETHYL]CYCLOPENTANE-1- | 6-(2-IODOPHENYL)-6- |
| CARBOXYLIC ACID | OXOHEXANOIC ACID |
| TRANS-2-[2-(4- | 7-(2-IODOPHENYL)-7- |
| IODOPHENYL)-2- | OXOHEPTANOIC ACID |
| OXOETHYL]CYCLOPENTANE-1- | 8-(2-IODOPHENYL)-8- |
| CARBOXYLIC ACID | OCTANOIC ACID |
| CIS-2-[2-(2-IODOPHENYL)- | 4-(3-IODOPHENYL)-4- |
| 2-OXOETHYL]CYCLOHEXANE- | OXOBUTYRIC ACID |
| 1-CARBOXYLIC ACID | 5-(3-IODOPHENYL)-5- |
| CIS-2-[2-(3-IODOPHENYL)- | OXOVALERIC ACID |
| 2-OXOETHYL]CYCLOHEXANE- | 6-(3-IODOPHENYL)-6- |
| OXOHEXANOIC ACID | |
| 7-(3-IODOPHENYL)-7- | |
| OXOHEPTANOIC ACID | |
| 8-(3-IODOPHENYL)-8- | |
| OCTANOIC ACID | |
| 4-(4-IODOPHENYL)-4- | |
| OXOBUTYRIC ACID | |
| 5-(4-IODOPHENYL)-5- | |
| OXOVALERIC ACID | |
| 6-(4-IODOPHENYL)-6- | |
| OXOHEXANOIC ACI | |
| 7-(4-IODOPHENYL)-7- | |
| OXOHEPTANOIC ACID | |
| 8-(4-IODOPHENYL)-8- | |
| OCTANOIC ACID | |
| 4-(4-METHYL-3- | |
| IODOPHENYL)-4-OXOBUTYRIC | |
| ACID | |
| 5-(4-METHYL-3- | |
| TODOPHENYL)-5-OXOVALERIC | |
| ACID | |
| 6-(4-METHYL-3- | |
| IODOPHENYL)-6- | |
| OXOHEXANOIC ACID | |
| 7-(4-METHYL-3- | |
| IODOPHENYL)-7- | |
| OXOHEPTANOIC ACID | |
| 8-(4-METHYL-3- | |
| IODOPHENYL)-8- | |
| OXOOCTANOIC ACID | |

TABLE 4
Amino acids, derivatives and R groups for use as
monomers (avai1ab1e as hydroxyacids)

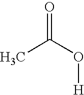

| | | |
|---|---|---|
| MOLNAME | SALICYLIC ACID | |
| FLURENOL | SALICYLIC ACID | |
| ALIZARIN COMPLEXONE | SALICYLIC ACID | |
| 4′-HYDROXYAZOBENZENE-2- | SALICYLIC ACID | |
| CARBOXYLIC ACID | SALICYLIC ACID | |
| [4′-HYDROXYAZOBENZENE-2- | SALICYLIC ACID] | |
| CARBOXYLIC ACID | 3,5-DIBROMOSALICYLIC ACID | |
| 4′-HYDROXYAZOBENZENE-2- | | |
| CAPEOXYLIC ACID | 3,5-DICHLOROSALICYLIC | |
| 4′-HYDROXYAZOBENZENE-2- | ACID | |
| | 3,5-DICHLOROSALICYLIC | |
| CARBOXYLIC ACID | | |
| 4′-HYDROXYAZOBENZENE-2- | ACID | |
| | 3,5-DIIODOSALICYLIC ACID | |
| CAPBOXYLIC ACID | | |
| 4′-HYDROXYAZOBENZENE-2- | 3-METHOXYSALICYLIC ACID | |
| CAPBOXYLIC ACID | | |
| 4′-HYDROXYAZOBENZENE-2- | 2,3-DIHYDROXYBENZOIC ACID | |
| CARBOXYLIC ACID | | |
| 4′-HYDROXYAZOBENZENE-2- | 2,3-DIHYDROXYBENZOIC ACID | |
| CAPEOXYLIC ACID] | | |
| 2-PHENOXYBENZOIC ACID | 2,3,4-TRIHYDROXYBENZOIC | |
| [2-PHENOXYBENZOIC ACID | ACID | |
| 2-PHENOXYBENZOIC ACID] | 3-METHYLSALICYLIC ACID | |
| SALICYLIC ACID | [3-METHYLSALICYLIC ACID | |
| [SALICYLIC ACID | 3-METHYLSALICYLIC ACID] | |
| SALICYLIC ACID | 4-CHLOROSALICYLIC ACID | |
| SALICYLIC ACID | 4-METHOXYSALICYLIC ACID | |
| SALICYLIC ACID | | |
| SALICYLIC ACID | 2,4-DIHYDROXYBENZOIC ACID | |
| SALICYLIC ACID | | |
| 2,4-DIHYDROXYBENZOIC ACID | ACID] | |
| | 2-ACETYLBENZOIC ACID | |
| 5-BROMO-2, 4- | ’-ACETYLBENZOIC ACID] | |
| DIHYDROXYBENZOIC ACID | 3-PHENOXYBENZOIC ACID | |
| PHLOROGLUCINOLCARBOXYLIC | [3-PHENOXYBENZOIC ACID | |
| ACID | 3-PHENOXYBENZOIC ACID | |
| 4-METHYLSALICYLIC ACID | 3-HYDROXYBENZOIC ACID | |
| 5-BROMOSALICYLIC ACID | 3-HYDROXYBENZOIC ACID | |
| [5-BROMOSALICYLIC ACID] | 3-HYDROXYBENZOIC ACID | |
| 5-FLUOROSALICYLIC ACID | 3-HYDROXYBENZOIC ACID] | |
| [5-FLUOROSALICYLIC ACID | 3-HYDROXY-4- | |
| 5-FLUOROSALICYLIC ACID | METHOXYBENZOIC ACID | |
| 5-FLUOROSALICYLIC ACID | 3-HYDROXY-4,5-DIMETHOXY- | |
| 5-FLUOROSALICYLIC ACID | BENZOIC ACID | |
| 5-FLUOROSALICYLIC ACID] | 3,4-DIHYDROXYBENZOIC ACID | |
| 5-CHLOROSALICYLIC ACID | | |
| 5-IODOSALICYLIC ACID | [3,4-DIHYDROXYBENZOIC | |
| [5-IODOSALICYLIC ACID | ACID | |
| 5-IODOSALICYLIC ACID | 3,4-DIHYDROXYBENZOIC | |
| 5-IODOSALICYLIC ACID] | ACID] | |
| 5-METHOXYSALICYLIC ACID | GALLIC ACID | |
| | [GALLIC ACID] | |
| [5-METHOXYSALICYLIC ACID | 3-HYDROXY-4-METHYLBENZOIC | |
| | ACID | |
| 5-METHOXYSALICYLIC ACID | 3,5-DIHYDROXYBENZOIC ACID | |
| 5-METHOXYSALICYLIC ACID] | [3,5-DIHYDROXYBENZOIC | |
| | ACID] | |
| 2,5-DIHYDROXYBENZOIC ACID | 4-BROMO-3,5- | |
| | DIHYDROXYBENZOIC ACID | |
| [2,5-DIHYDROXYBENZOIC | 3,5-DIHYDROXY-4- | |
| ACID] | METHYLBENZOIC ACID | |
| 5-METHYLSALICYLIC ACID | 5-HYDROXYISOPHTHALIC ACID | |
| | 2,6-DIHYDROXYBENZOIC ACID | |
| | 4-CHLOROMERCURIBENZOIC | |
| [2,6-DIHYDROXYBENZOIC | ACID | |
| ACID | [4-CHLOROMERCURIBENZOIC | |
| 2,6-DIHYDROXYBENZOIC | ACID | |
| 4-CHLOROMERCURIBENZOIC | [O-PHOSPHO-L-TYROSINE | |
| ACID | O-PHOSPHO-L-TYROSINE] | |
| 4-CHLOROMERCURIBENZOIC | L-TYROSINE | |
| ACID] | [L-TYROSINE | |
| 4-HYDROXYEENZOIC ACID | L-TYROSINE | |
| [4-HYDROXYBENZOIC ACID | L-TYROSINE | |
| 4-HYDROXYEENZOIC ACID | L-TYROSINE | |
| 4-HYDROXYBENZOIC ACID | L-TYROSINE | |
| 4-HYDROXYBENZOIC ACID] | L-TYROSINE | |
| 3,5-DIBROMO-4- | L-TYROSINE] | |
| | 3-IODO-L-TYROSINE | |
| HYDROXYBENZOIC ACID | | |
| 3-CHLORO-4-HYDROXYBENZOIC | [3-IODO-L-TYROSINE | |
| | 3-IODO-L-TYROSINE | |
| ACID | 3-IODO-L-TYROSINE | |
| [3-CHLORO-4- | | |
| | 3-IODO-L-TYROSINE | |
| HYDROXYBENZOIC ACID | 3-IODO-L-TYROSINE | |
| 3-CHLORO-4 -HYDROXYBENZOIC | 3-IODO-L-TYROSINE | |
| ACID] | 3-IODO-L-TYROSINE | |
| 3,5-DICHLORO-4- | 3-IODO-L-TYROSINE] | |
| HYDROXYBENZOIC ACID | DL-HOMOSERINE | |
| VANILLIC ACID | | |
| [VANILLIC ACID] | HYDROXYPHENYL)GLYCINE | |
| SYRINGIC ACID | [N-(4- | |
| KETOMALONIC ACID | | |
| 4-HYDROXYPHENYLPYRUVIC | HYDROXYPHENYL)GLYCINE | |
| ACID | | |
| [4-HYDROXYPHENYLPYRUVIC | HYDROXYPHENYL)GLYCINE] | |
| | O-HYDROXYHIPPURIC ACID | |
| ACID | 3-HYDROXY-3- | |
| 4-HYDROXYPHENYLPYRUVIC | | |
| | METHYLGLUTARIC ACID | |
| ACID] | 12-HYDROXYDODECANOIC ACID | |
| 3,3′,5-TRIIODO-L- | | |
| THYRONINE | 16-HYDROXYHEXADECANOIC | |
| L-THYROXINE | | |
| | ACID | |
| DL-M-TYROSINE | 3-(3,4- | |
| [DL-M-TYROSINE | DIHYDROXYPHENYL)PROPIONIC | |
| DL-M-TYROSINE] | | |
| L-DOPA | ACID | |
| [L-DOPA] | | |
| O-PHOSPHO-L-TYROSINE | HYDROXYPHENYL)PROPIONIC | |
| ACID | ACID | |
| [3-(4- | 2 -HYDROXY-1-NAPHTHOIC | |
| HYDROXYPHENYL)PROPIONIC | ACID | |
| | [2-HYDROXY-1-NAPHTHOIC | |
| ACID | | |
| | ACID | |
| | 2-HYDROXY-1-NAPHTHOIC | |
| HYDROXYPHENYL)PROPIONIC | | |
| | ACID | |
| ACID] | 2-HYDROXY-1-NAPHTHOIC | |
| DIPHENOLIC ACID | | |
| 2-CARBOXYBENZALDEHYDE | ACID] | |
| [2-CARBOXYBENZALDEHYDE] | CALCONCARBOXYLIC ACID | |
| APOCHOLIC ACID | PAMOIC ACID | |
| CHOLIC ACID | 3-HYDROXY-2-NAPHTHOIC | |
| DEOXYCHOLIC ACID | ACID | |
| URSODEOXYCHOLIC ACID | [3-HYDROXY-2-NAPHTHOIC | |
| HYODEOXYCHOLIC ACID | ACID | |
| LITHOCHOLIC ACID | 3-HYDROXY-2-NAPHTHOIC | |
| 18-BETA-GLYCYRRHETINIC | ACID] | |
| ACID | 2-ETHYL-2-HYDROXYBUTYRIC | |
| CHLOROGENIC ACID | ACID | |
| D-(−)-QUINIC ACID | L-ALPHA-METHYL-DOPA | |
| [D-(−)-QUINIC ACID] | DL-ALPHA-METHYLTYROSINE | |
| 1-HYDROXY-2-NAPHTHOIC | | |
| ACID | 2,2- | |
| [1-HYDROXY-2-NAPHTHOIC | BIS(HYDROXYMETHYL)PROPION | |
| ACID | IC ACID | |
| 1-HYDROXY-2-NAPHTHOIC | [2,2- | |
| ACID | BIS(HYDROXYMETHYL)PROPION | |
| 1-HYDROXY-2-NAPHTHOIC | IC ACID] | |
| ACID | ALPHA-CYANO-3- | |
| 1-HYDROXY-2-NAPHTHOIC | HYDROXYCINNAMIC ACID | |
| ACID] | ALPHA-CYANO-4- | |
| 4-FLUOROSULFONYL-1- | HYDROXYCINNAMIC ACID | |
| HYDROXY-2-NAPHTHOIC ACID | [ALPHA-CYANO-4- | |
| | HYDROXYCINNAMIC ACID | |
| 4-AMINOSULFONYL-1- | ALPHA-CYANO-4- | |
| HYDROXY-2-NAPHTHOIC ACID | HYDROXYCINNAMIC ACID | |
| | ALPHA-CYANO-4- | |
| 3,5-DIHYDROXY-2-NAPHTHOIC | | |
| HYDROXYCINNAMIC ACID] | [L-(−)-3-PHENYLLACTIC | |
| ALPHA-HYDROXYHIPPURIC | | |
| | ACID | |
| ACID | L-(−)-3-PHENYLLACTIC | |
| DL-3-METHOXYMANDELIC ACID | | |
| | ACID] | |
| D-(+)-MALIC ACID | | |
| 3-HYDROXY-4- | [D-(+)-MALIC ACID | |
| METHOXYMANDELIC ACID | D-(+)-MALIC ACID | |
| [3-HYDROXY-4- | D-(+)-MALIC ACID | |
| METHOXYMANDELIC ACID | D-(+)-MALIC ACID | |
| 3-HYDROXY-4- | D-(+)-MALIC ACID | |
| METHOXYMANDELIC ACID] | D-(+)-MALIC ACID | |
| DL-3,4-DIHYDROXYMANDELIC | D-(+) -MALIC ACID | |
| ACID | D-(+)-MALIC ACID] | |
| 4-BROMOMANDELIC ACID | DL-LEUCIC ACID | |
| 4-METHOXYMANDELIC ACID | [DL-LEUCIC ACID | |
| [4-METHOXYMANDELIC ACID] | DL-LEUCIC ACID | |
| DL-4-HYDROXYMANDELIC ACID | DL-LEUCIC ACID | |
| | DL-LEUCIC ACID] | |
| DL-4-HYDROXY-3- | DL-TROPIC ACID | |
| METHOXYMANDELIC ACID | [DL-TROPIC ACID] | |
| DIHYDROXYFUMARIC ACID | PHOSPHOENOLPYRUVATE | |
| TARTRONIC ACID | TRI(CYCLOHEXYLAMMONIUM) | |
| D-(−)-TARTARIC ACID | SALT | |
| [D-(−)-TARTARIC ACID | | |
| D-(−)-TARTARIC ACID | HYDROXYPHENYLGLYCINE | |
| D-(−)-TARTARIC ACID | | |
| D-(−)-TARTARIC ACID | | |
| | HYDROXYPHENYLGLYCINE | |
| D-(−)-TARTARIC ACID | DL-THREO-BETA-(3,4- | |
| D-(−)-TARTARIC ACID | | |
| D-(−)-TARTARIC ACID | DIHYDROXYPHENYL)SERINE | |
| | D-SERINE | |
| D-(−)-TARTARIC ACID | [D-SERINE | |
| D-(−)-TARTARIC ACID | D-SERINE | |
| D-(−)-TARTARIC ACID] | D-SERINE] | |
| MUCIC ACID | 6-HYDROXYDOPA | |
| GLUCONIC ACID | TRICINE | |
| 2-HYDROXY-3-METHYLBUTYRIC | [TRICINE | |
| ACID | TRICINE | |
| L-(−)-3-PHENYLLACTIC ACID | TRICINE | |
| TRICINE | ACID] | |
| TRICINE | HOMOGENTISIC ACID | |
| TRICINE | 2,5-DIHYDROXY-1,4- | |
| TRICINE | BENZENEDIACETIC ACID | |
| TRICINE | 3-HYDROXYPHENYLACETIC | |
| TRICINE] | ACID | |
| 1,3-DIAMINO-2-PROPANOL- | [3-HYDROXYPHENYLACETIC | |
| N,N,N′,N′-TETRAACETIC | ACID | |
| ACID | 3-HYDROXYPHENYLACETIC | |
| N-(2- | ACID | |
| HYDROXYETHYL)IMINODIACETI | 3-HYDROXYPHENYLACETIC | |
| C ACID | ACID | |
| N-(2- | 3-HYDROXYPHENYLACETIC | |
| HYDROXYETHYL)ETHYLENEDIAM | ACID] | |
| INETRIACETIC ACID | 3,4-DIHYDROXYPHENYLACETIC | |
| BICINE | ACID | |
| [BICINE | | |
| BICINE | 4-HYDROXYPHENYLACETIC | |
| BICINE | ACID | |
| BICINE | 3-FLUORO-4- | |
| BICINE | HYDROXYPHENYLACETIC ACID | |
| BICINE | | |
| B1CINE | [3-FLUORO-4- | |
| BICINE | HYDROXYPHENYLACETIC ACID | |
| BICINE | | |
| BICINE] | 3-FLUORO-4- | |
| MERSALYL ACID | HYDROXYPHENYLACETIC ACID | |
| [MERSALYL ACID] | | |
| GLYCOLIC ACID | 3-FLUORO-4- | |
| [GLYCOLIC ACID | HYDROXYPHENYLACETIC ACID | |
| GLYCOLIC ACID | | |
| GLYCOLIC ACID | 3-FLUORO-4- | |
| GLYCOLIC ACID] | HYDROXYPHENYLACETIC ACID] | |
| 2-HYDROXYPHENYLACETIC | | |
| ACID | 3-CHLORO-4- | |
| [2-HYDROXYPHENYLACETIC | HYDROXYPHENYLACETIC ACID | |
| ACID | | |
| 2-HYDROXYPHENYLACETIC | | |
| [3-CHLORO-4- | 2-HYDROXY-2-METHYLBUTYRIC | |
| HYDROXYPHENYLACETIC ACID] | ACID | |
| | L-(+)-MANDELIC ACID | |
| HOMOVANILLIC ACID | [L-(+)-MANDELIC ACID | |
| 2-HYDROXYCINNAMIC ACID | L-(+)-MANDELIC ACID | |
| [2-HYDROXYCINNAMIC ACID] | L-(+)-MANDELIC ACID | |
| 3-HYDROXYCINNAMIC ACID | L-(+)-MANDELIC ACID | |
| [3-HYDROXYCINNAMIC ACID | L-(+)-MANDELIC ACID] | |
| 3-HYDROXYCINNAMIC ACID] | LACTIC ACID | |
| 3-HYDROXY-4- | [LACTIC ACID | |
| METHOXYCINNAMIC ACID | LACTIC ACID | |
| CAFFEIC ACID | LACTIC ACID | |
| [CAFFEIC ACID | LACTIC ACID | |
| CAFFEIC ACID | LACTIC ACID | |
| CAFFEIC ACID | LACTIC ACID] | |
| CAFFEIC ACID] | D-ALLO-THREONINE | |
| 4-HYDROXYCINNAMIC ACID | 3-HYDROXYBUTYRIC ACID | |
| [4-HYDROXYCINNAMIC ACID] | DL-ALPHA-HYDROXYCAPROIC | |
| FERULIC ACID | ACID | |
| [FERULIC ACID] | [DL-ALPHA-HYDROXYCAPROIC | |
| 3,5-DIMETHOXY-4- | ACID | |
| HYDROXYCINNAMIC ACID | DL-ALPHA-HYDROXYCAPROIC | |
| [3,5-D-METHOXY-4- | ACID | |
| HYDROXYCINNAMIC ACID | DL-ALPHA-HYDROXYCAPROIC | |
| 3,5-DIMETHOXY-4- | ACID] | |
| HYDROXYCINNAMIC ACID | 12-HYDROXYSTEARIC ACID | |
| 3,5-DIMETHOXY-4- | CALCEIN | |
| HYDROXYCINNAMIC ACID | HEMATOPORPHYRIN | |
| 3,5-DIMETHOXY-4- | CIS-4-HYDROXY-D-PROLINE | |
| HYDROXYCINNAMIC ACID | | |
| 3,5-DIMETHOXY-4- | [CIS-4-HYDROXY-D-PROLINE] | |
| HYDROXYCINNAMIC ACID | | |
| 3,5-DIMETHOXY-4- | 5-HYDROXYINDOLE-2- | |
| | CARBOXYLIC ACID | |
| HYDROXYCINNAMIC ACID] | 5-HYDROXYINDOLE-3-ACETIC | |
| BENZILIC ACID | | |
| ATROLACTIC ACID | ACID | |
| 2-HYDROXYISOBUTYRIC ACID | DL-INDOLE-3-LACTIC ACID | |
| DL-INDOLE-3-LACTIC ACID | 3,5-DINITROSALICYLIC ACID | |
| DL-5-HYDROXYTRYPTOPHAN | 3-HYDROXY-4-METHYL-2- | |
| 2,4-DIHYDROXYPYRIMIDINE- | NITROBENZOIC ACID | |
| 5-CARBOXYLIC ACID | [3-HYDROXY-4-METHYL-2- | |
| | NITROBENZOIC ACID | |
| OROTIC ACID | 3-HYDROXY-4-METHYL-2- | |
| 3,5-DIIODO-4-PYRIDONE-1- | | |
| | NITROBENZOIC ACID | |
| ACETIC ACID | 3-HYDROXY-4-METHYL-2- | |
| 2-HYDROXY-6- | | |
| | NITROBENZOIC ACID | |
| METHYLNICOTINIC ACID | 3-HYDROXY-4-METHYL-2- | |
| [2-HYDROXY-6- | | |
| | NITROBENZOIC ACID | |
| METHYIMICOTINIC ACID] | 3-HYDROXY-4-METHYL-2- | |
| CITRAZINIC ACID | | |
| 6-HYDROXYNICOTINIC ACID | NITROBENZOIC ACID | |
| | 3-HYDROXY-4-METHYL-2- | |
| 3-HYDROXYPICOLINIC ACID | NITROBENZOIC ACID | |
| | 3-HYDROXY-4-METHYL-2- | |
| [3-HYDROXYPICOLINIC ACID | NITROBENZOIC ACID | |
| | 3-HYDROXY-4-METHYL-2- | |
| 3-HYDROXYPICOLINIC ACID | NITROBENZOIC ACID | |
| | 3-HYDROXY-4-METHYL-2- | |
| 3-HYDROXYPICOLINIC ACID] | NITROBENZOIC ACID] | |
| | 3-HYDROXY-4-NITROBENZOIC | |
| 4-PYRIDOXIC ACID | | |
| | ACID | |
| 3-HYDROXY-2- | [3-HYDROXY-4-NITROBENZOIC | |
| QUINOXALINECARBOXYLIC | ACID | |
| ACID | 3-HYDROXY-4-NITROBENZOIC | |
| KYNURENIC ACID | | |
| XANTHURENIC ACID | ACID] | |
| | 4-HYDROXY-3-NITROBENZOIC | |
| 4-HYDROXY-7- | | |
| | ACID | |
| (TRIFLUOROMETHYL)-3- | 4-HYDROXY-3- | |
| QUINOLINECARBOXYLIC ACID | NITROPHENYLACETIC ACID | |
| | [4-HYDROXY-3- | |
| TROLOX (R) | | |
| CALCEIN BLUE | NITROPHENYLACETIC ACID] | |
| | 3-NITRO-L-TYROSINE | |
| CITRININ | | |
| 5-FORMYLSALICYLIC ACID | [3-NITRO-L-TYROSINE | |
| | 3-NITRO-L-TYROSINE | |
| 3-NITRO-L-TYROSINE | [5-AMINOSALICYLIC ACID | |
| 3-NITRO-L-TYROSINE] | 5-AMINOSALICYLIC ACID | |
| 5-NITROSALICYLIC ACID | 5-AMINOSALICYLIC ACID] | |
| [5-NITROSALICYLIC ACID | DL-ISOSERINE | |
| 5-NITROSALICYLIC ACID] | DL-4-AMINO-3- | |
| ZINCON | HYDROXYBUTYRIC ACID | |
| SULFOSALICYLIC ACID | 3,5-DI-TERT-BUTYL-4- | |
| [SULFOSALICYLIC ACID | HYDROXYBENZOIC ACID | |
| SULFOSALICYLIC ACID | 3,5-DIISOPROPYLSALICYLIC | |
| SULFOSALICYLIC ACID | ACID | |
| SULFOSALICYLIC ACID] | | |
| 3-ANINO-4-HYDROXYBENZOIC | RHEIN | |
| ACID | BETULINIC ACID | |
| [3-AMINO-4-HYDROXYBENZOIC | URSOLIC ACID | |
| ACID | (R)-(−)-HEXAHYDROMANDELIC | |
| 3-AMINO-4-HYDROXYBENZOIC | ACID | |
| ACID] | | |
| 3-HYDROXYANTHRANILIC ACID | HEXAHYDROMANDELIC ACID | |
| | (R)-(−)-HEXAHYDROMANDELIC | |
| [3-HYDROXYANTHRANILIC | ACID] | |
| ACID | 2-HYDROXYNICOTINIC ACID | |
| 3-HYDROXYANTHEANILIC ACID | | |
| | [2-HYDROXYNICOTINIC ACID] | |
| 3-HYDROXYANTHRANILIC ACID | | |
| | ACRYLAMIDO BUFFER | |
| 3-HYDROXYANTHRANILIC ACID | 3,7-DIHYDROXY-2-NAPHTHOIC | |
| | ACID | |
| 3-HYDROXYANTHRANILIC ACID | [3,7-DIHYDROXY-2- | |
| | NAPHTHOIC ACID] | |
| 3-HYDROXYANTHRANILIC ACID | 3-AMINOSALICYLIC ACID | |
| | [3-AMINOSALICYLIC ACID] | |
| 3-HYDROXYANTHRANILIC | 1,4-DIHYDROXY-2-NAPHTHOIC | |
| ACID] | ACID | |
| 4-AMINOSALICYLIC ACID | 3-CHLOROSALICYLIC ACID | |
| [4-AMINOSALICYLIC ACID] | 4-HYDROXYISOPHTHALIC ACID | |
| 2-AMINO-5-HYDROXYBENZOIC | | |
| ACID | [4-HYDROXYISOPHTHALIC | |
| 5-AMINOSALICYLIC ACID | ACID | |
| 4-HYDROXYISOPHTHALIC | CITRIC ACID | |
| ACID] | CITRIC ACID | |
| 10-HYDROXYDECANOIC ACID | CITRIC ACID | |
| | CITRIC ACID | |
| DL-4-HYDROXYPHENYLLACTIC | CITRIC ACID | |
| ACID | CITRIC ACID | |
| | CITRIC ACID | |
| DL-BETA-HYDROXYNORVALINE | CITRIC ACID | |
| | CITRIC ACID | |
| 5-AMINOOROTIC ACID | CITRIC ACID | |
| PTERIN-6-CARBOXYLIC ACID | CITRIC ACID | |
| | CITRIC ACID | |
| 9-HYDROXY-9H-FLUORENE-1- | CITRIC ACID | |
| CARBOXYLIC ACID | CITRIC ACID | |
| 1-HYDROXY-1- | CITRIC ACID | |
| CYCLOPROPANECARBOXYLIC | CITRIC ACID | |
| | CITRIC ACID | |
| ACID | | |
| [1-HYDROXY-1- | CITRIC ACID | |
| | CITRIC ACID | |
| CYCLOPROPANECARBOXYLIC | CITRIC ACID | |
| ACID | CITRIC ACID | |
| 1-HYDROXY-1- | CITRIC ACID | |
| CYCLOPROPANECARBOXYLIC | CITRIC ACID | |
| ACID] | CITRIC ACID | |
| D-(−)-M- | CITRIC ACID | |
| | CITRIC ACID | |
| HYDROXY PHENYLGLYCINE | | |
| HEMATIN | CITRIC ACID | |
| CITRIC ACID | CITRIC ACID | |
| [CITRIC ACID | CITRIC ACID | |
| CITRIC ACID | CITRIC ACID | |
| CITRIC ACID | CITRIC ACID | |
| CITRIC ACID | CITRIC ACID | |
| CITRIC ACID | CITRIC ACID | |
| CITRIC ACID | CITRIC ACID | |
| CITRIC ACID | CITRIC ACID | |
| CITRIC ACID | CITRIC ACID | |
| CITRIC ACID | CITRIC ACID | |
| CITRIC ACID | CITRIC ACID | |
| | CITRIC ACID | |
| CITRIC ACID | 5-HYDROXY-DL-LYSINE | |
| CITRIC ACID | HYDROCHLORIDE | |
| CITRIC ACID | CARBOXYMETHOXYLAMINE | |
| CITRIC ACID | HEMIHYDROCHLORIDE | |
| CITRIC ACID | 3-HYDROXYANTHRANILIC ACID | |
| CITRIC ACID | HYDROCHLORIDE | |
| CITRIC ACID | N-OMEGA-METHYL-5- | |
| CITRIC ACID | HYDROXYTRYPTAMINE OXALATE | |
| CITRIC ACID | | |
| | SALT | |
| CITRIC ACID | HEMATOPORPHYRIN | |
| CITRIC ACID | | |
| | DIHYDROCHLORIDE | |
| CITRIC ACID | 2-HYDROXY-4,6- | |
| CITRIC ACID | | |
| | DIMETHOXYBENZOIC ACID | |
| CITRIC ACID | 5-ACETYLSALICYLIC ACID | |
| CITRIC ACID | 4-HYDROXYPHTHALIC ACID | |
| CITRIC ACID | 3-(2- | |
| CITRIC ACID | | |
| CITRIC ACID | HYDROXYPHENYL)PROPIONIC | |
| CITRIC ACID | ACID | |
| CITRIC ACID | 2-HYDROXYDODECANOIC ACID | |
| CITRIC ACID | | |
| CITRIC ACID | 2-HYDROXYMYRISTIC ACID | |
| CITRIC ACID | 2-HYDROXYHEXADECANOIC | |
| CITRIC ACID | ACID | |
| CITRIC ACID | CATECHOL-O,O-DIACETIC | |
| CITRIC ACID | ACID | |
| CITRIC ACID | 4-HYDROXYPHENOXYACETIC | |
| CITRIC ACID | ACID | |
| CITRIC ACID | 2-HYDROXYOCTANOIC ACID | |
| CITRIC ACID | 2-HYDROXYPHENOXYACETIC | |
| CITRIC ACID] | ACID | |
| DL-CARNITINE | [2-HYDROXYPHENOXYACETIC | |
| HYDROCHLORIDE | ACID] | |
| GALLOCYANINE | 3,4-DIMETHOXYSALICYLIC | |
| 2-(4- | ACID | |
| HYDROXYPHENOXY)PROPIONIC | 2,3-DIHYDROXY-4- | |
| ACID | METHOXYBENZOIC ACID | |
| L-TYROSINE HYDROCHLORIDE | 4-ETHOXY-2-HYDROXYBENZOIC | |
| | ACID | |
| 5-ETHOXY-2-HYDROXYBENZOIC | 3-HYDROXYHOMOPHTHALIC | |
| ACID | ACID | |
| 5-CARBOXYVANILLIC ACID | 3-HYDROXY-4- | |
| 5,5′-METHYLENEDISALICYLIC | METHOXYPHENYLACETIC ACID | |
| ACID | | |
| | (3-BROMO-4-HYDROXY- | |
| 2-(P- | PHENYL)-ACETIC ACID | |
| HYDROXYBENZOYL)BENZOIC | 3,5-DIMETHOXY-4- | |
| ACID | 1-HYDROXYPHENYLACETIC ACID | |
| 3,5,3′,5′- | | |
| TETRAIODOTHYROFORMIC ACID | 3-ETHOXY-4- | |
| | HYDROXYPHENYLACETIC ACID | |
| 3-O-METHYLGALLIC ACID | | |
| 5-HYDROXYISOVANILLIC ACID | 2,3-DIHYDROXY-4- | |
| | METHOXYCINNAMIC ACID | |
| 5-CHLOROVANILLIC ACID | 5-CARBOXYVANILLIN | |
| 3,5-DIIODO-4- | 3,5-DINITRO-4- | |
| HYDROXYBENZOIC ACID | HYDROXYPHENYLACETIC ACID | |
| 3-TERT-BUTYL-4- | | |
| HYDROXYBENZOIC ACID | 3,5-DINITRO-4- | |
| [3-TERT-BUTYL-4- | HYDROXYBENZOIC ACID | |
| | 3-HYDROXY-2-NITROBENZOIC | |
| HYDROXYBENZOIC ACID | | |
| 3-TERT-BUTYL-4- | ACID | |
| | (3-HYDROXY-2-NITROBENZOIC | |
| HYDROXYBENZOIC ACID] | | |
| 4-HYDROXY-3,5- | ACID | |
| | 3-HYDROXY-2-NITROBENZOIC | |
| DIMETHYLBENZOIC ACID | | |
| 3,5-DIIODO-L-TYROSINE | ACID | |
| 3-(3- | 3-HYDROXY-2-NITROBENZOIC | |
| HYDROXYPHENYL)PROPIONIC | ACID | |
| | 3-HYDROXY-2-NITROBENZOIC | |
| ACID | | |
| HYDROFERULIC ACID | ACID | |
| [HYDROFERULIC ACID] | 3-HYDROXY-2-NITROBENZOIC | |
| HYDROXINAPINIC ACID | ACID | |
| 3-FORMYL-4-HYDROXYBENZOIC | 3-HYDROXY-2-NITROBENZOIC | |
| ACID | ACID | |
| 3,5-DIBROMO-4- | 3-HYDROXY-2-NITROBENZOIC | |
| HYDROXYPHENOXY ACETIC | ACID | |
| ACID | | |
| 3-HYDROXY-2-NITROBENZOIC | TETRABROMODIPHENOLIC ACID | |
| ACID] | | |
| 4-AMINO-3-HYDROXYBENZOIC | 4,4-BIS-(3,5-DICHLORO-4- | |
| ACID | HYDROXYPHENYL)-VALERIC | |
| [4-AMINO-3-HYDROXYBENZOIC | ACID | |
| ACID] | 3-ACETAMIDO-2,4,6- | |
| 3,5-DI-TERT-BUTYL-4- | TRIIODOBENZOIC ACID, | |
| HYDROXYCINNAMIC ACID | BIS(2-HYDROXYETHYL)- | |
| 3-BROMO-4-HYDROXY-5- | | |
| | AMMONIUM SALT | |
| METHOXYCINNAMIC ACID | SALICYLSALICYLIC ACID | |
| [3-BROMO-4-HYDROXY-5- | 3-PHENYLSALICYLIC ACID | |
| METHOXYCINNAMIC ACID] | 4-ACETAIVIIDO SALICYLIC | |
| 7-HYDROXYCOUMARIN-3- | ACID | |
| CARBOXYLIC ACID | 5-(ALPHA,ALPHA-DIMETHYL- | |
| 3,5-DI-TERT-BUTYL-4- | 4-HYDROXYBENZYL) SALICYLIC | |
| HYDROXYPHENYLPROPIONIC | ACID | |
| ACID | | |
| 3-BROMO-4-HYDROXYBENZOIC | 2-HYDROXY-3-ISOPROPYL-6- | |
| ACID | METHYLBENZOIC ACID | |
| 5-HYDROXY-2-NITROBENZOIC | 2′-CARBOXY-2-HYDROXY-4- | |
| ACID | METHOXY-BENZOPHENONE | |
| 4-HYDROXYMETHYLBENZOIC | 3-HYDROXY-2,4,6- | |
| ACID | TRIIODOBENZOIC ACID | |
| MORPHOLINE-P- | N,N-BIS(2-HYDROXYETHYL)- | |
| HYDROXYBENZOATE | 3- | |
| BAICALIN | CARBOXYBENZENESULFONAMIDE | |
| ROSMARINIC ACID | | |
| 5-(TERT-BUTYL)-2- | 1,2-DIMETHYL-4-(2- | |
| HYDROXYCYCLOHEXANECARBOXY | CARBOXY-PHENYLAZO)-5- | |
| LIC ACID | HYDROXYBENZENE | |
| 2-(8-CARBOXYOCTYL)-3- | | |
| HYDROXY-1,4- | HYDROXYETHOXY)BENZOIC | |
| NAPHTHOQUINONE | ACID | |
| 2-(9-CARBOXYNONYL)-3- | 5-(TERT-BUTYL)-4-HYDROXY- | |
| HYDROXY-1,4- | M-TOLUIC ACID | |
| | 3′, 5′-DICHLORO-2 | |
| NAPHTHOQUINONE | | |
| | HYDROXY-4 | |
| METHYLMALEANILIC ACID | PHENYLPROPIONIC ACID | |
| 4′-BENZAMIDO-3′- | DL-O-PHOSPHOSERINE | |
| HYDROXYSUCCINANILATE | DL-O-TYROSINE | |
| 3-(2-HYDROXY-5- | [DL-O-TYROSINE] | |
| METHYLBENZOYL)PROPIONIC | 4-(6-HYDROXY-3-OXO-3H- | |
| ACID | XANTHEN-9-YL)BENZOIC ACID | |
| H-GLY-HYP-OH | | |
| 4′-(2-FUROYLAMINO)-3′- | 2-IMINO-4-OXYTHIAZOLE-5- | |
| HYDROXYMALONANILIC ACID | ACETIC ACID | |
| | 2,4-DIHYDROXYTHIAZOLE-5- | |
| 5-ANDROSTEN-3BETA-OL- | ACETIC ACID | |
| 17BETA-CARBOXYLIC ACID | [2,4-DIHYDROXYTHIAZOLE-5- | |
| TESTOSTERONE | ACETIC ACID] | |
| HEMISUCCINATE | N-(4-CARBOXY-3- | |
| 4-BROMO-1-HYDROXY-2- | HYDROXYPHENYL)MALEIMIDE | |
| NAPHTHOIC ACID | | |
| 2-(2-HYDROXY-1- | 5-CHLORO-2,6-DIHYDROXY-4- | |
| NAPHTHYLMETHYLENEAMINO)BE | PYRIMIDINECARBOXYLIC ACID | |
| NZOIC ACID | | |
| 3-(2-HYDROXY-1- | 6-HYDROXY-2-(METHYLTHIO)- | |
| NAPHTHYLMETHYLENEAMINO)BE | 4-PYRIMIDINECARBOXYLIC | |
| NZOIC ACID | ACID | |
| 4-(2-HYDROXY-1- | 2-AMINO-4- | |
| NAPHTHYLMETHYLENEAMINO)BE | HYDROXYPYRIMIDINE-6- | |
| NZOIC ACID | CARBOXYLIC ACID | |
| 1- | 6-HYDROXY-4- | |
| HYDROXYCYCLOHEPTANECARBOX | PYRIMIDINECARBOXYLIC ACID | |
| YLIC ACID | | |
| DL-2-METHYLSERINE | [6-HYDROXY-4- | |
| ALPHA-METHYL-DL-M- | | |
| | PYRIMIDINECARBOXYLIC | |
| TYROSINE | | |
| 4-HYDROXY-ALPHA- | ACID] | |
| | 3-CARBOXY-4- | |
| PHENYLCINNAMIC ACID | | |
| 3-(P-HYDROXYPHENYL)-2- | HYDROXYBENZENESULFONIC | |
| PHENYLPROPIONIC ACID | (METHYL-PENTADECYL | |
| 3-(3,5-DIIODO-4- | PYRIMIDINYLIDENE)HYDRAZID | |
| HYDROXYPHENYL)-2- | E | |
| CARBOXY- | 1,4,4A,5,8,8A-HEXAHYDRO- | |
| HYDROXYBENZENESULFONIC | 5-HYDROXY-8-OXO-1- | |
| HEXADECYLOXYPHENYL- | NAPHTHALENECARBOXYLIC | |
| METHYL- | ACID | |
| SULFOQUINOLYLIDENHYDRAZ | 4-(2-HYDROXY-1- | |
| 2-HYDROXYQUINOLINE-4- | NAPHTHYLMETHYLENEAMINO)SA | |
| CARBOXYLIC ACID | LICYLIC ACID | |
| 2-(CARBOXY-4- | 4-(2-CHLORO-5- | |
| HYDROXYPHENYL)QUINOLINE- | (TRIFLUOROMETHYL)PHENYLAZ | |
| 4-CARBOXYLIC ACID | O)-3-HYDROXY-2-NAPHTHOIC | |
| 3-HYDROXY-2- | | |
| | ACID | |
| PHENYLCINCHONINIC ACID | | |
| 4-HYDROXY-6- | | |
| | HYDROXYBENZYLIDENEAMINO)S | |
| METHOXYQUINOLINE-3- | | |
| | ALICYLIC ACID | |
| CARBOXYLIC ACID | ALPHA-(4-CARBOXY-3- | |
| BUTTPARK 20\09-64 | | |
| 7,8-DIMETHYL-4-HYDROXY-3- | HYDROXYPHENYLIMINO)-6- | |
| QUINOLINECARBOXYLIC ACID | METHOXY-O-CRESOL | |
| 4-HYDROXY-8- | (DIMETHYLAMINO)BENZYLIDEN | |
| METHOXYQUINOLINE-3- | EAMINO)SALICYLIC ACID | |
| CARBOXYLIC ACID | | |
| 3-NITROSALICYLIC ACID | | |
| 4,4-BIS-(4-HYDROXY-3- | HYDROXYBENZYLIDENEAIYIINO)S | |
| NITROPHENYL)-VALERIC ACID | ALICYLIC ACID | |
| | 2-(3-HYDROXY-4- | |
| 3-CARBOXY-4-HYDROXY-5- | METHOXYPHENYL)GLYOXYLIC | |
| SULFOANILINE | ACID | |
| 4,4-BIS-(3-AMINO-4- | 2-(2-(2-HYDROXY-1- | |
| HYDROXYPHENYL)-VALERIC | NAPHTHOYL)VINYL)BENZOIC | |
| ACID | ACID | |
| 4′-AMINO-2′- | 3-HYDROXY-4,4,4- | |
| HYDROXYSUCCINANILIC ACID | TRICHLOROBUTYRIC ACID | |
| | 2-(2-(4- | |
| | HYDROXYPHENYL)PROPYL)BENZ | |
| | QIC ACID | |
| 4′- (2-HYDROXYETHYL) - | 2-PHOSPHOENOL PYRUVATE | |
| 3,4,5,6- | CHA-SALT | |
| TETRABROMOPHTHALANILIC | L-NORADRENALINE | |
| ACID | BITARTRATE | |
| BETA-(2-HYDROXY-3- | AMMONIUM LACTATE | |
| | PROSTAGLANDIN B1 | |
| METHYLPHENYL)ALANINE | 15-EPI PROSTAGLANDIN A1 | |
| BETA-(2-HYDROXY-4- | | |
| METHYLPHENYL)ALANINE | 15(R)-PROSTAGLANDIN E1 | |
| BETA-(2-HYDROXY-5- | DL-THREO-BETA | |
| METHYLPHENYL)ALANINE | | |
| 4-HYDROXY-ALPHA-MERCAPTO- | HYDROXYASPARTIC ACID | |
| | MYCOPHENOLIC ACID | |
| 3-METHOXYCINNAMIC ACID | [MYCOPHENOLIC ACID | |
| 3-HYDROXY-ALPHA-MERCAPTO- | MYCOPHENOLIC ACID | |
| | MYCOPHENOLIC ACID | |
| BETA-METHYLCINNAMIC ACID | MYCOPHENOLIC ACID] | |
| 6,8-DICHLORO-2-HYDROXY-4- | THROMBOXANE B2 | |
| | 3-CARBOXYPHENYLBORONIC | |
| QUINOLINECARBOXYLIC ACID | | |
| | ACID | |
| N,N-DIETHYLHYDROXYLAMINE | [3-CARBOXYPHENYLBORONIC | |
| HEMIOXALATE | ACID | |
| | 3-CARBOXYPHENYLBORONIC | |
| EPINEPHRINE BITARTRATE | ACID] | |
| N-METHYLHYDROXYLAMINE | LEUKOTRIENE D4 | |
| OXALATE | NG-MONOMETHYL-L-ARGININE, | |
| 8-HYDROXYQUINOLINE | P-HYDROXYAZOBENZENE-P′- | |
| BENZOATE | SULFONATE SALT, | |
| 3-AMINO-4- | | |
| | MONOHYDRATE | |
| HYDROXYDIPHENYLMETHANE | | |
| | 7-ALPHA, 12-ALPHA- | |
| OXALATE | | |
| 2-HYDROXYDIBENZOFURAN-3- | DIHYDROXY-5-BETA-CHOLAN- | |
| CARBOXYLIC ACID | 24-OIC ACID | |
| M-NITROBENZENE ACID | FORSKOLIN, 7-DEACETYL-7- | |
| SULFONE | O-HEMISUCCINYL- | |
| METANILSULFONYL SALICYLIC | 12(S)-HETE | |
| ACID | 11(S)-HETE | |
| PHENOLPHTHALIN | 11-DEHYDROTHROMBOXANE B2 | |
| CHOLINE BITARTRATE | | |
| PROSTAGLANDIN H2 | CITRATE | |
| H-TRP(5-OH)-OH HCL | 4-HYDROXYPHENYLMALONIC | |
| Z-TYR-OH | ACID | |
| [Z-TYR-OH | PHYDROXYHIPPURYL-HIS- | |
| Z-TYR-OH | LEU-OH | |
| Z-TYR-OH | TRIS ACETATE | |
| Z-TYR-OH | 2-(HYDROXY MERCURI) | |
| Z-TYR-OH | BENZOIC ACID | |
| Z-TYR-OHJ | 3-(TRIFLUOROMETHYL)-3- | |
| 3-METHOXY-L-TYROSINE | HYDROXYBUTYRIC ACID | |
| MONOHYDRATE | 11-HYDROXYPENTADECANOIC | |
| AC-DL-SER-OH | ACID | |
| STATINE | | |
| DNP-HYDROXY-L-PROLINE | 4-HYDROXY-3-PYRIDINE | |
| BOC-HYP(BZL)-OH | CARBOXYLIC ACID | |
| [BOC-HYP(BZL)-OH] | 3,3-BIS(TRIFLUOROMETHYL)- | |
| Z-HYP-OH | 3-HYDROXYPROPIONIC ACID | |
| [Z-HYP-OH] | | |
| AC-HYP-OH | 2-HYDROXY-2-(3- | |
| H-GLY-PRO-HYP-OH | NITROPHENYL)ACETIC ACID | |
| H-GLY-HYP-ALA-OH | | |
| H-PRO-HYP-OH | 11-HYDROXYUNDECANOIC ACID | |
| 7-HYDROXYCOUMARIN-4- | | |
| ACETIC ACID | 3-HYDROXY-2- | |
| L-ARGININIC ACID | TRIFLUOROMETHYLPROPIONIC | |
| H-HYP-GLY-OH | ACID | |
| 2-THIOURACIL-5-CARBOXYLIC | (−)-3BETA-ACETOXY-5- | |
| ACID | ETIENIC ACID | |
| 7-CHLORO-4-HYDROXY-3- | 3-HYDROXYMANDELIC ACID | |
| QUINOLINECARBOXYLIC ACID | 4-CHLOROMANDELIC ACID | |
| | D-LUCIFERIN | |
| AMASTATIN | TRIS-SUCCINATE | |
| N-HEPTYL-HYP-OH | 5-HYDROXYTRYPTAIAINE | |
| N-O-NITROPHENYLSULFENYL- | MALEATE SALT | |
| L-HYDROXYPROLINE | 5-HYDROXYTRYPTAMINE | |
| DI(CYCLOHEXYL)AMMONIUM | OXALATE SALT | |
| | N-ISOPROPYLHYDROXYLAMINE | |
| SALT | | |
| OXALATE | | |
| CHOLINE DIHYDROGEN | | |
| 4-F′LUOROMANDELIC ACID | MONOSODIUM SALT | |
| PHOSPHOENOLPYRUVIC ACID, | PHOSPHOENOLPYRUVATE | |
| MONOPOTASSIUM SALT | MONOSODIUM SALT | |
| | PHOSPHOENOLPYRUVATE | |
| [PHOSPHOENOLPYRUVIC ACID, | MONOSODIUM SALT | |
| MONOPOTASSIUM SALT | PHOSPHOENOLPYRUVATE | |
| | MONOSODIUM SALT | |
| PHOSPHOENOLPYRUVIC | PHOSPHOENOLPYRUVATE | |
| MONOPOTASSIUM SALT | MONOSODIUM SALT | |
| | PHOSPHOENOLPYRUVATE | |
| PHOSPHOENOLPYRUVIC ACID, | | |
| | MONOSODIUM SALT | |
| MONOPOTASSIUM SALT] | PHOSPHOENOLPYRUVATE | |
| 3-CHLORO-4- | MONOSODIUM SALT | |
| HYDROXYMANDELIC ACID | PHOSPHOENOLPYRUVATE | |
| 3-HYDROXY-2-METHYL-4- | MONOSODIUM SALT | |
| QUINOLINECARBOXYLIC ACID | PHOSPHOENOLPYRUVATE | |
| | MONOSODIUM SALT] | |
| PHOSPHOENOLPYRUVATE | BISMUTH SUBGALLATE | |
| MONOSODIUM SALT | 1,2-DIHYDROXY CYCLOBUTANE | |
| [PHOSPHOENOLPYRUVATE | CARBOXYLIC ACID | |
| MONOSODIUM SALT | | |
| PHOSPHOENOLPYRUVATE | 1-CYCLOPENTANOL-1- | |
| MONOSODIUM SALT | CARBOXYLIC ACID | |
| PHOSPHOENOLPYRUVATE | 1-HYDROXY CYCLOHEXANE | |
| MONOSODIUM SALT | CARBOXYLIC ACID | |
| PHOSPHOENOLPYRUVATE | EVERNIC ACID | |
| | 3,5-DIIODO-4′-(4- | |
| MONOSODIUM SALT | | |
| PHOSPHOENOLPYRUVATE | HYDROXYPHENOXY)BENZOIC | |
| MONOSODIUM SALT | ACID | |
| PHOSPHOENOLPYRUVATE | 2-CHLORO-4-HYDROXYBENZOIC | |
| | ACID | |
| MONOSODIUM SALT | | |
| PHOSPHOENOLPYRUVATE | [2-CHLORO-4- | |
| | HYDROXYBENZOIC ACID] | |
| MONOSODIUM SALT | DL-THYRONINE | |
| PHOSPHOENOLPYRUVATE | 3-CHLORO-L-TYROSINE | |
| MONOSODIUM SALT | | |
| PHOSPHOENOLPYRUVATE | | |
| 3,5-DIIODOTHYROPROPIONIC | BETA-HYDROXY-DL-LEUCINE | |
| ACID | | |
| | 2-HYDROXY-3-(2′- | |
| 3-(4-HYDROXY-3- | AMINOPHENYLTHIO)-3-(4″- | |
| NITROPHENYL)PROPANOIC | METHOXYPHENYL)PROPIONIC | |
| ACID | ACID | |
| [3-(4-HXDROXY-3- | | |
| | 3,3,3-TRIFLUOROLACTIC | |
| NITROPHENYL)PROPANOIC | | |
| | ACID | |
| ACID] | 2-FLUORO-6-HYDROXYBENZOIC | |
| HYDROCORTISONE | | |
| | ACID | |
| HEMISUCCINATE | 5-CHLORO-6- | |
| 1,6-DIBROMO-2- | | |
| | HYDROXYNICOTINIC ACID | |
| HYDROXYNAPHTHALENE-3- | 5-CHLOROSULFONYL-2- | |
| CARBOXYLIC ACID | HYDROXYBENZOIC ACID | |
| 2-HYDROXYOCTADECANOIC | 5-CHLOROSULFONYL-2- | |
| ACID | HYDROXYBENZOIC ACID | |
| 2-HYDROXYTETRACOSANOIC | 2-PROPIONYLBENZOIC ACID | |
| ACID | | |
| 6-HYDROXYCAPROIC ACID | 2,5-BIS(2- | |
| 9-(10)- | HYDROXYETHYLAMINO)TEREPHT | |
| HYDROXYPHENYLSTEARIC ACID | HALIC ACID | |
| | BOC-HYP-OH | |
| (+/−)-2-HYDROXYNONANOIC | [BOC-HYP-OH | |
| ACID | BOC-HYP-OH] | |
| 9,10- | 2-HYDROXY-11H- | |
| DIHYDROXYOCTADECANOIC | BENZO(A)CARBAZOLE-3- | |
| ACID | CARBOXYLIC ACID | |
| 4-HYDROXY-8- | PTERIN-7-CARBOXYLIC ACID | |
| (TRIFLUOROMETHYL)-3- | | |
| QUINOLINECARBOXYLIC ACID | TRIETHANOLAMINE STEARATE | |
| 3-(3,5-DINITRO-4- | TRIETHANOLAMINE OLEATE | |
| HYDROXYPHENYL)PROPIONIC | 8-HYDROXYQUINOLINE | |
| ACID | CITRATE | |
| 3-HYDROXY-DL-KYNURENINE | (+/−)-ALPHA-AMINO-3- | |
| | HYDROXY-5- | |
| N-HYDROXY-D-ASPARAGINE | | |
| | METHYLISOXAZOLE-4- | |
| PROPIONIC ACID | TESTOSTERONE BETA-D | |
| HYDROBROMIDE | GLUCURONIDE | |
| 3-HYDROXY-2,4,6- | HYDROCORTISONE-3-(O- | |
| TRIBROMOBENZOIC ACID | CARBOXYMETHYL)OXIME | |
| 4-IODOACETAMIDOSALICYLIC | 4-ANDROSTEN-17BETA-OL-3- | |
| ACID | ONE 3-O- | |
| | CARBOXYMETHYLOXIME | |
| 4-HYDROXYBENZOIC ACID- | 5ALPHA-ANDROSTAN-3BETA- | |
| RING-UL-14C | OL-17BETA-CARBOXYLIC ACID | |
| H-[15N]TYR-OH | | |
| 3,3′,5 | HYOCHOLIC ACID | |
| TRIIODOTHYROPROPIONIC | 5BETA-PREGNANE- | |
| ACID | 3ALPHA,20ALPHA-DIOL | |
| 4′-HYDROXY-3,5,3′-TRI | GLUCURONIDE | |
| IODO PHENOXY-4-PHENYL | ESTRIOL 16ALPHA-(BETA-D- | |
| ACETIC ACID | GLUCURONIDE) | |
| 3,3′,5,5′-TETRAIODOTHYRO- | 3,16-ALPHA- | |
| ACETIC ACID | DIHYDROXYESTRA-1,3,5(10)- | |
| | TRIENE-17-BETA-YL-BETA-D- | |
| 4-AMINO-5-CARBOXY-2- | | |
| | GLUCURONIDE | |
| HYDROXYPYRIMIDINE | | |
| 5-IODOOROTIC ACID | 4-HYDROXY-3- | |
| 17BETA-ESTRADIOL 17- | METHOXYPHENYLPYRUVIC ACID | |
| HEMI SUCCINATE | | |
| CHLORFLURENOL | [4-HYDROXY-3- | |
| ESTRONE 3-HEMISUCCINATE | METHOXYPHENYLPYRUVIC ACID | |
| 5ALPHA-CHOLANIC ACID- | 4-HYDROXY-3- | |
| 3ALPHA-OL-6-ONE | METHOXYPHENYLPYRUVIC | |
| 6-KETOESTRIOL 6-(O- | | |
| | ACID] | |
| CARBOXYMETHYL)OXIME | L-GLUTAMIC ACID GAMMA-(3- | |
| 6-KETOESTRADIOL 6-(O- | | |
| | CARBOXY-4-HYDROXYANILIDE) | |
| CARBOXYMETHYL)OXIME | | |
| PREDNISOLONE 21- | CEFADROXIL | |
| HEMISUCCINATE | D-ALDOSTERONE 21-ACETATE | |
| 11ALPHA- | | |
| HYDROXYPROGESTERONE | D-ALDOSTERONE 21- | |
| HEMISUCCINATE | HEMISUCCINATE | |
| L-BETA-IMIDAZOLELACTIC | HYDROCHLORIDE | |
| | 5-ANDROSTEN-3BETA-OL-17- | |
| ACID | | |
| N-DECYL-HYP-OH | ONE HEMISUCCINATE | |
| (+/−) HOMOCITRIC ACID | CHOLESTERYL HEMISUCCINATE | |
| LACTONE | TRIS SALT | |
| 4-HYDROXY-3-IODO-5-NITRO- | | |
| PHENYLACETIC ACID | CHOLESTERYL HEMISUCCINATE | |
| | MORPHOLINE SALT | |
| 4-HYDROXY-3-IODO-5- | | |
| NITROBENZOIC ACID | TRIZMA BENZOATE | |
| (5)-(+)-HEXAHYDROMANDELIC | TRIS SUCCINATE | |
| | TRIS OXALATE | |
| ACID | | |
| | HYDROXYPYRUVIC ACID | |
| HEXAHYDROMANDELIC ACID | DIMETHYLKETAL PHOSPHATE | |
| (S)-(+)-HEXAHYDROMANDELIC | TRI(CYCLOHEXYLANMONIUM) | |
| ACID] | SALT | |
| | BUFOTENINE MONOOXALATE | |
| (HYDROXYMETHYL)PHENOXYACE | MEVALDIC ACID PRECURSOR | |
| TIC ACID | HEMI(N,N′- | |
| 12(5)-HYDROXY- | | |
| | DIBENZYLETHYLENE- | |
| (5Z,8E,10E)- | | |
| | DIAMMONIUM SALT | |
| HEPTADECATRIENOIC ACID | METARAMINOL BITARTRATE | |
| N-CBZ-(2R,3R)-3-AMINO-2- | | |
| | SALT | |
| HYDROXY-4-PHENYL-BUTYRIC | (2S,3R)-3-AMINO-2- | |
| ACID | HYDROXY-5-METHYLHEXANOIC | |
| [N-CBZ-(2R,3R)-3-AMINO-2- | | |
| | ACID HYDROCHLORIDE | |
| HYDROXY-4-PHENYL-BUTYRIC | | |
| ACID] | (2S,3R)-3-AMINO-2- | |
| N-T-BOC-(2S,3R)-3-AMINO- | HYDROXY-4-(4- | |
| 2-HYDROXY-4-PHENYLBUTYRIC | NITROPHENYL)BUTYRIC ACID | |
| ACID | HYDROCHLORIDE | |
| BESTATIN | N-T-BOC-(2S,3R)-3-AMINO- | |
| HYDROXYZINE PAMOATE | 2-HYDROXY-4-(4- | |
| ANASTATIN HYDROCHLORIDE | NITROPHENYL)BUTYRIC ACID | |
| L-LEUCINE 3-CARBOXY-4- | 3-HYDROXYPROPIONIC ACID | |
| HYDROXYANILIDE | | |
| 3,5-DI-TERT- | PEPSTATIN A | |
| BUTYLSALICYLIC ACID | PEPSTATIN A | |
| 4′-HYDROXY-4- | PEPSTATIN A | |
| BIPHENYLCARBOXYLIC ACID | PEPSTATIN A | |
| | PEPSTATIN A] | |
| BETA-HYDROXYISOVALERIC | 2,3-DIFLUOROMANDELIC ACID | |
| ACID | | |
| 3-HYDROXY-7-METHOXY-2- | 2,4-DIFLUOROMANDELIC ACID | |
| NAPHTHOIC ACID | | |
| GENTISURIC ACID | 2,5-DIFLUOROMANDELIC ACID | |
| 3-HYDROXYMYRISTIC ACID | | |
| 4-(3- | 2,6-DIFLUOROMANDELIC ACID | |
| HYDROXYPHENOXY)BENZOIC | | |
| ACID | 3,4-DIFLUOROMANDELIC ACID | |
| 4-(4- | | |
| | 3,5-DIFLUOROMANDELIC ACID | |
| HYDROXYPHENOXY)BENZOIC | | |
| ACID | | |
| 3-HYDROXY-3-METHYL-N- | 3,5-DIFLUOROMANDELIC ACID | |
| VALERIC ACID | (Z)-2-HYDROXYIMINO-2-(2- | |
| 2,2-DIMETHYL-3- | | |
| | AMINOTHIAZOLE-4-YL) | |
| HYDROXYPROPIONIC ACID | | |
| 6-HYDROXY-2-NAPHTHOIC | ACETIC ACID | |
| | 2-(2-CHLORO-ACETYLAMINO)- | |
| ACID | | |
| [6-HYDROXY-2-NAPHTHOIC | 3-(4-HYDROXY-PHENYL)- | |
| ACID | PROPIONIC ACID | |
| 6-HYDROXY-2-NAPHTHOIC | 3,3′,5-TRIIODO-D- | |
| ACID | THYRONINE | |
| 6-HYDROXY-2-NAPHTHOIC | D-THYROXINE | |
| ACID | DL-THYROXINE | |
| 6-HYDROXY-2-NAPHTHOIC | L-M-TYR | |
| ACID] | DL-3-(3,4- | |
| 3-BROMO-4-HYDROXY-5- | DIHYDROXYPHENYL)ALANINE | |
| METHOXY-PHENYLACETIC ACID | | |
| | D-3,4- | |
| PEPSTATIN A | DIHYDROXYPHENYLALANINE | |
| [PEPSTATIN A | O-PHOSPHO-DL-TYROSINE | |
| PEPSTATIN A | O-PHOSPHO-D-TYROSINE | |
| PEPSTATIN A | L-THYRONINE | |
| D-TYROSINE | D-SACCHARIC ACID | |
| DL-TYROSINE | POTASSIUM SALT | |
| 3,5-DIIODO-DL-TYROSINE | DL-MALIC ACID | |
| 3-METHOXY-DL-TYROSINE | [DL-MALIC ACID | |
| L-HOMOSERINE | DL-MALIC ACID | |
| [L-HOMOSERINE] | DL-MALIC ACID | |
| BOC-NST-OH | DL-MALIC ACID | |
| BOC-STATINE | DL-MALIC ACID | |
| PHALLACIDIN | DL-MALIC ACID] | |
| 17ALPHA- | L-(−)-MALIC ACID | |
| METHYLTESTOSTERONE 3-CO- | [L-(−)-MALIC ACID | |
| CARBOXYMETHYL)OXIME | L-(−)-MALIC ACID | |
| 5(S),6(R)-DIHETE | L-(−)-MALIC ACID | |
| 9(5)-HETE | L-(−)-MALIC ACID | |
| 8(5)-HETE | L-(−)-MALIC ACID | |
| 8(S),15(S)-DIHETE | L-(−)-MALIC ACID | |
| 5(S),15(S)-DIHETE | L-(−)-MALIC ACID | |
| 15(5)-HETE | L-(−)-MALIC ACID | |
| DL-THREONINE | L-(−)-MALIC ACID] | |
| 8-HYDROXYQUINOLINE | L-ALPHA-HYDROXYISOCAPROIC | |
| GLUCURONIDE | ACID | |
| 2-HYDROXY-2-PHENYLACETIC | [L-ALPHA- | |
| ACID | HYDROXYISOCAPROIC ACID | |
| D-CARNITINE HYDROCHLORIDE | L-ALPHA-HYDROXYISOCAPROIC | |
| | ACID | |
| CHENODEOXYCHOLIC ACID | L-ALPHA-HYDROXYISOCAPROIC | |
| ALPHA-METHYL-L-P-TYROSINE | ACID] | |
| | DL-SERINE | |
| L-(+)-TARTARIC ACID | L-SERINE | |
| [L-(+)-TARTARIC ACID | [L-SERINE | |
| L-(+)-TARTARIC ACID | L-SERINE | |
| L-(+)-TARTARIC ACID | L-SERINE | |
| L-(+)-TARTARIC ACID | L-SERINE | |
| L-(+)-TARTARIC ACID | L-SERINE | |
| L-(+)-TARTARIC ACID | L-SERINE | |
| L-(+)-TARTARIC ACID] | L-SERINE | |
| D-SACCHARIC ACID | L-SERINE | |
| POTASSIUM SALT | L-SERINE | |
| L-SERINE | L-HYDROXYPROLINE | |
| L-SERINE | L-HYDROXYPROLINE | |
| L-SERINE] | L-HYDROXYPROLINE] | |
| DL-MANDELIC ACID | L-5-HYDROXYTRYPTOPHAN | |
| [DL-MANDELIC ACID | [L-5-HYDROXYTRYPTOPHAN | |
| DL-MANDELIC ACID] | L-5-HYDROXYTRYPTOPHAN | |
| D-(−)-MANDELIC ACID | L-5-HYDROXYTRYPTOPHAN | |
| [D-(−)-MANDELIC ACID | L-5-HYDROXYTRYPTOPHAN | |
| D-(−)-MANDELIC ACID] | L-5-HYDROXYTRYPTOPHAN | |
| L-(+)-LACTIC ACID | L-5-HYDROXYTRYPTOPHAN] | |
| [L-(+)-LACTIC ACID | ZINCON, MONOSODIUM SALT | |
| L-(+)-LACTIC ACID9 | | |
| L-ALLO-THREONINE | 18-ALPHA-GLYCYRRHETINIC | |
| D-THREONINE | ACID | |
| [D-THREONINE | OLEANOLIC ACID | |
| D-THREONINE | 3,5-DIIODO-L-THYRONINE | |
| D-THREONINE | 2-HYDROXYVALERIC ACID | |
| D-THREONINE | BUTTPARK 18\09-66 | |
| D-THREONINE | 5-HYDROXYINDOLE-3-ACETIC | |
| D-THEEONINE] | ACID DIETHYLAMMONIUM SALT | |
| L-THREONINE | | |
| [L-THREONINE | BOC-(3S,4S)-4-AMINO-3- | |
| L-THREONINE | HYDROXY-5-PHENYL- | |
| L-THREONINE | PENTANOIC ACID | |
| L-THREONINE | FMOC-HYP-OH | |
| L-THREONINE | FMOC-HYP(TBU)-OH | |
| L-THEEONINE | FMOC-STA-OH | |
| L-THEEONINE | 4- | |
| L-THREONINE] | HYDROXYMETHYLPHENYLACETIC | |
| CIS-4-HYDROXY-L-PROLINE | | |
| | ACID | |
| L-HYDROXYPROLINE | 16,16-DIMETHYL | |
| [L-HYDROXYPROLINE | PROSTAGLANDIN A1 | |
| L-HYDROXYPROLINE | 16,16-DIMETHYL | |
| L-HYDROXYPROLINE | PROSTAGLANDIN A2 | |
| L-HYDROXYPROLINE | PROSTAGLANDIN A3 | |
| | PROSTAGLANDIN B2 | |
| L-HYDROXYPROLINE | | |
| L-HYDROXYPROLINE | 13,14-DIHYDRO-15-KETO | |
| L-HYDROXYPROLINE | PROSTAGLANDIN D2 | |
| 8-ISO PROSTAGLANDIN E1 | TRANS-6-LEUKOTRIENE B4 | |
| 11-DEOXY PROSTAGLANDIN E1 | (5S,12S)-DIHYDROXY- | |
| | (6E, 8E, 10E, 14Z)- | |
| 15-KETO PROSTAGLANDIN E1 | EICOSATETRAENOIC ACID | |
| | 6,7-DIHYDRO LEUKOTRIENE | |
| 8-ISO PROSTAGLANDIN E2 | B4 | |
| 15-KETO-PROSTAGLANDIN E2 | 20-CARBOXY-LEUKOTRIENE B4 | |
| PROSTAGLANDIN E3 | 20-HYDROXY LEUKOTRIENE B4 | |
| PROSTAGLANDIN J2 | | |
| DELTA12-PROSTAGLANDIN J2 | 20-TRIFLUORO LEUKOTRIENE | |
| | B4 | |
| CARBOCYCLIC THROMBOXANE | LEUKOTRIENE B5 | |
| A2 | LEUKOTRIENE C5 | |
| 5(S)-HEPE | LEUKOTRIENE D5 | |
| 9(5)-HEPE | PROSTAGLANDIN B2-D4 | |
| 11(S)-HEPE | GLYCOCHOLIC ACID | |
| (+/−)12-HEPE | 2-METHYL-3-(3,4- | |
| 15(S)-HEPE | DIHYDROXYPHENYL)-DL- | |
| (+/−)5-HETE | | |
| 5(S)-HETE | ALANINE | |
| (+/−)8-HETE | DL-GLYCERIC ACID | |
| | DL-BETA-PHENYLLACTIC ACID | |
| 8(R)-HYDROXY | | |
| (5Z,9E,11Z,14Z)- | HYDROXYPHENYLGLYCINE | |
| EICOSATETRAENOIC ACID | L-NORTYR | |
| (+/−)-9-HYDROXY- | O-PHOSPHO-L-SERINE | |
| (5Z,7E,11Z,14Z)- | L-CARNITINE HYDROCHLORIDE | |
| EICOSATETRAENOIC ACID | | |
| (+/−)11-HETE | L-ADRENALINE-D- | |
| (+/−)12-HETE | HYDROGENTARTRATE | |
| 12(R)-HETE | (R)-(−)-3-HYDROXYBUTYRIC | |
| 12-EPILEUKOTRIENE B4 | ACID | |
| (+/−)9-HODE | SHIKIMIC ACID | |
| 9(5)-HODE | (S)-(+)-CITRAMALIC ACID | |
| (+/−)13-HODE | | |
| 13(5)-HODE | (R)-(−)-CITRAMALIC ACID | |
| LIPOXIN A4 | | |
| LIPOXIN B4 | D-ALPHA-HYDROXYISOVALERIC | |
| ACID | (R)-(−)-2-HYDROXY-2- | |
| D-ALPHA-HYDROXYISOVALERIC | PHENYLPROPIONIC ACID | |
| ACID | (S)-(+)-2-HYDROXY-2- | |
| L-ALPHA-HYDROXYISOVALERIC | PHENYLPROPIONIC ACID | |
| ACID | D-LACTIC ACID | |
| CHELIDAMIC ACID | (RS)-ALPHA-ANINO-3- | |
| [CHELIDAMIC ACID | HYDROXY-5-METHYL-4 - | |
| CHELID~MIC ACID] | | |
| ABSCISIC ACID RACEMIC | ISOXAZOLE-PROPIONIC ACID | |
| R-(−)-ISOPROTERENOL(+)- | 7-CHLOROKYNURENIC ACID | |
| BITARTRATE SALT | 7-CHLOROKYNURENIC ACID | |
| 2-AMINO-3-HYDROXYBUTANOIC | 7-CHLOROKYNURENIC ACID | |
| ACID | S(−)-CARBIDOPA | |
| H-DL-SER(3,4- | HYDROXYTACRINE MALEATE | |
| DIHYDROXYPHENYL)-OH | SALT | |
| DESOXYCHOLIC ACID | IBOTENIC ACID | |
| 11-KETOETIOCHOLANOLONE | 11-KETOANDROSTERONE BETA- | |
| GLUCURONIDE SODIUM SALT | D-GLUCURONIDE | |
| | ANDROSTERONE GLUCURONIDE | |
| ETIOCHOLAN-3ALPHA-OL-17- | | |
| ONE GLUCURONIDE | 5-CHOLENIC ACID-3BETA-OL | |
| GLYCODEOXYCHOLIC ACID | | |
| 3-ALPHA-HYDROXY-5-BETA- | 3,4,5-TRIHYDROXYCYCLOHEX- | |
| CHOLAN-24-[N- | 1-ENE-1-CARBOXYLIC ACID | |
| (CARBOXYMETHYL)AMIDE] | | |
| 2-AMINO-3,4,5,6- | THREO-DS(+)ISOCITRIC ACID | |
| TETRAHYDROXYHEXANOIC ACID | MONOPOTASSIUM SALT | |
| CADMIUM HYDROGEN | O-PHOSPHO-L-THREONINE | |
| HYDROXYETHYL | H-D-SER(PO3H2)-OH | |
| | RICINELAIDIC ACID | |
| ETHYLENEDIANINE | | |
| | ERGOTAMINE TARTRATE | |
| TRIACETATE | 5-HYDROXYINDOLE-3-ACETIC | |
| DL-ALLO-THREONINE | | |
| BETA-(2-THIENYL)-DL- | ACID DICYCLOHEXYLAMMONIUM | |
| SERINE | SALT | |
| N-HYDROXY-L-ASPARAGINE | 5-HYDROXY-D-TRYPTOPHAN | |
| (R)-(−)-2-HYDROXY-2- | FUSIDIC ACID | |
| PHENYLPROPIONIC ACID | 5BETA-CHOLANIC ACID- | |
| 3ALPHA-OL-6-ONE | METHYLBENZOIC ACID | |
| NG, NG-DIMETHYL-L- | 3,5,6-TRICHLOROSALTCYLIC | |
| ARGININE DI(P- | ACID | |
| HYDROXYAZOBENZENE-P′- | | |
| | 2-HYDROXY-3- | |
| SULFONATE) | | |
| N-HEXADECYL-L- | ISOPROPYLBENZOIC ACID | |
| | 1,3-DIHYDROXY-9- | |
| HYDROXYPROLINE | | |
| (2S,3R)-3-(BOO-AMINO)-2- | ACRIDINECARBOXYLIC ACID | |
| HYDROXY-5-METHYLHEXANOIC | ABSOISIC ACID | |
| ACID | (S)-2-HYDROXYPENTANEDIOIC | |
| 11BETA- | | |
| | ACID | |
| HYDROXYETIOCHOLANOLONE | | |
| GLUCURONIDE | 5-IODOFERULIC ACID | |
| (2R,3S)-3-AMINO-2- | 4-CHLORO-3,5- | |
| HYDROXY-4-PHENYL-BUTANOTC | DIHYDROXYBENZOIC ACID | |
| ACID | 2,5-DIHYDROXYCINNANIC | |
| EPIBESTATIN HYDROCHLORIDE | ACID | |
| | 2,5-DIHYDROXYCINNAMIC | |
| EPIAMASTATIN HCL | ACID | |
| DL-2-HYDROXY-4- | L-MIMOSINE | |
| (METHYLTHIO) BUTYRIC ACID | (L-MIMOSINE | |
| | L-MIMOSINE] | |
| DL-2-HYDROXY-N-BUTYRIC | NOR-DESOXYCHOLIC ACID | |
| ACID | (D-ARGO,HYP3,D-PHE7)- | |
| [DL-2-HYDROXY-N-BUTYRIC | BRADYKININ | |
| ACID | (D-ARGO,HYP3,BETA-(2- | |
| DL-2-HYDROXY-N-BUTYRIC | THIENYL)-ALA5,8,D-PHE7)- | |
| ACID] | BRADYKININ | |
| CREATINE MONOHYDRATE | BOC-HYP-OH DCHA | |
| DL-TARTARIC ACID | 2- | |
| TITANIUM(IV) BIS(AMMONIUM | (BENZOYLOXYMETHYL)BENZOIC | |
| LACTATO)DIHYDROXIDE | ACID | |
| | D-HOMOSERINE | |
| PODOCARPIC ACID | PROSTAGLANDIN D2 | |
| 2-ISOPROPYLMALIC ACID | PROSTAGLANDIN A1 | |
| L-DOPA (RING-D3) | PROSTAGLANDIN A2 | |
| 2-HYDROXY-6-ISOPROPYL-3- | PROSTAGLANDIN E1 | |
| PROSTAGLANDIN E2 | N-CBZ-GAMMA-T-BUTYL-L- | |
| PROSTAGLANDIN F1ALPHA | GLUTANIC ACID N- | |
| PROSTAGLANDIN F2ALPHA | HYDROXYSUCCINIMIDE ESTER | |
| TRIS SALT | | |
| N-HYDROXYANILINE OXALATE | N-CBZ-BETA-T-BUTYL-L | |
| | ASPARTIC ACID N- | |
| | HYDROXYSUCCINIMIDE ESTER | |
| HYDROXYCYCLOHEXANECARBOXY | | |
| LIC ACID | [N-CBZ-BETA-T-BUTYL-L- | |
| D-BETA-PHENYLLACTIC ACID | ASPARTIC ACID N | |
| | HYDROXYSUCCINIMIDE ESTER] | |
| [D-BETA-PHENYLLACTIC ACID | | |
| | ERYTHRO-9,10- | |
| D-BETA-PHENYLLACTIC ACID | DIHYDROXYSTEARIC ACID | |
| | THREO-9,10- | |
| D-BETA-PHENYLLACTIC ACID | DIHYDROXYSTEARIC ACID | |
| | PYRANTEL PAMOATE | |
| D-BETA-PHENYLLACTIC ACID | FOLIC ACID | |
| | [FOLIC ACID | |
| D-BETA-PHENYLLACTIC ACID | FOLIC ACID | |
| | FOLIC ACID | |
| D-BETA-PHENYLLACTIC ACID | FOLIC ACID | |
| | FOLIC ACID | |
| D-BETA-PHENYLLACTIC ACID | FOLIC ACID | |
| | FOLIC ACID | |
| D-EETA-PHENYLLACTIC ACID] | FOLIC ACID] | |
| | GIBBERELLIC ACID | |
| OCHRATOXIN A | 11ALPHA-HYDROXYESTRADIOL | |
| IFENPRODIL TARTRATE SALT | 11-HEMISUCCINATE | |
| METHYLERGONOVINE MALEATE | 5(R)-HETE | |
| SALT | 11(R)-HYDROXY | |
| (2S,3R)-3-AMINO-2- | (5Z,8Z,12E,14Z)- | |
| HYDROXY-4-(4- | EICOSATETRAENOIC ACID | |
| NITROPHENYL)-BUTANOYL-L- | SUXIBUZONE | |
| | POTASSIUM HYDROGEN D- | |
| LEUCINE HYDROCHLORIDE | | |
| | TARTRATE | |
| | L-TYROSINE-2,3,5,6-D4 | |
| 2-BROMO-4-HYDROXY-5- | N-T-BOC-L-3,4-DIHYDROXY- | |
| METHOXY-PHENYLACETIC ACID | PHENYLALANINE | |
| | DICYCLOHEXYLAMMONIUM SALT | |
| ALA-ARG-GLY-ILE-LYS-GLY | | |
| ILE-ARG-GLY-PHE-SER-GLY | | |
| 2.5 ACOH 4.5 H2O | BESTATIN | |
| | (8R,15S)-DIHYDROXY | |
| PHOSPHORAIVIIDON | (5Z,9E,11Z,13E)- | |
| 2-(4,5,6-TRIHYDROXY-3- | EICOSATETRAENOIC ACID | |
| OXO-3H-XANTHEN-9-YL)- | 2-HYDROXYEICOSANOIC ACID | |
| BENZOIC ACID | | |
| N-(2-HYDROXYETHYL)GLYCINE | 2-HYDROXYDOCOSANOIC ACID | |
| TRIS MALEATE | 2-HYDROXYDECANOIC ACID | |
| [TRIS MALEATE | BETA-HYDROXYPYRUVIC ACID | |
| TRIS MALEATE] | | |
| ERGOMETRINE MALEATE | 9(R)-HETE | |
| 2-METHYL-5- | (+/−)-O-METHOXYMANDELIC | |
| HYDROXYTRYPTAMINE MALEATE | ACID DICYCLOHEXYLAMMONIUM | |
| | SALT | |
| ALPHA-METHYL-5- | | |
| HYDROXYTRYPTAMINE MALEATE | DL-4- | |
| | HYDROXYPHENYLALANINE-1- | |
| 4-(1,8-DIHYDROXY-3,6- | 13C | |
| DISULFO-2-NAPHTHYLAZO)- | L-TYROSINE (1-13C) | |
| SALICYLIC ACID | DL-4- | |
| 1-HYDROXY-2-INDANACETIC | HYDROXYPHENYLALANINE-2- | |
| ACID | 13C | |
| 5,7-DICHLOROKYNURENIC | DL-TYROSINE-BETA-13C | |
| ACID | L-TYROSINE-BETA-13C | |
| LY-53,857 MALEATE | DL-4- | |
| METHYSERGIDE MALEATE | HYDROXYPHENYLALANINE-15N | |
| PPHT-FLUORESCEIN ACETATE | | |
| | 2-(ACETYLAMINO)-3-(4- | |
| | HYDROXY-3,5- | |
| | DIIODOPHENYL)PROPANOIC | |
| | ACID | |
| 8-ISO PROSTAGLANDIN | 4-HEXADECYLOXY-3-(2- | |
| F2ALPHA | (HYDROXYIMINO)- | |
| 125I-BOP | ACETOACETAMIDO)-BENZOIC | |
| 1-BOP | | |
| PROSTAGLANDIN H1 | ACID | |
| | BETA-(HYDROXYAMINO)- | |
| 5(S)-HETE-D8 | | |
| 12(S)-HETE-D8 | HYDROCINNAMIC ACID | |
| 15(S)-HETE-D8 | 2-(N- | |
| THROMBOXANE B2-D4 | DODECYLCARBAMOYLMETHYL)- | |
| 9(R)-RODE | 2-HYDROXYSUCCINIC ACID | |
| 12(R)-HEPE | 2-ETHYL-2-(2- | |
| 13(R)-RODE | | |
| 15(S)-HETRE | HYDROXYETHYL)-GLUTARIC | |
| RICINOLEIC ACID | ACID GAMMA-LACTONE | |
| 2-CHLOROMANDELIC ACID | 2-(2-HYDROXYETHYL)-2- | |
| KETOMALONIC ACID | ISOPENTYLGLUTARIC ACID | |
| MONOHYDRATE | GANMA-LACTONE | |
| 2-HYDROXY-1- | 2-(2-HYDROXYETHYL)-2- | |
| NAPHTHALENEACETIC ACID | PROPYLGLUTARIC ACID | |
| 2-(3,6-DIHYDROXY-9H- | GAMMA-LACTONE | |
| XANTHEN-9-YL)-3,4,5,6- | 3-HYDROXY-2-OXOBUTYRIC | |
| TETRABROMOBENZOIC ACID | ACID 2,4- | |
| 2-(3,6-DIHYDROXY-9H- | DINITROPHENYLHYDRAZONE | |
| XANTHEN-9-YL)-3,4,5,6- | N-(3-AMINO-4- | |
| TETRACHLOROBENZOIC ACID | HYDROXYPHENYLSULFONYL)- | |
| | ANTHRANILIC ACID | |
| 6-(6-HYDROXY-3-OXO-3H- | 3-HYDROXY-4,4,4- | |
| XANTHEN-9-YL)-3- | TRIFLUOROBUTYRIC ACID | |
| CYCLOHEXENE-1-CARBOXYLIC | 3-HYDROXYBUTYL | |
| ACID | TETRACHLOROPHTHALATE | |
| 8-(3-OXO-4,5,6- | 2-(CARBOXYMETHOXY)-3′,5′- | |
| TRIHYDROXY-3H-XANTHEN-9- | DICHLORO-2′-HYDROXY-4′- | |
| YL)-1-NAPHTHOIC ACID | METHYLACETANILIDE | |
| 6-HYDROXY-3-OXO-3H- | | |
| | MALEIC ACID (3- | |
| XANTHENE-9-CARBOXYLIC | | |
| | HYDROXYBENZYLIDENE)HYDRAZ | |
| ACID | | |
| 5-AMINO-3-CHLORO-2,4- | IDE | |
| | 2′-HYDROXY-4′-NITRO-2- | |
| DIHYDROXYBENZOIC ACID | | |
| OCTADECYLSUCCINANILIC | 2-(4-(6-BROMO-1-HYDROXY- | |
| ACID | 2- | |
| ALPHA-CYANO-4-HYDROXY-3- | NAPHTHYLAZO)PHENOXY)ACETI | |
| METHOXYCINNAMIC ACID | | |
| 2-BROMO-4,6-DIMETHYL-3- | C ACID | |
| | 2-CYANO-3-(2- | |
| HYDROXYBENZOIC ACID | | |
| 2-(CARBOXYMETHYLTHIO)- | HYDROXYPHENYL)PROPIONIC | |
| 5,6-DIAMINO-4- | ACID | |
| | 5-BROMO-3,4- | |
| HYDROXYPYRIMIDINE | DIHYDROXYBENZOIC ACID | |
| | 2,6-DIHYDROXY-3-(3- | |
| HYDROXYHEXAHYDROPHTHALANI | (TRIFLUOROMETHYL)PHENYLAZ | |
| LIC ACID | | |
| 5-HYDROXY-2-(1-OCTYNYL)- | O)BENZOIC ACID | |
| | 3,6-DIHYDROXYPHTHALIC | |
| 3-OXO-1-CYCLOPENTENE-1- | ACID | |
| HEPTANOIC ACID | 2- | |
| N-(2- | [[(HEXYLAMINO)CARBONYL]AM | |
| HYDROXYBENZOYL)GLUTAMIC | INO]-3-HYDROXYPROPANOIC | |
| ACID | ACID | |
| 4-(2-HYDROXY-6- | 2-[[2-(HYDROXYMETHYL)-4- | |
| (TRIMETHYLSILYL)-1- | METHOXYPHENYL]THIG]BENZOI | |
| NAPHTHYLAZO)BENZOIC ACID | C ACID | |
| 2-(4-(2-HYDROXY-1- | 7-AMINO-6-HYDROXYIMINO-7- | |
| | OXOHEPTANOIC ACID | |
| NAPHTHYLAZO)PHENOXY)ACETI | 2,3-DICHLORO-4- | |
| C ACID | | |
| 2-(4-HYDROXYPHENYL)-6- | HYDROXYIMINOBUT-2-ENOIC | |
| METHOXY-4 | ACID | |
| | 7-HYDROXY-4-OXO-8-PROPYL- | |
| QUINOLINECARBOXYLIC ACID | 4H-CHROMENE-2-CARBOXYLIC | |
| 4-BROMO-3-HYDROXY- | ACID | |
| NAPHTHALENE-2-CARBOXYLIC | 1-ETHYL-6-HYDROXY-4-OXO- | |
| ACID | 1,4-DIHYDROQUINOLINE-2- | |
| 8-HYDROXYQUINOLINE-4- | CARBOXYLIC ACID | |
| CARBOXYLIC ACID | HYDROBROMIDE | |
| 2′-HYDROXY-2- | | |
| BIPHENYLCARBOXYLIC ACID | | |
| 2-(3-HYDROXY-5-METHOXY-2- | DI-(METHOXYCARBONYL)BICYCL | |
| PROPYLPHENYL)ACETIC ACID | O[3.3.1]NONA-2,6-DIENE- | |
| | 1,5-DICARBOXYL | |
| 3-HYDROXY-6- | 4-(4-ACETYL-3-HYDROXY-2- | |
| PROPYLPHTHALIC ACID | PROPYLPHENOXY)ISOPHTHALIC | |
| 4-(2-HYDROXY-5- | | |
| | ACID | |
| PROPYLPHENYL)-4- | | |
| OXOBUTANOIC ACID | 2-HYDROXY-4- | |
| 6-HYDROXY-4-OXO-5-(2- | BENZYLOXYCARBONYLAMINO | |
| PHENYLDIAZ-1-ENYL)-4H- | BUTANIC ACID | |
| CHROMENE-2-CARBOXYLIC | 2-(1,1-DIOXO-1LAMBDA-6- | |
| ACID | ,4-THIAZINAN-4-YL)-3-(4- | |
| 5-HYDROXY-4-OXO-4- | HYDROXYPHENYL)PROPANOIC | |
| CHROMENE-2-CARBOXYLIC | ACID | |
| ACID | 2-(1,1-DIOXO-1LAMBDA-6- | |
| 2-[(4- | | |
| HYDROXYPHENYL)THIO]BUT-2- | ,4-THIAZINAN-4-YL)-3-(4- | |
| ENEDIOIC ACID | HYDROXYPHENYL)PROPANOIC | |
| 6- | ACID | |
| HYDROXYBICYCLO[2.2.1]HEPT | 2-(1,1-DIOXO-1LAMBDA-6- | |
| ANE-2-CARBOXYLIC ACID | ,4-THIAZINAN-4-YL)-3-(4- | |
| 5 | HYDROXYPHENYL)PROPANOIC | |
| HYDROXYBICYCLO[2.2.1]HEPT | ACID | |
| ANE-2-CARBOXYLIC ACID | 2-HYDROXY-2- | |
| | PHENYLBUTANOIC ACID | |
| 3-HYDROXY-2-[[(4 | 4-(2-HYDROXY-3,4- | |
| METHYLPHENYL)SULFONYLIAMI | DIMETHYLPHENYL)-4- | |
| NO]PROPANOIC ACID | OXOBUTANOIC ACID | |
| 6- | 3-(3-AMINO-4- | |
| HYDROXYBICYCLO[2.2.2]OCTA | HYDROXYPHENYL)PROPANOIC | |
| NE-2-CARBOXYLIC ACID | ACID | |
| 6- | 2-CHLORO-3- | |
| HYDROXYBICYCLO[2.2.2]OCTA | HYDROXYPENTANOIC ACID | |
| NE-2-CARBOXYLIC ACID | | |
| 2,6-DIHYDROXY-3,7- | | |
| 2,3-DICHLORO-4-[2-[3-(4- | 5(6)-CARBOXYTETRAMETHYL- | |
| HYDROXYPHENYL)PROPANOYL]H | RHODAMINE N-HYDROXY | |
| YDRAZONO]BUT-2-ENOIC ACID | SUCCINIMIDE ESTER | |
| | DEOXYCORTICOSTERONE 21- | |
| 3-([2-[3-(4- | HEMISUCCINATE | |
| HYDROXYPHENYL)PROPANOYL]H | DL-BETA-HYDROXYCAPRIC | |
| YDRAZINO]CARBONYL)BICYCLO | ACID | |
| | DL-BETA-HYDROXYCAPRYLIC | |
| [2.2.1]HEPT-5-ENE- | | |
| | ACID | |
| 4-(4-HYDROXYANILINO)-4- | DL-BETA-HYDROXYLAURIC | |
| OXOBUTANOIC ACID | ACID | |
| 2,6-DIHYDROXY-4- | FLUO 3 AMMONIUM SALT | |
| METHYLNICOTINIC ACID | FUMONISIN B1 | |
| 6-BROMO-2- | ALPHA-ETHYL-3-HYDROXY- | |
| HYDROXYQUINQLINE-4- | 2,4,6- | |
| CARBOXYLIC ACID | TRIIODOHYDROCINNAMIC ACID | |
| 2-[2-[4-(TERT- | | |
| BUTYL)PHENYL]-4-HYDROXY- | LAVENDUSTIN B | |
| | LEUHISTIN | |
| 1,3-THIAZOL-5-YLIACETIC | (R)-ALPHA-HYDROXYMETHYL | |
| ACID | TYROSINE | |
| 2-[2-(4-CHLOROPHENYL)-4- | (5)-ALPHA- | |
| HYDROXY-1,3-THIAZOL-5- | (HYDROXYMETHYL)-TYROSINE | |
| YLIACETIC ACID | | |
| 3-HYDROXYASPARTIC ACID | 17-ALPHA, 21-DIHYDROXY- | |
| 2,3-DIHYDROXYQUINOXALINE- | 11,20-DIOXO-5-BETA- | |
| 6-CARBOXYLIC ACID | PREGNAN-3-ALPHA-YL-BETA- | |
| 2-(4- | D-GLUCURONIDE | |
| | 3- | |
| HYDROXYPHENYL)PROPIONIC | | |
| | (CARBOXYMETHYLAMINOMETHYL | |
| ACID | | |
| (+/−)-ARTERENOL | )-4-HYDROXYBENZOIC ACID | |
| BITARTRATE SALT | 2-(4-DIETHYLAMINO-2- | |
| 12(S)-HEPE | | |
| 2,5-DIHYDROXYTEREPHTHALIC | HYDROXYBENZOYL)BENZOIC | |
| | ACID | |
| ACID | 2-[4-(DIBUTYLAMINO)-2- | |
| | HYDROXYBENZOYL]BENZOIC | |
| ACID | 2,3-DINOR-6-KETO- | |
| 2′-HYDROXY-5′- | PROSTAGLANDIN F1ALPHA | |
| METHYLNALEANILIC ACID | 6-KETO-PROSTAGLANDIN | |
| 12-OXOCHENODEOXYCHOLIC | F1ALPHA | |
| ACID | PROSTAGLANDIN F1BETA | |
| 4-HYDROXYMETHYL-3- | 11BETA-PROSTAGLANDIN | |
| METHOXY-PHENOXYACETIC | F1BETA | |
| ACID | 13,14-DIHYDRO | |
| 17-PHENYL TRINOR-13,14- | PROSTAGLANDIN F1ALPHA | |
| DIHYDRO PROSTAGLANDIN A2 | 13,14-DIHYDRO-15-KETO | |
| | PROSTAGLANDIN F1ALPHA | |
| 11BETA-PROSTAGLANDIN E1 | 15-KETO PROSTAGLANDIN | |
| | F1ALPHA | |
| 13,14-DIHYDRO- | 6,15-DIKETO-13,14-DIHYDRO | |
| PROSTAGLANDIN E1 | PROSTAGLANDIN F1ALPHA | |
| 13,14-DIHYDRO-15(R)- | | |
| PROSTAGLANDIN E1 | 19(R)HYDROXY | |
| 13,14-DIHYDRO-15-KETO | PROSTAGLANDIN F1ALPHA | |
| PROSTAGLANDIN E1 | PROSTAGLANDIN F2ALPHA | |
| 15(S)-15-METHYL | 5-TRANS PROSTAGLANDIN | |
| PROSTAGLANDIN E1 | F2ALPHA | |
| 19(R)-HYDROXY | 5-TRANS PROSTAGLANDIN | |
| PROSTAGLANDIN E1 | F2ALPHA (TROMETHAMINE | |
| 5-TRANS PROSTAGLANDIN E2 | SALT) | |
| | 9BETA,11ALPHA-PGF2 | |
| 9-DEOXY-9-METHYLENE | PROSTAGLANDIN F2BETA | |
| PROSTAGLANDIN E2 | (TROMETHAMINE SALT) | |
| 11BETA-PROSTAGLANDIN E2 | 16,16-DIMETHYL | |
| | PROSTAGLANDIN F2BETA | |
| 11-DEOXY-16,16-DIMETHYL | 11-DEOXY PROSTAGLANDIN | |
| PROSTAGLANDIN E2 | F2BETA | |
| | 13,14-DIHYDRO-15-KETO | |
| 13,14-DIHYDRO-15-KETO | | |
| | PROSTAGLANDIN F2ALPHA | |
| PROSTAGLANDIN E2 | 15-CYCLOHEXYL PENTANOR | |
| 15(R)-PROSTAGLANDIN E2 | | |
| | PROSTAGLANDIN F2ALPHA | |
| 17-PHENYL-TRINOR- | 15-KETO-PROSTAGLANDIN | |
| PROSTAGLANDIN E2 | F2ALPHA | |
| 19(R)-HYDROXY- | 15(R)-15-METHYL | |
| PROSTAGLANDIN E2 | | |
| PROSTAGLANDIN F2ALPHA | ALEURITIC ACID | |
| 15(R)-PROSTAGLANDIN | LEUKOTRIENE B4 | |
| F2ALPHA | CHOLESTERYL HEMISUCCINATE | |
| LATANOPROST | | |
| 16,16-DIMETHYL | O-PHOSPHO-DL-THREONINE | |
| PROSTAGLANDIN F2ALPHA | ARPHAMENINE B | |
| 16-PHENOXY TETRANOR | 6-KETO PROSTAGLANDIN E1 | |
| PROSTAGLANDIN F2ALPHA | | |
| 16-PHENYL TETRANOR | PINANE THROMBOXANE A2 | |
| PROSTAGLANDIN F2ALPHA | CHORISMIC ACID | |
| 17-PHENYL TRINOR | 11-NOR-DELTAB | |
| PROSTAGLANDIN F2ALPHA | TETRAHYDROCANNABINOL-9- | |
| 19(R)-HYDROXY- | CARBOXYLIC ACID | |
| PROSTAGLANDIN F2ALPHA | 11BETA-PROSTAGLANDIN | |
| PROSTAGLANDIN F3ALPHA | F2ALPHA | |
| 6BETA-PROSTAGLANDIN I1 | 3-HYDROXY-3- | |
| 10,11-DIHYDRO LEUKOTRIENE | | |
| | PHENYLPROPYLHYDROXYLAMINE | |
| B4 | | |
| 18-CARBOXY DINOR | HEMIOXALATE | |
| | (2S,3S)-2-HYDROXY-3- | |
| LEUKOTRIENE B4 | | |
| N-ACETYL-LEUKOTRIENE E4 | METHYLPENTANOIC ACID | |
| | (S)-(+)-3-HYDROXYBUTYRIC | |
| N-ACETYL-16-CARBOXY | ACID | |
| | 7-BROMO-3-HYDROXY-2- | |
| TETRANOR LEUKOTRIENE E3 | | |
| | NAPHTHOIC ACID | |
| N-ACETYL-18-CARBOXY DINOR | | |
| LEUKOTRIENE E4 | HYDROXYMETHYLPHENOXY)PROP | |
| N-ACETYL-20-CARBOXY | IONIC ACID | |
| LEUKOTRIENE E4 | 3-(4- | |
| 20,20,20- | HYDROXYMETHYLPHENOXY)PROP | |
| TRIFLUOROLEUKOTRIENE E4 | IONIC ACID | |
| PROSTAGLANDIN A2-D4 | [(HYDROXYIMINO)METHYL]-6- | |
| PROSTAGLANDIN E2-D4 | METHOXYPHENOXY]ACETIC | |
| 6-KETO PROSTAGLANDIN | | |
| | ACID | |
| F1ALPHA-D4 | | |
| PROSTAGLANDIN F2ALPHA-D4 | 6-HYDROXY-5-(2- | |
| | HYDROXYBENZOYL)-2- | |
| 8(S)-HEPE | (TRIFLUOROMETHYL)NICOTINI | |
| C ACID | (3,5-DICARBOXY-PHENYL)-2- | |
| 4-HYDROXY-1-(4 | NAPHTHAMIDE | |
| METHYLPHENYL)-6-OXO-1,6- | (1R,2S)-1-CARBOXY-1,2- | |
| DIHYDRO-3- | DIHYDROXYCYCLOHEXA-3,5- | |
| PYRIDAZINECARBOXYLIC ACID | DIENE | |
| | 2-HYDROXY-3-BUTENOIC ACID | |
| 1-(2,4-DICHLOROPHENYL)-4- | | |
| HYDROXY-6-OXO-1,6- | (2R,3R)-1-CARBOXY-2,3- | |
| DIHYDRO-3- | DIHYDROXY-4- | |
| PYRIDAZINECARBOXYLIC ACID | METHYLCYCLOHEXA-3,5-DIENE | |
| 4-HYDROXY-6-OXO-1-[3- | (2R,3R)-1-CARBOXY-4,5- | |
| (TRIFLUOROMETHYL)PHENYL]- | DICHLORO-2,3- | |
| 1,6-DIHYDRO-3-PYRIDAZINE | DIHYDROXYCYCLOHEXA-4,6- | |
| CARBOXYLI | | |
| 4-HYDROXY-6-OXO-1-PHENYL- | DIENE | |
| | (2R,3R)-1-CARBOXY-4- | |
| 1,6-DIHYDRO-3- | CHLORO-2,3- | |
| PYRIDAZINECARBOXYLIC ACID | DIHYDROXYCYCLOHEXA-4,6- | |
| 1-(3-CHLOROPHENYL)-4- | DIENE | |
| HYDROXY-6-OXO-1,6- | (2R,3R)-1-CARBOXY-4-IODO- | |
| DIHYDRO-3- | 2,3-DIHYDROXYCYCLOHEXA- | |
| PYRIDAZINECARBOXYLIC ACID | 4,6-DIENE | |
| 1,2-DICARBOXY-CIS-4,5- | (2R,3R)-4-BROMO-1- | |
| DIHYDROXYCYCLOHEXA-2,6- | CARBOXY-2,3- | |
| DIENE | DIHYDROXYCYCLOHEXA-4,6- | |
| (1-ADAMANTANEACETYL-D- | DIENE | |
| ARGO,HYP3,BETA-(2- | (2R,3S)-(−)-2-AMINO-3- | |
| THIENYL)-ALA5,8,D-PHE7)- | HYDROXY-4-METHYLPENTANOIC | |
| BRADYKININ | ACID | |
| (CARBOXY- | (2R,3S)-1-CARBOXY-4- | |
| HYDROXYPHENYLAZO-OH- | TRIFLUOROMETHYL-2,3- | |
| NAPHTHAMIDO)-(ME- | DIHYDROXYCYCLOHEXA-4,6- | |
| OCTADECYLAMINO)BENZENESUL | DIENE | |
| FONICACID | (2R,3S)-1-CARBOXY-5-IODO- | |
| 1-HYDROXY-N-OCTADECYL-N- | 4-METHYL-2,3- | |
| DIHYDROXYCYCLOHEXA-4,6- | L-TYROSINE (PHENOL-4-13C) | |
| DIENE | | |
| (2S,3R)-2-AMINO-3- | L-TYROSINE (RING-13C6) | |
| HYDROXY-4-METHYLPENTANOIC | L-4-HYDROXYPHENYL-3,5-D2- | |
| ACID | ALANINE | |
| (2S,3S),(2S,3R)-2-AMINO- | L-TYROSINE-13C9 | |
| 3-HYDROXY-4- | L-TYROSINE (U-13C9,15N) | |
| METHYLPENTANOIC ACID | (R)-(−)-2-AMINO-2-METHYL- | |
| 3-(2,4- | | |
| | 3-HYDROXYPROPANOIC ACID | |
| DIHYDROXYPHENYL)PROPIONIC | | |
| ACID | (R)-(−)-2-AMINO-3- | |
| 3-FLUORO-4-HYDROXYBENZOIC | HYDROXY-3-METHYLBUTANOIC | |
| ACID | | |
| 4,(2- | ACID | |
| | (R)-3-HYDROXY-3- | |
| HYDROXYETHOXY)SALICYLIC | PHENYLPROPANOIC ACID | |
| ACID | (R,S)-′2-AMINO-3-HYDROXY- | |
| ACETYLSALICYLSALICYLIC | 3-METHYLBUTANOIC ACID | |
| ACID | | |
| CIS-2-HYDROXY-1- | (S)-(+)-2-AMINO-2-METHYL- | |
| CYCLOPENTANECARBOXYLIC | 3-HYDROXYPROPANOIC ACID | |
| ACID | | |
| CIS-3-HYDROXY-DL-PROLINE | (5)-3-HYDROXY-3- | |
| | PHENYLPROPANOIC ACID | |
| DL-4-HYDROXYPHENYL-D4- | VANILLIC ACID (CARBOXYL- | |
| ALANINE-2,3,3-D3 | 13C) | |
| ISOVANILLIC ACID (RING- | ALIZARIN COMPLEXONE | |
| 13C6) | DIHYDRATE | |
| L-7-HYDROXY-1,2,3,4- | 5-BROMO-2,4- | |
| TETRAHYDROISOQUINOLINE-3- | DIHYDROXYBENZOIC ACID | |
| CARBOXYLIC ACID | MONOHYDRATE | |
| L-DOPA (RING-13C6) | 2,4,6-TRIHYDROXYBENZOIC | |
| L-MALIC ACID NA-SALT | ACID MONOHYDRATE | |
| L-TYROSINE-CARBOXY-13C | | |
| L-4-HYDROXYPHENYLALANINE- | GALLIC ACID MONOHYDRATE | |
| 3,3-D2 | | |
| | 3,5-DIHYDROXY-4- | |
| | METHYLBENZOIC ACID | |
| HEMIHYDRATE | 5-SULFOSALICYLIC ACID | |
| | 3-CHLORO-4-HYDROXYBENZOIC | |
| | DIHYDRATE | |
| ACID HEMIHYDRATE | 5-SULFOSALICYLIC ACID | |
| 2,2-DIHYDROXY-3,3,3 | DIHYDRATE | |
| | 5-SULFOSALICYLIC ACID | |
| TRICHLOROPROPIONIC ACID | | |
| | DIHYDRATE | |
| (−)-3-(3,4- | 5-SULFOSALICYLIC ACID | |
| DIHYDROXYPHENYL)-2- | DIHYDRATE | |
| | 5-SULFOSALICYLIC ACID | |
| METHYL-L-ALANINE | | |
| | DIHYDRATE | |
| SESQUIHYDRATE | | |
| 4-HYDROXYMANDELIC ACID | 5-SULFOSALICYLIC ACID | |
| MONOHYDRATE | DIHYDRATE] | |
| DIHYDROXYFUMARIC ACID | CITRIC ACID MONOHYDRATE | |
| HYDRATE | | |
| DIHYDROXYFUMARIC ACID | [CITRIC ACID MONOHYDRATE | |
| DIHYDRATE | | |
| DL-ATROLACTIC ACID | CITRIC ACID MONOHYDRATE | |
| HEMIHYDRATE | | |
| DL-THREO-3-PHENYLSERINE | CITRIC ACID MONOHYDRATE | |
| HYDRATE | | |
| URACIL-5-CARBOXYLIC ACID | CITRIC ACID MONOHYDRATE | |
| MONOHYDRATE | CITRIC ACID MONOHYDRATE | |
| OROTIC ACID MONOHYDRATE | | |
| 4-HYDROXYQUINOLINE-2- | CITRIC ACID MONOHYDRATE | |
| CARBOXYLIC ACID HYDRATE | CITRIC ACID MONOHYDRATE | |
| 5-SULFOSALICYLIC ACID | | |
| | CITRIC ACID MONOHYDRATE | |
| DIHYDRATE | | |
| [5-SULFOSALICYLIC ACID | | |
| | CITRIC ACID MONOHYDRATE | |
| DIHYDRATE | | |
| 5-SULFOSALICYLIC ACID | CITRIC ACID MONOHYDRATE | |
| DIHYDRATE | | |
| 5-SULFOSALICYLIC ACID | CITRIC ACID MONOHYDRATE | |
| DIHYDRATE | | |
| 5-SULFOSALICYLIC ACID | CITRIC ACID MONOHYDRATE | |
| DIHYDRATE | | |
| CITRIC ACID MONOHYDRATE | PROSTAGLANDIN E2 | |
| | 2,3-DINOR THROMBOXANE B2 | |
| | CITRIC ACID MONOHYDRATE] | |
| | DL-TARTARIC ACID HYDRATE | |
| | 3,5-DIIODO-L-TYROSINE | |
| DIHYDRATE | TRANS-6-LEUKOTRIENE B4- | |
| DL-ALPHA-METHYL-M- | 1,2-13C2 | |
| TYROSINE | LEUKOTRIENE B4-1,2-13C2 | |
| (R)-(−)-NOREPINEPHRINE L | | |
| BITARTRATE MONOHYDRATE | DEXTRORPHAN D-TARTRATE | |
| | [DEXTRORPHAN D -TARTEATE | |
| 3-METHOXY-L-TYROSINE | DEXTRORPHAN D-TARTRATE] | |
| | LEVORPHANOL TARTRATE SALT | |
| HYDRATE | | |
| HEPTYL-HYP-OH H2O | | |
| N-[(2S,3R)-3-AMINO-2- | MUG | |
| | 4-CARBOXYPHENYLBORONIC | |
| HYDROXY-5- | | |
| | ACID | |
| METHYLHEXANOYL]-L-VALYL- | [4-CARBOXYPHENYLBORONIC | |
| L-VALYL-L-ASPARTIC ACID H | | |
| | ACID | |
| 5-FLUOROOROTIC ACID | 4-CARBOXYPHENYLBORONIC | |
| MONOHYDRATE | ACID] | |
| MESO-TARTARIC ACID | FMOC-TRANS-3-HYDROXY-PRO- | |
| MONOHYDRATE | OH | |
| (DL-3-HYDROXY-3- | 3-FLUORO-2-HYDROXYBENZOIC | |
| METHYLGLUTARYL)COENZYME | ACID | |
| A DISODIUM SALT | 4-FLUORO-2-HYDROXYBENZOIC | |
| TRIHYDRATE | ACID | |
| IBOTENIC ACID MONOHYDRATE | Z-HYP(TBU)-OH | |
| | H-HYP(TBU)-OH | |
| (R)-(−)-2-HYDROXY-2- | CYCLOHEXYLSTATINE | |
| | AHPPA | |
| PHENYLPROPIONIC ACID | | |
| GLYCODEOXYCHOLIC ACID | 4-(4-HYDROXYMETHYL-3- | |
| MONOHYDRATE | METHOXYPHENOXY)-BUTYRIC | |
| LEUKOTRIENE C4 | ACID | |
| LEUKOTRIENE E4 | 4-(4-HYDROXYMETHYL-3- | |
| P-SYMPATOL TARTRATE | METHOXYPHENOXY)-BUTYRIC | |
| LEUKOTRIENE F4 | ACID | |
| 16,16-DIMETHYL | (RS)-4-BROMO-HOMO- | |
| IBOTENIC ACID | EXANOIC ACID | |
| NIGROSINE, WS | 3,5-DIBROMO-2,4- | |
| LAVENDUSTIN A | DIHYDROXYBENZOIC ACID | |
| 4-CHLORO-3-HYDROXYBENZOIC | 2,4-DIHYDROXY-3- | |
| ACID | METHYLBENZOIC ACID | |
| 4-CHLORO-3-HYDROXYBENZOIC | 2′-(HYDROXYMETHYL)-2- | |
| ACID | BIPHENYLCARBOXYLIC ACID | |
| 5-(2,5- | | |
| DIHYDROXYBENZYLAMINO)-2- | 4,5-DIHYDROXY-2- | |
| HYDROXYBENZOIC ACID | METHYLBENZOIC ACID | |
| 9-HYDROXYDECANOIC ACID | 5-HYDROXY-2-METHYL-4- | |
| (−)-3-HYDROXYBUTYRIC | NITROBENZOIC ACID | |
| ACID, QUININE SALT | N-(2,4-DICHLOROPHENYL)- | |
| 3,3-DIMETHYL-3-(1,1- | 2,3-DIHYDROXYSUCCINAMIC | |
| DIMETHYL-2- | ACID | |
| HYDROXYETHYLAMINO)PROPION | 4′-HYDROXYGLUTARANILIC | |
| IC ACID | | |
| N-(2-HYDROXYETHYL)-BETA- | ACID | |
| | [4′-HYDROXYGLUTARANILIC | |
| ALANINE | | |
| 5-HYDROXYDICAMBA | ACID] | |
| 3,6-DICHLORO-2-HYDROXY | 4′(2 | |
| | HYDROXYETHYL)GLUTARANILIC | |
| BENZOIC ACID | | |
| BOC-ACHPA-OH | ACID | |
| F′MOC-AHPPA | [4′-(2- | |
| FMOC-ACHPA | HYDROXYETHYL)GLUTARANILIC | |
| 3,5-DI-TERT-BUTYL-4- | ACID] | |
| HYDROXYPHENYLACETIC ACID | N-(1,1- | |
| | BIS(HYDROXYMETHYL)ETHYL)- | |
| 1-BENZYL-4-HYDROXY-3- | | |
| | 3,4,5,6- | |
| PIPERIDINEMETHANOL | | |
| | TETRACHLOROPHTHALAMIC | |
| FUMARATE | | |
| DL-2-(4-HYDROXYPHENYL)-2- | ACID | |
| | N-(2-HYDROXYBENZOYL)-L- | |
| PHENYLACETIC ACID | | |
| 5-(3- | ASPARTIC ACID | |
| | 3′-HYDROXYPHTHALANILIC | |
| HYDROXYPHENYL)VALERIC | | |
| | ACID | |
| ACID | 1-(4-CARBOXY-3- | |
| 6-(2- | | |
| | HYDROXYPHENYL)-3-(3- | |
| HYDROXYBENZYLIDENEAMINO)H | | |
| FLUOROPHENYL)UREA | HEXACHLORO-5-(6-HYDROXY- | |
| N-(2-HYDROXY-3-METHOXY-5- | 3-OXO-3H-XANTHEN-9-YL)- | |
| NITROBENZYLIDENE)HISTIDIN | TRICYCLOUNDEC-9-ENE-4- | |
| E | COOH | |
| 5-(HYDROXYIMINO)AZELAIC | 2- | |
| ACID | HYDROXYAMINOCYCLOHEXANONE | |
| 5,5-DIMETHYL-4-HYDROXY-4- | OXIME ACETATE | |
| | 1,3- | |
| (4-PHENOXYPHENYL)-2- | DIHYDROXYPERHYDROBENZIMID | |
| HEXYNOIC ACID | | |
| N-(2-HYDROXY-3-METHOXY-5- | AZOL-2-CARBOXYLIC ACID | |
| NITROBENZYLIDENE)-BETA- | ETHANOLATE | |
| | 2-(HYDROXYAMINO)-2- | |
| ALANINE | | |
| METHYL CALCEIN BLUE | METHYLCYCLOHEXAN-1-ONE | |
| 3-MORPHOLINOMETHYL-4- | OXIME ACETATE | |
| | 8-HYDROXYQUINOLINE-2- | |
| HYDROXY-BENZOIC ACID- | | |
| | CARBOXYLIC ACID | |
| DIHYDRATE | [-HYDROXYQUINOLINE-2 - | |
| N,N′-DI(2- | | |
| | CARBOXYLIC ACID | |
| HYDROXYBENZYL)ETHYLENEDIA | 8-HYDROXYQUINOLINE-2- | |
| MINE-N,N′-DIACETIC ACID | | |
| | CARBOXYLIC ACID | |
| DIHYDROCHLORIDE DIHYD | 8-HYDROXYQUINOLINE-2- | |
| 2,3,5,6-TETRAFLUORO-4- | CARBOXYLIC ACID] | |
| HYDROXYBENZOIC ACID | D-7-HYDROXY-1,2,3,4- | |
| HYDRATE | TETRAHYDROISOQUINOLINE-3- | |
| (2-HYDROXYNORBORNAN-2- | CARBOXYLIC ACID | |
| YL)-PHENYLACETIC ACID | FMOC-TYR(3-NO2)-OH | |
| 5-HYDROXY-2,3- | [FMOC-TYR(3-NO2)-OH] | |
| NORBORNANEDICARBOXYLIC | (+/−)-5-HEPE | |
| | 20-HYDROXY- | |
| ACID GAMMA-LACTONE | | |
| 3-(3,6-DIHYDROXY-9H- | (5Z,8Z,11Z,14Z)- | |
| XANTHEN-9-YL)-HEXACHLORO- | EICOSATETRAENOIC ACID | |
| | 5-HYDROXY-L-TRYPTOPHAN | |
| 5-NORBORNENE-2-CARBOXYLIC | | |
| | HYDRATE | |
| ACID | DL-BETA-HYDROXYPALMITIC | |
| 3-HYDROXY-1- | ACID | |
| ADAMANTANEACETIC ACID | 2-(2- | |
| HYDROXYPHENYL)SUCCINIC | 4-AMINO-3-BROMO-5-CYANO- | |
| ACID | 2-HYDROXYBENZOIC ACID | |
| 3-[(2-HYDROXY-1,1- | | |
| DIMETHYLETHYL)AMINO]THIOP | 2- | |
| HENE-2-CARBOXYLIC ACID | HYDROXYBICYCLO[3.2.1]OCTA | |
| | NE-6-CARBOXYLIC ACID | |
| 2-[(1- | | |
| HYDROXYCYCLOPENTYL)THIO]A | 2- | |
| CETIC ACID | HYDROXYBICYCLO[2.2.1]HEPT | |
| 2-[[(17-HYDROXY-10,13,17- | ANE-7-CARBOXYLIC ACID | |
| TRIMETHYL | | |
| | 3-(1,3-BENZOTHIAZOL-2- | |
| 1,2,6,7,8,9,10,11,12,13,1 | | |
| YL)-2- | | |
| 4,15,16,17-TETRADECA | HYDROXYIMINOPROPANOIC | |
| 2-(1,2,3,4- | ACID | |
| TETRAHYDROXYBUTYL)-1,3- | (R)-(+)-6-HYDROXY- | |
| THIAZOLANE-4-CARBOXYLIC | 2,5,7,8- | |
| ACID | TETRAMETHYLCHROMAN-2- | |
| 2-(1,2,3,4- | CARBOXYLIC ACID | |
| TETRAHYDROXYBUTYL)-1,3- | (S)-(−)-6-HYDROXY- | |
| THIAZOLANE-4-CARBOXYLIC | 2,5,7,8- | |
| ACID | TETRAMETHYLCHROMAN-2- | |
| 2-(3-CHLORO-4- | CARBOXYLIC ACID | |
| HYDROXYPHENYL)-5,5- | 2-HYDROXY-2- | |
| DIMETHYL-1,3-THIAZOLANE- | TRIFLUOROMETHYL-HEXADEC | |
| 4-CARBOXYLIC ACID | 4-ENOIC ACID | |
| | 2-HYDROXY-2- | |
| 5-HYDROXYBENZENE-1,2,4- | | |
| | TRI FLUOROMETHYL-EICOS-4- | |
| TRICARBOXYLIC ACID | | |
| ASINEX-REAG BAS 0873810 | ENOIC ACID | |
| | 2-HYDROXY-2- | |
| 2-(3,5-DIBROMO-4- | TRIFLUOROMETHYL-DEC-4- | |
| HYDROXYPHENYL)-5,5- | ENOIC ACID | |
| | 2-HYDROXY-2- | |
| DIMETHYL-1,3-THIAZOLANE- | | |
| | TRIFLUOROMETHYL-DOCOS-4- | |
| 4-CARBOXYLIC ACID | | |
| | ENOIC ACID | |
| 4-(3-(3,5-DI-TERT-BUTYL- | ((((DIHYDROXY | |
| 4-HYDROXY-PHENYL)-3-OXO- | (GLUCOPYRANOSYL)-PH)-OXO- | |
| PROPENYL)-BENZOIC ACID | PR)-HO-PHENYLCARBAMOYL)- | |
| HYDROXY-(2,3,5,6- | MEO)-ACETIC ACID | |
| TETRAMETHYL-PHENYL)- | (1-(DIHYDROXY | |
| ACETIC ACID | (GLUCOPYRANOSYL)-PH)-3- | |
| (4-HYDROXY-3-METHOXY- | (4-HO-PH)- | |
| PHENYL)-OXO-ACETIC ACID | PROPYLIDENEAMINOOXY)- | |
| | ACETIC ACID | |
| 2-BENZOYLAMINO-3-(4- | 2-(1,2,3,4,5- | |
| HYDROXY-3-METHOXY- | PENTAHYDROXY-PENTYL)- | |
| PHENYL)-PROPIONIC ACID | THIAZOLIDINE-4-CARBOXYLIC | |
| 3-HYDROXY-2-(TRITYL | | |
| AMINO)-PROPIONIC ACID | ACID | |
| 2-(2-HYDROXYIMINO- | 1-HYDROXY-4-(1-PHENYL-1H- | |
| PROPIONYLAMINO)-SUCCINIC | TETRAZOL-5-YL-THIO)-2- | |
| ACID | NAPHTHOIC ACID | |
| (2-HYDROXYIMINO- | 1-HYDROXY-4-(4- | |
| PROPIONYLAIVIINO)-ACETIC | NITROPHENOXY)-2-NAPHTHOIC | |
| ACID | ACID | |
| 4-((2-HYDROXY-5-NITRO- | 1-HYDROXY-4- | |
| BENZYLIDENE)-AMINO)- | (METHOXYCARBONYLMETHOXY)- | |
| BENZOIC ACID | 2-NAPHTHOIC ACID | |
| BIS-(4-BR-PH)-HYDROXY- | 7-HYDROXY-1,3,4- | |
| ACETIC ACID, 2-AMINO-1- | TRIAZINDOLIZINE-6- | |
| (4-NITRO-PHENYL) -PROPANE- | CARBOXYLIC ACID | |
| | 4- | |
| 1,3-DIOL | | |
| 2,6-DIHYDROXY-4-METHYL- | (ETHOXYCARBONYLMETHOXY)- | |
| BENZOIC ACID | 1 -HYDROXY-2-NAPHTHOIC | |
| 3-(5-((DIHYDROXY- | ACID | |
| (GLUCOPYRANOSYL)-PH)-OXO | | |
| PR)-2-HO- | (ETHOXYCARBONYLMETHOXY)- | |
| PHENYLCARBAMOYL)-ACRYLIC | 1-HYDROXY-2-NAPHTHOIC | |
| ACID | ACID | |
| | 7-HYDROXY-5-METHYL-1,3,4- | |
| | TRIAZAINDOLIZINE-2- | |
| CARBOXYLIC ACID | 4-((2-HYDROXY-3-NITRO- | |
| MONOHYDRATE | BENZYLIDENE)-AMINO)- | |
| 1-(DIBUTYLAMINO)-3-(4- | BENZOIC ACID | |
| HYDROXYANILINO)-2- | 4-((2,4-DIHYDROXY- | |
| PROPANOL DIOXALATE | BENZYLIDENE)-AMINO)- | |
| 1-HYDROXY-2-OXO-1,2- | BENZOIC ACID | |
| DIHYDRO-PYRIDINE-3- | 2-((2-HYDROXY-3-NITRO- | |
| CARBOXYLIC ACID | BENZYLIDENE)-AMINO)-3- | |
| (5-CYANO-6-HYDROXY-4- | (1H-IMIDAZOL-2-YL)- | |
| METHYL-2-OXO-2H-PYRIDIN- | PROPIONIC ACID | |
| 1-YL)-ACETIC ACID | 2-((2-HYDROXY- | |
| 2-(1,2,3,4,5- | BENZYLIDENE)-AMINO)-3- | |
| PENTAHYDROXY-PENTYL)- | (3H-IMIDAZOL-4-YL)- | |
| THIAZOLIDINE-4-CARBOXYLIC | PROPIONIC ACID | |
| | 4-((5-BROMO-2-HYDROXY- | |
| ACID | | |
| 2-(1,2,3,4,5- | BENZYLIDENE)-AMINO)- | |
| PENTAHYDROXY-PENTYL)- | BENZOIC ACID | |
| | 4-((2-HYDROXY- | |
| THIAZOLIDINE-4-CARBOXYLIC | | |
| | BENZYLIDENE)-AMINO)- | |
| ACID | | |
| 2-(1,2,3,4-TETRAHYDROXY- | BENZOIC ACID | |
| | DIBROMOBENZILIC ACID | |
| BUTYL)-THIAZOLIDINE-4- | 4-(2-(2-HYDROXY-PHENOXY)- | |
| CARBOXYLIC ACID | ACETYLAMINO)-BENZOIC ACID | |
| (2,3,4,5,6-PENTAHYDROXY- | 3-FORMYL-2-HYDROXY- | |
| HEXANOYLAMINO)-ACETIC | | |
| | BENZOIC ACID, COMPOUND | |
| ACID | WITH MORPHOLINE | |
| ((2-HYDROXY-BENZYLIDENE)- | 2-HYDROXY-3-(P- | |
| AMINO)-PHENYL-ACETIC ACID | TOLYLIMINO-METHYL)- | |
| | BENZOIC ACID | |
| 4-((2-HYDROXY-5-METHOXY- | 1-BICYCLO[2.2.1]HEPT-2- | |
| BENZYLIDENE)-AMINO)- | YL-ETHYLAMINE, COMPOUND | |
| BENZOIC ACID | WITH HYDROXY-ACETIC ACID | |
| 2-((2-HYDROXY-5-METHOXY- | | |
| BENZYLIDENE)-AMINO)- | | |
| BENZOIC ACID | | |
| 2-HYDROXY-3-IMINOMETHYL- | 2,2-BIS(TRIFLUOROMETHYL)- | |
| BENZOIC ACID, COMPOUND | 2-HYDROXYACETIC ACID | |
| WITH THIOUREA | | |
| | 2-HYDROXY-2- | |
| ALPHA-CARBOXY-4-HYDROXY- | (TRIFLUOROMETHYL)BUTYRIC | |
| 3′-METHOXYSTILBENE | ACID | |
| | 2-HYDROXY-2- | |
| 4-(3-FLUORO-4- | (TRIFLUOROMETHYL)PROPIONI | |
| HYDROXYPHENYL) BUTYRIC | | |
| | C ACID | |
| ACID | BOC-DL-SER-OH | |
| SEROTONIN HYDROGEN | Z-4-AMINO-3- | |
| ACETATE | HYDROXYBUTYRIC ACID | |
| HYDROXYVALERENIC ACID | | |
| DIHYDROXYPHENYLALANINE, | HYDROXYPHENYL)ISOVALERIC | |
| DL-3,4-[ALANINE-1-14C] | | |
| | ACID | |
| 2-(4- | | |
| DIHYDROXYPHENYLALANINE, | | |
| L-3,4-[RING 2,5,6-3H] | HYDROXYPHENYL)ISOVALERIC | |
| | ACID | |
| HYDROXYBENZOIC ACID-P | (1ALPHA, 2ALPHA, 3BETA, 4BET | |
| [CARBOXYL-14C] | A)-2,4-BIS(4- | |
| 3-HYDROXYMETHYL SERINE, | HYDROXYPHENYL)-1,3- | |
| [1-14C] | CYCLOBUTANEDICARBOXYLIC | |
| HYDROXYPROLINE, L-4 | | |
| [3H(G)] | 4-(2,3-DIHYDROXYPROPYL) | |
| HYDROXYTRYPTAMINE | 2-ISONONENYLSUCCINATE, | |
| BINOXALATE,5-[2-14C]] | POTASSIUM SALT | |
| HYDROXYTRYPTAMINE | | |
| | (S)-(−)-2-HYDROXY-3,3- | |
| BINOXALATE, 5-[1,2-3H] | | |
| 2,2-BIS(HYDROXYMETHYL)-N- | DIMETHYLBUTYRIC ACID | |
| | (S)-(−)-2-HYDROXY-3,3- | |
| BUTYRIC ACID | | |
| 4-HYDROXYBENZOIC ACID- | DIMETHYLBUTYRIC ACID | |
| (S)-(+)-2-HYDROXY-4- | | |
| 13C7 | | |
| L-TYROSINE-2,6-D2 | PHTHALIMIDOBUTYRIC ACID | |
| BOC-[15N]TYR-OH | | |
| 5-HYDROXYPYRAZINE-3- | (S)-(+)-2-HYDROXY-4- | |
| CARBOXYLIC ACID | PHTHALIMIDOBUTYRIC ACID | |
| 4-HYDROXY-3- | | |
| (MORPHOLINOMETHYL)BENZOIC | 1-BICYCLO(2.2.1)HEPT-2- | |
| ACID HYDRATE | YL-ETHYLAMINE, 2-HYDROXY- | |
| (R)-2-HYDROXY-4- | 3,5-DINITRO-BENZOATE | |
| PHENYLBUTYRIC ACID | | |
| 6-HYDROXYPICOLINIC ACID | 4-HYDROXY-BENZOIC ACID, | |
| | COMPOUND WITH 1- | |
| 6-HYDROXYPICOLINIC ACID | BICYCLO(2.2.1)HEPT-2-YL- | |
| 9-HYDROXY-OCTADECANOIC | ETHYLAMINE | |
| | CYCLOHEXYL-HYDROXY | |
| ACID | | |
| 3,3,3-TRIFLUORO-2,2- | PHENYL-ACETIC ACID | |
| | 5-CHLORO-6-HYDROXY-2- | |
| DIHYDROXY-PROPIONIC ACID | METHYL-PYRIMIDINE-4- | |
| HYDROXY-PHENYL-P-TOLYL- | CARBOXYLIC ACID | |
| | (3,4,5,6-TETRAHYDROXY- | |
| ACETIC ACID | | |
| 2-AMINO-3-(4-HYDROXY-3- | TETRAHYDRO-PYRAN-2-YL)- | |
| METHOXY-PHENYL)-PROPIONIC | ACETIC ACID | |
| | 2-TERT- | |
| ACID, HYDROCHLORIDE | | |
| | BUTOXYCARBONYLAMINO-3-(4- | |
| 3-HYDROXYADAMANTANE-1- | HYDROXY-PHENYL)-PROPIONIC | |
| CARBOXYLIC ACID | ACID | |
| BIS-(4-BR-PH)HYDROXY- | 4-[(4- | |
| ACETIC ACID, 2- | HYDROXYPHENYL)SULFONYL]BE | |
| ISOPROPYLAMINO-1-(4- | NZOIC ACID | |
| NITRO-PHENYL)-ETHANOL | ((2-HYDROXY-5-NITRO- | |
| BIS-(4-BR-PH)-HYDROXY- | BENZYLIDENE)-AMINO) | |
| ACETIC ACID, 3-AMINO-1- | PHENYL-ACETIC ACID | |
| (4-NITRO-PHENYL)-BUTANE- | 3-PHENYL-DL-SERINE | |
| 1,4-DIOL | 3-PHENYL-DL-SERINE | |
| 4-HYDROXY-BENZOIC ACID, | 17-OXOANDROST-5-EN-3- | |
| COMPOUND WITH C- | BETA-YL-BETA-D | |
| BICYCLO(2.2.1)HEPT-5-EN- | GLUCURONIDE | |
| | 17 -BETA-HYDROXY-5-OXO- | |
| 2-YL-METHYLAMINE | | |
| BIS-(4-ER-PHENYL)- | 3,5-SECO-A-NORANDROSTAN- | |
| HYDROXY-ACETIC ACID, 2,6- | 3-OIC ACID | |
| DIMETHYL-PYRIDINE-N-OXIDE | 3-HYDROXYESTRA-1,3,5(10)- | |
| | TRIEN-17BETA-YL BETA-D | |
| GLUCURONIDE | 3,20-DIONE 11-(BETA-D- | |
| 17-BETA-HYDROXYESTRA- | GLUCURONIDE) | |
| 1,3,5(10),6-TETRAEN-3-YL- | 11BETA-HYDROXY-3,20- | |
| BETA-D-GLUCURONIDE | DIOXOPREGN-4-EN-21-YL | |
| 3,20-DIOXOPREGN-4-EN- | 21-BETA-D-GLUCURONIDE (1:1 | |
| YL BETA-D-GLUCURONIDE | SODIUM SALT + | |
| 3,11,20-TRIOXOPREGN-4-EN- | 17-ALPHA-HYDROXY-3,20- | |
| | DIOXOPREGN-4-EN-21-YL- | |
| | 21-YL BETA-D-GLUCURONIDE | |
| | BETA-D-GLUCURONIDE | |
| 21-HYDROXY-20-OXO-5-BETA- | ALDOSTERONE 18-(-ALPHA-D | |
| PREGNAN-3-ALPHA-YL-BETA- | GLUCURONIDE) | |
| | 11BETA, 18-EPOXY-21- | |
| D-GLUCURONIDE | | |
| 3-ALPHA-HYDROXY-20-OXO-5- | HYDROXY-3,20-DIOXO- | |
| BETA-PREGNAN-21-YL-BETA- | 17ALPHA-PREGN-4-EN-18-YL | |
| D-GLUCURONIDE | ALPHA-D-GLUCURONI | |
| 3-ALPHA,20-BETA- | 17-ALPHA-HYDROXY-3,11,20- | |
| DIACETOXY-11-OXO-5-BETA- | TRIOXOPREGN-4-EN-21-YL- | |
| PREGNAN-21-OIC ACID | BETA-D-GLUCURONIDE | |
| 21-HYDROXY-11,20-DIOXO-5- | 17ALPHA-HYDROXY-3,11,20- | |
| BETA-PREGNAN-3-ALPHA-YL- | TRIOXOPREGN-4-EN-21-YL | |
| BETA-D-GLUCURONIDE | BETA-D-GLUCURONIDE | |
| 21-HYDROXY-11,20-DIOXO- | AMMONIUM SALT | |
| 5BETA-PREGNAN-3ALPHA-YL | PREDNISONE 21-(-BETA-D- | |
| BETA-D-GLUCURONIDE, | GLUCURONIDE) | |
| AMMONIUM SALT | 3-ALPHA,11-BETA- | |
| 11,20-DIOXO-5-BETA- | DIHYDROXY-20-OXO-5-BETA- | |
| PREGNANE-3-ALPHA,21-DIYL | PREGNAN-21-YL-BETA-D- | |
| BIS-(BETA-D-GLUCURONIDE) | GLUCURONIDE | |
| | TETRAHYDROCORTICOSTERONE | |
| 11,20-DIOXO-5BETA- | | |
| PREGNANE-3ALPHA,21-DIYL | 3,21BIS(-BETA-D- | |
| | GLUCURONIDE) | |
| BIS-(BETA-D-GLUCURONIDE | 20-BETA,21-DIHYDROXY-11- | |
| AMMONIUM SALT) | OXO-5-BETA-PREGNAN-3- | |
| 11-BETA,21- | ALPHA-YL-BETA-D- | |
| DIHYDROXYPREGN-4-ENE | | |
| GLUCURONIDE | BETA,14-BETA,17-ALPHA- | |
| 3-ALPHA,17-ALPHA- | PREGNAN-21-OIC ACID | |
| DIHYDROXY-11,20-DIOXO-5- | 3-BETA,5,14-TRIHYDROXY- | |
| BETA-PREGNAN-21-YL-BETA- | 20-OXO-5-BETA,14-BETA,17- | |
| D-GLUCURONIDE | ALPHA-PREGNANE-19,21- | |
| HYDROCORTISONE 21-(-BETA- | DIOIC ACID | |
| D-GLUCURONIDE) | 3-ALPHA,5-DIHYDROXY-20- | |
| HYDROCORTISONE 21-(-BETA- | OXO-5-BETA,17-ALPHA- | |
| D-GLUCURONIDE,AMMONIUM | PREGNANO-21,14-LACTON-19- | |
| SALT) | OIC ACID | |
| PREDNISOLONE 21-(-BETA-D- | 3BETA,5-DIHYDROXY-20-OXO- | |
| GLUCURONIDE) | 5BETA,14BETA-PREGNANO- | |
| 5BETA-PREGNAN- | 21,14-LACTON-19-OIC ACID | |
| 3ALPHA,11BETA,17,21- | 2-HYDROXY-3,3-DIMETHYL-A- | |
| TETROL-20-ONE 21- | NORCHOLEST-5-ENE-2- | |
| GLUCOSIDURONATE | | |
| 17ALPHA,20BETA,21- | CARBOXYLIC ACID | |
| | 5ALPHA-CHOLANIC ACID | |
| TRIHYDROXY-11-OXO-5BETA- | 3BETA-OL | |
| PREGNAN-3ALPHA-YL BETA-D- | 3-BETA-ACETOXYCHOL-5-EN- | |
| GLUCURONIDE | 24-OIC ACID | |
| 11BETA,17ALPHA,20BETA,21- | 3-ALPHA-HYDROXY-5-ALPHA- | |
| TETRAHYDROXY-5BETA- | CHOL-11-EN-24-OIC ACID | |
| PREGNAN-3ALPHA-YL BETA-D | | |
| | 5BETA-CHOLANIC ACID- | |
| GLUCURONIDE | | |
| 20-BETA-HYDROXY-5-OXO- | 3ALPHA,6ALPHA-DIOL N- | |
| 3,5-SECO-A-NORPREGNAN-3- | (CARBOXYMETHYL)-AMIDE | |
| | 5BETA-CHOLANIC ACID- | |
| CIC ACID | | |
| 20-BETA-ACETOXY-5-OXO- | 3ALPHA,6BETA-DIOL | |
| | METHYL 3-ALPHA-(3- | |
| 3,5-SECO-A-NORPREGNAN-3- | | |
| | CARBOXYPROPANOYLOXY)-12- | |
| OIC ACID | | |
| 3-BETA,17-BETA-DIHYDROXY- | ALPHA-HYDROXY-5-BETA- | |
| 17-ALPHA-PREGN-5-EN-21- | CHOLAN-24-OATE | |
| | 3-ALPHA,12-BETA- | |
| OIC ACID | | |
| 3-BETA,5,14,19- | DIHYDROXY-5-BETA-CHOLAN- | |
| TETRAHYDR0XY-20-OXO-5- | 24-OIC ACID | |
| 3-BETA-HYDROXY-7-BETA- | 3-BETA-HYDROXY-5-BETA- | |
| METHOXYCHOL-5-EN-24-OIC | ANDROSTANE-17-BETA- | |
| ACID | CARBOXYLIC ACID | |
| 3-ALPHA,7-ALPHA- | 3-BETA-HYDROXYANDROSTA- | |
| DIACETOXY-12-ALPHA- | 5,16-DIENE-17-CARBOXYLIC | |
| HYDROXY-5-BETA-CHOLAN-24- | ACID | |
| OIC ACID | 3-BETA,7-DIACETOXY-5- | |
| 3-ALPHA-HYDROXY-11-OXO-5- | ALPHA-ANDROSTANE-17-BETA- | |
| BETA-CHOLAN-24-OIC ACID | CARBOXYLIC ACID | |
| | 3-ALPHA, 12-BETA- | |
| 5BETA-CHOLANIC ACID- | DIHYDROXY-5-ALPHA- | |
| 3ALPHA-OL-12-ONE | ANDROSTANE-17-BETA- | |
| 11-BROMO-3ALPHA-HYDROXY- | | |
| | CARBOXYLIC ACID | |
| 12-OXO-CHOLAN-24-OIC ACID | 3-BETA,17-BETA-DIACETOXY- | |
| 5BETA-CHOLANIC ACID- | 5-ALPHA-ANDROSTANE-17- | |
| 3ALPHA-OL-7,12-DIONE | ALPHA-CARBOXYLIC ACID | |
| 3-ALPHA-HYDROXY-11,12- | 8,19-EPOXY-3-BETA,5- | |
| DIOXO-5-BETA-CHOLAN-24- | | |
| | DIHYDROXY-5-BETA- | |
| OIC ACID | | |
| 3-ALPHA,12-BETA- | ANDROSTANE-17-BETA- | |
| DIHYDROXY-11-OXO-5-BETA- | CARBOXYLIC ACID | |
| | 3BETA-ACETOXY-8,19-EPOXY- | |
| CHOLAN-24-OIC ACID | 5-HYDROXY-5BETA- | |
| (20R)-3-ALPHA,12-ALPHA- | ANDROSTANE-17 BETA | |
| DIHYDROXY-23,24-DINOR-5- | CARBOXYLIC ACID | |
| BETA-CHOLAN-22-OIC ACID | METHYL 3ALPHA-(3- | |
| | CARBOXYPROPANOYLOXY)- | |
| 3-ALPHA-HYDROXY-11-OXO- | | |
| | 12ALPHA-HYDROXY-5BETA- | |
| 23,24-DINOR-5-BETA- | ANDROSTANE-17BETA-CA | |
| CHOLAN-22-OIC ACID | 3-BETA-HYDROXY-7-ALPHA- | |
| 3-ALPHA-ACETOXY-5-ALPHA- | | |
| | METHOXYANDROST-5-ENE-17- | |
| ANDROSTANE-17-BETA- | | |
| | BETA-CARBOXYLIC ACID | |
| CARBOXYLIC ACID | | |
| 3-ALPHA-HYDROXY-5-BETA- | | |
| ANDROSTANE-17-BETA- | | |
| CARBOXYLIC ACID | | |
| 3-BETA,17-BETA- | 3BETA,5-DIHYDROXY- | |
| DIACETOXYANDROST-5-ENE- | 5BETA,14BETA-ANDROSTANO- | |
| 17-ALPHA-CARBOXYLIC ACID | 19,8-LACTONE-17ALPHA- | |
| 3-BETA,19- | CARBOXYLIC ACID | |
| DIHYDROXYANDROST-4-ENE- | 3-BETA,5,19-TRIHYDROXY-5- | |
| 17-BETA-CARBOXYLIC ACID | BETA-ANDROSTANE-17-BETA- | |
| | CARBOXYLIC ACID | |
| 17-ALPHA-CARBOXY-3- | 3-BETA,19-DIACETOXY-5- | |
| BETA,5-DIHYDROXY-5- | HYDROXY-5-BETA- | |
| BETA,14-BETA-ANDROSTAN- | ANDROSTANE-17-BETA- | |
| 19-OIC ACID | CARBOXYLIC ACID | |
| 17-ALPHA-ETHOXYCARBONYL- | 8,19-EPOXY-3BETA,5- | |
| 3-BETA,5-DIHYDROXY-5- | DIHYDROXY-19-METHOXY- | |
| BETA,14-BETA-ANDROSTAN- | 5BETA-ANDROSTANE-17BETA- | |
| 19-OIC ACID | CARBOXYLIC ACID | |
| 3BETA-ACETOXY-17ALPHA- | 3BETA-ACETOXY-8,19-EPOXY- | |
| ETHOXYCARBONYL-5-HYDROXY- | 5-HYDROXY-19-METHOXY- | |
| 5BETA,14BETA-ANDROSTAN- | 5BETA-ANDROSTANE-17BETA- | |
| 19-OIC ACI | CARBOXYLIC | |
| 3-BETA,5-DIACETOXY-17- | 3-BETA,5,19-TRIHYDROXY-5- | |
| ALPHA-ETHOXYCARBONYL-5- | BETA,14-BETA,17-BETA(H)- | |
| BETA,14-BETA-ANDROSTAN- | ETIANIC ACID | |
| 19-OIC ACID | 3-BETA,5,19-TRIHYDROXY-5- | |
| 17-ALPHA-CARBOXY-3- | BETA-ANDROST-8(14)-ENE | |
| BETA,5-DIHYDROXY-5-BETA- | 17-BETA-CARBOXYLIC ACID | |
| ANDROST-14-EN-19-OIC ACID | | |
| | 3-BETA,5-DIHYDROXY-19,8- | |
| 17-ALPHA-ETHOXYCARBONYL- | BETA-LACTONE-5-BETA,17- | |
| 3-BETA,5-DIHYDROXY-5- | ALPHA(H)-ETIANIC ACID | |
| BETA-ANDROST-14-EN-19-OIC | | |
| | 3-BETA-ACETOXY-5-HYDROXY- | |
| ACID | | |
| | 19,8-BETA-LACTONE-5- | |
| | BETA,17-ALPHA(H)-ETIANIC | |
| | ACID | |
| 3-BETA,5-DIHYDROXY-19,8- | 19-HYDROXY-3-OXOANDROST- | |
| BETA-LACTONE-5-BETA,17- | 4-ENE-17-BETA-CARBOXYLIC | |
| BETA(H)-ETIANIC ACID | ACID | |
| | 19-ACETOXY-3-OXOANDROST- | |
| 3-BETA-ACETOXY-5-HYDROXY- | 4-ENE-17-BETA-CARBOXYLIC | |
| 19,8-BETA-LACTONE-5- | ACID | |
| BETA,17-BETA(H)-ETIANIC | 19-HYDROXY-3-OXO-14-BETA- | |
| ACID | ANDROST-4-ENE-17-ALPHA- | |
| 17-ALPHA-CARBOXY-3- | CARBOXYLIC ACID | |
| BETA,5,14-TRIHYDROXY-5- | 19-ACETOXY-3-OXO-14-BETA- | |
| BETA,14-BETA-ANDROSTAN- | ANDROST-4-ENE-17-ALPHA- | |
| 19-OIC ACID | CARBOXYLIC ACID | |
| 5,14-DIACETOXY-3BETA,19- | 17-ALPHA-ETHOXYCARBONYL- | |
| DIHYDROXY-5BETA,14BETA- | 5-HYDROXY-3-OXO-5- | |
| ANDROSTANE-17BETA- | BETA,14-BETA-ANDROSTAN- | |
| CARBOXYLIC ACID | 19-OIC ACID | |
| 3-BETA,5,14,19- | METHYL 3ALPHA-(3- | |
| TETRAHYDROXY-5-BETA,14- | CARBOXYPROPANOYLOXY)- | |
| BETA,17-BETA(H)-ETIANIC | 12ALPHA-HYDROXY-7-OXO- | |
| ACID | 5BETA-ANDROSTANE-17B | |
| 3BETA,11ALPHA,19- | 5,19-DIHYDROXY-3-OXO-5- | |
| TRIACETOXY-1BETA,5,14- | BETA,14-BETA-ANDROSTANE- | |
| TRIHYDROXY-5BETA,14BETA- | 17-ALPHA-CARBOXYLIC ACID | |
| ANDROSTANE-17BETA | 14,19-DIHYDROXY-3-OXO-14- | |
| 1BETA,3BETA,11ALPHA,19- | BETA-ANDROST-4-ENE-17- | |
| TETRAACETOXY-5,14- | BETA-CARBOXYLIC ACID | |
| DIHYDROXY-5BETA,14 BETA- | | |
| | 14-ANHYDRO-17-ALPHA- | |
| ANDROSTANE-17BET | | |
| 8,19-EPOXY-5-HYDROXY-3- | STROPHANTHIDINIC ACID | |
| | 3BETA-ACETOXY-5-HYDROXY- | |
| OXO-5-BETA-ANDROSTANE-17- | | |
| | 5BETA,17ALPHA-CARDA- | |
| BETA-CARBOXYLIC ACID | | |
| | 14,20(22)-DIENOLID-19-OIC | |
| 6-BETA-HYDROXY-3- | ACID | |
| OXOANDROST-4-ENE-17-BETA- | 14-CHLORO-3-BETA,5- | |
| CARBOXYLIC ACID | DIHYDROXY-5-BETA,17- | |
| ALPHA-CARD-20(22)-ENOLID- | 2-([2-[2-([2-HYDROXY-3- | |
| 19-OIC ACID | (3-(TRIFLUOROMETHYL)-1H- | |
| STROPHANTHIDINIC ACID | PYRAZOL-1- | |
| 17-ALPHA-STROPHANTHIDINIC | | |
| | YL]PROPYL]TRIO)ANILINO] | |
| ACID | | |
| 3-BETA-HYDROXYCHOLEST-5- | 5-CHLORO-2- | |
| ENE-7-BETA-YL | HYDROXYNICOTINIC ACID | |
| CARBOXYMETHYL ETHER | 2-(1,2,3,4- | |
| 1-[(3,4- | TETRAHYDROXYBUTYL)-1,3- | |
| DIMETHOXYPHENYL)SULFONYL | THIAZOLANE-4-CARBOXYLIC | |
| ]-4-HYDROXY-2- | ACID | |
| | 2-(1,2,4,5-TETRAHYDROXY- | |
| PYRROLIDINECARBOXYLIC | | |
| ACID | 3-11[3,4,5-TRIHYDROXY-6- | |
| 4-HYDROXY-1-(2- | (HYDROXYMETHYL)TETRAHYDRO | |
| THIENYLSULFONYL)-2- | -2H-PYRA | |
| PYRROLIDINECARBOXYLIC | ROYAL JELLY ACID | |
| ACID | | |
| 1-(2-FURYLCARBONYL)-4- | (HYDROXYMETHYL)PHENYL]THI | |
| HYDROXY-2- | O]-1-NAPHTHOIC ACID | |
| PYRROLI DINECARBOXYLIC | 2-[[8-(HYDROXYMETHYL)-1- | |
| ACID | NAPHTHYL]THIO]BENZOIC | |
| 1-(4-FLUOROBENZOYL)-4- | ACID | |
| HYDROXY-2- | | |
| | 8-[[8-(HYDROXYMETHYL)-1- | |
| PYRROLIDINECARBOXYLIC | | |
| | NAPHTHYL]TRIO]-1- | |
| ACID | | |
| 1-(4-FLUOROBENZOYL)-4- | NAPHTHOIC ACID | |
| | 2-[[2-(2- | |
| HYDROXY-2- | | |
| | HYDROXYETHYL)PHENYL]THIO] | |
| PYRROLIDINECARBOXYLIC | | |
| | BENZOIC ACID | |
| ACID | 8-[[2- | |
| 4-[2-([2-HYDROXY-3-[3- | | |
| | (HYDROXYMETHYL)PHENYL]SUL | |
| (TRIFLUOROMETHYL)-1H- | | |
| | FINYL]-1-NAPHTHOIC ACID | |
| PYRAZOL-1- | | |
| YL]PROPYL]THIO)ANILINO]- | 8-[[8-(HYDROXYMETHYL)-1- | |
| 4-O | NAPHTHYL]SULFINYL]-1- | |
| NAPHTHOIC ACID | | |
| 2-[[8-(HYDROXYMETHYL)-1- | ACID | |
| NAPHTHYL]SULFINYL]BENZOIC | 4-[(3- | |
| ACID ACETATE | CARBOXYACRYLOYL)AMINO]-2- | |
| | HYDROXYBENZOIC ACID | |
| 4-[(3- | | |
| HYDROXYETHYL)PHENYL]SULFI | CARBOXYPROPANOYL)AMINO]- | |
| NYL]BENZOIC ACID | 2-HYDROXYBENZOIC ACID | |
| 2-HYDROXY-4-OXO-4- | | |
| PHENYLBUT-2-ENOIC ACID | | |
| 4-[2-[(2-HYDROXY-3-([5- | 1-HYDROXYBENZYLIDENE)AMINO | |
| (TRIFLUOROMETHYL)-2- | ]-2-(2- | |
| PYRIDYL]OXY]PROPYL)THIOIA | METHOXYETHOXY)BENZOIC | |
| NILINO]-4-OX | ACID | |
| 2-[(5-CHLORO-2- | 3,4-DIHYDROXYPHENYLACETIC | |
| HYDROXYBENZYL)AMINO]PROPA | ACID (2,2-D2) | |
| NOIC ACID | | |
| 2-[[(3-HYDROXY-2- | 3,4-DIHYDROXYPHENYLACETIC | |
| NAPHTHYL)METHYL]AMINO]PRO | ACID (RING-D3,2,2-D2) | |
| PANOIC ACID | 3,4-DIHYDROXYPHENYLACETIC | |
| 2-[(2- | | |
| | ACID (RING-13C6, 4- | |
| HYDROXYBENZYL)AMINO]PROPA | | |
| | HYDROXY-18O) | |
| NOIC ACID | | |
| 2-[(2-HYDROXY-3- | L-DOPA (2, 3-13C2, 4- | |
| METHOXYBENZYL)AMINO]PROPA | HYDROXY-18O) | |
| NOIC ACID | 2-HEDTA(2-HYDROXYETHYL- | |
| 2-[(2- | D4) | |
| HYDROXYBENZYL)AMINO]-4- | HOMOVANILLIC ACID (1,2- | |
| METHYLPENTANOIC ACID | 13C2) | |
| 2-[(2- | HOMOVANILLIC ACID (RING | |
| HYDROXYBENZYL)AMINO]-4- | 13C6, 4-HYDROXY-18O) | |
| (METHYLTHIO) BUTANOIC ACID | (S)-N-CARBOBENZYLOXY-4- | |
| 5-ACETYL-2-AMINO-4- | AMINO-2-HYDROXYBUTYRIC | |
| HYDROXYBENZOIC ACID | ACID | |
| 4-[3-[(2- | 4-(HYDROXYMETHYL)-3- | |
| HYDROXYBENZOYL)AMINO]ANIL | PYRIDINECARBOXYLIC ACID | |
| INO]-4-OXOBUT-2-ENOIC | | |
| 6-HYDROXY-1-NAPHTHOIC | CARBACYCLIN | |
| ACID | DANSYL HYDROXY-L-PROLINE, | |
| (5R)-5-HYDROXY-L-LYSINE | CYCLOHEXYLAMMONIUM SALT | |
| DIHYDROCHLORIDE | | |
| MONOHYDRATE | | |
| TETRODOTOXIN CITRATE | (+/−)11,12-DIHETRE | |
| L-3-HYDROXYKYNURENINE | (+/−)8,9-DIHETRE | |
| 3-HYDROXYNONAOIC ACID | FLUPROSTENOL | |
| 3-HYDROXYUNDECANOIC ACID | GLY-PRO-HYDROXY-PRO | |
| | ACETATE SALT | |
| 3-HYDROXYTRIDECANOIC ACID | HEPOXILIN A3 | |
| | HEPOXILIN B3 | |
| 3-HYDROXYPENTADECANOIC | 15(S)-HEDE | |
| | (+/−)5-HETRE | |
| ACID | | |
| (+/−)-2-HYDROXYUNDECANOIC | 13(S)-ROTE | |
| | LTB3 | |
| ACID | | |
| (+/−)-2- | (R)-2-HYDROXY-4- | |
| HYDROXYTRIDECANOIC ACID | METHYLPENTANOIC ACID | |
| | L-6-HYDROXYNORLEUCINE | |
| (+/−)-2- | R-(3)-HYDROXYMYRISTIC | |
| HYDROXYPENTADECANOIC ACID | ACID | |
| | HYDROXYLAMMONIUM OXALATE | |
| 3-AMINO-4-HYDROXYBENZOIC | | |
| ACID HYDROCHLORIDE | (+/−)-ALPHA-AMINO-3- | |
| | HYDROXY-5- | |
| 5,6-DICHLOROVANILLIC ACID | METHYLISOXAZOLE-4- | |
| | PROPIONIC ACID HYDRATE | |
| 4-HYDROXY-5-IODO-3- | 4-HYDROXYADAMANTANE-2- | |
| METHOXYBENZOIC ACID | CARBOXYLIC ACID | |
| LECANORIC ACID | | |
| ORSELLINIC ACID | HYDROXYBICYCLO[3.2.1]OCTA | |
| MONOHYDRATE | NE-6-CARBOXYLIC ACID | |
| STEVIOL | | |
| ALA-ARG-GLY-ILE-LYS-GLY- | 12(S),20-DIHETE | |
| ILE-ARG-GLY-PHE-SER-GLY | 8(S),15(S)-DIHETE | |
| 3ACOH . 5H2O | 8(R),15(S)-DIHETE | |
| 3,4-DIHYDROXYBENZOIC ACID | HOE 140 | |
| MONOHYDRATE | (−)-CIS, TRANS-ABSCISIC | |
| ACID | PROSTAGLANDIN D1 | |
| N-T-BOC-(2R,3R)-3-AMINO- | PROSTAGLANDIN D2-D4 | |
| 2-HYDROXY-4-PHENYLBUTYRIC | BW 245C | |
| ACID | 15(R)-15-METHYL | |
| BUTORPHANOL TARTRATE SALT | PROSTAGLANDIN D2 | |
| | 15(S)-15-METHYL | |
| NAZUMAMIDE A | PROSTAGLANDIN D2 | |
| 2-HYDROXY-6-PHENYL-3,5- | 16,16-DIMETHYL | |
| PYRIDINEDICARBOXYLIC ACID | PROSTAGLANDIN D2 | |
| | TETRANOR PGDM | |
| 6-(4-CHLOROPHENYL)-2- | PROSTAGLANDIN D3 | |
| HYDROXY-3,5- | PROSTAGLANDIN E1-D4 | |
| PYRIDINEDICARBOXYLIC ACID | DELTA17-PROSTAGLANDIN E1 | |
| 2-HYDROXY-6-[3- | 16,16-DIMETHYL-6-KETO | |
| (TRIFLUOROMETHYL)PHENYL]- | PROSTAGLANDIN E1 | |
| 3,5-PYRIDINEDICARBOXYLIC | 15(R)-15-METHYL | |
| ACID | PROSTAGLANDIN E2 | |
| | 15(S)-15-METHYL | |
| 2-HYDROXY-6-[4- | PROSTAGLANDIN E2 | |
| (TRIFLUOROMETHYL)PHENYL]- | HEXANOR PGEM | |
| 3,5-PYRIDINEDICARBOXYLIC | 20-HYDROXY PROSTAGLANDIN | |
| ACID | E2 | |
| | 17-TRANS PROSTAGLANDIN E3 | |
| 2-HYDROXY-6-(3,4,5- | | |
| TRIMETHOXYPHENYL)-3,5- | DELTA17-6-KETO | |
| PYRIDINEDICARBOXYLIC ACID | PROSTAGLANDIN F1ALPHA | |
| | 8-ISO PROSTAGLANDIN | |
| 2-(6-HYDROXY-3- | F1ALPHA | |
| PYRIDAZINYL)-2-(4- | 8-ISO PROSTAGLANDIN | |
| METHOXYPHENYL)ACETIC ACID | F1BETA | |
| | S-TRANS PROSTAGLANDIN | |
| 4-HYDROXY-1-[(4- | F2BETA | |
| METHYLPHENYL)SULFONYL]-2- | 8-ISOPROSTAGLANDIN | |
| PYRROLIDINECARBOXYLIC | F2BETA | |
| ACID | 8-ISO-13,14-DIHYDRO-15- | |
| | KETO PROSTAGLANDIN | |
| 15(R)-15-METHYL | | |
| | F2ALPHA | |
| PROSTAGLANDIN A2 | | |
| 8-ISO-15-KETO | 9(S)-HODE-D4 | |
| PROSTAGLANDIN F2ALPHA | 13(S)-HODE-D4 | |
| 11BETA-13,14-DIHYDRO-15- | (+/−)11-HEDE | |
| KETO PROSTAGLANDIN | 11(R)-HEDE | |
| F2ALPHA | 11(S)-HEDE | |
| 13,14-DIHYDRO | (+/−)15-HEDE | |
| | 15(R)-HEDE | |
| PROSTAGLANDIN F2ALPHA | | |
| 20-HYDROXY PROSTAGLANDIN | 5(S)-HETRE | |
| F2ALPHA | 8(S)-HETRE | |
| 8-ISOPROSTAGLANDIN | (+/−)15-HETE | |
| F3ALPHA | (+−)8-HEPE | |
| 17-TRANS PROSTAGLANDIN | (+/−)9-HEPE | |
| F3ALPHA | (+/−)11-HEPE | |
| 13,14-DEHYDRO-15- | 11(R)-HEPE | |
| | (+/−)15-HEPE | |
| CYCLOHEXYL | 12(S)-HYDROXY-16- | |
| CARBAPROSTACYCLIN | HEPTADECYNOIC ACID | |
| ILOPROST | (+/−)5,6-DIHETRE | |
| PROSTAGLANDIN K1 | (+/−)14,15-DIHETRE | |
| PROSTAGLANDIN K2 | NG-HYDROXY-L-ARGININE | |
| 11-DEHYDRO THROMBOXANE | | |
| | MONOACETATE | |
| B2-D4 | CUCURBIC ACID | |
| 11-DEHYDRO-2,3-DINOR | 4-HYDROXYCYCLOHEXANE | |
| THROMBOXANE B2 | | |
| THROMBOXANE B3 | CARBOXYLIC ACID | |
| 11-DEHYDRO THROMBOXANE B3 | [CARBOXYL-14C] | |
| | ALLO-HYDROXYPROLINE, L-4 | |
| LEUKOTRIENE B4-D4 | [3H(G)] | |
| 11-TRANS LEUKOTRIENE C4 | L-3-HYDROXYPROLINE | |
| | 2-(4-HYDROXYCARBONYL-2- | |
| 11-TRANS LEUKOTRIENE D4 | NITROPHENYL)MALONDIALDEHY | |
| | DE | |
| 11-TRANS LEUKOTRIENE E4 | 2-(3-HYDROXYCARBONYL-2- | |
| | NITROPHENYL) MALONDIALDEHY | |
| 20-HYDROXY LEUKOTRIENE E4 | DE | |
| | 2-(5-HYDROXYCARBONYL-2- | |
| N-ACETYL-20-HYDROXY | NITROPHENYL)MALONDIALDEHY | |
| LEUKOTRIENE E4 | DE MONOHYDRATE | |
| LEUKOTRIENE G4 | (4(S)-TRANS)-4,5- | |
| DIHYDROXY-3-OXO-1- | 2-HYDROXY-2-(4- | |
| CYCLOHEXENE-1-CARBOXYLIC | METHOXYPHENYL)-2- | |
| ACID | PHENYLACETIC ACID | |
| 2-(2-HYDROXYCARBONYL-6- | 3-(2-HYDROXY-4- | |
| PYRIDYL)MALONDIALDEHYDE | METHOXYPHENYL)PROPANOIC | |
| | ACID | |
| 2-(3-HYDROXYCARBONYL-6- | 6-(3-HYDROXYPHENYL)-4- | |
| PYRIDYL)MALONDIALDEHYDE | OXOHEX-5-ENOIC ACID | |
| | 15(R)-HETE | |
| DEUTEROPORPHYRIN IX 2- | 20-CARBOXY LEUKOTRIENE E4 | |
| VINYL 4-HYDROXYMETHYL | | |
| (RS)-4-CARBOXY-3- | 16-CARBOXY TETRANOR | |
| 1-HYDROXYPHENYLOLYCINE | LEUKOTRIENE E3 | |
| (S)-4-CARBOXY-3- | (S)-(+)-NOREPINEPHRINE L- | |
| HYDROXYPHENYLGLYCINE | BITARTRATE | |
| CLOPROSTENOL | (+/−) -NOREPINEPHRINE L | |
| 6-CHLORO-4-HYDROXY-8- | | |
| | BITARTRATE HYDRATE | |
| METHYLQUINOLINE-3- | HYDROXY-6,8,11,14 | |
| CARBOXYLIC ACID | EICOSATETRAENOIC ACID, 5- | |
| 8-FLUORO-4- | | |
| | S-[5,6,8,9,11,12,14,15- | |
| HYDROXYQUINOLINE-3- | 3H(N)]- | |
| CARBOXYLIC ACID | | |
| 2-(4- | HYDROXY-5,8,10,14- | |
| 1-HYDROXYPHENYL) BUTANOIC | EICOSATETRAENOIC ACID, | |
| ACID | 12S | |
| AURORA KA-836 | [5,6,8,9,11,12,14,15- | |
| N′-(3-CHLORO-4- | 3H(N)]- | |
| HYDROXYBENZYLIDENE)AIVIINOM | ANPA, [5-METHYL-3H]- | |
| ETHANEHYDRAZONAMIDE | 9-HYDROXYIMINO-9H- | |
| ACETATE | FLUORENE-4-CARBOXYLIC | |
| 5,7-DICHLORO-4- | ACID | |
| HYDROXYQUINOLINE-3- | 2-((3-ALLYL-2-HYDROXY- | |
| CARBOXYLIC ACID | BENZYLIDENE)-AMINO)-3- | |
| 4-HYDROXY-6- | (1H-IMIDAZOL-4-YL)- | |
| (TRIFLUOROMETHOXY)QUINOLI | PROPIONIC ACID | |
| NE-3-CARBOXYLIC ACID | 3-(4-HYDROXY-PHENYL)-2- | |
| | ((NAPHTHALEN-2- | |
| YLMETHYLENE)-AMINO)- | ACID | |
| PROPIONIC ACID | 4-(4-HYDROXY-PHENYLAZO)- | |
| N-(3-HYDROXY-PHENYL)-5- | BENZOIC ACID | |
| NITRO-PHTHALAMIC ACID | | |
| 9-HYDROXYIMINO-9,10- | 4-HYDROXY-1-PHENYL- | |
| DIHYDRO-ANTHRACENE-1- | NAPHTHALENE-2-CARBOXYLIC | |
| CARBOXYLIC ACID | ACID | |
| 2-ETHYLSULFANYL-5-FLUORO- | 6-HEPTADECYLOXY-1- | |
| 6-HYDROXY-PYRIMIDINE-4- | HYDROXY-NAPHTHALENE-2- | |
| CARBOXYLIC ACID | CARBOXYLIC ACID | |
| | 4-(3,4,5-TRIHYDROXY-6- | |
| (4,6-DIHYDROXY-5-PHENYL- | HYDROXYMETHYL-TETRAHYDRO- | |
| PYRIMIDIN-2-YLSULFANYL)- | PYRAN-2-YLAMINO)-BENZOIC | |
| ACETIC ACID | ACID | |
| | 2-CARBOXYAMINO-3-(2- | |
| 5-FLUORO-6-HYDROXY-2- | | |
| | HYDROXY-PHENYL)-ACRYLIC | |
| METHYL-PYRIMIDINE-4 - | | |
| | ACID | |
| CARBOXYLIC ACID | | |
| 5-FLUORO-2,6-DIHYDROXY- | (DIHYDROXY-DI-ME-OXO- | |
| | HEXADECAHYDRO- | |
| PYRIMIDINE-4-CARBOXYLIC | | |
| | CYCLOPENTA(A)PHENANTHREN- | |
| ACID | | |
| 5-FLUORO-6-HYDROXY- | YL)-PENTANOIC ACID | |
| | 5-ACETYL-4-HYDROXY-3- | |
| PYRIMIDINE-4-CARBOXYLIC | | |
| | METHOXY-FURAN-2- | |
| ACID | | |
| 5-BROMO-6-HYDROXY- | CARBOXYLIC ACID | |
| PYRIMIDINE-4-CARBOXYLIC | (DIHYDROXY-DIMETHYL- | |
| | HEXADECAHYDRO- | |
| ACID | | |
| (5-ETHYL-4-HYDROXY-6- | CYCLOPENTA(A)PHENANTHREN | |
| METHYL-PYRIMIDIN-2- | 17-YL)-PROPIONIC ACID | |
| YLSULFANYL)-ACETIC ACID | 3-HYDROXY-7-OXO-7H- | |
| 1-(2-HYDROXYMETHYL-4-(HO- | BENZO(DE)ANTHRACENE-2- | |
| | CARBOXYLIC ACID | |
| PIPERIDIN-YL-PROPOXY)- | 2-(2,7-DICHLORO-6- | |
| PH)-PROPAN-ONE, BUT- | HYDROXY-3-OXO-2,3- | |
| ENEDIOIC ACID | DIHYDRO-1H-XANTHEN-9-YL)- | |
| 2-HYDROXY | | |
| CYCLOHEXANECARBOXYLIC | BENZOIC ACID | |
| 4-(2-HYDROXY-ETHYLAIMIINO)- | PHENYL-NAPHTHALENE-2- | |
| 3-NITRO-BENZOIC ACID | CARBOXYLIC ACID | |
| | 6A-(2-CYANO-ET)-5,7,8- | |
| (4-HYDROXY-3-SULFO- | TRIHYDROXY-4A-ME- | |
| PHENYLAMINO)-ACETIC ACID | OCTADECAHYDRO-CHRYSENE-2- | |
| | CARBOXYLIC ACID | |
| 4-(3-ETHYL-5-(2-HYDROXY- | 2-(6-HYDROXY-2,5,7- | |
| PHENYL)-4,5-DIHYDRO- | TRIIODO-3-OXO-3H-XANTHEN- | |
| PYRAZOL-1-YL)-BENZOIC | 9-YL)-BENZOIC ACID | |
| ACID | | |
| 5-(5-(4-HYDROXY-PHENYL)- | 2-HYDROXYAMINO-2-METHYL- | |
| 3-PHENYL-4,5-DIHYDRO- | PROPIONIC ACID | |
| PYRAZOL-1-YL)-ISOPHTHALIC | 2-HYDROXYAMINO-BUTYRIC | |
| ACID | ACID | |
| 2-(3-ACETYL-4-HYDROXY- | 2-HYDROXYIMINO-BUTYRIC | |
| PHENYLCARBAMOYL)- | ACID | |
| | 2-1-IYDROXYAMINO-4-METHYL- | |
| CYCLOHEX-1-ENECARBOXYLIC | PENTANOIC ACID | |
| ACID | 2-HYDROXYANINO-3-METHYL- | |
| 5-BENZO(1,3)DIOXOL-5-YL- | BUTYRIC ACID | |
| 8-HYDROXY-NAPHTHO(2,3- | 2-HYDROXYAMINO-3-METHYL- | |
| D)(1,3)DIOXOLE-6- | PENTANOIC ACID | |
| CARBOXYLIC ACID | 1-HYDROXYAMINO- | |
| | CYCLOHEXANECARBOXYLIC | |
| 1-(3-HYDROXY-PROPYL)- | | |
| | ACID | |
| 2,3,4,9-TETRAHYDRO-1H- | 5-HYDROXYMETHYL-2,6- | |
| BETA-CARBQLINE-3- | DIMETHYL-NICOTINIC ACID | |
| CARBOXYLIC ACID | | |
| 4-HYDROXY-7-METHOXY-1-(3- | 2-HYDROXYIMINO-3-(4- | |
| METHOXY-PHENYL)- | METHOXY-PHENYL)-PROPIONIC | |
| NAPHTHALENE-2-CARBOXYLIC | ACID | |
| ACID | 4-(3-HYDROXY- | |
| 1-HYDROXYMETHYL-2,3,4,9- | PROPYLAMINO)-3-NITRO- | |
| TETRAHYDRO-1H-BETA- | BENZOIC ACID | |
| CARBOLINE-3-CARBOXYLIC | | |
| ACID | | |
| 4-HYDROXY-5-METHOXY-1- | | |
| 4-HYDROXY-7-IODO-10-OXO- | 1-(3,4-DIMETHOXY-PH)-4- | |
| 9-OXA- | HYDROXY-5,6,7-TRIMETHOXY- | |
| TRICYCLO(6.2.1.0(1,5))UND | NAPHTHALENE-2-CARBOXYLIC | |
| ECANE-2-CARBOXYLIC ACID | ACID | |
| 1-BENZYL-3-HYDROXY- | 9,9-DIHYDROXY-5-OXO- | |
| | NONANOIC ACID | |
| 1,2,3,3A,4,7-HEXAHYDRO- | 3-(4-HYDROXY-2-METHYL- | |
| INDENE-1,7A-DICARBOXYLIC | CYCLOHEX-2-ENYL)- | |
| ACID | PROPIONIC ACID | |
| (5-HYDROXY-5H- | 2-(2-HYDROXY-5-METHYL- | |
| DIBENZO(A,D)CYCLOHEPTEN- | BENZOYL)-BENZOIC ACID | |
| 5-YL)-ACETIC ACID | 2-(2-HYDROXY-BENZOYL)- | |
| 2-CYANO-3-(3,4-DIHYDROXY- | BENZOIC ACID | |
| 5-METHOXY-PHENYL)-ACRYLIC | 6-HYDROXY-3-OXO-7A- | |
| ACID | PROPIONYL-OCTAHYDRO- | |
| 2-BENZYLAMINO-3-HYDROXY- | INDENE-1-CARBOXYLIC ACID | |
| PROPIONIC ACID | | |
| 4-HYDROXY-6-METHOXY-1-(4- | 4-HYDROXY-10-OXO-9-OXA- | |
| METHOXY-PHENYL)- | TRICYCLO(6.2.1.O(1,5))UND | |
| NAPHTHALENE-2-CARBOXYLIC | ECANE-2-CARBOXYLIC ACID | |
| ACID | 3-HYDROXY-6-METHOXY- | |
| 20-ISO-3,12- | | |
| | INDAN-1-CARBOXYLIC ACID | |
| DIHYDROXYBISNORCHOLANIC | | |
| ACID | 3-(5-HYDROXY-4-METHOXY-2- | |
| 4-(DIHYDROXY-DIMETHYL- | NITRO-PHENYL)-ACRYLIC | |
| HEXADECAHYDRO- | ACID | |
| CYCLOPENTA(A)PHENANTHREN- | 2-[(2- | |
| YL)-PENTANOIC ACID | CHLOROACETYL)AMINO]-3-(4- | |
| 3-(TRIHYDROXY-DI-ME- | HYDROXYPHENYL)PROPANOIC | |
| HEXADECAHYDRO- | ACID | |
| CYCLOPENTA(A)PHENANTHREN- | 4,6-DIHYDROXY-5- | |
| 17-YL)-BUTYRIC ACID | | |
| (TRIHYDROXY-DIMETHYL- | METHANESULFONYL-1-PH- | |
| HEXADECAHYDRO- | 3A,6-METHANO-AZULENE-3- | |
| | CARBOXYLIC ACID | |
| CYCLOPENTA(A)PHENANTHREN- | BUTTPARK 22\07-100 | |
| YL)-PROPIONIC ACID | 2-HYDROXY-4,6- | |
| DIMETHYLNICOTINIC ACID | ASIATIC ACID | |
| 4-HYDROXY-3,5- | 23-HYDROXYBETULINIC ACID | |
| | PYRIDINEDICARBOXYLIC ACID | |
| | [1R-(2-ENDO,3-EXO)]-3- | |
| HYDROXY-5,8,11,13- | HYDROXY-4,7,7- | |
| EICOSATETRAENOIC ACID, | TRIMETHYLBICYCLO[2.2.1]HE | |
| 15-5- | PTANE-2-ACETIC ACID | |
| [5,6,8,9,11,12,14,15- | 4-(DIMETHYLAMINO)-3- | |
| 3H(N)]- | HYDROXYBUTANOIC ACID | |
| HYDROXY-3-METHYLGLUTARYL | 2-[(2-HYDROXYIMINO-2-(3- | |
| COENZYME A, DL-3- | NITROPHENYL)ETHYL]THIO]BE | |
| [GLUTARYL-3-14C1- | NZOIC ACID | |
| EMOC-ASPARTAMINOL(OBUT) | 2-[[2-HYDROXYIMINO-2-(4- | |
| | NITROPHENYL)ETHYL]THIO]BE | |
| FMOC-GLUTAMINOL(OBUT) | NZOIC ACID | |
| ACETODIPHOSPHONIC ACID | | |
| 4-(2- | 5-HYDROXY-2-IODOBENZOIC | |
| HYDROXYHEXAFLUORCISOPROPY | ACID | |
| L)BENZOIC ACID | 2,4-DIHYDROXY-3,6- | |
| 2,3,4-TRIHYDROXY-5-OXO-2- | DIMETHYLBENZOIC ACID | |
| TETRAHYDROFUROIC ACID | 4-(1′, 1′-DIMETHYL-1′- | |
| | HYDROXYPROPYL)-PHENOXY- | |
| HYDROXYLAMINE-EDTA | ACETIC ACID | |
| 4-HYDROXY-6- | 6-HYDROXY-1-METHYL-2-OXO- | |
| (TRIFLUOROMETHYL)-3- | 1,2-DIHYDRO-4- | |
| QUINOLINECARBOXYLIC ACID | PYRIMIDINECARBOXYLIC ACID | |
| FMOC-HYP(BZL)-OH | 1-BENZYL-6-HYDROXY-2-OXO- | |
| N-ALPHA-HIPPURYL-L- | 1,2-DIHYDRO-4- | |
| ARGININIC ACID HCL | PYRIMIDINECARBOXYLIC ACID | |
| P-HYDROXY-BZ -ALA-PHE-OH | | |
| | 2-AMINO-3-[4- | |
| P-HYDROXYHIPPURIC ACID | (HYDROXYMETHYL)PHENYL]PRO | |
| H-DL-DELTA-HYDROXY-DL | | |
| | PANOIC ACID | |
| LYS(BOC)-OH | 3-(3-BROMO-4-HYDROXY- | |
| 3,5-DIHYDROXY-BZ-TYR-OH | PHENYL)-PROPIONIC ACID | |
| | (3-BENZYLOXY-4-METHOXY- | |
| PHENYL)-HYDROXY-ACETIC | 3-(3-HYDROXY-PHENYL)-2- | |
| ACID | METHYL-ACRYLIC ACID | |
| 2-HYDROXYIMINO- | 9,10-DIHYDROXY-18- | |
| PENTANEDIOIC ACID | (TOLUENE-4-SULFONYLOXY)- | |
| HYDROXYIMINO-PHENYL- | OCTADECANOIC ACID | |
| ACETIC ACID | | |
| 3-HYDROXY-3-(2-OXO-1,2- | 2-HYDROXYIMINO-PROPIONIC | |
| DIHYDRO-INDOL-3-YLIDENE)- | ACID | |
| | 2-HYDROXYIMINO-3-PHENYL- | |
| PROPIONIC ACID | | |
| | PROPIONIC ACID | |
| 1-(3,4-DIMETHOXY-PHENYL)- | 5,7-BIS(TRIFLUOROMETHYL)- | |
| 4-HYDROXY-6,7-DIMETHOXY- | 4-HYDROXYQUINOLINE-3- | |
| NAPHTHALENE-2-CARBOXYLIC | CARBOXYLIC ACID | |
| ACID | | |
| 2-(3,5-DI-TERT-BUTYL-4- | 4-FLUORO-3-HYDROXYBENZOIC | |
| HYDROXY-BENZYLIDENE)- | ACID | |
| | [4-FLUORO-3- | |
| MALONIC ACID | | |
| 10,11-DIHYDROXY- | HYDROXYBENZOIC ACID | |
| | 4-FLUORO-3-HYDROXYBENZOIC | |
| UNDECANOIC ACID | | |
| 9,10-DIHYDROXY- | ACID | |
| | 4-FLUORO-3-HYDROXYBENZOIC | |
| OCTADECANEDIOIC ACID | | |
| | ACID | |
| MONOETHYL ESTER | | |
| 9,10,18-TRIHYDROXY- | 4-FLUORO-3-HYDROXYBENZOIC | |
| OCTADECANOIC ACID | ACID | |
| 2-(1-HYDROXY-OCTADECYL)- | 4-FLUORO-3-HYDROXYBENZOIC | |
| HEXACOSANOIC ACID | ACID] | |
| | 3,5-DIIODO-P- | |
| 3-HYDROXY-2-METHYL- | HYDROXYPHENYLPYRUVIC ACID | |
| EICOSANOIC ACID | | |
| 2-HEXADECYL-3-HYDROXY- | ALLOCHOLIC ACID | |
| EICOSANOIC ACID | 7,7-AZO-3-ALPHA,12-ALPHA- | |
| 3-(4-HYDROXY-3-METHOXY- | DIHYDROXYCHOLANIC ACID | |
| PHENYL)-2-METHYL-ACRYLIC | | |
| | TRANS-1-(3′-CARBOXY-4′- | |
| ACID | | |
| 3-HYDROXY-2-METHYL-3-(3 | HYDROXYPHENYL)-2- | |
| NITRO-PHENYL)-PROPIONIC | (2″, 5″, -DIHYDROXY- | |
| ACID | PHENYL)ETHANE | |
| 1-HYDROXYCHLORODIENE | 3-HYDROXY-5-METHYL- | |
| HEMISUCCINATE | HEPTANOIC ACID | |
| 4-HYDROXY-3-(F- | N-HYDROXYMETHYL SARCOSINE | |
| DIAZOPHENYLPHOSPHORYLOHOL | | |
| INE)-PHENYLACETIC ACID | BOC-L-5-HYDROXY-TRP | |
| | NITROSALICYLIC ACID | |
| NW-HYDROXYL-L-ARGININE, | 5-AMINO-N-(2,3-DIHYDROXY- | |
| DIHYDROCHLORIDE | 1-PROPYL)ISOPHTHALANIC | |
| | ACID | |
| 6-HYDROXYTRYPTAMINE, | | |
| CREATINE SULFATE | NG-MONOMETHYL-L-ARGININE, | |
| 4-HYDROXY-3-METHYLBENZOIC | DI-P-HYDROXYAZOBENZENE | |
| ACID | P′-SULFONATE SALT | |
| BOC-(3S,4S)-4-AMINO-3- | | |
| HYDROXY-5-METHYLHEXANOIC | 4-ANDROSTEN | |
| ACID DICYCLOHEXYLAMMONIUM | 11ALPHA,17BETA-DIOL-3-ONE | |
| SALT | 11-HEMISUCCINATE | |
| | 4-ANDROSTEN-11BETA-OL- | |
| BOC-(3S,4S)-4-AMINO-3- | 3,17-DIONE3-CMO | |
| HYDROXY-5-(4- | 5-ANDROSTEN- | |
| BENZYLOXYPHENYL)- | 3BETA,16ALPHA-DIOL-17-ONE | |
| PENTANOIC ACID | DIHEMISUCCINATE | |
| BOC-(35,4S)-4-AMINO-3- | 5ALPHA-CHOLANIC ACID | |
| HYDROXY-5-(2-INDOLYL)- | 22,23-BISNOR-3BETA-OL | |
| PENTANOIC ACID | 3-HYDROXY-BIS-NORCHOLENIC | |
| FMOC-(35,45)-4-AMINO-3- | ACID | |
| HYDROXY-5-METHYL HEXANOIC | 5BETA-CHOLANIC ACID- | |
| ACID DICYCLOHEXYLAMMONIUM | 3ALPHA-OL-7-ONE | |
| | PROGESTERONE-6BETA-OL-6- | |
| SALT | HEMISUCCINATE | |
| FMOC-(3S,4S)-4-AMINO-3- | PROGESTERONE-11ALPHA- | |
| HYDROXY-6-METHYLTHIO- | [BETA-D-GLUCURONIDE] | |
| | 4-PREGNEN-17-OL-3,20- | |
| HEXANOIC ACID | | |
| FMOC-(3S,4S)-4-AMINO-3- | DIONE 3-CMO:BSA | |
| | 5-PREGNEN-3BETA,17-DIOL- | |
| HYDROXY-PENTANOIC ACID | | |
| | 20-ONE 3-HEMISUCCINATE | |
| DICYCLOHEXYLAMMONIUM SALT | | |
| FMOC-(3S,4S,5S)-4-AMINO- | DL-ALLO-HYDROXYLYSINE | |
| HYDROCHLORIDE | (2S,3R)-3-AMINO-2- | |
| D-(3,4-DIHYDROXY)- | HYDROXY-4-PHENYLBUTYRIC | |
| PHENYLGLYCINE | | |
| 4-[(3-HYDROXY-2- | ACID HYDROCHLORIDE | |
| NAPHTHYL)CARBONYL]AMINO]B | 9-([E]-2-CARBOXY-2- | |
| ENZOIC ACID | CYANOVINYL)JULOLIDINE | |
| (RS)-3,5-DHPG | 10-HYDROXYCAMPTOTHECIN | |
| ERYTHRO-BETA-HYDROXY-L- | ACETATE SALT | |
| ASPARTIC ACID | 10-HYDROXYCAMPTOTHECIN | |
| HYDROXYETHYL | ACETATE SALT | |
| ETHYLENEDIALVITNE TRIACETIC | 3-NITRO-5-SULFOSALICYLIC | |
| ACID | ACID | |
| DELTA-DL(+)ALLO HYDROXY- | 2-(1,2,3,4- | |
| L-LYS HCL | TETRAHYDROXYBUTYL)-1,3- | |
| DL-3-HYDROXYGLUTAMIC ACID | THIAZOLANE-4-CARBOXYLIC | |
| | ACID | |
| 19-HYDROXYTESTOSTERONE- | | |
| 19-CARBOXYMETHYLETHER | HYDROXYBICYCLO[2.2.2]OCTA | |
| 5-ANDROSTEN- | | |
| | NE-2-CARBOXYLIC ACID | |
| 3BETA,16ALPHA-DIOL-17-ONE | | |
| 16-HEMISUCCINATE | 1-ACETYL-4- | |
| 17ALPHA- | HYDROXYPYRROLIDINE-2- | |
| HYDROXYPROGESTERONE3-(O- | CARBOXYLIC ACID | |
| CARBOXYMETHYL)OXIME | 4-(3,12-DIHYDROXY-10,13- | |
| JMV 390-1 | DIMETHYLPERHYDROCYCLOPENT | |
| [DES-ARG10]HOE 140 | A[A]PHENANTHREN-17- | |
| M-HYDROXYBENZOYLECGONINE | | |
| | YL)PENTANOIC | |
| P-HYDROXYBENZOYLECGONINE | 2-(1,2,3,4,5- | |
| | PENTAHYDROXYPENTYL)-1,3- | |
| (R)-(+)-2-(4 | THIAZOLANE-4-CARBOXYLIC | |
| HYDROXYPHENOXY)PROPIONIC | ACID | |
| ACID | 5-[2-[[2-(3-CARBOXY-4- | |
| (2R,3R)-3-AMINO-2- | HYDROXYPHENYL)DIAZ-1- | |
| HYDROXY-4-PHENYLBUTYRIC | ENYL](CYANO)METHYLIDENE]H | |
| ACID HYDROCHLORIDE | YDRAZINO]-2- | |
| | 2-[2-(2- | |
| HYDROXYPHENYL)HYDRAZONO]- | HYDROXYVINYL)-2- | |
| 2-PHENYLACETIC ACID | NITROBENZOIC ACID | |
| 2-[[2-(4-FLUOROPHENYL)-2- | 2-[(2- | |
| HYDROXYIMINOETHYL]THIO]BE | HYDROXYBENZYL)AMINO]-4- | |
| NZOIC ACID | METHYLPENT-3-ENOIC ACID | |
| 5-CHLORO-2-[[2- | | |
| (HYDROXYMETHYL)PHENYL]THI | (ACETYLAMINO)PHENYL]SULFO | |
| O]BENZOIC ACID | NYL]AMINO)-2- | |
| 2-[[2- | QUINOXALINYL]AMINO]-2- | |
| (HYDROXYMETHYL)PHENYL]THI | HYDROXYBE | |
| O]-5-NITROBENZOIC ACID | 2-HYDROXY-4-([3- | |
| 2-[[2- | [(PHENYLSULFONYL)AMINO]- | |
| (HYDROXYMETHYL)PHENYL]THI | 2- | |
| O]-5-METHYLBENZOIC ACID | QUINOXALINYL]AMINO)BENZOI | |
| | C ACID | |
| 2-[[2- | N-(4-HYDROXY-PHENYL)-5- | |
| (HYDROXYMETHYL)PHENYL]THI | NITRO-PHTHALAMIC ACID | |
| O]-5-METHOXYBENZOIC ACID | 4-(4-CHLOROANILINO)-2,3- | |
| | DIHYDROXY-4-OXOBUTANOIC | |
| 4-(2,6-DIMETHYLPHENYL)-2- | | |
| | ACID | |
| HYDROXY-4-OXOBUT-2-ENOIC | 2-(2-HYDROXYPHENYL)-1,3- | |
| ACID | THIAZOLANE-4-CARBOXYLIC | |
| 2-[(2- | | |
| | ACID | |
| HYDROXYBENZYL)AMINO]-3- | ASINEX-REAG BAS 0873809 | |
| PHENYLPROPANOIC ACID | | |
| 4-AMINO-2-[(2- | 5-(ACETYLAMINO)-2- | |
| HYDROXYBENZYL)AMINO]-4- | HYDROXYBENZOIC ACID | |
| OXOBUTANOIC ACID | 3,4,5-TRICHLORO-6- | |
| 6-BUTYL-4-HYDROXY-8- | HYDROXY-PYRIDINE-2- | |
| METHYLQUINOLINE-3- | CARBOXYLIC ACID | |
| CARBOXYLIC ACID | NITR 7 | |
| 5-CHLORO-2-[[2- | 4-ANDROSTEN- | |
| (HYDROXYMETHYL)PHENYL]SUL | 11ALPHA,17BETA-DIOL-3-ONE | |
| FINYL]BENZOIC ACID | 11-HEMISUCCINATE:BSA | |
| 3-(1-FORMYL-2- | | |
| 5-PREGNEN-3BETA,17ALPHA- | HYDROXYBUTANOIC ACID | |
| DIOL-20-ONE-7-CM:BSA | 6-HYDROXYPALMITIC ACID | |
| | 2-(ACETYLAMINO)-3-(4- | |
| 6-AMINOQUINOLINE, L- | HYDROXY-3,5- | |
| HYDROXYPROLINE AMIDE, | DINITROPHENYL)PROPANOIC | |
| DI(TRIFLUOROACETATE) SALT | ACID | |
| | 3-[6-ETHYL-7-HYDROXY-3- | |
| 7-HYDROXY-4- | | |
| | (1-METHYL-1H- | |
| METHYLCOUMARIN-3-ACETIC | | |
| | BENZO[D]IMIDAZOL-2-YL)-4- | |
| ACID | | |
| FMOC-APNS | OXO-4H-CHROMEN-2-YL | |
| | 4-HYDROXYQUINOLINE-3- | |
| HYDROXYETHYLETHYLENEDIAMI | | |
| | CARBOXYLIC ACID | |
| NETRIACETIC ACID, | PHILANTHOTOXIN 343 TRIS- | |
| TRISODIUM SALT | TRIFLUOROACETATE | |
| GAMMA-GLUTAMINYL-3,4- | PHILANTHOTOXIN 433 TRIS- | |
| DIHYDROXYBENZENE | TRIFLUOROACETATE | |
| (E)-3-HYDROXYTAMOXIFEN | XAMOTEROL HEMIFUMARATE | |
| CITRATE | 4-[4-[(2- | |
| GHB | | |
| 3-HYDROXY-2,2-DIMETHYL-3- | HYDROXYBENZOYL)AMINO]ANIL | |
| | INO-4-OXOBUT-2-ENOIC | |
| PHENYLPROPIONIC-ACID | | |
| 2-(9H-FLUOREN-9- | ACID | |
| | 1-[4-[(5-CARBOXY-2- | |
| YLMETHOXYCARBONYLAMINO)- | | |
| 3-HYDROXY-BUTYRIC ACID | HYDROXYANILINO)CARBONYL]B | |
| | ENZYLI PYRIDINIUM CHLORIDE | |
| 4-HYDROXY-3,5- | | |
| | 2-[(4-HYDROXY-5-METHOXY- | |
| DIISOPROPYL-BENZOIC ACID | | |
| | 2- | |
| 3-(3-HYDROXY-1- | PYRIMIDINYL)SULFANYL]ACET | |
| ADAMANTYL)PROPANOIC ACID | IC ACID | |
| | BUTTPARK 15\01-32 | |
| 4-[(3,5-DIBROMO-4- | BUTTPARK 16\02-06 | |
| HYDROXYPHENYL)SULFONYL]BE | 6-CARBOXYMETHYL-2,3- | |
| NZOIC ACID | DIHYDROXY-BENZOIC ACID | |
| 17-HYDROXYHEPTADECANOIC | 5-HYDROXY-CYCLOHEXANE- | |
| ACID | 1,3-DICARBOXYLIC ACID | |
| | 4-AMINO-1-HYDROXY- | |
| 3-(1-ADAMANTYL)-3- | 2,2,6,6-TETRAMETHYL- | |
| PIPERIDINE-4-CARBOXYLIC | 2-METHOXY-3,5-DIMETHYL- | |
| ACID | BENZOIC ACID,2-HYDROXY- | |
| 3-HYDROXY-5-(1-HYDROXY-1- | 3,5-DIMETHYL-BENZOIC ACID | |
| METHYL-ETHYL)- | | |
| | 3-HYDROXY-12-OXO-CHOLANIC | |
| CYCLOHEXANECARBOXYLIC | | |
| | ACID | |
| ACID | | |
| 2-MEO-4-METHYL-6-(2,2,2- | 11-BROMO-3-ALPHA-HYDROXY- | |
| TRICHLORO-1-HYDROXY- | 12-OXO-5-CHOLAN-24-OIC | |
| | ACID | |
| ETHYL)- | 3-BETA-ACETOXY-17-ALPHA- | |
| CYCLOHEXANECARBOXYLIC | ETHOXYCARBONYL-5-HYDROXY- | |
| ACID | | |
| 2-(CARBOXY-HYDROXY- | 5-BETA,14-BETA-ANDROSTAN- | |
| | 19-OIC | |
| METHYL)-6-HYDROXY-4- | 8,19-EPOXY-3-BETA,5- | |
| METHYL- | DIHYDROXY-19-MEO-5-BETA- | |
| CYCLOHEXANECARBOXYLIC | ANDROSTANE-17-BETA- | |
| ACID | | |
| 2-ETHYL-3-(3-HYDROXY- | CARBOXYLIC ACID | |
| | 3-BETA-ACETOXY-5-HYDROXY- | |
| PHENYL)-ACRYLIC ACID | 5-BETA,17-ALPHA-CARDA- | |
| 2-(4-CHLORO- | | |
| BENZOYLAMINO)-3-HYDROXY- | 14,20(22)-DIENOLID-19-OIC | |
| PROPIONIC ACID | 2-HYDROXYOCTANOIC ACID, | |
| 2-(3-CHLORO- | [1-14C] | |
| BENZOYLAMINO)-3-HYDROXY- | (S)-ALPHA-AMINO-3- | |
| PROPIONIC ACID | HYDROXY-5-[3H]-METHYL-4- | |
| 5-(2-HYDROXY-3-METHOXY- | ISOXAZOLEPROPIONIC ACID | |
| PHENYL)-PENTANOIC ACID | | |
| 5-(3-HYDROXY-4-METHOXY- | N-ACETYL-HYDROXY-PROLINE, | |
| PHENYL)-PENTANOIC ACID | L-4[3H(G)] | |
| 5-(4-HYDROXY-3-METHOXY- | 14-HYDROXY-10,12- | |
| PHENYL)-PENTANOIC ACID | TETRADECADIYNOIC ACID | |
| 2-HYDROXY-3,5-DIMETHYL- | 8-HYDROXY-7- | |
| BENZOIC ACID | QUINOLINECARBOXYLIC ACID | |
| 2,6-DIHYDROXY-3,5- | | |
| DIMETHYL-BENZOIC ACID | 3-HYDROXY-2-METHYLBENZOIC | |
| | ACID | |
| | 2-HYDROXY-3-BUTYNOIC ACID | |
| (2R,3S)-3-PHENYLISOSERINE | ACID | |
| | 2-HYDROXYTRICOSANOIC ACID | |
| BOC-L-7-HYDROXY-1,2,3,4- | | |
| TETRAHYDROISOQUINOLINE-3- | (2R,3R)-1-CARBOXY-4,5- | |
| CARBOXYLIC ACID | DIHYDROXYCYCLOHEXA-4,6- | |
| | DIENE | |
| 4-HYDROXY-3-METHYL(ALPHA- | (S)-3,5- | |
| AMINOACETIC ACID)BENZOIC | DIHYDROXYPHENYLGLYCINE | |
| ACID | D(+)-THREO-3- | |
| | HYDROXYASPARTIC ACID | |
| DEUTEROPORPHYRIN IX 2,4 | L(−)-THREO-3- | |
| (4,2) HYDROXYETHYL VINYL | HYDROXYASPARTIC ACID | |
| | 6,7-DICHLORO-3-HYDROXY-2- | |
| FMOC-CYS(2-HYDROXYETHYL)- | QUINOXALINECARBOXYLIC | |
| OH | ACID | |
| H-(2S,4S)-GAMMA-HYDROXY | | |
| GLU-OH | 2,4- | |
| H-(2S,4R)-GANMA-HYDROXY- | DIHYDROXYPHENYLACETYL-L | |
| GLU-OH | ASPARAGINE | |
| (R)-AMPA | (R)-4-BROMO-HOMOIBOTENIC | |
| (S)-AMPA | ACID | |
| 5-CHLOROKYNURENIC ACID | (S)-4-BROMO-HOMOIBOTENIC | |
| HYDROCHLORIDE | | |
| (RS)-ALPHA-METHYL-3- | ACID | |
| | 6-CHLOROKYNURENIC ACID | |
| CARBOXY-4-HYDROXYPHENYL- | (S)-4-METHYLHOMOIBOTENIC | |
| GLYCINE | ACID | |
| (RS)-3-CARBOXY-4- | | |
| HYDROXYPHENYLOLYCINE | (+/−)-3- | |
| (S)-3-CARBOXY-4- | HYDROXYPHENYLGLYCINE | |
| HYDROXYPHENYLGLYCINE | (S)-3- | |
| (R)-4-CARBOXY-3- | HYDROXYPHENYLGLYCINE | |
| HYDROXYPHENYLGLYCINE | (R)-3-CARBOXY-4- | |
| (S)-4-AMINO-3- | HYDROXYPHENYLGLYCINE | |
| HYDROXYBUTYRIC ACID | TRIFLUOROAMPA | |
| 5-HYDROXYTRYPTAMINE | TRANS-4-HYDROXYCROTONIC | |
| BINOXALATE, [ETHYLAMINE- | ACID | |
| 1,2-3H]- | (E)-4-HYDROXY-4-(4- | |
| 3-HYDROXYOCTADECANOIC | NITROPHENYL)-2-BUTENOIC | |
| ACID | 5,12-DIHYDROXY-11-METHYL- | |
| 3,5-DIIODO-4- | 6-METHYLENE-16-OXO-15- | |
| HYDROXYPHENYLPROPIONIC | OXAPENTACYCLO[9.3.2.1- | |
| ACID | | |
| CIS-1-AMINO-3-(2- | 5,8-.0-1,10 | |
| PHOSPHONO-1- | FMOC-TIC(OH)-OH | |
| HYDROXYETHYL)- | BOC-D-7-HYDROXY-1,2,3,4- | |
| CYCLOBUTANE-1-CARBOXYLIC | TETRAHYDROISOQUINOLINE-3- | |
| | CARBOXYLIC ACID | |
| ACID | | |
| ICI 185,282 | FMOC-D-TIC(OH)-OH | |
| ICI 192,605 | 9-HYDROXYNONANOIC ACID | |
| ICI 215,001 HYDROCHLORIDE | 8-HYDROXYOCTANOIC ACID | |
| | L-(−)-N-BENZOXYLCARBONYL- | |
| 6-METHOXYSALICYLIC ACID | | |
| | ALPHA-HYDROXY-GAMMA-AMINO | |
| (S)-(−)-4-AMINO-2- | BUTYRIC ACID AMIKACIN | |
| HYDROXYBUTYRIC ACID | (2R)-2-[(4-ETHYL-2,3- | |
| TRANS-7-HYDROXY-PIPAT | DIOXOPIPERAZINYL)CARBONYL | |
| MALEATE | | |
| 2-(4-HYDROXYMETHYL-3- | AMINO]-2-(4- | |
| METHOXYPHENOXY)BUTYRIC | HYDROXYPHENYL)ACETIC A | |
| | R-PHTHALIMIDO-ALPHA- | |
| ACID | HYDROXY-N-BUTYLIC ACID | |
| L-(+)-7- | M-HYDROXYBENZOIC ACID, | |
| HYDROXYAMETHOPTERIN | | |
| 7-CHLOROKYNURENIC ACID | [CARBOXYL-14C]- | |
| | N-T-BOC-CIS-4-HYDROXY-D- | |
| HYDROCHLORIDE | | |
| ASINEX-REAG BAS 0367332 | PROLINE | |
| | 2-(HYDROXY-PHENYL | |
| 2-[(5-HYDROXY-4-OXO-2- | PHOSPHINOYL)-BENZOIC ACID | |
| PHENYL-4H-CHROMEN-7- | N-(1-ETHOXYCARBONYL-2- | |
| YL)OXY]PROPANOIC ACID | HYDROXY-2-PHENYL-ETHYL)- | |
| H-D-TIC(OH)-OH 2H2O | | |
| | SUCCINAMIC ACID | |
| 2-HYDROXY-4,6- | 2-NITRO-4-(2,2,2- | |
| DI(TRIFLUOROMETHYL)BENZOI | TRIFLUORO-1-HYDROXY-1- | |
| C ACID | | |
| (RS)-3,4,5-THPG | TRIFLUOROMETHYL-ETHYL)- | |
| | BENZOIC ACID | |
| 1-ET-6-FLUORO-7-HYDROXY- | F1BETA | |
| 4-OXO-1,4-DIHYDRO- | 11-DEOXY PROSTAGLANDIN | |
| | F1ALPHA | |
| (1,8)NAPRTHYRIDINE-3- | 1A,1B-DIHOMO | |
| CARBOXYLIC ACID | PROSTAGLANDIN F2ALPHA | |
| 4-(2-HYDROXY-PHENYL)- | 8-ISO-15(R)-PROSTAGLANDIN | |
| BUTYRIC ACID | F2ALPHA | |
| N-AMIDINO-N-(2,3- | 11-DEOXY PROSTAGLANDIN | |
| DIHYDROXY PROPYL)GLYCINE | F2ALPHA | |
| | TETRANOR PGFM | |
| Z-TIC(7-OH)-OH | 6ALPHA-PROSTAGLANDIN I1 | |
| 8-ISOPROSTAGLANDIN A1 | | |
| 16-PHENOXY TETRANOR | 5-CISCARBAPROSTACYCLIN | |
| PROSTAGLANDIN A2 | | |
| 19(R)-HYDROXY | [3H]-PROSTAGLANDIN H2 | |
| PROSTAGLANDIN A2 | 8-ISOPROSTAGLANDIN | |
| 19(R)-HYDROXY | F2ALPHA-D4 | |
| PROSTAGLANDIN B2 | TETRANOR 12(R)-HETE | |
| PROSTAGLANDIN B3 | 2-HYDROXY-4-AMINOBUTYRIC | |
| 15-DEOXY-DELTA12,14- | ACID | |
| PROSTAGLANDIN D2 | 4-(PHOSPHONOMETHYL)-D,L | |
| 1A,1B-DIHOMO | PHENYLALANINE | |
| PROSTAGLANDIN E1 | 2-CHLORO-4-HYDROXYBENZOIC | |
| AH 13205 | ACID HYDRATE | |
| 13,14-DIHYDRO-19(R)- | | |
| HYDROXY PROSTAGLANDIN E1 | (2R,3R)-2-BENZYL-3- | |
| | HYDROXYBUTYRIC ACID | |
| 16,16-DIMETHYL | (+/−)-7-HYDROXY-1,2,3,4- | |
| PROSTAGLANDIN E1 | TETRAHYDRO-3- | |
| 16-PHENYL TETRANOR | ISOQUINOLINE-4-CARBOXYLIC | |
| PROSTAGLANDIN E1 | ACID METHYL ESTER | |
| 11-DEOXY PROSTAGLANDIN E2 | | |
| | TETRANOR-12(R)-HETE | |
| 16-PHENOXY TETRANOR | R-4-HYDROXYMANDELIC ACID | |
| PROSTAGLANDIN E2 | | |
| 16-PHENYL TETRANOR | S-4-HYDROXYMANDELIC ACID | |
| PROSTAGLANDIN E2 | | |
| TETRANOR PGEM | FMOC-D-HYP(TBU)-OH | |
| 11-DEOXY PROSTAGLANDIN | CYANOPINDOLOL | |
| HEMIFUMARATE | N(G)-HYDROXY-L-ARGININE | |
| 7 -HYDROXY-4-METHYL-3- | | |
| COUMARINYLACETIC ACID | 3- | |
| 3-(2-CARBOXYETHYL)-2-(3- | HYDROXYMETHYLPYRIDINIUMHY | |
| ((3- 2-HYDROXYETHYL)- | DROGEN-L(+)-TARTARIC ACID | |
| METHYL-BENZOTHIAZOL- | SALT | |
| 2(1H)-YLIDENE | 6-AMINOHEXANOIC ACID | |
| (R)-(+)-3-HYDROXY-5-OXO- | MATRIX | |
| | 6-AMINOHEXANOIC ACID | |
| 1-CYCLOPENTENE-1- | | |
| | MATRIX | |
| HEPTANOIC ACID | | |
| 4-HYDROXYMETHYL-3- | | |
| | HYDROXYBENZYL)BUTANOIC | |
| METHOXY-PHENOXY-VALERIC | | |
| | ACID | |
| ACID | 3-(3,5-DIBROMO-4- | |
| BOC-(3S,4S)-4-AMINO-3- | | |
| | HYDROXYPHENYL)PROPANOIC | |
| HYDROXY-5-(3-INDOLYL)- | | |
| | ACID | |
| PENTANOIC ACID | 2-[(4-HYDROXY-2-METHYL-3- | |
| BOC-AHMHPA-OH DCHA | | |
| 3-HYDROXY-2,4,5- | PYRIDYL)OXY]ACETIC ACID | |
| TRIFLUOROBENZOIC ACID | 2-[(5-BROMO-4-HYDROXY-2- | |
| (S)-(+)-2-AMINO-3- | | |
| | METHYL-3- | |
| HYDROXY-3-METHYLBUTANOIC | | |
| | PYRIDYL)OXY]ACETIC ACID | |
| ACID | | |
| (S)-(+)-2-AMINO-3- | H-TIC(OH)-OH 2H2O | |
| HYDROXY-3-METHYLBUTANOIC | 2-(2,5-DIMETHYLPHENYL)-2- | |
| ACID | HYDROXYACETIC ACID | |
| (S)-(+)-2-AMINO-3- | 2-(1,2,3,4- | |
| HYDROXY-3-METHYLBUTANOIC | TETRAHYDROXYBUTYL)-1,3- | |
| ACID | THIAZOLANE-4-CARBOXYLIC | |
| D,L-6-HYDROXY-1,2,3,4- | ACID | |
| TETRAHYDROISOQUINOLINE-3- | 2-(3-CHLOROPHENYL)-2- | |
| CARBOXYLIC ACID | HYDROXYACETIC ACID | |
| 2-ANINO-3-(3′- | 3-[(TERT- | |
| HYDROXYPHENYL)-BUTYRIC | BUTOXYCARBONYL)AMINO]-2- | |
| ACID | HYDROXY-4-PHENYLBUTANOIC | |
| KETOROLAC TROMETHAMINE | ACID | |
| 6- | 2- | |
| HYDROXYBICYCLO[2.2.2]OCTA | HYDROXYBICYCLO[2.2.1]HEPT | |
| NE-2-CARBOXYLIC ACID | ANE-1-CARBOXYLIC ACID | |
| 3-L-[3-HYDROXY-4- | 2-(1,2,3,4,5- | |
| (HYDROXYMETHYL)TETRAHYDRO | PENTAHYDROXYPENTYL)-1,3- | |
| FURAN-2-YL]-2,4-DIOXO- | THIAZOLANE-4-CARBOXYLIC | |
| 1,2,3,4-TETRAHY | ACID | |
| 4-HYDROXY-5-OXO-2,3- | 2-(1,2,3,4- | |
| DIPHENYL-2,5- | TETRAHYDROXYBUTYL)-1,3- | |
| DIHYDROFURAN-2-CARBOXYLIC | THIAZOLANE-4-CARBOXYLIC | |
| ACID | ACID | |
| 7-HYDROXY-2,4B-DIMETHYL- | 3-(AMINOCARBONYL)-3- | |
| 1-[(3-OXO-2,3-DIHYDRO-1H- | HYDROXY-4-METHYLPENTANOIC | |
| 2- | ACID | |
| INDOLYLIDEN)METHYL]PERHYD | 2-[(3- | |
| RO-2 | CHLOROBENZOYL)AMINO]-3- | |
| 2,4-DI(4- | HYDROXYPROPANOIC ACID | |
| HYDROXYPHENYL)CYCLOBUTANE | HYDRATE | |
| 1,3-DICARBOXYLIC ACID | | |
| | HYDROXYPHENYL)-1H- | |
| 10-[[3-(ACETYLAMINO)-4,5- | 1,2,3,4-TETRAAZOL-5- | |
| DIHYDROXY-6- | | |
| | YL]THIO]ACETYL)AMINO]- | |
| (HYDROXYMETHYL)TETRAHYDRO | 1,3- | |
| -2H-PYRAN-2-YL]OXY | 9-DEOXY-9-METHYLENE- | |
| 10-[[3-(ACETYLAMINO)-4,5- | 16,16-DIMETHYL | |
| DIHYDROXY-6 | PROSTAGLANDIN E2 | |
| (HYDROXYMETHYL)TETRAHYDRO | LIMAPROST | |
| -2H-PYRAN-2-YL]oxy | 3-HYDROXYVALPROIC ACID | |
| 5,5-DIMETHYL-2-(1,2,3,4- | MISOPROSTOL | |
| TETRAHYDROXYBUTYL)-1,3- | ISOCARBACYCLIN | |
| THIAZOLANE-4-CARBOXYLIC | N-[3,5-DIMETHYL-4-(4′- | |
| ACID | HYDROXY-3′- | |
| | ISOPROPYLPHENOXY)PHENYL]O | |
| | XAMIC ACID | |
| | 6-FLUORO-4- | |
| HYDROXYQUINOLINE-3- | PENTANEDIOIC ACID | |
| CARBOXYLIC ACID | 9-TRANS-12 HYDROXY- | |
| 3,4-DIHYDROXYBENZOIC ACID | OCTADECENOIC ACID | |
| [CARBOXYL-14C] | 4CL-3,5-DHPG | |
| 4-HYDROXY-3- | HOMOAMPA | |
| METHOXYBENZOIC ACID [1- | L-4-HYDROXYPHENYLALANINE- | |
| 14C] | 2-13C, 15N | |
| 2-HYDROXYNICOTINIC ACID | | |
| | DL-3-HYDROXYDODECANOIC- | |
| [2-14C | | |
| HYDROXY-6,8,11,14- | 2,2,3,4,4,-D5 ACID | |
| | 3-HYDROXYTETRADECANOIC- | |
| EICOSATETRAENOIC ACID | | |
| | 2,2,3,4,4-D5 ACID | |
| 5(S)- | L-4-HYDROXYPHENYLALANINE | |
| (5,6,8,9,11,12,14,15-3H] | 1,2,3-13C3 | |
| HYDROXY-5,8,10,14- | 4-HYDROXYBENZOIC-CARBOXY | |
| EICOSATETRAENOIC ACID | | |
| | 13C ACID | |
| 12(S)- | 4-HYDROXYBENZOIC-13C6 | |
| [5,6,B,9,11,12,14,15-3H] | ACID (RING-13C6) | |
| 2-HYDROXYBENZOIC ACID- | L-4-HYDROXY-ISO- | |
| ALPHA-13C | PHENYLALANINE | |
| 3-HYDROXYBENZOIC ACID-A- | 5-HYDROXYTRYPTAMINE | |
| 13C | BINOXALATE, [1,2-3H(N)] | |
| L-4-HYDROXYPHENYL-D4- | | |
| | GAMMA-HYDROXYBUTYRIC | |
| ALANINE-2,3,3-D3 | | |
| 5-HYDROXYINDOLE-3-ACETIC- | ACID, [2,3-3H]- | |
| | (3R,4S)N-BOC-4-AMINO-5- | |
| 2,2-D2 ACID | | |
| DL-4- | PHENYL-3-HYDROXY VALERIC | |
| HYDROXYPHENYLALANINE-3,3- | ACID | |
| | (2R,3S)-3- | |
| D2 | | |
| DL-4-HYDROXYPHENYL-2,6- | (ETHOXYCARBONYL)-AMINO-2- | |
| D2-ALANINE-2-D1 | HYDROXY-4-CYCLOHEXYL- | |
| DL-DOPA-ALPHA-D1 HBR | BUTANOIC ACID | |
| L-4-HYDROXYPHENYL-2,6-D2- | 4-HYDROXYMETHYL-3-NITRO- | |
| ALANINE-2-D1 | BENZOIC ACID | |
| 4-HYDROXYBENZOIC-2,3,5,6- | MAP KINASE SUBSTRATE, | |
| D4 ACID | TYROSINE HYDROXYLASE 24- | |
| 3-HYDROXY-3-METHYL-D3- | 33 | |
| L-5-HYDROXYLYSINE | 4-PREGNEN- | |
| DIHYDROCHLORIDE | 6BETA,11BETA,17,21- | |
| MONOHYDRATE | TETROL-3,20-DIONE 21- | |
| 2-HYDROXY-5- | HEMISUCCINATE | |
| (TRIFLUOROMETHOXY)BENZOIC | 3,5-DIIODOTHYROACETIC | |
| ACID | ACID | |
| 4-HYDROXY-2- | APSTATIN TRIFLUOROACETATE | |
| (TRIFLUOROMETHYL)BENZOIC | SALT | |
| ACID | 2-FLUORO-4-HYDROXYBENZOIC | |
| 4-HYDROXY-3- | ACID | |
| (TRIFLUOROMETHYL)BENZOIC | (+/−)-ALPHA-AMINO-3- | |
| ACID | HYDROXY-5-METHYL- | |
| 3-NITRO-5-HYDROXYBENZOIC | ISOXAZOLE-4-PROPIONIC | |
| ACID | ACID DIHYDRATE | |
| 2,4,2′-TRINITRO-5- | 4-HYDROXY-3-METHOXY- | |
| HYDROXY-6,6′-DIMETHYL- | PHENYLACETIC-2,2-D2 ACID | |
| | DL-4-HYDROXY-3- | |
| DIHYDROXYCARBONYLDIPHENYL | | |
| | METHOXYMANDELIC-2-D1 ACID | |
| SULPHIDE | | |
| 2,4,2′-TRINITRO-5- | 3,4,5-TRIHYDROXYBENZOIC- | |
| BENZYLOXY-3,3′ | 2,6-D2 ACID | |
| | DIHYDROXYCARBONYLDIPHENYL | |
| SULPHIDE | 3-(AND-4)-((ALPHA- | |
| 2,4,2′-TRINITRO-5- | CARBOXY-2- | |
| METHOXY-6,6′-DIMETHYL-3- | NITROBENZYL)OXY)-4-(AND- | |
| METHOXYCARBONYL-3′- | 3)-HYDROXYPHENETHYLAMINE, | |
| HYDROXYCARBONYLDIPH | TR | |
| 2,2′,4′-TRINITRO-5′ | HBED | |
| HYDROXY-5-AMINO-3- | 7-HYDROXYCOUMARIN | |
| HYDROXYCARBONYLDIPHENYL | GLUCURONIDE | |
| SULPHIDE | 2-(1-HYDROXY-1,2,3,4- | |
| D-(+)-B-[(2-AMINOPHENYL)- | TETRAHYDRONAPHTHALEN-1- | |
| THIO]-A-HYDROXY-4- | YL)PROPANOIC ACID | |
| METHOXYPHENYLPROPIONIC | | |
| ACID | | |
| 5,8-DIHYDROXY-9,10-DIOXO- | 2-AMINO-1- | |
| 9,10-DIHYDROANTHRACENE-1- | HYDROXYCARBONYL-3H-3- | |
| CARBOXYLIC ACID | OXOPHENOTHIAZINE | |
| | 2-AMINO-4,6-DIMETHYL-1,9- | |
| 2-[(2- | DIHYDROXYCARBONYL-3H-3- | |
| [[(BENZYLOXY)CARBONYL]AMI | OXOPHENOTHIAZINE | |
| NO]-3- | 2-AMINO-4-METHYL-1- | |
| METHYLBUTANOYL)AMINO]-3- | HYDROXYCARBONYL-3H-3- | |
| (4-HYDROXYPHENYL)PR | OXOPHENOTHIAZINE | |
| 2-[[(9H-9- | 2-AMINO-7- | |
| FLUORENYLMETHOXY)CARBONYL | HYDROXYCARBONYL-3H-3- | |
| ]AMINO]-3- | OXOPHENOTHIAZINE | |
| HYDROXYPROPANOIC ACID | 2-AMINO-8- | |
| 4-(BENZOYLAMINO)-3- | HYDROXYCARBONYL-3H-3- | |
| HYDROXYBUTANOIC ACID | OXOPHENOTHIAZINE | |
| 2-[(2-HYDROXY-6- | N-(2-AMINO-2- | |
| METHOXYBENZOYL)AMINOIACET | HYDROXYCARBONYL | |
| IC ACID | ETHYL)BENZYLPHOSPHONIC | |
| 17ALPHA- | | |
| ACID | | |
| HYDROXYPROGESTERONE 17- | O-(2-AMINO-2- | |
| ACETATE 3-(O- | HYDROXYCARBONYL | |
| CARBOXYMETHYL)OXIME | ETHYL)PHENYLPHOSPHONIC | |
| FMOC-HPG (TBU)-OH | ACID | |
| L-3,4-DIHYDROXYPHENYL[3- | M-(2-AMINO-2- | |
| 14C]ALANINE | HYDROXYCARBONYL | |
| 5-HYDROXY[3H]TRYPTAMINE | ETHYL)PHENYLPHOSPHONIC | |
| TRIFLUOROACETATE | ACID | |
| P-(2-AMINO-2- | | |
| BOC-(3S,4S)-4-AMINO-3- | HYDROXYCARBONYL | |
| HYDROXY-5-(4′- | ETHYL)PHENYLPHOSPHONIC | |
| PHENYL)PHENYLPENTANOIC | ACID | |
| ACID | 2-(3′-AMINO-3′- | |
| 2-AMINO-1,9- | HYDROXYCARBONYL | |
| DIHYDROXYCARBONYL-3H-3- | PROPYLOXY)ETHYLPHOSPHONIC | |
| OXOPHENOTHIAZINE | ACID | |
| 2-[3′-AMINO-(3′- | M-[2-AMINO-2-METHYL-2- | |
| HYDROXYCARBONYL)- | (HYDROXYCARBONYL)ETHYL]BE | |
| PROPYLOXY]ETHYLMETHYL | NZYLPHOSPHONIC ACID | |
| PHOSPHINIC ACID | P-[2-AMINO-2-METHYL-2- | |
| O-[2-AMINO-2- | (HYDROXYCARBONYL)ETHYL]PH | |
| (HYDROXYCARBONYL)- | ENYLPHOSPHONIC ACID | |
| ETHYL]BENZYLMETHYLPHOS PHI | O-[2-AMINO-2-METHYL-2- | |
| NIC ACID | (HYDROXYCARBONYL)ETHYL]PH | |
| P-[2-AMINO-2- | ENYLPHOSPHONIC ACID | |
| (HYDROXYCARBONYL)- | M-[2-AMINO-2-METHYL-2- | |
| ETHYL]BENZYLMETHYLPHOSPHI | (HYDROXYCARBONYL)ETHYL]PH | |
| NIC ACID | ENYLPHOSPHONIC ACID | |
| M-[2-AMINO-2- | 2-[3′-AMINO-3′-METHYL-3′- | |
| (HYDROXYCARBONYL)- | (HYDROXYCARBONYL)PROPYLOX | |
| ETHYL]BENZYLMETHYLPHOSPHI | Y]ETHYLPHOSPHONIC ACID | |
| NIC ACID | | |
| 2-AMINO-7- | BIS[5-AMINO-5- | |
| HYDROXYCARBONYL-7- | (HYDROXYCARBONYL)AMIL]PHO | |
| PHOSPHONOHEPTANOIC ACID | SPHINIC ACID | |
| | BIS [O-(2-AMINO-2- | |
| 2-AMINO-9- | | |
| | (HYDROXYCARBONYL)ETHYL)BE | |
| HYDROXYCARBONYL-9- | | |
| NZYL]PHOSPHINIC ACID | | |
| PHOSPHONONONYLIC ACID | | |
| 2-AMINO-8- | BIS[M-(2-AMINO-2- | |
| HYDROXYCARBONYL-8- | (HYDROXYCARBONYL)ETHYL)BE | |
| PHOSPHONOOCTANOIC ACID | NZYL]PHOSPHINIC ACID | |
| (2-[3′-AMINO-3′- | | |
| (HYDROXYCARBONYL)- | BIS[P-(2-AMINO-2- | |
| PROPYLOXY]ETHYL)-PHENYL | (HYDROXYCARBONYL)ETHYL)BE | |
| PHOSPHINIC ACID | NZYL]PHOSPHINIC ACID | |
| O-[2-AMIN0-2-METHYL-2- | BIS[7-AMINO-7- | |
| (HYDROXYCARBONYL)ETHYL]BE | (HYDROXYCARBONYL)HEPTYL]P | |
| NZYLPHOSPHONIC ACID | HOSPHINIC ACID | |
| P-[2-AMINO-2-METHYL-2- | BIS[6-AMINO-6- | |
| (HYDROXYCARBONYL)ETHYL]BE | (HYDROXYCARBONYL)HEXYL]PH | |
| NZYLPHOSPHONIC ACID | | |
| OSPHINIC ACID | (HYDROXYCARBONYL)-(O- | |
| BIS[2-(3′-AMINO-3- | AMINOMETHYL)- | |
| (HYDROXYCARBONYL)PROPYLOX | BENZYLMETHYLPHOSPHONIC | |
| Y)ETHYL]PHOSPHINIC ACID | ACID | |
| 6-HYDROXYCARBONYL- | (HYDROXYCARBONYL)-(M- | |
| [1,4]BENZOTHIAZINO[2,3- | AMINOMETHYL)BENZYLMETHYLP | |
| B]PHENOTHIAZINE | HOSPHONIC ACID | |
| 1-(HYDROXYCARBONYL)-5- | (HYDROXYCARBONYL)-(P- | |
| AMINOAMYL PHOSPHONIC ACID | AMINOMETHYL)BENZYLMETHYLP | |
| | HOSPHONIC ACID | |
| 1-(HYDROXYCARBONYL)-4- | 1-(HYDROXYCARBONYL)-3- | |
| AMINOBUTYL PHOSPHONIC | AMINOPROPYL PHOSPHONIC | |
| ACID | ACID | |
| 1-(HYDROXYCARBONYL)-3- | 2-(HYDROXYCARBONYL)ETHYL- | |
| (2′- | O-[2-AMINO-2- | |
| AMINOETHYLOXY)PROPYLPHOSP | (HYDROXYCARBONYL)ETHYL] | |
| HONIC ACID | BENZYLPHOSPHINIC A | |
| 1-(HYDROXYCARBONYL)-7- | 2-(HYDROXYCARBONYL)ETHYL- | |
| AMINOHEPTYL PHOSPHONIC | | |
| | P-[2-AMINO-2- | |
| ACID | | |
| 1-(HYDROXYCARBONYL)-6- | (HYDROXYCARBONYL)ETHYL] | |
| AMINOHEXYL PHOSPHONIC | BENZYLPHOSPHINIC A | |
| ACID | 2-(HYDROXYCARBONYL)ETHYL- | |
| 1-HYDROXYCARBONYL-2-(P- | 3-AMINO-3- | |
| [2′-AMINO-2′ | (HYDROXYCARBONYL)PROPYLPH | |
| (HYDROXYCARBONYL)ETHYL] | OS PHONIC ACID | |
| PHENYL)-ETHYL PHOSPH | 2-(HYDROXYCARBONYL)ETHYL- | |
| 1-HYDROXYCARBONYL-2-(M- | | |
| | 5-AMINOAMYLPHOSPHINIC | |
| [2′-AMINO-2′ | | |
| ACID | | |
| (HYDROXYCARBONYL)ETHYL]- | | |
| PHENYL)ETHYL PHOSPHO | 2-(HYDROXYCARBONYL)ETHYL- | |
| 1-HYDROXYCARBONYL-3-[3′- | 4-AMINOBUTYLPHOSPHINIC | |
| AMINO-3- | ACID | |
| (HYDROXYCARBONYL)PROPYLOX | | |
| Y]PROPYLPHOSPHONIC ACI | | |
| 2-(HYDROXYCARBONYL)ETHYL- | 5-(HYDROXY- | |
| 2-AMINOETHYLPHOSPHINIC | CARBONYL)ANYLPHOSPHINIC | |
| ACID | ACID | |
| | 2-(PHENYL)ETHYL-7-AMINO- | |
| 2-(HYDROXYCARBONYL)ETHYL- | 7 | |
| 6-AMINOHEXYLPHOSPHINIC | (HYDROXYCARBONYL)HEPTYLPH | |
| ACID | OSPHINIC ACID | |
| 2-(HYDROXYCARBONYL) ETHYL- | 2-(PHENYL)ETHYL-6-AMINO- | |
| 5-AMINO-5- | | |
| (HYDROXYCARBONYL)AMYLPHOS | (HYDROXYCARBONYL)HEXYLPHO | |
| PHINIC ACID | SPRINIC ACID | |
| | 2-(PHENYL)ETHYL-3-AMINO- | |
| 2-(HYDROXYCARBONYL)ETHYL- | 3 | |
| 7-AMINO-7- | (HYDROXYCARBONYL)PROPYLPH | |
| (HYDROXYCARBONYL)HEPTYLPH | OSPHINIC ACID | |
| OSPIIINIC ACID | 2-(PHENYL)ETHYL-2-[3′- | |
| 2-(HYDROXYCARBONYL)ETHYL- | AMINO-3′- | |
| 6-AMINO-6- | (HYDROXYCARBONYL)PROPYLOX | |
| (HYDROXYCARBONYL)HEXYLPHO | Y]ETHYL PHOSPHINIC ACID | |
| SPRINIC ACID | 2,2′,4′-TRINITRO-5′- | |
| 2-(HYDROXYCARBONYL)ETHYL- | BENZYLOXY-5-ACETYLAMINO- | |
| 5-AMINO-5- | 3-HYDROXYCARBONYL- | |
| (HYDROXYCARBONYL)PENT-2- | DIPHENYL SULPHIDE | |
| ENYLPHOSPHINIC ACID | 2,4,2′-TRINITRO-5- | |
| 2-(HYDROXYCARBONYL)ETHYL- | BENZYLOXY-3- | |
| 2-[3′-AMINO-3′- | BENZYLOXYCARBONYL-3 | |
| (HYDROXYCARBONYL)- | HYDROXYCARBONYLDIPHENYL | |
| PROPYLOXY]ETHYLPHOSPH | SULPH | |
| | 2,4,2′-TRINITRO-5- | |
| 2-(HYDROXYCARBONYL)ETHYL- | | |
| | BENZYLOXY-3- | |
| 3-AMINOPROPYLPHOSPHINIC | | |
| | HYDROXYCARBONYLDIPHENYL | |
| ACID | | |
| | SULPHIDE | |
| | 2,4,2′-TRINITRO-5- | |
| 2-(PHENYL)ETHYL-5-AMINO- | BENZYLOXY-6-METHYL-3- | |
| BENZYLOXYCARBONYL-3′- | ACID | |
| HYDROXYCARBONYLDIPHE | N-CBZ-4-AMINO-5-PHENYL-3- | |
| 2,4,2′-TRINITRO-5- | HYDROXYPENTANOIC ACID | |
| HYDROXY-3,3′ | (2S,3R)-2,3- | |
| DIHYDROXYCARBONYLDIPHENYL | | |
| | DIHYDROXYBUTANOIC ACID | |
| SULPHIDE | (R)-2-HYDROXY-3-(2′ | |
| 2,2′,4′-TRINITRO-5′ | NITROPHENYL)-PROPIONIC | |
| HYDROXY-4- | | |
| | ACID | |
| HYDROXYCARBONYLDI PHENYL | (S)-2-HYDROXY-3-(2′ | |
| SULPHIDE | NITROPHENYL)-PROPIONIC | |
| 2,2′,4′-TRINITRO-5′- | ACID | |
| METHOXY-5-ACETYLAMINO-3- | 2-HYDROXY-4-PYRIDINE | |
| HYDROXYCARBONYLDIPHENYL | CARBOXYLIC ACID | |
| SULPHIDE | (S)-2-HYDROXYBUTYRIC ACID | |
| 2,4,2′-TRINITRO-5- | (R)-2-HYDROXYBUTYRIC ACID | |
| METHOXY-3- | | |
| METHOXYCARBONYL-3′- | 4-(HYDROXYETHYL)-2- | |
| HYDROXYCARBONYLDIPHENYL | METHOXY-PHENYLOXY ACETIC | |
| SULPHIDE | ACID | |
| 2,4,2′-TRINITRO-5- | (R)-2-HYDROXYHEXANOIC | |
| HYDROXY-6-METHYL-3- | ACID | |
| HYDROXYCARBONYLDIPHENYL | (S)-2-HYDROXYHEXANOIC | |
| | ACID | |
| SULPHIDE | | |
| (+/−)-CIS-1-AMINO-2-(4- | 4-(HYDROXYMETHYL)-2- | |
| HYDROXYPHENYL) | METHOXYPHENOXYACETIC ACID | |
| CYCLOPROPANE CARBOXYLIC | 2,3,4,5-TETRAHYDRO-4- | |
| ACID | HYDROXY-3-PYRIDAZINE | |
| (1R,2S)-1-AMINO-2- | CARBOXYLIC ACID | |
| (HYDROXYMETHYL) | FMOC-D-CIS-HYP-OH | |
| CYCLOPROPANE CARBOXYLIC | N-OMEGA-HYDROXY-NOR-L- | |
| ACID | ARGININE | |
| (+/−)-TRANS-1-AMINO-3- | 4-ACETYL-3,5-DIOXO-1- | |
| (HYDROXYMETHYL) | METHYLCYCLOHEXANE- | |
| CYCLOBUTANE CARBOXYLIC | CARBOXYLIC ACID | |
| 4-(4-(1-HYDROXYETHYL)-2- | METHYL(2S,3R)-(−)-2,3- | |
| METHOXY-5-NITRO-PHENOXY) | DIHYDROXY-3- | |
| BUTYRIC ACID | PHENYLPROPIONATE | |
| 2-AMINO-5-HYDROXY-4- | 2-HYDROXY-4-PHTHALIMINO | |
| OXOPENTANOIC ACID | BUTANOIC ACID | |
| O-NPS-L-HYDROXYPROLINE | | |
| DICYCLOHEXYLANINE SALT | 3,4,5,6- | |
| DL-2-(TRIFLUOROMETHYL)-3- | TETRAHYDROXYTETRAHYDRO | |
| (3′,4′- | 2H-PYRAN-2-CARBOXYLIC | |
| DIHYDROXYPHENYL)ALANINE | ACID | |
| | 3-[(4-[[(2-AMINO-4- | |
| S(−)-CYANOPINDOLOL | | |
| | HYDROXYPTERIDIN-6- | |
| HEMI FUMARATE | | |
| CHPG | YL)METHYL]AMINO]BENZOYL)A | |
| 3-CARBOXY-4 - | MINO]HEXANEDIOIC A | |
| HYDROXYPHENYLACETONE | 5-(TERT-BUTYL)-1-[2-(2- | |
| 3-CARBOXY-4-HYDROXYBENZYL | HYDROXYETHYLTHIO)ETHYL]- | |
| ALCOHOL | 2-METHYLPYRROLE-3- | |
| N-(2-CARBOXYPROPIONYL)-5- | CARBOXYLIC ACID | |
| HYDROXYTRYPTAMINE | 1-[2-(2- | |
| 2,3-DIFLUORO-4- | HYDROXYETHYLTHIO)ETHYL]- | |
| HYDROXYBENZOIC ACID | 2-METHYL-5-PHENYLPYRROLE- | |
| D-(−)-ALPHA- | | |
| DIHYDROXYPHENYLGLYCINE | 3-CARBOXYLIC ACID | |
| (2R,3R)-TARTRANILIC ACID | 2-(4-HYDROXY-3,5- | |
| | DIMETHYLBENZOYL)BENZOIC | |
| 2-HYDROXY-5-(1H-PYRROL- | | |
| | ACID | |
| 1-YL)BENZOIC ACID | 5-(TERT-BUTYL)-2- | |
| 2-HYDROXY-5-(1H-PYRROL- | HYDROXYISOPHTHALIC ACID | |
| 1-YL)BENZOIC ACID | BUTTPARK 21\09-50 | |
| | 2,6-DIHYDROXYTEREPHTHALIC | |
| N- | (TERT- | |
| ACID | | |
| BUTYL)HYDROXYLAMINE | | |
| ACETATE | 3-HYDROXY-2-METHYL-3- | |
| METHYL (2R,3S)-(+)-2,3- | PHENYLPROPANOIC ACID | |
| DIHYDROXY-3- | 3-HYDROXY-3-ISOPROPYL-4- | |
| PHENYLPROPIONATE | METHYLPENTANOIC ACID | |
| 2,3-DIHYDROXY-3- | HYDROXYPHENYLALANINE | |
| PHENYLPROPANOIC ACID | 17-PHENYL TRINOR | |
| 2-(1-HYDROXY-1,2,3,4- | PROSTAGLANDIN A2 | |
| TETRAHYDRONAPHTHALEN-1- | BW 2460 | |
| [3H]-8-ISO PROSTAGLANDIN | | |
| YL)-2-METHYLPROPANOIC | | |
| F2ALPHA | | |
| ACID | MYRIOCIN | |
| 2-(BENZOYLAMINO)-3- | 2-HYDROXYAMINO-3- | |
| HYDROXYPROPANOIC ACID | HYDROXYIMINO-2- | |
| 5-CYANO-2-HYDROXY-4- | | |
| METHYLBENZOIC ACID | METHYLBUTANE ACETIC ACID | |
| 3-[3-(3-HYDROXY-3- | SALT | |
| OXOPROPYL)-2- | 4-HYDROXY-1,3-DIMETHYL- | |
| OXOCYCLOHEXYL]PROPANOIC | 1H-PYRAZOLO[3,4- | |
| ACID | B]PYRIDINE-5-CARBOXYLIC | |
| 4-HYDROXY-5,8- | ACID | |
| DIMETHYLQUINOLINE-3- | 2-(3,5-DICHLORO-2- | |
| CARBOXYLIC ACID | HYDROXYPHENYL)BENZO[H]QUI | |
| PHEBESTIN | NOLINE-4-CARBOXYLIC ACID | |
| 2-(4- | 2-[(TERT- | |
| DIHYDROXYBORANE)PHENYL-4- | | |
| | BUTOXYCARBONYL)AMINO]-3- | |
| CARBOXY-6-METHYLQUINOLINE | HYDROXYBUTANOIC ACID | |
| 2-(4- | 2-[2-[2-(TERT-BUTOXY)-2- | |
| DIHYDROXYBORANE)PHENYL-4- | OXOETHYL]ANILINO]-3- | |
| CARBOXYQUINOLINE | HYDROXYBUTANOIC ACID | |
| SIASTATIN B | 2-FLUORO-4′-[(5- | |
| HYDROXY-3-METHYLGLUTARIC | HYDROXYPENTYL)OXY][1,1′- | |
| ACID, [3-14C1 | BIPHENYL]-4-CARBOXYLIC | |
| | ACID | |
| BESTATIN, [LEUCINE-1- | (Z)-4-(4-HYDROXYPHENYL)- | |
| 14C], HYDROCHLORIDE | 4-OXO-2-BUTENOIC ACID | |
| FMOC-AHPA-OH DCHA | | |
| L-O-TYR | 2′-FLUORO-4′-(4- | |
| (S)-3-(4′-HYDROXYPHENYL)- | HYDROXYBUTOXY)[1, 1′- | |
| 2-HYDROXY-PROPANOIC ACID | BIPHENYL]-4-CARBOXYLIC | |
| N-ACETYL-3- | ACID | |
| 4- | 4-(CHLOROPHENYL)-3,3- | |
| (DIPHENYLHYDROXYMETHYL)BE | DIFLUORO-2- | |
| NZOIC ACID | HYDROXYPROPIONIC ACID | |
| FMOC-L-PHE(3,5- | 2-FLUORO-4-(2- | |
| DIHYDROXY) | HYDROXYETHYL)BENZOIC ACID | |
| ATPA | | |
| | 2-FLUORO-5-(2- | |
| (DIPHENYLHYDROXYMETHYL)PH | HYDROXYETHYL)BENZOIC ACID | |
| ENOXY]PROPIONIC ACID DCHA | | |
| | 3,3-DIFLUORO-3-(4- | |
| 3-[4-(DI-(4- | FLUOROPHENYL)-2- | |
| CHLOROPHENYL)HYDROXYMETHY | HYDROXYPROPIONIC ACID | |
| L)PHENOXY]PROPIONIC ACID | 2-FLUORO-3-HYDROXYBENZOIC | |
| DCHA | ACID | |
| 2,5-DIMETHOXY-4- | 3,3-DIFLUORO-2-HYDROXY-3- | |
| HYDROXYBENZOIC ACID | PHENYLPROPIONIC ACID | |
| BOC-D-PHG (4-OH)-OH | 2-FLUORO-5-HYDROXYBENZOIC | |
| FMOC-THY-OH | ACID | |
| BOC-D-HYP-OH | 2-FLUORO-3-(2- | |
| 4-BROMO-1- | HYDROXYETHYL)BENZOIC ACID | |
| HYDROXYANTHRAQUINONE-2- | | |
| | (2R,3S)-1-CARBOXY-4- | |
| CARBOXYLIC ACID | | |
| 7-HYDROXYISOFLAVONE-7-O- | CHLORO-2,3- | |
| GLUCURONIDE | DIHYDROXYCYCLOHEXA-4,6- | |
| 2-ETHYL-D5- | DIENE | |
| HYDROXYBUTYRIC-3,3,4,4,4. | (2R,3S)-1-CARBOXY-4,5- | |
| D5 ACID | DICHLORO-2,3- | |
| 2-FLUORO-5- | DIHYDROXYCYCLOHEXA-4,6- | |
| (HYDROXYMETHYL)BENZOIC | DIENE | |
| ACID | (2R,3S)-1-CARBOXY-4-IODO- | |
| 3,3-DIFLUORO-2-HYDROXY-3- | 2,3-DIHYDROXYCYCLOHEXA- | |
| (4- | 4,6-DIENE | |
| METHOXYPHENYL)PROPIONIC | | |
| | BOC-L-6-HYDROXYNORLEUCINE | |
| ACID | | |
| 2-FLUORO-3- | | |
| | CP-H HYDROCHLORIDE | |
| (HYDROXYMETHYL)BENZOIC | 2-HYDROXY-6- | |
| ACID | | |
| (TRIFLUOROMETHYL)NICOTINI | 2,2-DIMETHYL-3-HYDROXY-3- | |
| C ACID | (P-TOLYL)PROPIONIC ACID | |
| (R,S)-ALPHA-AMINO-3- | | |
| HYDROXY-4-METHYL-5- | 2,2-DIMETHYL-3-HYDROXY-3- | |
| ISOXAZOLE PROPIONIC ACID, | (P- | |
| MONOHYDRATE | METHOXYPHENYL)PROPIONIC | |
| FMOC-D-HYP-OH | ACID | |
| HYDROXYPALMITIC ACID | DL 2-ISOPROPYLSERINE | |
| ALPHA-DL [1-14C] | DL-2-ISOBUTYLSERINE | |
| DIHYDROXYPHENYLALANINE- | DL-2-BENZYLSERINE | |
| D-3,4-[ALANINE-1-14C] | DL-N-BENZOYL-2- | |
| BOC-O-BENZYL-L-BETA- | METHYLSERINE | |
| HOMOHYDROXYPROLINE | DL-N-BENZOYL-2- | |
| DICYCLOHEXYLAMINE SALT | ISOPROPYLSERINE | |
| L-BETA-HOMOSERINE | DL-N-BENZOYL-2- | |
| HYDROCHLORIDE | ISOBUTYLSERINE | |
| L-BETA-HOMOHYDROXY PROLINE | DL-N-BENZOYL-2- | |
| HYDROCHLORIDE | BENZYLSERINE | |
| | 4′-ACETAMIDO-2′-CARBOXY | |
| FMOC-O-TERT-BUTYL-L-BETA- | 4-DIMETHYLAMINO-2- | |
| HOMOHYDROXYPROLINE | HYDROXYBENZOPHENONE | |
| L-BETA-HOMOTYROSINE | 5′-ACETOMIDO-2′-CARBOXY | |
| HYDROCHLORIDE | 4-DIMETHYLAMINO-2- | |
| L-BETA-HOMOTHREONINE | HYDROXYBENZOPHENONE | |
| HYDROCHLORIDE | | |
| 3-(2- | | |
| BENZOTHIAZOLYL)UMBELLIFER | (ACETAMIDO)ETHYL]-N | |
| ONE-4-CARBOXYLIC ACID | METHYLAMINO]-2′-CARBOXY- | |
| | 2-HYDROXYBENZOPHENONE | |
| 6,7-DIHYDROXY-4- | | |
| | 2′CARBOXY-4- | |
| COUMARINYLACETIC ACID | | |
| HYDROXYECTOINE | DIMETHYLAMINO-2-HYDROXY- | |
| FMOC-(R,S)-3-AMINO-3-(3- | 4′NITROBENZOPHENONE | |
| HYDROXY-PHENYL)-PROPIONIC | | |
| ACID | | |
| 2′CARBOXY-4- | CAFTARIC ACID | |
| DIMETHYLAMINO-2-HYDROXY- | TRANS-3′- | |
| 5′NITROBENZOPHENONE | HYDROXYMETHYLNICOTINE, | |
| | HEMISUCCINATE | |
| (S)-3-HYDROXYMYRISTIC | H-CIS-MEHYP-OH | |
| ACID | BOC-D-HPG(BR-Z)-OH | |
| 9-HYDROXY-9-(4- | BOC-CISHYP(BZL)-OH | |
| CARBOXYPHENYL)XANTHENE | BOC-TYR(HEXAHYDRO)-OH | |
| 4-HYDROXYMETHYL-2- | FMOC-CIS-HYP(BZL)-OH | |
| METHOXY-5- | FMOC-HYP(3)-OH | |
| NITROPHENOXYBUTYRIC ACID | 3-FLUORO-4-HYDROXYBENZOIC | |
| | ACID, HYDRATE | |
| 5-HYDROXY-1,3- | | |
| OXATHIOLANE-2-CARBOXYLIC | 4-HYDROXY-3- | |
| ACID | NITROPHENYLACETYL- | |
| 4-[(2-[[2-(4 | EPSILON-ANINOCAPROIC ACID | |
| HYDROXYPHENYL)ACETYL]AMIN | | |
| | 4-HYDROXY-3-IODO-5- | |
| O]ETHYL)AMINO]-4-OXOBUT- | | |
| | NITROPHENYLACETYL- | |
| 2-ENOIC ACID | | |
| 2-AMINO-3-HYDROXY-2- | EPSILON-AMINOCAPROIC ACID | |
| METHYLBUTANOIC ACID | N-BENZOXYCARBONYL-ALPHA- | |
| 4′-[(10- | | |
| | HYDROXYGLYCINE | |
| HYDROXYDECYL)OXY][1,1′- | CHLOROGENIC ACID | |
| BIPHENYL]-4-CARBOXYLIC | HEMIHYDRATE | |
| ACID | 4-HYDROXY-8-METHOXY-6- | |
| 4-(1-HYDROXYETHYL)- | NITROQUINOLINE-3- | |
| METHYL-5- | CARBOXYLIC ACID | |
| (METHYLTHIO)THIOPHENE-2- | 7-CHLORO-4-HYDROXY-8- | |
| CARBOXYLIC ACID | METHYLQUINOLINE-3- | |
| 2-HYDROXYCITRIC ACID | CARBOXYLIC ACID | |
| TRANS-4- | 6,8-DIMETHYL-4- | |
| HYDROXYCYCLOHEXANECARBOXY | HYDROXYQUINOLINE-3- | |
| LIC ACID | CARBOXYLIC ACID | |
| (2R,3R)-1-CARBOXY-2,3- | 8-BROMO-4- | |
| DIHYDROXY-4- | HYDROXYQUINOLINE-3- | |
| METHYLCYCLOHEX-4,6-DIENE | CARBOXYLIC ACID | |
| 6-HYDROXY-5- | HYDROXYPHENYL)METHYL]PROP | |
| NITRONICOTINIC ACID | IONIC ACID | |
| AMPA,S-(−)[5-METHYL-3H]- | 5-HYDROXYVALPROIC ACID | |
| | 2-PROPYL-3- | |
| 5,6-DIHYDROXY-1H-INDOLE- | HYDROXYPENTENOIC ACID | |
| 2-CARBOXYLIC ACID | 2-PROPYL-4- | |
| | HYDROXYPENTENOIC ACID | |
| 2,3-DIHYDROXYTERPHTHALIC | 2-PROPYL-5- | |
| ACID | HYDROXYPENTENOIC ACID | |
| | PHENYL (4- | |
| 4-HYDROXY-2- | ISOTHIOCYANATO)SALICYLATE | |
| PYRROLECARBOXYLIC ACID | | |
| 3-HYDROXY-2- | 3-CARBOXY-4- | |
| PYRROLIDINECARBOXYLIC | HYDROXYPHENYLISOTHIOCYANA | |
| ACID | TE | |
| 5-HYDROXY-3- | 2-HYDROXY-5-NITROCINNAMIC | |
| PIPERIDINECARBOXYLIC ACID | ACID | |
| | 5-BROMO-2-HYDROXY-3- | |
| 4-HYDROXY-5- | METHOXYBENZOIC ACID | |
| HYDROXYMETHYL-2- | 2[(1E)-2- | |
| PYRROLIDINE-CARBOXYLIC | (METHOXYCARBONYL)-1- | |
| ACID | METHYLVINYL]AMINO]-(2R)- | |
| | 2-(4-HYDROXYPHENYL)ACETIC | |
| HYDROXYBENZO[B]THIOPHENE- | 3-HYDROXYHEXANOIC ACID | |
| 2-CARBOXYLIC ACID | 3-HYDROXY-15- | |
| 4-HYDROXY-L-ISOLEUCINE | | |
| | METHYLPALMITIC ACID | |
| FMOC-(R,S)-3-AMINO-3-(4 | THREO-2,3- | |
| HYDROXY-PHENYL)-PROPIONIC | DIHYDROXYPALMITIC ACID | |
| ACID | ERYTHRO-2,3- | |
| 3-[(FLUOREN-9- | DIHYDROXYPALMITIC ACID | |
| YLMETHOXY)CARBONYLAMINO]- | 3-HYDROXYHEPTADECANOIC | |
| 3-(4-HYDROXY-3- | ACID | |
| METHOXYPHENYL)PROPANOIC | 20-HYDROXYEICOSANOIC ACID | |
| AC | | |
| 3-[(FLUOREN-9- | 21-HYDROXYHENEICOSANOIC | |
| YLMETHOXY)CARBONYLAMINO]- | ACID | |
| 2-[(4- | | |
| 22-HYDROXYDOCOSANOIC ACID | PHENYLISOSERINE | |
| | (2R,3S)-FMOC-3- | |
| LESQUEROLIC ACID | PHENYLISOSERINE | |
| (R)-ATPA | (2S,4S)-N-BOC-4-HYDROXY | |
| DELTA2-PROSTAGLANDIN A1 | PIPERIDINE-2-CARBOXYLIC | |
| | ACID BENZYLAMINE SALT | |
| 17,20-DIMETHYL-TRANS- | | |
| DELTA2-PROSTAGLANDIN A1 | | (2S,4S)-N-BOC-4-HYDROXY |
| | PIPERIDINE-2-CARBOXYLIC | |
| DELTA2-PROSTAGLANDIN E1 | ACID BENZYLAMINE SALT | |
| 15(R),19(R)-HYDROXY | (2R,4R)-N-BOC-4-HYDROXY | |
| PROSTAGLANDIN E1 | PIPERIDINE-2-CARBOXYLIC | |
| 15(R),19(R)HYDROXY | ACID BENZYLAMINE SALT | |
| PROSTAGLANDIN E2 | | |
| 3-METHOXY PROSTAGLANDIN | (S)-4-HYDROXY-1- | |
| F1ALPHA | CYCLOPENTENE-1-CARBOXYLIC | |
| 2,3-DINOR-8-ISO | ACID | |
| PROSTAGLANDIN F1ALPHA | (1S,3R)-3-HYDROXY- | |
| 17,20-DIMETHYL | CYCLOPENTANECARBOXYLIC | |
| PROSTAGLANDIN F1ALPHA | ACID | |
| 17,20-DIMETHYL-TRANS- | (1S,3S)-3-HYDROXY- | |
| DELTA2-PROSTAGLANDIN | CYCLOPENTANECARBOXYLIC | |
| F1ALPHA | ACID | |
| 15(R),19(R) -HYDROXY | (R)-4-HYDROXY-1- | |
| PROSTAGLANDIN F1 ALPHA | CYCLOPENTENE-1-CARBOXYLIC | |
| IPF2ALPHA-I | | |
| 8-EPI PGFM | ACID | |
| | (R)-4-AMINO-3- | |
| 17,20-DIMETHYL | | |
| | HYDROXYBUTANOIC ACID | |
| PROSTAGLANDIN F2ALPHA | SDZ 220-040 | |
| 15(R),19(R)-HYDROXY | 2-N-PROPYL-4- | |
| PROSTAGLANDIN F2ALPHA | HYDROXYPENTANOIC ACID | |
| FLUPROSTENOL-D4 | | |
| 3(R)-HETE | | |
| BAEOMYCESIC ACID | | |
| CARNOSIC ACID | | |
| 15(R)-LIPOXIN A4 | | |
| (2R,3S)-BOC-3- | | |

### EXAMPLE I

### Preparation of Holliday Junction-Trapping Macrocyclic Peptides

This example shows a method for preparing Holliday junction-trapping cyclic peptides based on known lead compounds.

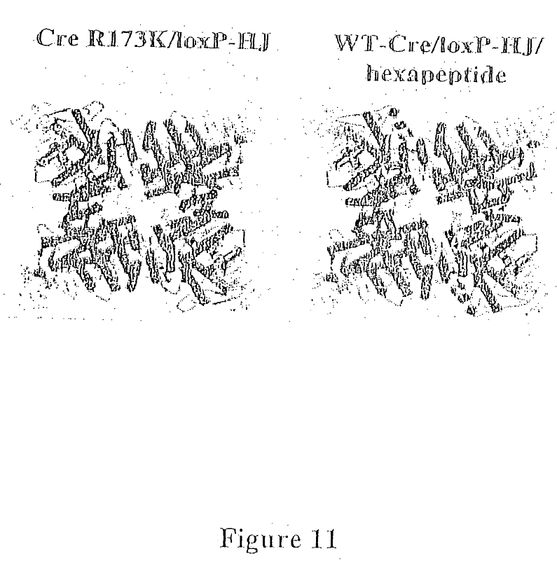

Eight macrocycle peptides for trapping Holliday junctions (HJs) were prepared based on the C-2 symmetrical HJ binding site seen in a co-crystal structure of wild type Cre recombinase and on the residues in the active linear HJ trapping peptides described in Cassell et al. J. Mol Bio. 299:1193-1202 (2000) and Klemm et al. J. Mol Bio. 299:1203-1212(2000), shown in FIG. 11. The macrocycles were also designed to fit the approximate size of the HJ binding site, which is estimated from the crystal structure to be ˜25 Å by 10 Å (Klemm et al., 2000, supra; and Gopaul, et al. EMBO J. 17: 4175-4187 (1998)). Because the lead linear peptides were C-2 symmetric, several C-2 symmetric cyclic peptides were prepared, as shown in FIGS. 55.

The cyclic peptides were designed with consideration of the structural characteristics of the HJ. The minimal number of residues involved in binding to the HJ is not known, but several important interactions have been identified (Cassell, 2000, supra, and Klemm et al., 2000, supra). Hydrophobic residues such as tryptophan and phenylalanine are known to be important for binding to the DNA because they appear in all of the lead compounds. It is thought that these residues stack with nucleotides that surround the center of the HJ. Hydrophilic residues such as arginine and lysine likely form hydrogen bonds with the proteins that assemble the HJ, with the DNA substrate itself, or both.

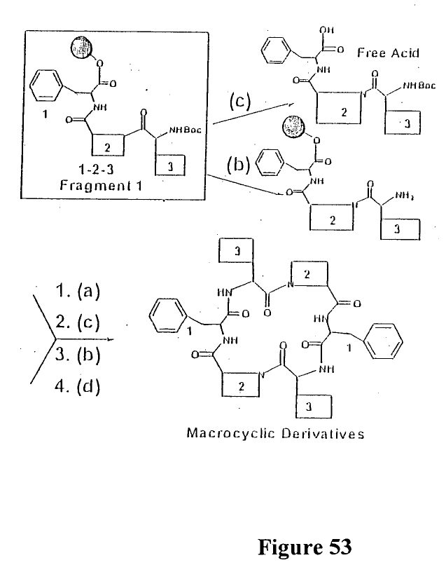

Starting from commercially available natural and unnatural amino acids, cyclic hexameric peptides were synthesized. Initially, a number of hydrophobic residues were selected for inclusion in the macrocycles, with the goal of sequentially exchanging the hydrophobic residues with hydrophilic residues as needed. L-phenylalanine methyl ester and N-Boc protected residue 2 (FIGS. 53) were coupled using 2-(1-H-Benzotriazole-1-yl)-1,1,3,3 tetramethyluronium tetrafluoroborate (TBTU) (1.2 equivalents) as the coupling reagent and Hunig's base (3 equivalents), yielding the dipeptide 1-2-Boc (80-94% yield). Deprotection of the amine on residue 2 using 20% TFA and two equivalents of anisole in methylene chloride yielded the free amine 1-2 (˜quantitative yields). Coupling of this dipeptide to monomer 3a or 3b gave the desired tripeptide (Fragment 1) in high yields (65%-94%). Coupling of the amino acids using these reagents alleviates racemization of stereocenters and was more effective for secondary amines when compared to more common coupling agents (for example, HOBT).

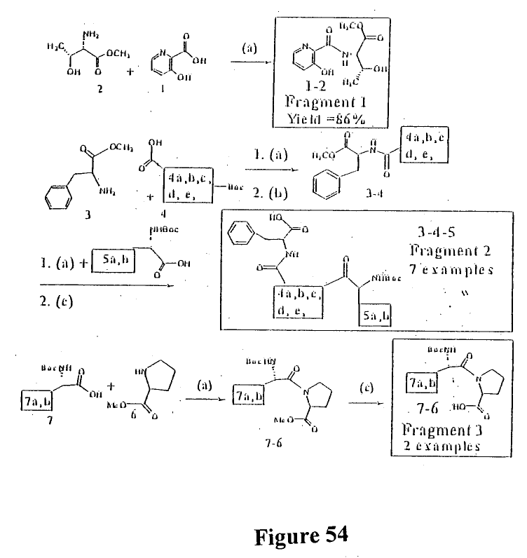

Fragment 1 was separated into two equal aliquots (FIGS. 54). The acid was deprotected in the first aliquot using four equivalents of barium hydroxide, while the amine was deprotected in the second aliquot using TFA/Anisole. These two trimer peptides were coupled using multiple coupling agents to give the linear hexapeptides. The resultant hexapeptides were cyclized by deprotecting the acid using four equivalents of barium hydroxide. Upon workup of the free acid the compounds were subjected to 20% TFA and two equivalents of anisole in methylene chloride. Following deprotection of the free amine, the crude, dry product was subjected to HATU, TBTU, PyBop, and/or DEPBT coupling reagents (1.2 equivalents each), and Hunig's base (3 equivalents) in methylene chloride. The final macrocyclizations took approximately four days due to the low concentration (0.005-0.01 M) that was required to maximize the yield. The one-pot ring-closing yields varied from 21% to 48%. The peptides were purified using reverse-phase HPLC and the structures were confirmed using LCMS, High Resolution Mass Spectroscopy and proton NMR.

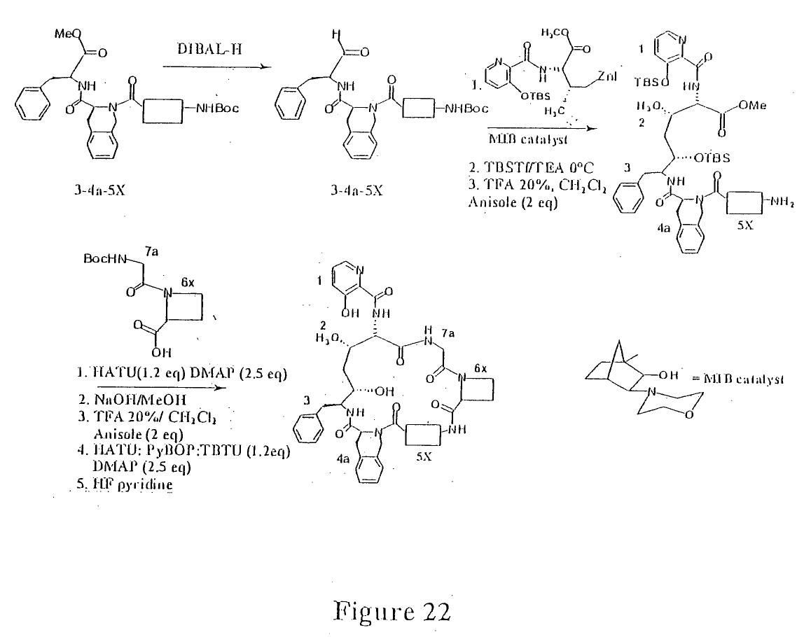

Four macrocyclic hexapeptides that incorporate tyrosine (2f) and either Tryptophan (3b) or arginine (3c) (FIG. 22) also have been synthesized. The methods described herein also are useful for synthesis of octameric macrocycles, such as those that incorporate Tyrosine (2f), and Arginine (4a), as shown in FIGS. 59.

Solid-phase synthesis was used to synthesize tripeptides and tetrapeptides in parallel using standard coupling conditions, such as those described above. The monomers included 1, 2a-f, 3a-c, 4a-c, as shown in FIGS. 59, and were coupled sequentially to give Boc-protected trimers or tetramers, in a similar approach to that shown in FIGS. 53. Only the methyl ester of residue 1 was attached to solid-phase as an ester linkage, as shown in FIGS. 60. Merrifield resin and a standard linker, which cleaves to give a free acid, was used for this purpose. The beads of the tri- and tetra-peptides were divided into two batches, with half of the beads for each compound being placed in new wells. The beads in each well were cleaved to give the free acid of each tri- or tetrapeptide in a parallel fashion, as shown in FIGS. 60. The remaining beads underwent amine deprotection also in parallel. The free acids were then coupled to their matching free amine tri- or tetrapeptide, which was attached to solid-phase. This yielded the linear, symmetrical hexa- and octa-peptides. This solid-phase approach is analogous to the route taken in solution, shown in FIGS. 54. The resultant linear peptides were cleaved from solid phase giving the free acid, and purity of the peptides is determined using LCMS. Purification was performed using reverse-phase HPLC, as needed. Deprotection of the amines and subsequent cyclization in parallel using the conditions described above yielded the desired C-2 symmetrical macrocycles. Tyrosine, arginine, histadine, and lysine residues were used with appropriate protecting groups that were cleaved upon cyclization. D-amino acids also were incorporated into the syntheses of these macrocycles.

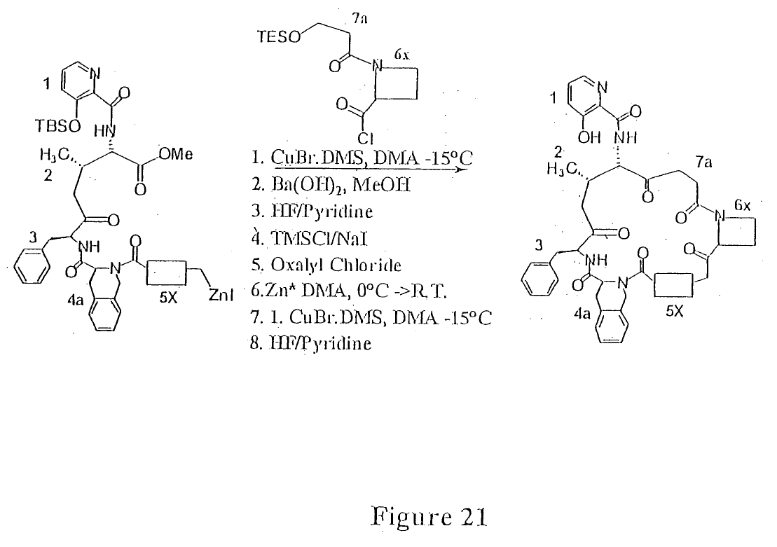

This example shows methods for preparing cyclic peptides, including those shown in FIG. 21.

### EXAMPLE II

In Vitro Trapping of Holliday Junctions Using Macrocyclic Peptides

This example shows that macrocyclic peptides can be used to trap Holliday junctions using in vitro assays.

The eight macrocyclic compounds shown in FIGS. 55 were tested for their ability to bind to the HJ in an in vitro assay. The assay employs a recombination reaction between one radiolabeled double-stranded DNA substrate and an unlabeled partner DNA substrate. Successful recombination gives recombination products and very small amounts of HJ intermediate. A previously identified linear peptide dimer of Lys-Trp-Trp-Cys-Arg-Trp was used as a positive control because it was known to accumulate HJs, as described in Cassell et al., 2000, supra). During the recombination reaction, two initial DNA cleavage and ligation reactions must take place to form the junction, and two subsequent DNA cleavage and ligation reactions resolve the HJs into double stranded recombination products.

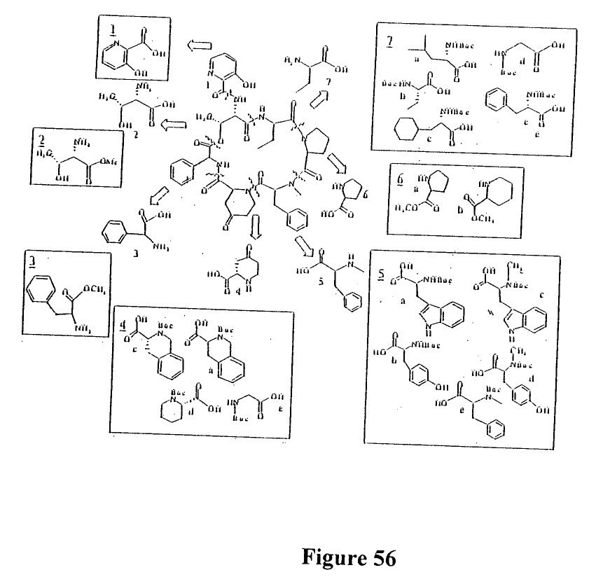

At 0.01 mg/ml concentrations of peptides, a significant amount of HJ intermediates (I) accumulated in the presence of four macrocyclic hexamers when compared to a control reaction not treated with peptide (FIGS. 56, lane 2). In FIGS. 56, which shows results of an experiment in which compounds were combined with one radiolabeled double stranded DNA and an unlabeled partner DNA, I=HJ Intermediate, II=Recombination Products, III=Free DNA hexamers. Four compounds having structures: 1-2a-3a (A), 1-2a-3b (B), 1-2c-3a (D), and 1-2c-3b (E) were particularly active, as can be observed in lanes 5-8. These four active compounds all contain phenylalanine coupled to the 1,2,3,4-tetrahydroisoquinoline-3-carboxylic acid moiety (lanes 5-8), and stabilizes the HJ almost as potently as the lead peptide (lane 3). This binding effect may be the result of the aromatic group in the tetrahydroisoquinoline π-stacking with DNA bases. The relatively low binding of the compounds containing glycine or the piperdine moiety in position 2 (lanes 9-11) may be explained by their inability to intercalate into the DNA. The flexibility of the amino octanoic chain may explain the lack of binding for the phenyl and indole moieties present in 1-2b-3b (C) macrocyclic hexamer (lane 4). Results from this in vitro assay indicate that the macrocyclic peptides can trap the Holliday junction.

### EXAMPLE III

### In Vivo Trapping of Holliday Junctions Using Macrocyclic Compounds

This example shows that macrocyclic peptides can be used to trap Holliday junctions using in vivo assays.

The eight macrocyclic compounds shown in FIGS. 55 were tested for their ability to bind to the HJ and inhibit bacterial growth in an in vivo assay. Growth inhibition assays were performed in microtiter plates, by comparing the growth of bacteria in the presence and absence of compounds. Cell density at 600 nm was determined for a series of bacterial strains, both gram positive and gram negative, in the presence and absence of macrocyclic compounds. The bacterial strains used in the assays include three gram+ species: methicillin-resistant Staphylococcus aureus (MRSA; gm+), Streptococcus pneumoniae (gm+), and Bacillus subtilis, and 6 gram negative species: Salmonella typhimurium, E. coli, Klebsiella pneumoniae, Haemophilus influenzae, Neisseria gonorrhoea, and Pseudomonas aeruginosa. Measurements were taken using a BioRad microtiter plate reader. Overnight cultures were diluted 1:20 into LB media and each test compound was added in no more than 10% of the volume of the final suspension. The compounds were dissolved in DMSO and added to the cultures at two concentrations (101 μg/ml and 100 μg/ml). The diluted cultures were shaken at 37° C. and optical densities were measured at 30-minute intervals in order to follow their growth. Growth curves were then compared for all compounds at these two concentrations.

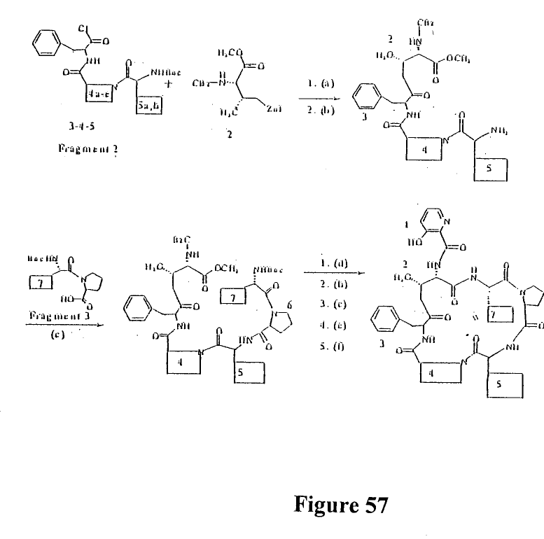

Although bacterial growth inhibition was seen for most of the compounds (FIGS. 57), one compound (H), which was made with monomers 1, 2d, 3b, had the greatest level of activity. This compound (H) contains two phenylalanines, two tryptophans, and two glycines. These results from this in vivo assay indicate that the macrocyclic peptides have antibiotic activity.

To optimize the activity of a macrocyclic compound, as the most active compounds are identified, individual monomers of the original peptidomimetic are substituted with a glycine or alanine, in order to determine the contribution of each chemical moiety. The potency of these substituted compounds is determined both for bacterial growth inhibition assays and recombination inhibition assays. If the substituted compounds have the same or similar profiles of inhibition for bacterial growth and for recombination, it is concluded that it is most likely that the ability of the compound to inhibit growth reflects inhibition of site-specific recombination or a related property. It is possible that the profile of inhibition of the substituted compounds can diverge greatly between inhibition of growth and inhibition of recombination. If this is the case, either that the substitutions affect transport of the compounds into cells or that the inhibition of growth and inhibition of recombination in vitro do not involve the same mechanism.

### EXAMPLE IV

### In Vivo Trapping of Holliday Junctions Using Macrocyclic Peptides

This example shows identification of linear peptides that stabilize Holliday junctions.

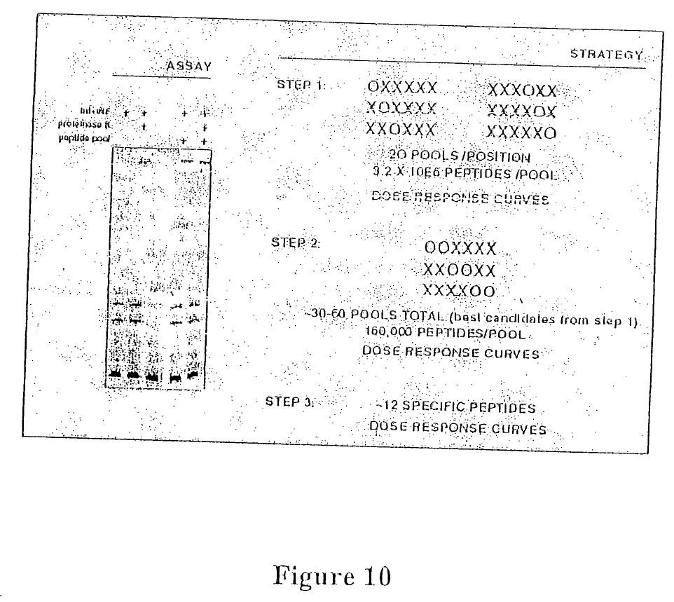

To determine whether candidate Holliday junction-trapping hexapeptides stabilized Holliday junctions, protein-DNA covalent complexes were identified by their sensitivity to proteinase K, while the Holliday junctions were identified by their slow mobility and resistance to proteinase K (FIG. 10). Two distinct families of peptides were observed, of which peptide LysTrpTrpCysArgTrp (KWWCRW) and peptide TrpLys(His or Ala)TyrAsnTyr (WK(HorA)YNY) are the most potent (Cassell et al., J. Mol. Bio. 299:1193-1202 (2000)). Results described herein suggest that KWWCRW blocks strand cleavage, either at the top or at the bottom strand, while peptide WKHYNY does not block strand cleavage but rather appears to stabilize Holliday junctions (Klemm et al., J. Mol. Bio. 299:1203-1216 (2000)). Because two rounds of strand cleavage, exchange, and ligation are necessary for this reaction, inhibiting the second round also results in accumulation of Holliday junctions. A similar screen for site-specific recombination inhibitors has yielded another set of peptides, the most potent being TrpArgTrpTyrCysArg (WRWYCR). This inhibitor is related in structure and mode of inhibition to the peptide that blocks strand cleavage (KWWCRW) The low concentration of these peptides required to inhibit recombination correlates with that required for the accumulation of Holliday junction intermediates and suggests that they inhibit recombination by trapping Holliday junctions.

Although some peptides bind DNA at very high concentrations, this binding can be outcompeted by nonspecific DNA. In addition, these peptides block recombination almost completely and are resistant to high levels of nonspecific DNA but they have no obvious effect on intermediates. Peptide KWWCRW also blocks DNA cleavage by Vaccinia topoisomerase (topo) with a potency very near that with which it acts on Int, possibly by binding to a complex between the topo and its DNA substrate. However, it does not block either assembly of topo-DNA complexes nor DNA ligation. This suggests that the peptide either binds DNA sequence-specifically (we disfavor this based on its nondescriminate inhibition of all 4 restriction enzymes we tested) or that it recognizes a specific conformation present in both the Int-DNA complex that is also present in the Vaccinia topo-DNA complex.

By trapping these transient intermediates, the peptides have permitted crystallization of a recombination structure between wild type Cre recombinase, a relative of the Int protein, and wild type loxP substrates (FIG. 11). The crystal structure of the peptide-trapped intermediate has higher electron density in the central hole of the structure suggesting that the peptide is bound to the Holliday junction in this space. This electron density is not present in the structure of the Cre mutant protein bound to a pre-made Holliday junction Ghosh and VanDuyne, Unpublished results, personal communication trapped without using the peptide. Moreover, the nucleotides near the central hole are unstacked in the presence of the peptide but are stacked on each other in the crystal structure not containing the peptide. This suggests that these residues are stacking with the bound hexapeptide.

The peptides inhibit cell growth of both gram-positive (Staph. aureus) and gram-negative (Salmonella typhimurium) bacteria in a dose-dependent way (Vannuffel et al., J. Biol. Chem. 267:16114-16120 (1992); Depardieu et al., Antimicrob. Agents Chemother. 45:319-323 (2001); Vannuffel et al., Nucleic Acids Res. 22:4449-4453 (1994)). Although it is possible that this activity comes from the peptides being dissolved in the membrane and forming channels that lead to eventual lysis of the cells, we believe it is rather due to trapping the Holliday Junction intermediate within cells. One reason for this is that a strain, Salmonella typhimurium Ames, which is more permeable than the wild type Salmonella typhimurium LT2 strain is 2-4 fold more sensitive to our peptide than the LT2 strain. A second reason is that when the peptides are added to dividing cells during the mid-log phase of growth, they block further growth but do not cause decreases in turbidity, suggesting that lysis is not occurring. Cell lysis was not observed, indicating that the membrane remains intact. Peptide transport was determined by using C-terminus rhodamine-labeled peptides that retain the inhibitory activity of the unlabeled peptide. To prevent degradation, D-amino acid peptides were synthesized; these retain the activity of the L-amino acid peptides in vitro, and sometimes have slightly higher activity than the L-amino acid peptides. These D-amino acid peptides also inhibit the growth of S. typhimurium, at concentrations about 2 fold lower than L-amino acid peptides. This indicates that degradation indeed can deplete the intracellular pool of peptides.

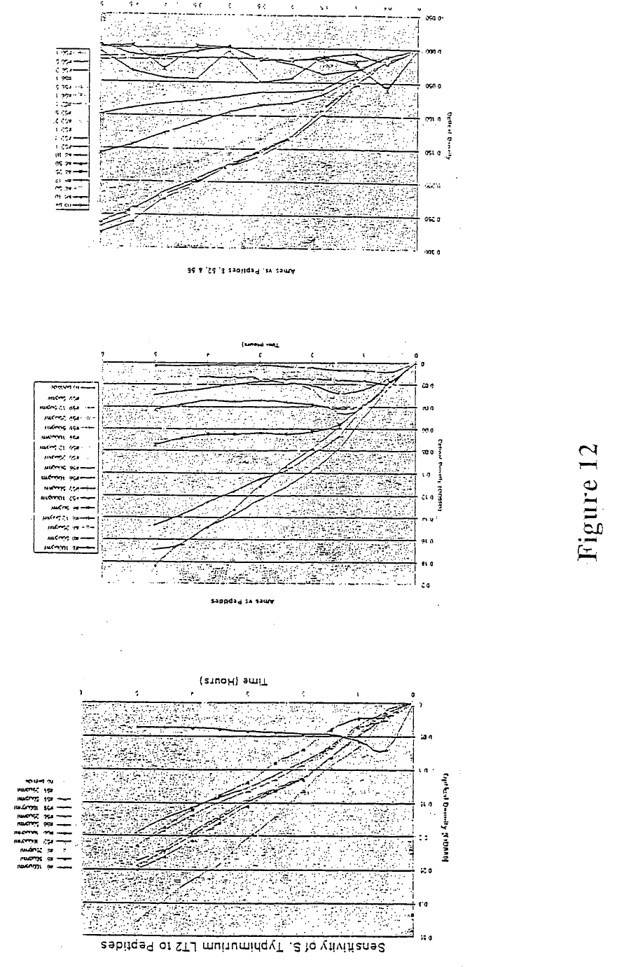

As shown in FIG. 12, the identified peptides inhibit the growth of both gram-negative and gram-positive bacterial species. In addition, these peptides have inhibitory activity against certain topoisomerase enzymes, which can also be a target of the peptides in vivo.

In order to test the inhibitory potency of the peptides, cell growth assays were performed. Growth in the presence and absence of compounds was compared by measuring the cell density of treated and untreated cultures using optical density at 600 nm (a measurement of cell turbidity). The organisms tested included four gram-positive species, methicillin-resistant Staphylococcus aureus (MRSA; gm+), Streptococcus pneumoniae (gm+), and Bacillus subtilis, and 6 gram-negative species: Salmonella typhimurium, E. coli, Klebsiella pneumoniae, Haemophilus influenzae, Neisseria gonorrhoea, and Pseudomonas aeruginosa. Additional species were used for testing of the most potent growth inhibitory compounds. The cell growth assays were performed in microtiter plates; measurements are taken using a BioRad microtiter plate reader.

Overnight bacterial cultures were diluted 1:20 into media and each test compound was added in no more than 10% of the volume of the final suspension. Bacteria are resistant to at least 5% DMSO, which was used to solubilize non-water soluble compounds. The diluted cultures were shaken at 37° C. and optical densities were measured at 30 minute intervals to follow their growth. Growth curves were then compared for all compounds at various concentrations, as shown in FIG. 12. The minimal inhibitory concentration (M.I.C.) for each of the active compounds was tested by serial 2-fold dilutions.

Another assay for used for testing the inhibitory potency of compounds was a disk diffusion assay. The disk diffusion assay involves spotting different concentrations on sterile paper disks that are then placed on appropriate agar plates containing a suspension of the target bacteria. The potencies of each compound at various concentrations are compared by measuring the zone of inhibition—that is, the diameter of the cleared zone around the disk in which no bacterial growth is seen. This test of inhibition indicates the solubility of each compound in agar in addition to potency. In addition, solubility in agar simplifies the isolation of drug-resistant mutant bacteria, as described below.

To test the ability of compounds to cause cell lysis, bacterial cultures were diluted and grown to an O.D.600 of 0.3 (early-mid logarithmic growth), and then active compounds is added at different concentrations. O.D. measurements are obtained for another 2 hours. When cell turbidity remains at the same level or increases at a lower rate, it was concluded that the culture has not been lysed. If, however, turbidity of the culture decreased, it was concluded that lysis was occurring. The ability of a compound to cause lysis is not necessarily desirable, since lysis releases toxic membrane components such as lipopolysaccharides, which can lead to shock in high doses. Fluorescence microscopy also was used to determine the ability of a compound to cause lysis. Fluorescence microscopy was performed using a vital stain such as the SYTOX Green Nucleic Acid stain from Molecular Probes, Inc,. This reagent does not cross the intact plasma membrane of cells, thus bacteria that are stained using SYTOX Green are determined to have a damaged membrane.

Toxicity of peptides KWWCRW and WKHYNY was determined by testing the inhibition of a eukaryotic cell line using a vital stain. The peptides were determined to not be cytotoxic in a non-adherent eukaryotic cell line, Bare Lymphocyte Syndrome (BLS), at concentrations below 250 mg/ml. (Allicotti et al, Manuscript in Preparation). The MTT cytotoxicity assay was used to measure cytotoxicity of the class B synergimycin compounds (Mosmann, Immunol. Methods 65:55-63 (1983)). In this assay, 3-(4,5-dimethylthiazol-2-yl)-2,5-diphenyl tetrazolium bromide (MTT) is converted by live cells into the purple compound formazan. The amount of formazan produced is measured at OD570. The absorbance of the sample wells is compared to the absorbance of the control wells. The readings are used to calculate an IC50 for each peptide. This value represents the concentration of each peptide that reduces the growth of the BLS cells to 50% of the maximum growth measured in the controls.

Because peptides have certain disadvantages as pharmaceutical agents, alternative peptidomimetics were developed which retain the active properties of the peptides but are more soluble and less susceptible to degradation by peptidases. Many tested peptides were found to be sensitive to DTT, but become DTT-resistant if pre-crosslinked with a homobifunctional crosslinking reagent (two maleimide active groups). This data, as well as other results' indicate that the active form of the peptides is a dimer. This means that their effective molecular weight is 1800-2000 daltons rather than 900-1000. Two problems arise from using such a large inhibitor: one is that the effective size of the compounds is significantly larger than most antibiotics (around 500-1000 daltons). Second, the intracellular environment rapidly reduces the disulfide bridges therefore requiring an increase in the peptide concentration for inhibition. Furthermore, alanine substitutions of each amino acid along the peptide indicated that the tryptophan, lysine, arginine and tyrosine residues are important for activity. Peptidomimetic cyclic compounds were designed to incorporate the most potent residues of the original peptides, as well as eliminate unnecessary residues, decrease the molecular weight of the final compound, and obviate the need for the transient disulfide bridges.

### EXAMPLE V

### Organozinc Reactions with Acid Chlorides Bound to Solid-Phase

This example describes organozinc reaction-based methods for preparing a macrocyclic cpmpound or library of the invention.

In this method, an acid chloride was attached to a solid support. Attachment of the acid chloride (via a linker) to the solid support was accomplished using commercially available monomers that contain alcohols and protected acids. Conversion of the acid to the acid chloride and subsequent addition of the organozinc reagent was performed in a manner similar to that described herein above for solution phase methodology, to provide the ketone. The initial work involved simple acid chlorides that contain the protected acid and the necessary alcohol used to attach the compounds to solid-phase. This ensures no other functional groups are complicating the reaction.

Commercially available organozinc reagents (i.e. diethylzinc) are added to a chosen acid chloride attached to solid phase. Twelve resin bound acid chlorides and eight organozinc reagents were reacted in a combinatorial fashion. The final products from these reactions are isolated on HPLC/MS system and the yields are compared to the synthesis of these same compounds in solution.

### EXAMPLE VI

### Organozinc Reactions with Aldehydes Bound to Solid-Phase

This example describes organozinc reaction-based methods for preparing a macrocylic compound or library of the invention. The methods involve organozinc additions to aldehydes.

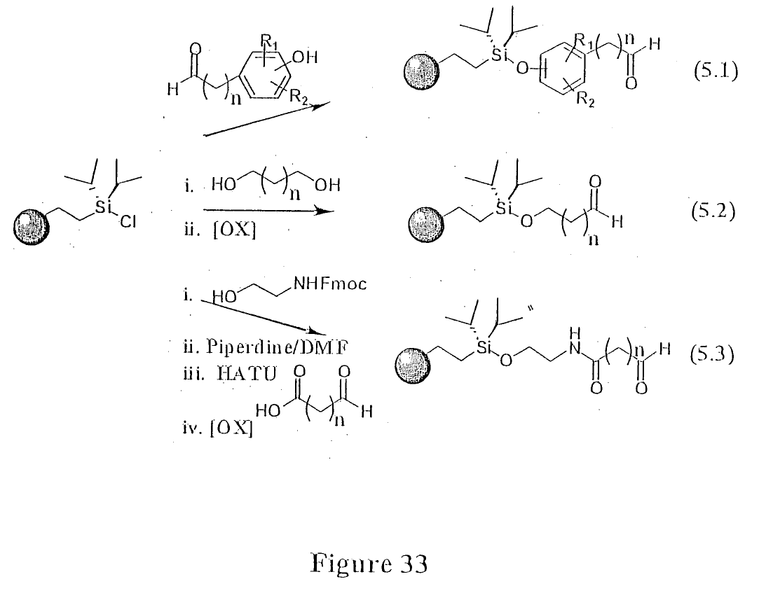

The successes of several solid support-bound chiral amino alcohols that are used to catalyze the enantioselective additions of organozincs to aldehydes are encouraging (Soai and Niwa, S. Chem. Rev. 92:833-856 (1992); Itsuno et al., J. Org. Chem. 55:304-310 (1990); Soai et al., J. Org. Chem. 53:927-928 (1988); Soai et al., J. Chem. Soc. Perkin Trans. I 109-113 (1989); Soai and Watanabe, Tetrahedron: Asymmetry 2:97-100 (1991); Itsuno and Fréchet, J. Org. Chem. 52:4140-4142 (1987). A number of aromatic aldehydes (>40) that contain the appropriate alcohol functionality necessary for attachment to the silyl linker, are commercially available. In order to use non-aromatic aldehydes in this library, other functional groups that could be transformed into an aldehyde were examined. The most successful and straightforward method for producing aldehydes on solid support is the oxidation of an alcohol Chen et al., J. Am. Chem. Soc. 116:2661-2662 (1994) and Reggelin et al., Tetrahedron Lett. 39:4801-4804 (1998)) (see FIG. 33). A protection-deprotection strategy is sometimes necessary to avoid an intramolecular coupling of the diol to the activated diisopropylsilane. With over 25 aliphatic diols commercially available, the possibility of examining multiple substitutent effects, and electronic effects on reactivity and enantioselectivity is feasible. Another route, which adds additional heteroatoms and steps to the synthesis, can be to couple ethanolamine to the activated chlorosilane (equation 5.3) (Hu et al., J. Org. Chem. 63:4518-4521 (1998)). Coupling a hydroxy acid and oxidization of the alcohol to the aldehyde provides access to a number of aldehydes otherwise unavailable.

Another route for synthesizing the organozinc on solid support involve reacting solutions of acid chlorides and aldehydes with solid-phase reagents. Synthesizing the organozinc halide on solid support can be unsuccessful, as demonstrated by Kondo et al, (Kondo et al., J. Comb. Chem. 1:123-126 (1999)) where the lithium halogen exchange was not successful using either n-butyl lithium or t-butyllithium. Upon addition of lithium tri-t-butylzincate the exchange was reasonable at 0° C. Subsequent transmetalation to the organocuprate gave 1,4 addition products in good yields. Typically the order of reactivity of organo-halides for halogen-zinc exchange from fastest to slowest is: allylic=benzylic>alkyl>Csp² hydridized (Knochel and Singer, Chem. Rev. 93:2117-2188 (1993)). As Kondo demonstrated, the aryl iodides are not ideal for synthesizing zinc reagents via the typical t-butyl lithium addition and required the soluble zincate reagent. However, allylic, benzylic, and alkyl halides can successfully undergo the lithium halogen exchange. The use of activated zinc metals such as zinc dust or Rieke's reagent (Hanson and Rieke, J. Am. Chem. Soc. 117:10775-10776 (1995)) are not ideal on solid phase due to the inherent problems of two solids reacting Itsuno et al., J. Org. Chem. 52:4644-4645 (1987)). However, there are examples where the organolithium reagent is formed on solid support via addition of n-butyllithium to bromopolystyrene (O'Brien and Rieke, J. Org. Chem. 55:788-790 (1990)).

Initially, experiments focus on lithiation via an organolithium using halo-alcohols as starting materials (the alcohol are attached to the linker prior to lithium halogen exchange), and comparing the rate of reaction of an aryl halide to that of an alkyl, benzylic, or allylic halide. There are a large number of both non-aromatic and aromatic halo-alcohols as well as non-aromatic and aromatic halo-carboxylic acids (including benzylic and allylic halides) that can be attached to solid-phase. These allow observation of the lithium-halogen exchange at sp² and Sp³ carbons. Transmetalation then provides the organozinc halide or diorganozinc. Particularly useful is the method described in by Rozema et al., J. Org. Chem. 57:1956-1958 (1992) where neat diethylzinc is heated with the alkylhalide, which leads to rapid exchange in solution and formation of the mixed dialkylzinc. At this point the resin is filtered and is added to solutions of commercially available aldehydes, ketones, and enones. The Rozema method is mild and tolerates a large number of functional groups including esters, nitrites, chlorides, and boronic esters. In addition, these diorganozinc reagents formed using this method are more reactive than the organozinc halides.

### EXAMPLE VII

### Organozinc Reactions Used to Form Chiral Alcohols

This example describes organozinc reaction-based methods for preparing macrocyclic compounds and libraries.

Similar to the synthesis using an acid chloride electrophile the organozinc chemistry, an aldehyde was used as an electrophile to generate chiral alcohols. The starting material is the tripeptide fragment (3-4x-5x, FIGS. 30) from the peptide synthesis, and reduction using DIBAL-H gives the desired aldehyde. Coupling of the organozinc reagent 1-2 in the presence of the MIB chiral catalyst in DMA, protection of the resulting chiral secondary alcohol and deprotection of the amine in 5 can provide the pentamer 1-2-3-4x-5x. Coupling of that to 6-7 using HATU and DMAP, then deprotection of the acid on 2, and the amine on 7 using sodium hydroxide and TFA respectively, can give the linear heptamer precursor. Addition of the three coupling agents in the presence of DMAP for the final cyclization followed by deprotection of the alcohols provides the final peptidomimetic, where the labile ester has been replaced with a chiral secondary alcohol. This alcohol mimics the hydrogen-bond donor of an amide and the synthesis of these compounds can depend on the biological assay results of the peptides and peptidomimetics described earlier.

In one experiment twelve aldehydes were distributed in columns of a 96-well plate. In a separate plate the organozinc reagents as described above. However, upon the addition of CuBr.DMS and drying it by heating the plate under vacuum until it green, the aldehyde was added with the 3-exo-Morpholinoisoborneol (MIB) catalyst (Nugent, W., Chem. Commun. 1369-1370 (1999)).

### EXAMPLE VIII

### Assay for Recombination Modulation

This example shows methods for testing compounds in vitro for their ability to inhibit the recombination reaction catalysed by the Int protein. Briefly, attL and attR linear DNA recombination substrates (one radioactively labeled with ³²P) is incubated in the presence of Int protein and the accessory proteins IHF (Integration Host Factor) and X is (Excisionase). The reaction is allowed to incubate for 1 hour and recombination are monitored after separation of the substrates and products after polyacrylamide gel electrophoresis. Both inhibition of the complete recombination reaction and the accumulation of reaction intermediates can be monitored on the same gel.

### EXAMPLE IX

### Assays for Inhibition of Bacterial Cell Growth

This example shows essays for determining if a compound of the invention inhibits bacterial growth.

Compounds that inhibit bacterial growth can induce DNA damage, defects in chromosome segregation, replication, and transcription. The damage-inducing activity of compounds can be tested in a variety of ways, two of which are described below: testing the induction of a DNA-damage inducible promoter, and quantitating intracellular few 3′ OH ends (TUNEL assay). The dinD gene is one of 20-30 DNA repair genes whose transcription is increased in response to DNA damage. We have a reporter gene, lacZ, fused to the promoter of the dinD gene. The lacZ gene encodes an activity, beta galactosidase, which converts the substrate ONPG (orthonitrophenyl galactose) into galactose and a free blue indicator dye. Cells carrying a dinD::lacZ fusion are treated with compounds that inhibit site-specific recombination and the amount of lacZ induction is measured by measuring the release of the yellow orthonitrophenol spectrophotometrically. This test, while very simple, is not very sensitive. Another quantitative assay is the TUNEL assay. Bacteria treated with inhibitory compounds as well as control untreated cultures are permeabilized by treatment with 4% formaldehyde and then mixed with biotin-labeled dUTP and the terminal-d-transferase (TdT) enzyme (Rohwer and Azam, Appl. Environ, Microbiol. 66:1001-1006 (2000)). TdT polymerizes the dUTP onto free 3′OH ends of broken DNA. The DNA is then isolated and reacted with fluorescein-conjugated avidin. The amount of labeled DNA in treated and untreated cultures is compared. If the compounds induce DNA damage, more labeling occurs in the treated cells. Finally, if the inhibitors induce DNA breaks, bacteria mutated in genes necessary for the repair of breaks are hypersensitive to the inhibitors. The sensitivity to peptides of recA mutants and wild type cells are compounded. The RecA protein is necessary for essentially all repair of DNA breaks.

Defects in chromosome segregation are tested directly by visualizing DAPI-stained cells with an epifluorescence microscope. DAPI is a double strand DNA-specific stain which emits light of about 488 nm wavelength when excited with 350 nm light, and thus allows visualization of the bacterial chromosome.

Defects in DNA replication are quantitated by adding ³H-thymidine to cells, treated or untreated, allowing incorporation for 30 minutes, precipitating the DNA with trichloroacetic acid, and counting the ³H-labeled DNA in a scintillation counter. Defects in transcription are measured by addition of ³H-labeled dUTP, as in the TUNEL assay but without addition of TdT, simultaneously with addition of inhibitors. The dUTP is incorporated into mRNA by the cellular RNA polymerase. Total RNA is isolated using a kit, and the labeled RNA is quantitated in a scintillation counter. In each case, comparisons between inhibitor-treated and untreated cells reveal the extent of the inhibitors' effects on the replication, transcription, repair, and segregation processes.

Peptides that inhibit the Int protein also can inhibit, to different extents, the mechanistically- and structurally-related vaccinia virus topoisomerase (vTopo) enzyme, which is necessary for replication of the virus. This enzyme is related to human topoisomerase I. Some of the peptides have a greater than 1000-fold difference in IC50 between Int and vTopo (Table 1). The degree to which the peptidomimetic compounds affect the activity of vTopo is tested by performing plasmid relaxation assays. A peptide known to inhibit vTopo and peptidomimetics were individually incubated with a reaction mixture containing a supercoiled plasmid substrate and vTopo, and the degree of inhibition was compared with respect to a reaction containing enzyme and DNA substrate but no test compound. In addition to effects on eukaryotic topoisomerases, the effects of the compound on prokaryotic topoisomerases, the E. coli topoisomerase I encoded by the topA gene and the E. coli DNA gyrase enzyme encoded by the gyrA and gyrB gene was tested using the assay described above for vTopo.

TABLE 1

| Peptide | KWWCRW^{a} | WRRWCR^{b} | WRYRCR | WRWYCR | YWCYWW |
|---|---|---|---|---|---|
| Int | 0.02-1.1^{C} | 0.01-0.032 | 0.05-0.15 | 0.005-0.021 | 0.018-0.11 |
| Cre | 0.3 | 0.05 | 0.3 | 0.2 | 0.2 |
| hTopol | ˜5^{d} | 38 | 36 | 64 | 23% @ 100 |
| vTopo | 3.5 | 32 | 31 | 48%@100 | 32 |
| EcTopol | 8 | 33% @ 100^{e} | 33% @ 100 | 25% @ 100 | none @ 100 |
| Hind III | 48^{f} | 23 | 27 | 56 | 18% @100 |

In vivo assays were also used to verify inhibition of topoisomerases. Inhibition of topoisomerases changes the superhelical density of plasmids within cells. Plasmid DNA was isolated from inhibitor-treated and untreated cells and electrophorese the uncut plasmid DNA on agarose gels in the presence of a low concentration of ethidium bromide or chloroquine, to better separate topoisomers. The different topoisomer forms of plasmid DNA isolated from the treated and untreated strains indicate whether the inhibitors affect topoisomerase activity within cells.

### EXAMPLE X

### Organozinc Substitution Strategy For Class B Antibiotics

This example shows organozinc reaction-based methods for preparing a macrocylic class B synergimycin derivative.

Up to all amide and ester groups in lead peptide libraries have been replaced with ketones and alcohols to improve their stability and bioavailability, thus increasing their usefulness as a bacterial inhibitor. Four macrocyclic libraries were used and three specific bonds were targeted (two amide and one ester bond) to be replaced by carbon-carbon bonds (see FIG. 17). The initial synthesis of these peptidomimetics occurred in a parallel synthesis fashion, using the biological results from the cyclic peptides as guidance for selecting particular monomers.

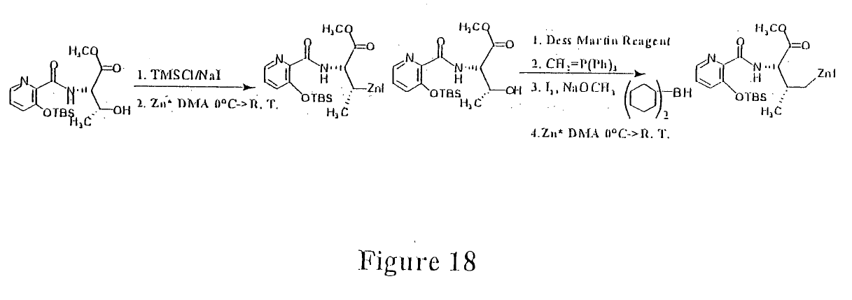

To prepare a class B synergimycin peptidomimetic macrocyclic compound as shown in FIG. 17, the two rings were synthesized using two different 1-2 fragments (FIG. 18 a and b). Both fragments start from the peptide dimer of 1-2. The only difference is that the hydroxy group on monomer 1 is protected as a silyl ether prior to coupling to 2. This protection is not necessary in the peptide synthesis. For synthesis of the first macrocycle (FIG. 18A) the free secondary alcohol is converted to the iodide using trimethylsilyl chloride and sodium iodide. The more severe conditions of iodide, triphenylphosphine, and imidazole also can be used. Conversion of the iodide into the organozinc reagent using one of several methods can provide the desired starting material (1-2A). The other three macrocycles require a longer synthesis to give fragment 1-2 B. The synthesis is performed by oxidation of the secondary alcohol to the ketone using the Dess Martin Reagent, then formation of the alkene by reaction of a methylene ylide with the ketone (Wittig reaction). The resulting alkene reacts with iodide and dicyclohexylborane in the presence of sodium methoxide to give the terminal iodo derivative, which is subsequently converted to the organozinc reagent using activated zinc in dimethylacetamide.

One the first fragment was synthesized, the peptidomimetic macrocyclic compounds shown in FIGS. 17A and B were approached using a similar method as that described for the peptide synthesis strategy. The starting material for this method is the second peptide fragment (FIG. 19). The method involved deprotection of the acid using barium hydroxide (yield=82%) and oxidation of this acid to the acid chloride to provide the trimer 3-4a-5x. Coupling of the trimer to the 1-2 A or B organozinc reagent was performed using the coupling procedures described by Jackson et al. (Deboves et al., J. Chem. Soc., Perkin Trans. 1(1) 1876-1884 (2001); (Dunn et al., J. Chem. Soc., Perkin Trans. 1(13) 1639-1640 (1995); (Dunn and Jackson, Tetrahedron, 53;13905-13914 (1997); (Jackson et al., J. Org. Chem., 57;3397-3404 (1992); (Jackson et al., J. Org. Chem., 63;7875-7884 (1998); (Deboves et al. J. Chem. Soc., Perkin Trans. 1; 4284-4292(2000)). Deprotection of the free amine using trifluoroacidic acid yielded the desired pentamer precursor 1-2-3-4a-5x. Coupling with the third fragment 6x-7a was performed using HATU and dimethylaminopyridine (PyBop or TBTU also can be used), deprotection of the acid on 2 was performed using barium hydroxide, deprotection of the amine on 7 was performed using trifluoroacetic acid and finally, the addition of three coupling agents provided the desired macrocyclic peptidomimetic ring.

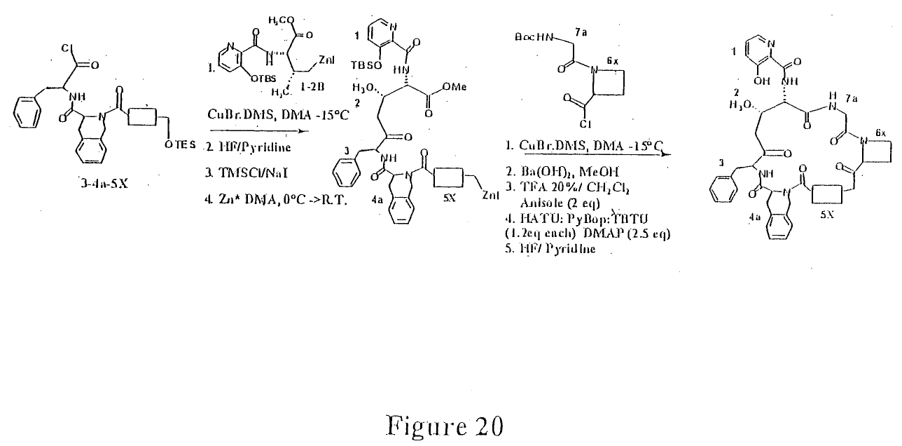

To prepare the peptidomimetic macrocyclic compounds shown in FIGS. 17C and D, the exchange of the appropriate monomer 5 to the protected alcohol was performed to provide the starting material for the 3-4a-5x fragment. Formation of the pentamer 1-2-3-4a-5x, via the method described above involves selective deprotection of the alcohol on 5x (insert reference 59); conversion into the iodide; and then into the organozinc reagent (FIG. 20). For the peptidomimetic macrocyclic compound shown in FIG. 17C, reaction of the organozinc reagent with the 6-7 acid chloride provided the heptamer. Finally, the deprotection of the acid on 2 using barium hydroxide and deprotection of the amine on 7 using TFA afforded the linear precursor for the final macrocyclization.

To prepare the peptidomimetic macrocyclic compounds shown in FIG. 17D, the organozinc pentamer precursor was the same as for FIG. 17C except that the third fragment 6-7 is different (FIG. 21). In this case the 6-7 fragment contains a protected alcohol on 7 rather than a protected amine. After coupling the pentamer with the 6-7 acid chloride, selective deprotection of the alcohol on 7, conversion into the iodide, provided the linear precursor. Deprotection of the acid on 2, and conversion into the acid chloride provided the macrocyclic precursor. Macrocyclization of this heptamer via organozinc coupling chemistry provided the desired peptidomimetic, where three functional groups (an ester and two amides) were exchanged for three ketones.

As shown in FIGS. 65, organozinc methodology is useful for synthesizing class B synergimycin peptidomimetics by replacing the lactone functionality in class B synergimycin derivatives with a ketone. The route involves organozinc chemistry for the formation of the ketone between residues 2 and 3, as shown in FIGS. 65. As shown in this reaction scheme, residue 1 is added after cyclization.

The iodo derivatized residue is converted into the organozinc reagent. Using the acid on fragment 2, addition of thionyl chloride and pyridine provides the acid chloride. Coupling of the organozinc reagent to the acid chloride using the coupling procedures described by Cassell et al., J. Mol. Bio., 299;1193-1202 (2000); Klemm et al., J. Mol. Bio., 299;1203-1216 (2000); Albericio and Carpino, In Methods in Enzymology; Fields, G. B., Ed.; Academic Press: New York, N.Y., pp 104-126 (1997); Humphrey and Chamberlin, Chem. Rev. 97;2243-2266 (1997); WO 2001002427, WO 2000_FR1818, FR_{—}1999-8375, Caba et al., Org. Chem., 66;7568-7574 (2001); Dunn et al., J. Chem. Soc., Perkin Trans. 1(13) 1639-1640 (1995); or Ward et al., J. Org. Chem. 66, 7832-7840 (2001), which uses a Nickel catalyst, provides the desired precursor. The organozinc reactions are often run in some percentage of THF, and the use of palladium catalysts rather than copper for the transmetalation is usually unsuccessful because the zinc (or zinc salt) induces a reaction of the acid chloride with THF. The transmetalation using a copper catalyst is a good alternative to this problem, as is the use of a nickel catalyst. To maintain the fidelity of the stereo center on residue 3 of the acid chloride a copper catalyst is used. Deprotection of the free amine on this intermediate using trifluoroacidic acid and coupling the free acid on dipeptide 6-7 using coupling agents and Hunig's base provides the linear precursor. Deprotection of the acid on 2 using barium hydroxide, followed by acidification of the solution using trifluoroacetic acid deprotects the amine on 7 in situ. Addition of coupling agents, as described above, provides the desired macrocycle. Subsequent deprotection of residue 2 and coupling of residue 1 provides the desired peptidomimetic macrocyclic compound.

In FIGS. 65, (a) is CuBr DMS (0.13 eq), DMF, −15 C; (b) is TFA (20%), CH₂Cl₂, Anisole (2 eq); (c) is HATU (1.2 eq), Hunigs base (3 eq), CH₂Cl₂; (d) is Ba(OH)₂ (4 eq), MeOH,; (e) is CH3CN; (f) is Residue 1, HATU (1.2 eq), Hunig's base (3 eq), CH₂Cl₂.

### EXAMPLE XI

### Organozinc Reactions Used to Form Chiral Alcohols for Compound and Combinatorial Library Preparation

This example shows a method for preparing a macrocyclic compound of the invention using organozinc reactions to form chiral alcohols.

An aldehyde was used as an electrophile to generate chiral alcohols (FIG. 22) in class B synergimycin derivatives. The starting material was a tripeptide fragment 3-4x-5x. Reduction using DIBAL-H yielded the desired aldehyde. Coupling of this aldehyde with the organozinc reagent 1-2 in the presence of the MIB chiral catalyst (Nugent, W. Chem. Commun, 1369-1370 (1999)); protection of the resulting chiral secondary alcohol; and deprotection of the amine in 5 provided the pentamer 1-2-3-4x-5x. Coupling of the pentamer to 6-7 using HATU and DMAP; and deprotection of the acid on 2, and the amine on 7 using sodium hydroxide and TFA respectively, provided the linear heptamer precursor. Addition of the coupling agent cocktail followed by deprotection of the alcohols provided the final peptidomimetic, where the labile ester was replaced with a chiral secondary alcohol. This alcohol mimics the hydrogen bonding donor of an amide.

### EXAMPLE XII

### Methods for Preparing Libraries of Macrocyclic Compounds

This example shows a general synthesis strategies for targeted libraries of macrocyclic compounds that are class B synergimycin derivatives or Holliday junction-trapping compounds.

For class B synergimycin derivatives, syntheses of all pentamers and dimers in each grid of pentamers shown in FIG. 14, ranging from 1-2-3-4a-5a to 1-2-3-4a-5e and dimers ranging from 6a-7a to 6e-7a are prepared. The compounds are then placed in a 96 well reactor, and the libraries synthesized.

For Holliday junction-trapping compounds, one method of synthesis involves coupling trimers together with spaces to form octamers, as shown in FIG. 16. To prepare a variety of compounds containing different spacers by combinatorial methods, a specific 4 monomer is selected; the 5 monomer is varied by row; and the spacer is varied by column. A variety of spacers are used, for example, glycine, tryptophan, arginine, histidine, and phenylalanine.

With a particular trimer sequence, large scale quantities (˜1 g) are made of the five trimers (3-4x-5A to 3-4x-5e). Trimers that have the free acid on monomer 3 are placed in the 96-well reactors and coupled to the appropriate spacers. Addition of the appropriate spacer (1 to 5) to each grid column and two separate coupling agents in a 1:1 ratio (HATU:PyBop) as well as DMAP in methylene chloride, provide the appropriate tetramers.

Tetramers are purified and divided into two portions where half of each tetramer are placed in a plate. In one plate, the acid is deprotected on the spacer using NaOH in MeOH and in the second plate the amine are deprotected on monomer 5 using TFA and anisole. As the appropriate deprotection of these two plates have been accomplished, the second plate of compounds are added to the first plate, being sure to keep the corresponding 5 monomers to the appropriate row. For example, all tetramers from plate two that have 5a are added to the first row in plate one. Coupling agents and DMAP are also added to the grid. This ensures the C-2 symmetrical synthesis of 25 linear octapeptides per plate, to obtain five different spacers and five different monomers of 5. Then these linear ocatamers compounds are purified and placed back in a 96-well reactor. The acid is deprotected using NaOH in MeOH. Addition of 20% TFA and anisole (2 eq) to the compounds deprotects the amine. The TFA is vacuumed off and the addition of the 1:1 mixture of the two coupling reagents (HATU:PyBop) along with DMAP can provide the cyclic peptides. Purification at this point via prep HPLC provides twenty-five C-2 symmetric cyclic peptides.

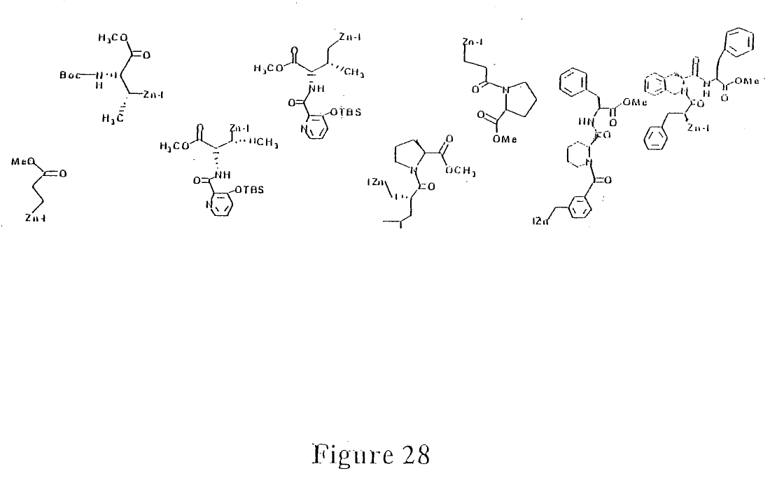

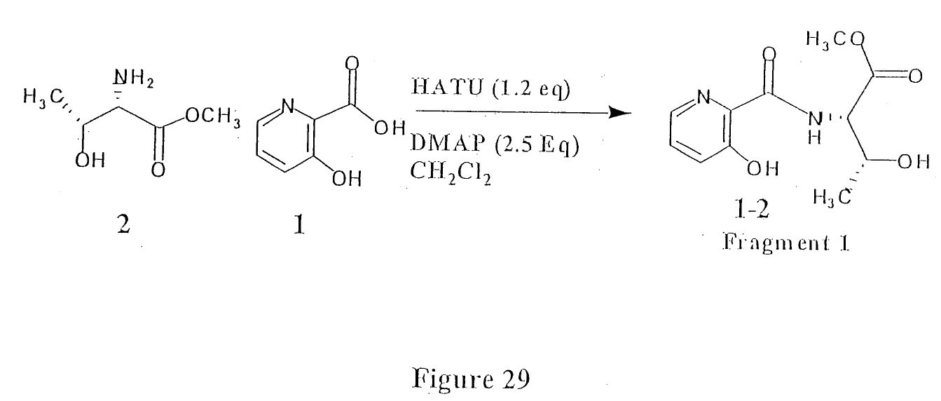

To prepare Holliday junction-trapping compounds using organozinc chemistry in a high throughput synthesis scheme, 96-well plates were prepared with the each of the reagents shown in FIG. 28 being distributed to every well in a single row. Each of the twelve acid chlorides shown in FIG. 27 were distributed to every well in each column. Zinc powder was added to each well and heated to dryness. After DMA with TMSCl was added, the slurry was stirred for 30 minutes (Deboves et al., J. Chem. Soc., Perkin Trans. 1 4284-4292 (2000)). The solution was then cooled to 0° C. and the iodo compound was added to the appropriate column. The reaction was stirred for an hour at room temperature. In a separate plate CuBr.DMS was added to each well first. This plate as heated under vacuum until the CuBr turned from brown to green. Dry DMA was added and cooling the plate to −15° C. followed subsequent addition of the acid chloride. The supernatant from the organozinc reagent plate was added to each row of acid chloride solutions at −15° C. Allowing the reactions to warm to room temperature overnight provided the desired ketones.

A second route involves conversion of the iodo-derivatized amino acid to the zinc reagent via addition of zinc-copper couple in toluene:DMA at 50° C. Addition of these organozinc reagents to bis(triphenylphosphine) dichloropalladium and the columns of acid chlorides, which are in toluene DMA at 50° C., provide the desired ketone.

Using either route, the work-up consisted of a small-scale extraction on the plate using methylene chloride and keeping the organic layer in each well. After drying and evaporating the solvent, the compounds were purified.

The specific acid chlorides and particular organozinc reagents were chosen to provide organozinc reactions with di- and tri-peptides, while delineating the maximum size of both the organozinc reagent and the acid chloride possible for any given coupling reaction.

### EXAMPLE XIII

### Molecular Modeling of Class B Synergimycin Derivatives

This example describes the use of molecular modeling to guide selection of monomers for inclusion in a macrocyclic compound of the invention.

Molecular modeling was performed using Macromodel™34 on four Class B derivatives: (A) 1-2-3-4a-5a6-7a; (C) 1-2-3-4c-5a-6-7a; (D) 1-2-3-4d-5a-6-7a; (E) 1-2-34e-5a-6-7a, with reference to FIG. 3. These four structures were chosen because they contained identical residues in every position except for residue 4. Residue 4 was varied in order to understand the role that pi-stacking plays between residue 4 and 5 in the conformation of these macrocycles. Comparison of compounds A-E low energy structures to those of VS1 and Quinopristin (Bodanszky and Bodanszky, “The practise of peptide synthesis”, 2nd ed.; Springer: New York, N.Y., (1994)). Both compound A and C, where residue 4 is replaced by 4a and the enantiomer 4c respectively, exhibited pi-stacking between residues 4 and 5. This was unexpected given that they have the opposite stereocenter at a critical carbon. They both demonstrated a hydrogen bond between the amide nitrogen on 3 and the carbonyl on 7. This is in contrast to the low energy conformations found for VS1 and to the active Class B compound Quinopristin (data not shown). According to these done in MacroMode™, and those done by others, (insert 4,27,31-33) VS1 exhibits a hydrogen bond (H-bond) between the amide nitrogen on 3 and the carbonyl on 5, and Quinopristin exhibits an H-bond between the amide nitrogen on 3 and the carbonyl on 6. Interestingly, compounds D and E exhibited H-bonds between the same residues as Quinopristin. The energy of each structure was dominated by the compounds ability to form a close intramolecular H-bond. Previous studies had proposed that the pi-stacking between 4 and 5 would have a large impact on the conformation of the macrocycle. (Insert refs 30,31). However, these molecular modeling studies suggest that the transannular hydrogen bonding has the greatest influence on the macrocyclic conformation. This suggests that compounds D and E maintain the same conformation as the active antibacterial compound Quinopristin and can be effective antibiotics, whereas, compounds A and C are predicted to have little or no activity in bacterial strains resistant to VS1.

### EXAMPLE XIV

### Methods for Preparing Class B Synergimycin Derivatives

This example shows synthetic methods for preparing class B synergimycin derivative macrocyclic compounds of the invention, as well as libraries of such compounds.

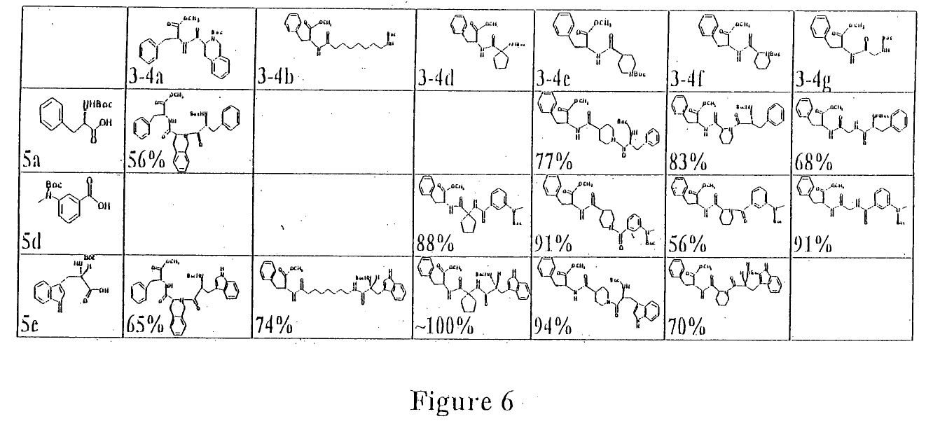

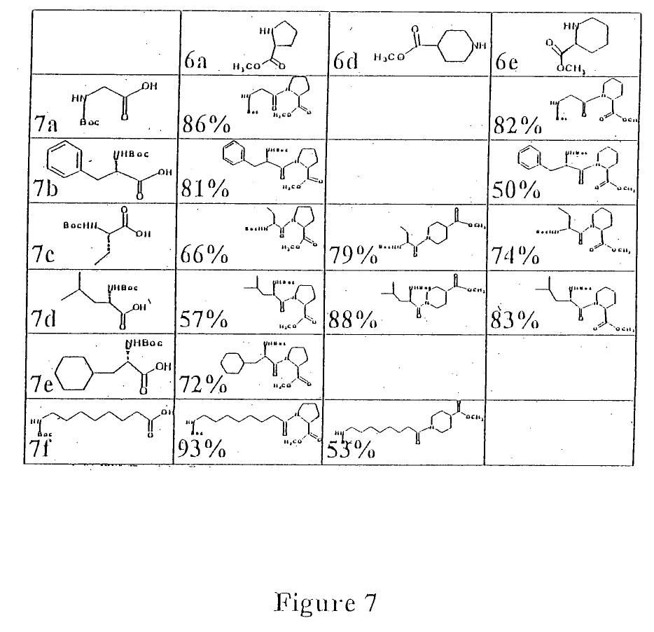

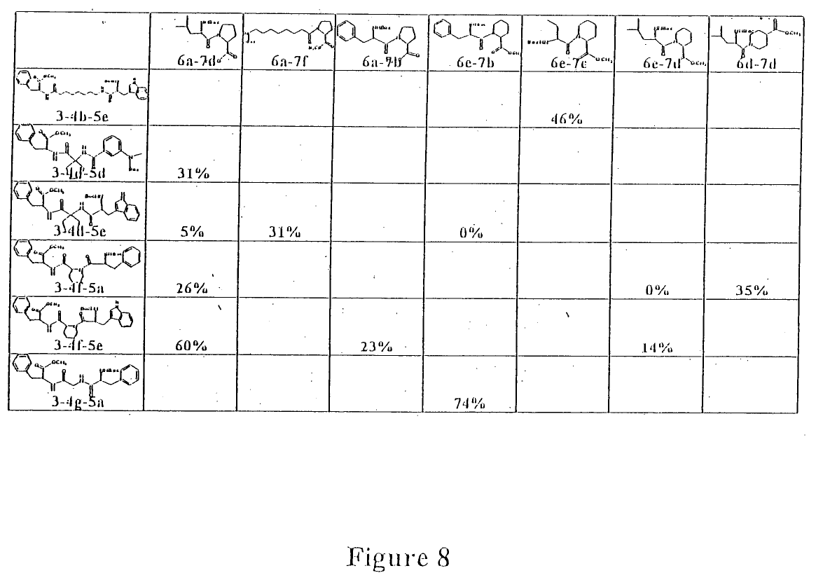

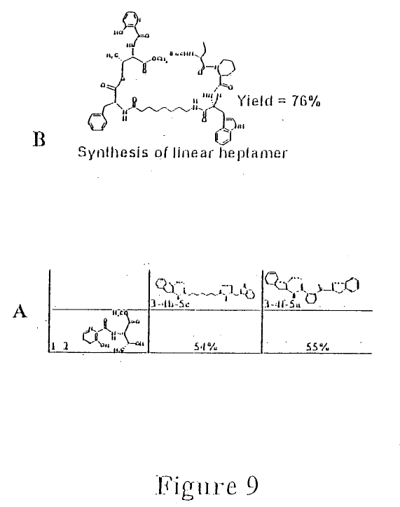

Several targeted peptide fragments were synthesized (FIG. 5b) in good to excellent yield (FIGS. 6-9). Coupling of the amino acids using HATU and DMAP were chosen because they alleviate racemization of stereocenters and are more effective for secondary amines when compared to most coupling reagents. However, a variety of coupling agents can be used. As shown in FIGS. 8 and 9a, several synthesized fragments were prepared in acceptable yields. Coupling of amino acids 1 and 2 occurred in an acceptable yield (75%) considering the steric hindrance of both the electrophile and nucleophile. Synthesis of the 3 and 4 dimers occur in high yields, and deprotection of the amines on 3-4 using TFA/Anisole2, gives the free amines (Greene and Wuts, Protective Groups in Organic Synthesis, third edition ed., John Wiley and Sons, Inc.: New York (1999)). Coupling of these amines to 5 gives the second fragment in relatively high yields (56-99%) (FIG. 6). Coupling of the amine protected 7 and the acid protected 6 and deprotection of acid using Ba(OH)₂ give the third fragment in high yields (57-93%), 6-7 (FIG. 7).

Fragments 3-4-5 and 6-7 were coupled with yields that vary from 0% to 74% (FIG. 8). Subsequent deprotection of the acid on 3, and coupling to 1-2 can give the desired linear precursor. A heptamer has also been prepared using this method (yield=56%). As shown in FIG. 9, a second method involved coupling of 1-2 to 3-4-5 first (FIG. 9a), and the resulting pentamer is coupled to 6-7 (76%) (FIG. 9b). The advantage is that the relatively non-nucleophilic alcohol on 2 couples better to a smaller fragment, thus improving the overall yields.

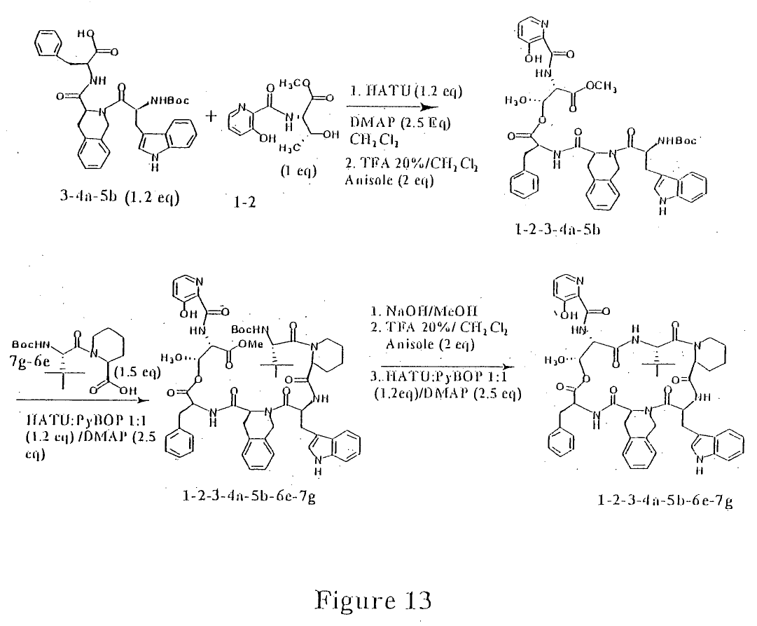

The most successful coupling order of the fragments is when the 1-2 fragment (the first fragment) is coupled to 3-4-5 (the second fragment) before the 6-7 (the third fragment) (FIG. 13). This is because the alcohol is only moderately nucleophilic, therefore coupling of the 1-2 alcohol to the 3-4-5 trimer is significantly more facile than coupling the alcohol to the more sterically congested pentamer (3-4-5-6-7). This approach accommodates the poor nucleophilicity of the alcohol by coupling it sooner in the sequence. A series of libraries in solution have been prepared, where monomer 4 and monomer 7 (FIG. 3) are assigned individually, and monomer 5 (column) and monomer 6 (row) are systematically varied to produce a total of 175 cyclic peptides.

Each of the references and U.S. Patents cited above is hereby incorporated herein by reference.

Although the present invention has been exemplified by the disclosed embodiment, those skilled in the are will readily appreciate that the specific examples are provided to illustrate, not to limit, the invention. It can therefore be understood that various modifications can be made without departing from the spirit of the invention.

### EXAMPLE XV

### Preparation of Macrocyclic Peptidomimetic Compounds that are Synergimycin Derivatives

This example shows a method for preparing macrocyclic peptidomimetic compounds that are Synergimycin derivatives.

A procedure for preparing macrocyclic peptidomimetic compounds that are Synergimycin derivatives is shown in FIG. 34. The macrocyclic compounds shown in FIG. 17 were prepared.

As shown in FIG. 34, to synthesize the two rings of the macrocycle, two different 1-2 fragments were prepared. Both fragments start from the peptide dimer of 1-2, and the yield for this reaction is 72%. The only difference is that the hydroxy group on monomer 1 is protected as a silyl ether prior to coupling to 2. This protection is not necessary in the peptide synthesis. For synthesis of the first macrocycle the free secondary alcohol is converted to the iodide using trimethylsilyl chloride and sodium iodide. Conversion of the iodide into the organozinc reagent using one of several methods described herein provides the desired starting material (1-2A). The other three macrocycles require a longer synthesis to give fragment 1-2 B. Oxidation of the secondary alcohol to the ketone using the Dess Martin Reagent then formation of the alkene occur by reacting a methylene ylide with the ketone (Wittig reaction). This alkene is reacted with iodide, dicyclohexylborane in the presence of sodium methoxide to give the terminal iodo derivative, which is subsequently converted to the organozinc reagent using activated zinc in dimethylacetamide at 0° C.

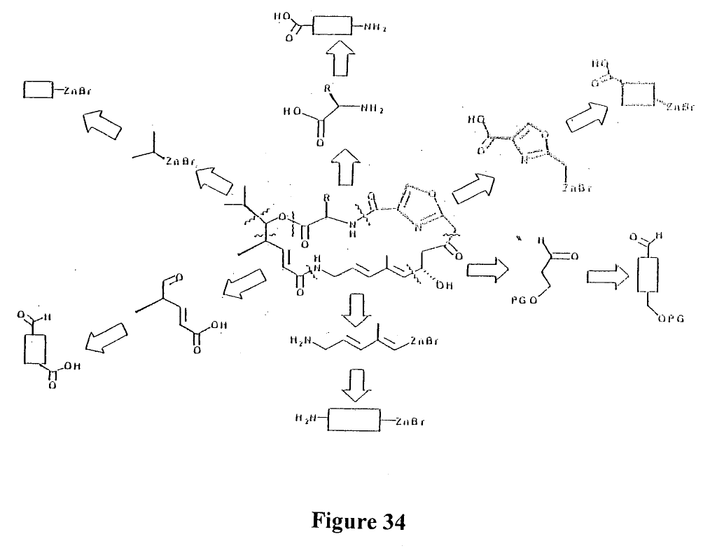

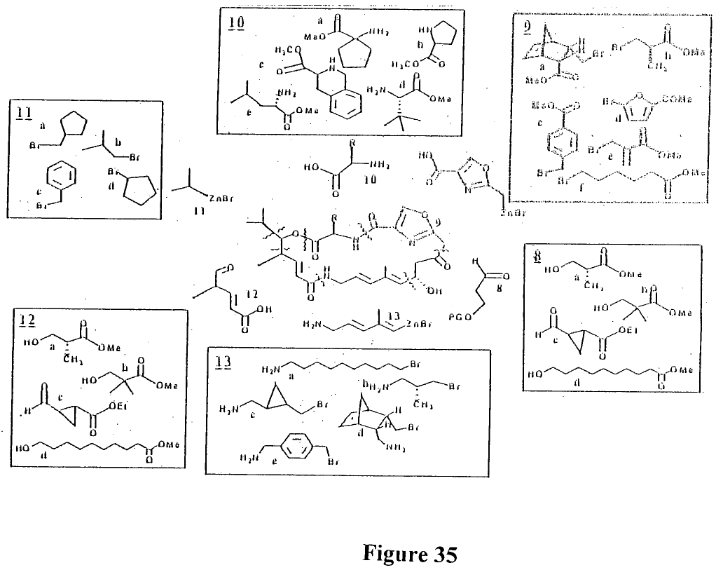

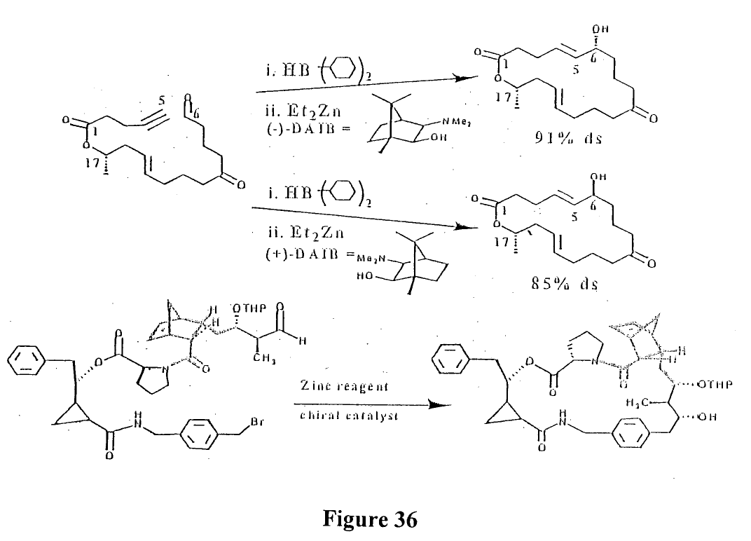

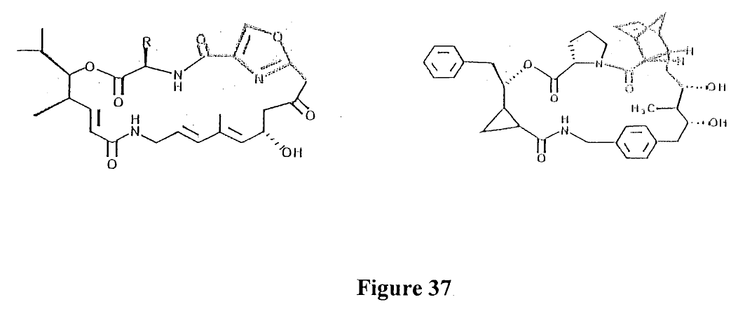

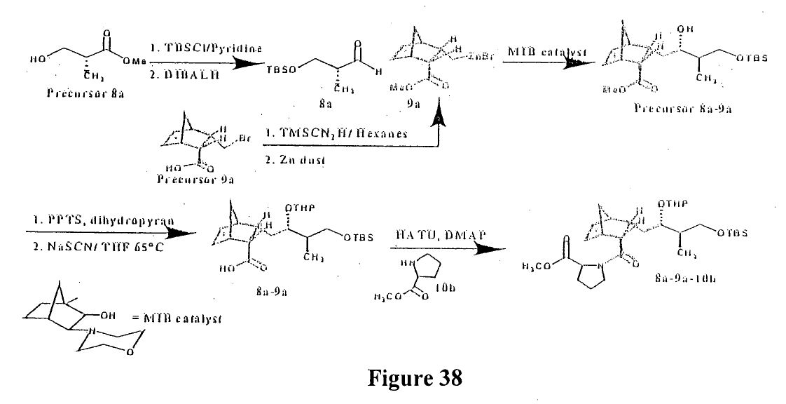

The first and second macrocycles (A and B) were both synthesized using a similar method as that described for the peptide synthesis methods described here above and is shown (FIGS. 35), where the starting material for this method is the second peptide fragment. Deprotection of the acid using barium hydroxide (yield=82%) and oxidation of this acid to the acid chloride provides the trimer 3-4a-5X. Coupling of this to the 1-2 A or B organozinc reagent using the coupling procedures described herein, and deprotection of the free amine using trifluoroacidic acid provides the desired pentamer precursor 1-2-3-4a-5X. Coupling with the third fragment 6x-7a using HATU and dimethylaminopyridine (as well as possibly PyBOP and TBTU), deprotection of the acid on 2 using barium hydroxide, deprotection of the amine on 7 using trifluoroacetic acid and finally the addition of three coupling agents provide the desired macrocyclic peptidomimetic ring.

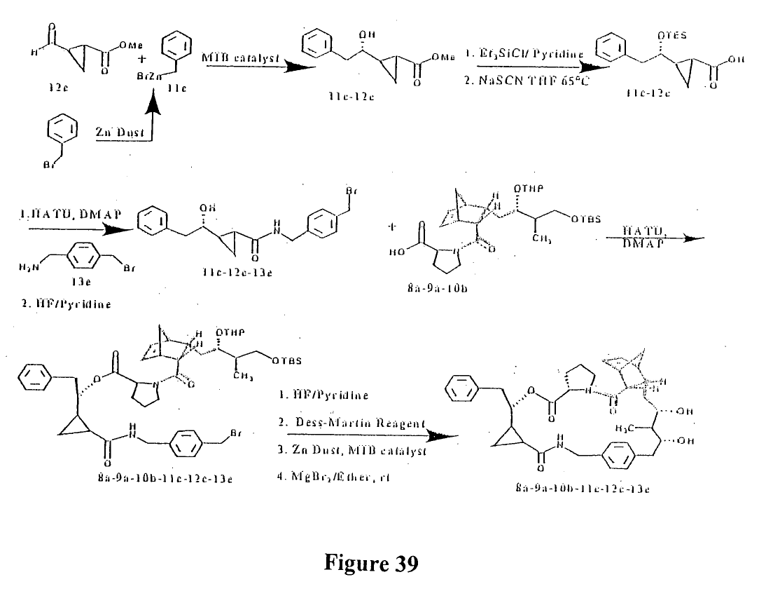

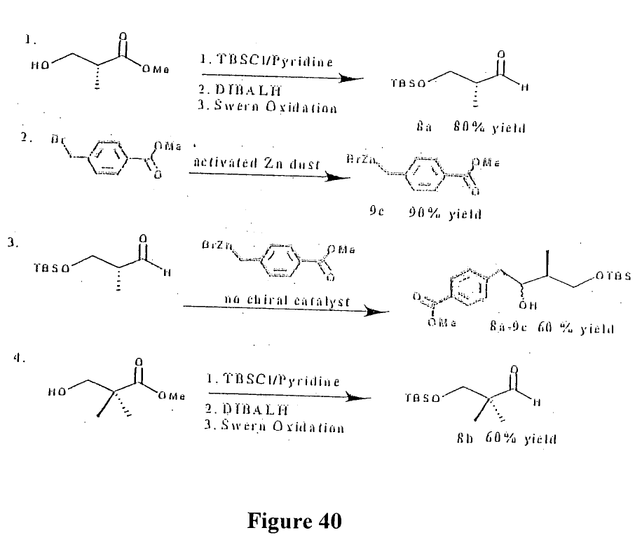

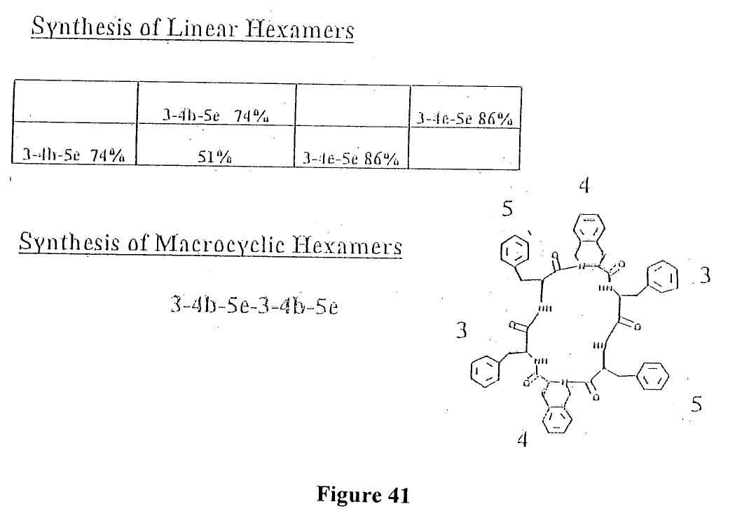

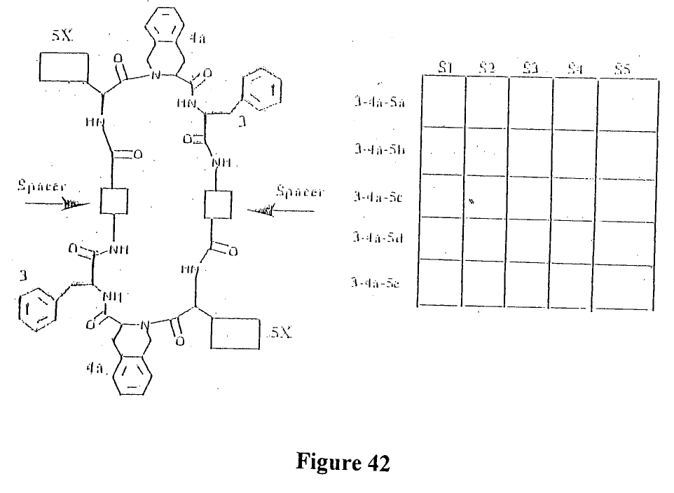

For the formation of the third (C) and fourth (D) macrocycles, the exchange of the appropriate monomer 5 to the protected alcohol is necessary to provide the starting material for the 3-4a-5x fragment. Formation of the pentamer 1-2-3-4a-5x, via the method described above involves, selective deprotection of the alcohol on 5x conversion into the iodide, and then into the organozinc reagent (FIGS. 39). For the third macrocycle the reaction of the organozinc reagent with the 6-7 acid chloride, again using the conditions described above, provide the heptamer. Finally, the deprotection of the acid on 2 using barium hydroxide and deprotection of the amine on 7 using TFA give the linear precursor for the final macrocyclization. The addition of three coupling reagents in the presence of DMAP give the desired peptidomimetic that has two groups, an amide and an ester moiety, exchanged for ketones.

For the formation of the fourth macrocycle (D), the organozinc pentamer precursor is the same as that described above, however the third fragment 6-7 is different. In this case the 6-7 fragment contains a protected alcohol on 7 rather than a protected amine. After coupling the pentamer with the 6-7 acid chloride, selective deprotection of the alcohol on 7, and conversion into the iodide give the desired heptamer. Deprotection of the acid on 2, and conversion into the acid chloride using the same conditions described above, provide the macrocyclic precursor. Macrocyclization of this heptamer via organozinc coupling chemistry give the desired peptidomimetic, where three functional groups (an ester and two amides) have been exchanged for three ketones.

### EXAMPLE XVI

### Inhibition of Bacterial Growth Using Class B Synergimycin Derivatives

This example shows that identified linear hexapeptides have antibiotic activity.

Four linear heptamers prepared according to FIGS. 63 were tested for their bacteria growth inhibition. Growth in the presence and absence of compounds were compared by measuring the cell density of the cultures at 600 nm (the optical density is a measure of cell turbidity). A set of bacterial pathogens, both gram− and gram+, were tested. These organisms included three gram+ species, methicillin-resistant Staphylococcus aureus (MRSA; gm+), Streptococcus pneumoniae (gm+), and Bacillus subtilis, and 6 gram negative species: Salmonella typhimurium, E. coli, Klebsiella pneumoniae, Haemophilus influenzae, Neisseria gonorrhoea, and Pseudomonas aeruginosa. Measurements were taken using a BioRad microtiter plate reader. Overnight cultures were diluted 1:20 into LB media and each test compound will be added in no more than 10% of the volume of the final suspension. The compounds were dissolved in DMSO and were added to the cultures at two concentrations (10 μg/ml and 100 μg/ml). The diluted cultures were shaken at 37° C. and optical densities were measured at 30-minute intervals in order to follow their growth. Growth curves were then compared for all compounds at two concentrations. In addition, Synercid and Class B Synergimycin were used as positive controls in these experiments. In most cases, Synercid demonstrated no measurable bacteria growth and Class B demonstrated some growth depending on the bacterial strain and the concentration. Some of the linear compounds demonstrated bacterial growth inhibition.

What is claimed is:

1. A method for making a combinatorial library of cyclic compounds that have the generic structure: 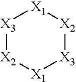
comprising the steps of
(a) obtaining a plurality of trimers according to the generic structure X₁X₂₋X₃, wherein X₁, X₂ and X₃ can be independently any naturally or nonnaturally occurring amino acids or peptidomimetics thereof;
(b) optionally coupling a spacer S to the trimer at either end.
(c) cyclizing two trimers or trimer-spacer conjugates in a head-to-tail orientation;
thereby obtaining a combinatorial library of compounds, wherein the library does not include an unmodifed or naturally occurring Holliday Junction-trapping compounds.

2. The method of claim 1, wherein the side group of X₁ or X₃ independently can have a C₁ to C₃ alkyl connecting a cycloalkyl or aryl group.

3. The method of claim 2, wherein said cycloalkyl is a substitute.

4. The method of claim 2, wherein said aryl is a hetro aryl substituted aryl or X₂ substituted.

5. The method of claim 1, wherein the side group of X₂ incorporates a mono or bicyclic moiety that can be a heterocycle.

6. The method of claim 5, wherein said moiety is a substituted mono or bicylic moiety.

7. The method of claim 5, wherein X₂ provides conformational constraint to the trimer.

8. The method of claim 1, wherein S is an molecular spacer of 5 to 25, 5 to 15, or 5 to 10 Å.

9. The method of claim 1, wherein S is from an organozinc spacer precursor.

10. The method of claim 8, wherein S is selected from glycine, tryptophan, arginine, histidine, phenylalanine, or any other naturally occurring or non-naturally occurring amino acid;

11. The method of claim 1, wherein at least one subunit is radiolabelled.

12. The method of claim 1, wherein synthesis occurs according to FIGS. [53] 46 and [54] 47.

13. A compound having a formula selected from the group consisting of:

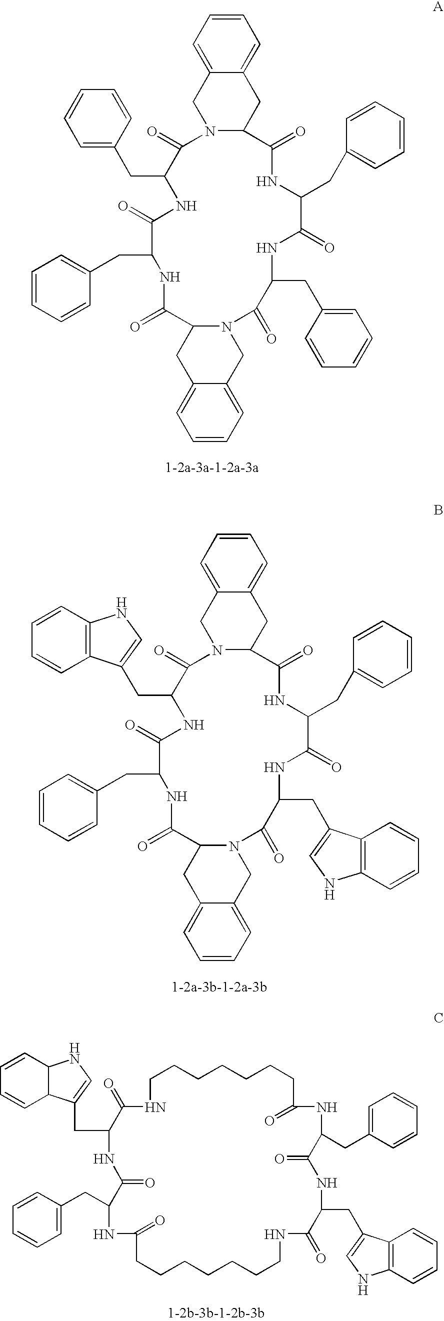

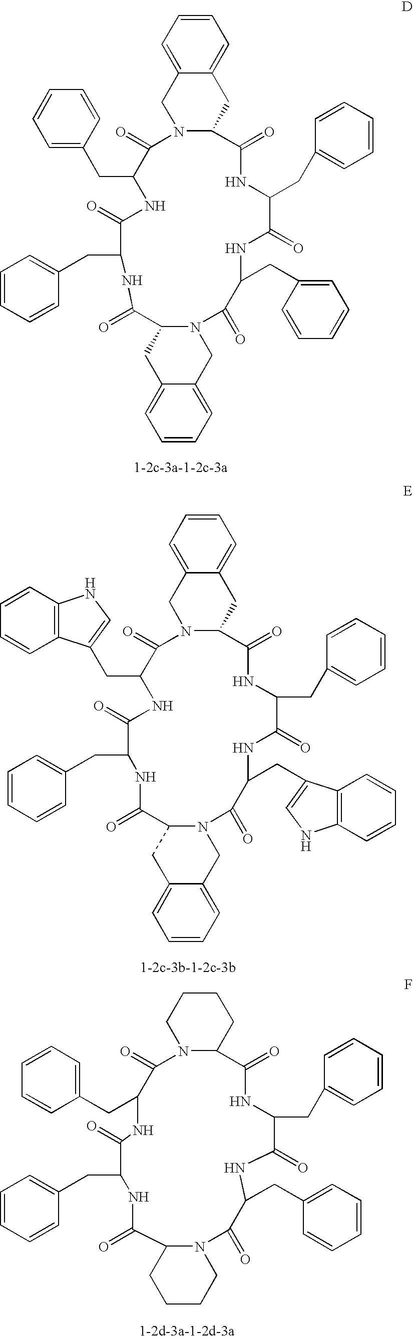

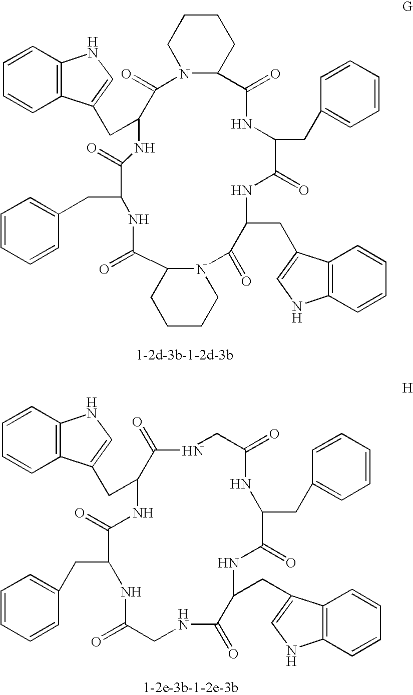

14. A peptidomimetic of a compound of claim 13.

15. A method for inhibiting bacterial growth, comprising contacting a bacterium with a compound of claim 1.

16. A method for making a combinatorial library of compounds having the formula, 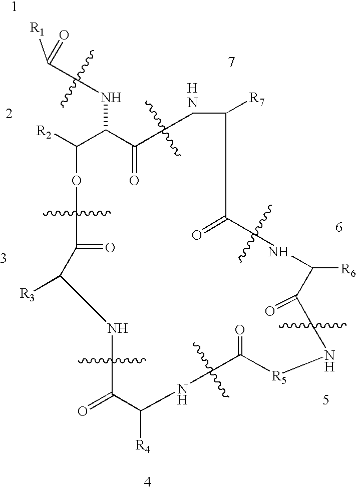
comprising,
(a) obtaining precursors of subunits 1, 2, 3, 4, 5, 6 and 7, or any combination of a plurality of contiguous subunits thereof and
(b) joining contiguous subunit precursors in a combinatorial manner; thereby obtaining a combinatorial library.

17. The method of claim 16, wherein 3 and 6 are capable of hydrogen bonding with each other.

18. The method of claim 17, wherein 1 is a hydroxy acid, wherein R is a linear or branched alkyl or aryl:

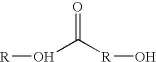

19. The method of claim 18, wherein 1 is an aromatic hydroxy acid.

20. The method of claim 18, wherein 1 is a hydroxy iodide, wherein R is a linear or branched alkyl or aryl:
HO—R—I

21. The method of claim 18, wherein 2 is a hydroxy amine, wherein R is a linear or branched alkyl or aryl:
HO—R—NH₂ or

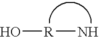

22. The method of claim 18, wherein 2 is a hydroxy acid, wherein R is a linear or branched alkyl or aryl:

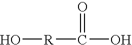

23. The method of claim 18, wherein 2 is a hydroxy iodide, wherein R is a linear or branched alkyl or aryl:
HO—R—I

24. The method of claim 18, wherein 2 is a hydroxy amino acid, wherein R is a linear or branched alkyl or aryl:

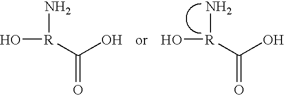

25. The method of claim 18, wherein 2 is an iodo amino acid, wherein R is a linear or branched alkyl or aryl:

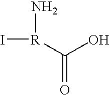

26. The method of claim 18, wherein 2 is an iodo hydroxy amino acid, wherein R is a linear or branched alkyl or aryl:

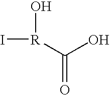

27. The method of claim 16, wherein 3 is an amino acid having the following structure, wherein R is a linear or branched alkyl or aryl:

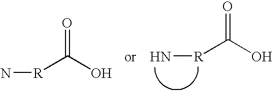

28. The method of claim 16, wherein 3 is an iodo acid, wherein R is a linear or branched alkyl or aryl:

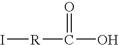

29. The method of claim 2, wherein 3 is a hydroxyacid, wherein R is a linear or branched alkyl or aryl:

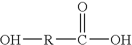

30. The method of claim 18, wherein 3 is a aromatic amino acid.

31. The method of claim 18, wherein 3 is an aromatic iodo acid.

32. The method of claim 18, wherein 3 is a aromatic hydroxy acid.

33. The method of claim 18, wherein 3 contains a hydrogen and 6 contains an electronegative atom.

34. The method of claim 18, wherein 6 contains a hydrogen and 3 contains an electronegative atom.

35. The method of claim 18, wherein 4 is an amino acid.

36. The method of claim 35, wherein 4 is an aromatic amino acid.

37. The method of claim 35, wherein 4 is a hydroxy acid.

38. The method of claim 35, wherein 4 is an iodo acid.

39. The method of claim 35, wherein 4 has a hydrophobic moiety.

40. The method of claim 39, wherein the hydrophobic moiety is an unsubstituted or substituted alkyl, aryl, napthyl or polycyclic group.

41. The method of claim 18, wherein 4 and 5 are capable of pi-stacking.

42. The method of claim 18, wherein 5 is an unsubstituted or substituted aryl, napthyl or polycyclic group.

43. The method of claim 18, wherein 5 is an amino acid.

44. The method of claim 43, wherein 5 is a hydroxy acid.

45. The method of claim 43, wherein 5 is a aromatic amino acid.

46. The method of claim 43, wherein 5 is a aromatic hydroxy amino acid.

47. The method of claim 18, wherein 5 is a hydroxy acid capable of pi-stacking.

48. The method of claim 47, wherein 5 is an iodo acid.

49. The method of claim 48, wherein 5 is an aromatic iodo acid.

50. The method of claim 48, wherein 5 is an iodo acid capable of pi-stacking.

51. The method of claim 48, wherein 5 is an L-isomer amino acid.

52. The method of claim 18, wherein 5 has an aromatic ring and the ring is oriented toward 4.

53. The method of claim 18, wherein 6 is a natural or non-natural amino acid.

54. The method of claim 53, wherein 6 is an iodo acid.

55. The method of claim 53, wherein 6 is a hydroxy acid.

56. The method of claim 18, wherein 6 provides a (type-VI) beta-turn element into the compound.

57. The method of claim 39, wherein the beta-turn element is a type-VI beta-turn element.

58. The method of claim 18, wherein 7 is a natural or non-natural amino acid.

59. The method of claim 58, wherein 7 is an iodo acid.

60. The method of claim 58, wherein 7 is a hydroxy acid.

61. The method of claim 18, wherein 7 has a linear or branched alkyl chain, or a large aromatic or hydrophobic group.

62. The method of claim 61, wherein said alkyl chain is substituted.

63. The method of claim 16, wherein 1-2 is a combination of contiguous subunits.

64. The method of claim 16, wherein X3-X4-X5 is a combination of contiguous subunits.

65. The method of claim 16, wherein 6-7 is a combination of contiguous subunits.

66. The method of claim 18, wherein the bond between 5 and 6 is a ketone.

67. The method of claim 16, wherein synthesis occurs according to FIGS. [62] 55 and [63] 56.

68. The method of claim 18, wherein at least one subunit is radiolabelled.

69. A method for inhibiting bacterial growth, comprising contacting a bacterium with a compound of claim 16.

70. A method for making a combinatorial library of compounds that have the generic structure 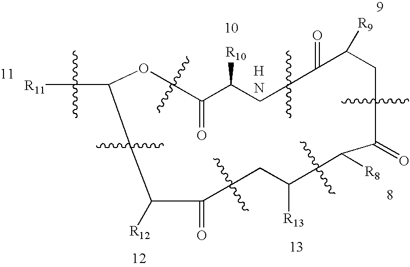
comprising the steps of
(a) obtaining precursors, of subunits 8, 9, 10, 11, 12 and 13, or any combination of a plurality of contiguous subunits or peptidomimetics thereof and
(b) joining contiguous subunit precursors in a combinatorial manner; thereby obtaining a combinatorial library.

71. The method of claim 70, wherein the library does not include unmodifed or naturally occurring Class A Synergimycin.

72. The method of claim 70, wherein R of 8, 9, 10, 11, 12 or 13 is a side group of a naturally or nonnatural amino acid.

73. The method of claim 70, wherein the side group of 11 contains one of or a combination of a C₁ to C₈ alkyl, cycloalkyl or aryl.

74. The method of claim 70, wherein 8, 9, 10, 11, 12 or 13 are independently a hydroxy acid, an aromatic hydroxy acid, a hydroxy iodide, a hydroxy amine, a hydroxy amino acid, an iodo amino acid or an iodo hydroxy amino acid.

75. The method of claim 70, wherein 8 is a hydryoxy acid, hydroxy ester or hydroxy aldehyde.

76. The method of claim 70, wherein 9 is an iodo acid or hydroxy acid.

77. The method of claim 70, wherein 10 is a hydroxy acid or iodo acid.

78. The method of claim 70, wherein 11 is a haloalkyl, halo aryl or hydrophobic halogen group.

79. The method of claim 70, wherein 12 is a hydroxy acid, hydroxy ester, hydroxy aldehyde, iodo acid, iodo ester or iodo aldehyde.

80. The method of claim 70, wherein 13 is a halo amine, hydroxy acid or iodo acid.

81. The method of claim 70, wherein macrocyclization occurs according to FIGS. [43] 36 or [45] 38.

82. The method of claim 70, wherein at least one subunit is radiolabelled.

83. A method for making a peptidomimetic of target molecule A-B, wherein the bond between A and B is an amide or ester, comprising the steps of
(a) obtaining a modified A, wherein A contains an acid chloride or aldehyde;
(b) obtaining a modified B, wherein B contains halide;
(c) coupling the modified A to the modified B using an organozinc reaction, resulting in a carbon-carbon bond;
thereby making a mimetic of A-B.

84. The method of claim 83, wherein the method is performed a plurality of cycles on a target molecule etc. to obtain a mimetic of said target molecule.

85. The method of claim 83, wherein an amino acid derivative is converted into an organozinc reagent.

86. The method of claim 83, wherein the target molecule has a peptide bond between A and B.

87. The method of claim 83, wherein the peptide is cyclic.

88. The method of claim 83, wherein the target peptide is a synergimycin.

89. The method of claim 83, wherein the target peptide is a Holliday Junction-trapping compound.

90. The method of claim 83, wherein the organozinc reaction couples the organozinc reagent to an acid chloride, replacing an amide or ester with a ketone.

91. The method of claim 83, wherein the organozinc reaction couples the organozinc reagent to an aldehyde, replacing an amide or ester with a chiral alcohol.

92. The method of claim 83, wherein the organozinc reaction is performed in liquid or solid phase.
